(19) United States
(12) Patent Application Publication
(10) Pub. No.: US 2025/0179544 A1
(43) Pub. Date: Jun. 5, 2025

(54) PRODUCTION OF CORYNANTHE-TYPE MONOTERPENE INDOLE ALKALOID COMPOUNDS IN A HETEROLOGOUS HOST

(71) Applicant: CB Therapeutics, Carlsbad, CA (US)

(72) Inventors: Phu Khat Nwe, San Diego, CA (US); Jacob M. Vogan, San Diego, CA (US); James Wade, San Diego, CA (US); Tyler Hueslman, San Diego, CA (US); Laura Flatauer Peiffer, San Diego, CA (US)

(21) Appl. No.: 18/965,429

(22) Filed: Dec. 2, 2024

Related U.S. Application Data
(60) Provisional application No. 63/605,289, filed on Dec. 1, 2023.

(51) Int. Cl. C12P 17/18; C12N 1/16; C12N 1/20; C12N 15/52

(52) U.S. Cl. CPC C12P 17/182; C12N 1/16; C12N 1/20; C12N 15/52; C12N 2500/30; C12N 2500/76; C12N 2510/02

(57) ABSTRACT

Provided are non-naturally occurring nucleic acids comprising a sequence encoding an enzyme, regulatory protein, or other functional protein that provides for biosynthesis of monoterpene indole alkaloids (e.g., mitragynine), as well as modified enzyme and protein sequences encoded by the nucleic acid sequences. Also provided are recombinant microorganisms, including novel strains, that comprise the nucleic acid sequences and/or express the enzyme or regulatory protein that provides for the biosynthesis of monoterpene indole alkaloids (e.g., mitragynine). Methods of expressing the enzyme or regulatory protein are provided, as are methods of generating monoterpene indole alkaloid mitragynine-type compounds, as well as precursors, intermediates, and analogs thereof.

### CROSS-REFERENCE TO RELATED APPLICATION

This application claims the benefit of U.S. Provisional Application No. 63/605,289, filed Dec. 1, 2023, and incorporated by reference herein in its entirety.

### SEQUENCE LISTING

The contents of the electronic sequence listing (CBTH-14-US.xml; Size: 584 kilobytes; and Date of Creation: Nov. 20, 2024) is herein incorporated by reference in its entirety.

### FIELD

The disclosure relates to polynucleotides, enzymes, biosynthetic methods, and recombinant microorganisms useful in the production of downstream monoterpene indole alkaloid compounds. The monoterpene indole alkaloids that can be synthesized according to the disclosure include plant-derived and/or fungal-derived compounds or metabolites that can exert a physiological effect, and are inclusive of compounds such as strictosidine, hydroxystrictosidine, methoxystrictosidine, strictosidine aglycone, hydroxystrictosidine aglycone, methoxystrictosidine aglycone, demethylcorynantheidine (20R, 20S), 9-hydroxydemethylcorynantheidine (20R, 20S), 9-methoxydemethylcorynantheidine (20R, 20S), corynantheidine, dihydrocorynantheine, 9-hydroxycorynantheidine, 9-hydroxydihydrocorynantheine, speciogynine, and/or mitragynine, as well as precursors, intermediates, metabolites, stereoisomers, and derivatives thereof. The monoterpene indole alkaloids herein can be generated from precursor compounds that are produced by upstream monoterpene indole alkaloid biosynthetic pathways, which can feed into the biosynthetic pathways (e.g., mitragynine pathways) useful in the production the target compounds as described herein.

### BACKGROUND

Plants, fungi, and bacteria are biochemical factories that can make complex alkaloids molecules, often to defend against herbivores and protect against pathogens. Many of these plant and fungal natural products have medicinal properties. There are multiple examples of these alkaloid compounds used to treat a wide range of conditions including, for example, drug addiction, depression, pain, cancer, and diabetes.

Indole alkaloid compounds occur in nature, typically in very small quantities in certain plants, fungi, and/or bacteria. Isolating such compounds from natural sources typically requires large amounts of starting material (e.g., biomass) as well as complicated separation techniques, and only provides the target compound(s) at low yield. Chemical synthesis strategies that target monoterpene indole alkaloids, particularly from readily available and low-cost precursors, are often highly complicated, involve toxic solvents, and also are associated with low yields.

Accordingly, there is a need for compositions and methods that provide for the production of indole alkaloid molecules from a renewable source such as recombinant microorganism(s). The disclosure provides polynucleotides, amino acid sequences (i.e., enzymes), recombinant cells and strains, and biosynthetic methods that produce indole alkaloids from low-cost feedstock or precursor compounds, as well as from cell lysates and/or in cell-free reactions. The methods and recombinant cells disclosed herein provide for synthesis of mitragyna- or corynanthe-type indole alkaloid compounds in good quantity and yield, providing advantages in scale, reliability, reproducibility and environmentally-friendly production, particularly when compared to existing production and isolation and/or purification methods.

### SUMMARY OF THE DISCLOSURE

In an aspect the disclosure provides for polynucleotide sequences that encode amino acid sequences (e.g., enzymes, transporters, etc.) that are useful in the biosynthesis of a downstream monoterpene indole alkaloid (e.g., from strictosidine or strictosidine aglycone to mitragynine, and intermediates, derivatives, and metabolites thereof). In embodiments of this aspect the polynucleotide sequence comprises a gene that encodes a polypeptide that is associated with monoterpene indole alkaloid synthesis.

In one aspect, the disclosure relates to a polynucleotide comprising a sequence as disclosed in the Sequence Listing provided herewith, or a sequence having about 90% sequence identity to any one of the disclosed sequences. In some embodiments, the disclosure relates to a polynucleotide sequence having about 95%, 96%, 97%, 98%, or 99% sequence identity to any one of the polynucleotide sequences disclosed in the Sequence Listing filed herewith.

In an aspect, the disclosure relates to an expression vector comprising a polynucleotide in accordance with disclosure and an operatively-linked promoter sequence that allows for expression of the one or more polynucleotide sequences in a cell.

In one aspect, the disclosure relates to a recombinant cell comprising a polynucleotide and/or an expression vector in accordance with disclosure. In some embodiments, the recombinant cell comprises a bacterial cell, a fungal cell, or a yeast cell. In some embodiments, the yeast cell comprises Saccharomyces, Candida, Pichia, Schizosaccharomyces, Scheffersomyces, Blakeslea, Rhodotorula, or Yarrowia. In some embodiments, the filamentous fungus cell comprises Aspergillus or Penicillium. In some embodiments, the bacterial cell comprises Escherichia, Corynebacterium, Caulobacter, Pseudomonas, Streptomyces, Bacillus, or Lactobacillus.

In yet further embodiments, the recombinant cell comprises at least one copy of the one or more polynucleotide sequences stably integrated into the genome of the cell. In further embodiments, a plurality of copies of the one or more polynucleotide sequences are stably integrated into the genome of the cell. In some further embodiments, the recombinant cell comprises a polynucleotide sequence or polynucleotide sequences in accordance with the disclosure that comprises the same sequence. In some further embodiments, the recombinant cell comprises a polynucleotide sequence or polynucleotide sequences in accordance with the disclosure that comprises different sequences. In yet further embodiments, the recombinant cell comprises a polynucleotide sequence or polynucleotide sequences in accordance with the disclosure that encode the same class of enzyme or amino acid sequence. In some further embodiments, the recombinant cell comprises a polynucleotide sequence or polynucleotide sequences in accordance with the disclosure that encode a different class of enzyme or amino acid sequence.

In another aspect, the disclosure provides a method for the biosynthesis of a downstream mitragyna-type or corynanthe-type monoterpene indole alkaloid (MIA) compound, or a precursor or metabolite thereof, wherein the method comprises culturing a recombinant cell in accordance with any of the aspects and embodiments of the disclosure under conditions that allow for the biosynthesis of the compound. In some embodiments, the method further comprises contacting the recombinant cell with a feedstock comprising one or more exogenous substrate compounds useful in the biosynthesis of one or more MIAs. In some further embodiments, the one or more exogenous substrate compounds may comprise an upstream precursor MIA compound such as, for example, nepetalactol, loganic acid, loganin, secologanin, L-tryptophan, tryptamine, serotonin, 5-methoxytryptamine, 4-methoxytryptamine, or 4-hydroxytryptamine, or the feed can comprise a plant extract that includes the one or more exogenous substrate compounds. In some further embodiments, the method can comprise one or more exogenous substrate compounds that are produced by a recombinant cell that can produce one or more upstream precursor MIA compounds, and which are fed into a recombinant cell according to any one of the aspects and embodiments disclosed herein.

In some embodiments, the biosynthetic method generates one or more of the following compounds: strictosidine, hydroxystrictosidine, methoxystrictosidine, strictosidine aglycone, hydroxystrictosidine aglycone, methoxystrictosidine aglycone, demethylcorynantheidine (20R, 20S), 9-hydroxydemethylcorynantheidine (20R, 20S), 9-methoxydemethylcorynantheidine (20R, 20S), corynantheidine, dihydrocorynantheine, 9-hydroxycorynantheidine, 9-hydroxydihydrocorynantheine, speciogynine, and/or mitragynine, among others, including halogenated analogs.

In a further aspect, the disclosure provides an amino acid sequence comprising a sequence as disclosed in the Sequence Listing provided herewith, or a sequence having about 90% sequence identity to any one of the disclosed sequences. In some embodiments, the disclosure relates to an amino acid sequence having about 95%, 96%, 97%, 98%, or 99% sequence identity to any one of the amino acid sequences disclosed in the Sequence Listing provided herewith, or a sequence having about 90% sequence identity to any one of the sequences. The disclosure also provides a polynucleotide encoding the amino acid sequences disclosed in the Sequence Listing provided herewith or those with 90%, 95%, 96%, 97%, 98% or 99% sequence identity to those amino acid sequences.

In some further embodiments, the polynucleotide that encodes a polypeptide associated with monoterpene indole alkaloid synthesis (e.g., arising from MIA precursors such as strictosidine, hydroxystrictosidine, methoxystrictosidine, and/or strictosidine aglycone, etc.) comprises a sequence that is a modified gene. In some embodiments, the polynucleotide sequence comprises a NADPH-dependent reductase (DCS); an enol-O-methyltransferase (enolMT); an indole ring hydroxylase (M9H); indole ring O-methyltranferase (M9OMT) or a combination of any two or more the genes.

In some further embodiments, the polynucleotide encodes a polypeptide that is useful in the biosynthesis of a monoterpene indole alkaloid that encodes for a polypeptide having a wide variety of biological activity including, for example, a polypeptide associated with electron transport, a polypeptide associated with metabolic processes, transporter protein (importer protein, efflux protein, etc.), an oxidase, a reductase, a dehydrogenase, a peptide synthetase, a halogenase, a transferase (e.g., glycosyltransferase), a synthetase (e.g., tryptophan synthetase) or other polypeptides having functional utility in the biosynthesis of a monoterpene indole alkaloid. In some embodiments the polynucleotide sequence encodes a cytochrome P450 (CYP), a cytochrome reductase (CPR), a cytochrome B (CYB), a tryptophan synthetase (TRPS), or combinations thereof. In such further embodiments, one or more of the polynucleotides can be used in combination with one or more polynucleotides, as described herein.

In some embodiments, the method comprises an NADPH-dependent reductase (DCS) that catalyzes the iminium reduction of an aglycone from compounds such as strictosidine aglycone to produce dihydrocorynantheine aldehyde. In some embodiments, the method comprises an NADPH-dependent reductase (DCS) gene in the Sequence Listing provided herewith, including DCS_In (SEQ ID NO:1), DCS_2n (SEQ ID NO:2), DCS_3n (SEQ ID NO: 3), DCS_4n (SEQ ID NO:4), DCS_5n (SEQ ID NO:5), DCS_6n (SEQ ID NO:6), DCS_7n (SEQ ID NO:7), DCS_8n (SEQ ID NO:8), DCS_9n (SEQ ID NO:9), DCS_10n ((SEQ ID NO: 10), DCS_1In (SEQ ID NO:11), DCS_12n (SEQ ID NO:12), or DCS_13n (SEQ ID NO: 13), DCS_14n (SEQ ID NO:14), DCS_15n (SEQ ID NO:15), DCS_16n ((SEQ ID NO:16), DCS_17n (SEQ ID NO:17), DCS_18n (SEQ ID NO:18), DCS_19n (SEQ ID NO:19), DCS_20n (SEQ ID NO:20), DCS_21n (SEQ ID NO:21), DCS_22n (SEQ ID NO:22), DCS_23n ((SEQ ID NO: 23), DCS_24n (SEQ ID NO:24), DCS_25n (SEQ ID NO:25), or DCS_26n (SEQ ID NO: 26), or a (DCS) amino acid sequence of the Sequence Listing provided herewith, including DCS_Ip (SEQ ID NO:53), DCS_2p (SEQ ID NO:54), DCS_3p (SEQ ID NO:55), DCS_4p (SEQ ID NO:56), DCS_5p (SEQ ID NO:57), DCS_6p (SEQ ID NO:58), DCS_7p (SEQ ID NO: 59), DCS_8p ((SEQ ID NO:60), DCS_9p (SEQ ID NO:61), DCS_10p (SEQ ID NO:62), DCS_11p (SEQ ID NO:63), DCS_12p (SEQ ID NO:64), or DCS_13p (SEQ ID NO:65), DCS_14p (SEQ ID NO:66), DCS_15p (SEQ ID NO:67), DCS_16p (SEQ ID NO:68), DCS_17p (SEQ ID NO:69), DCS_18p (SEQ ID NO:70), DCS_19p (SEQ ID NO:71), DCS_20p (SEQ ID NO: 72), DCS_21p (SEQ ID NO:73), DCS_22p (SEQ ID NO:74), DCS_23p (SEQ ID NO:75), DCS_24p (SEQ ID NO:76), DCS_25p (SEQ ID NO:77), or DCS_26p (SEQ ID NO:78) or a sequence having about 90%, 91%, 92%, 93%, 94%, 95%, 96%, 97%, 98%, or 99% sequence identity to any of the DCS sequences.

In some embodiments, the method comprises an indole ring O-methyltransferase (M9OMT) that catalyzes the methylation of oxygen bound to an indole ring, leading to the conversion of compounds such as 9-hydroxycorynantheidine to produce mitragynine. In some embodiments, the method comprises an indole-ring O-methyltransferase (M9OMT) gene in the Sequence Listing provided herewith, including M9OMT_In (SEQ ID NO:27), M9OMT_2n (SEQ ID NO:28), M9OMT_3n (SEQ ID NO:29), M9OMT_4n (SEQ ID NO:30), M9OMT_5n (SEQ ID NO:31), M9OMT_6n (SEQ ID NO:32), M9OMT_7n (SEQ ID NO:33), M9OMT_8n (SEQ ID NO:34), M9OMT_9n (SEQ ID NO:35), M9OMT_10n (SEQ ID NO:36), M9OMT_1In (SEQ ID NO:37), M9OMT_12n (SEQ ID NO:38), M9OMT_13n (SEQ ID NO:39), M9OMT_14n (SEQ ID NO:40), M9OMT_15n (SEQ ID NO:41), M9OMT_16n (SEQ ID NO: 42), M9OMT_17n (SEQ ID NO:43), M9OMT_18n (SEQ ID NO:44), M9OMT_19n (SEQ ID NO: 45), M9OMT_20n (SEQ ID NO:46), M9OMT_21n (SEQ ID NO:47), M9OMT_22n (SEQ ID NO:48), M9OMT_23n (SEQ ID NO:49), M9OMT_24n (SEQ ID NO:50), M9OMT_25n (SEQ ID NO:51), M9OMT_26n (SEQ ID NO:52), M9OMT_27n (SEQ ID NO: 233), M9OMT_28n (SEQ ID NO:234), M9OMT_29n (SEQ ID NO:235), M9OMT_30n (SEQ ID NO:236), M9OMT_3In (SEQ ID NO:237), M9OMT_32n (SEQ ID NO:238), M9OMT_33n (SEQ ID NO:239), M9OMT_34n (SEQ ID NO:240), M9OMT_35n (SEQ ID NO: 241), M9OMT_36n (SEQ ID NO:242), M9OMT_37n (SEQ ID NO:243), M9OMT_38n (SEQ ID NO:244), M9OMT_39n (SEQ ID NO:245), M9OMT_40n (SEQ ID NO:246), M9OMT_4In (SEQ ID NO:247), M9OMT_42n (SEQ ID NO:248), M9OMT_43n (SEQ ID NO: 249), M9OMT_44n (SEQ ID NO:250), M9OMT_45n (SEQ ID NO:251), M9OMT_46n (SEQ ID NO:252), M9OMT_47n (SEQ ID NO:253), M9OMT_48n (SEQ ID NO:254), M9OMT_49n (SEQ ID NO:255), M9OMT_50n (SEQ ID NO:256), M9OMT_5In (SEQ ID NO: 257), M9OMT_52n (SEQ ID NO:258), M9OMT_53n (SEQ ID NO:259), M9OMT_54n (SEQ ID NO:260), M9OMT_55n (SEQ ID NO:261), M9OMT_56n (SEQ ID NO:262), M9OMT_57n (SEQ ID NO:263), M9OMT_58n (SEQ ID NO:264), M9OMT_59n (SEQ ID NO: 265), M9OMT_60n (SEQ ID NO:266), M9OMT_6In (SEQ ID NO:267), M9OMT_62n (SEQ ID NO:268), M9OMT_63n (SEQ ID NO:269), M9OMT_64n (SEQ ID NO:270), or an indole-ring O-methyltransferase (M9OMT) amino acid sequence in the Sequence Listing provided herewith, including M9OMT_Ip (SEQ ID NO:79), M9OMT_2p (SEQ ID NO:80), M90MT_3p (SEQ ID NO:81), M9OMT_4p (SEQ ID NO:82), M9OMT_5p (SEQ ID NO:83), M9OMT_6p (SEQ ID NO:84), M9OMT_7p (SEQ ID NO:85), M9OMT_8p (SEQ ID NO:86), M9OMT_9p (SEQ ID NO:87), M9OMT_10p (SEQ ID NO:88), M9OMT_11p (SEQ ID NO:89), M9OMT_12p (SEQ ID NO:90), M9OMT_13p (SEQ ID NO:91), M9OMT_14p (SEQ ID NO: 92), M9OMT_15p (SEQ ID NO:93), M9OMT_16p (SEQ ID NO:94), M9OMT_17p (SEQ ID NO: 95), M9OMT_18p (SEQ ID NO:96), M9OMT_19p (SEQ ID NO:97), M9OMT_20p (SEQ ID NO:98), M9OMT_21p (SEQ ID NO:99), M9OMT_22p (SEQ ID NO:100), M9OMT_23p (SEQ ID NO:101), M9OMT_24p (SEQ ID NO: 102), M9OMT_25p (SEQ ID NO: 103), M9OMT_26p (SEQ ID NO: 104), M9OMT_27p (SEQ ID NO: 105), M9OMT_28p (SEQ ID NO:106), M9OMT_29p (SEQ ID NO:107), M9OMT_30p (SEQ ID NO:108), M9OMT_31p (SEQ ID NO:109), M9OMT_32p (SEQ ID NO: 110), M9OMT_33p (SEQ ID NO: 111), M9OMT_34p (SEQ ID NO:112), M9OMT_35p (SEQ ID NO:113), M90MT_36p (SEQ ID NO:114), M9OMT_37p (SEQ ID NO:115), M9OMT_38p (SEQ ID NO:116), M9OMT_39p (SEQ ID NO:117), M9OMT_40p (SEQ ID NO:118), M9OMT_41p (SEQ ID NO: 119), M9OMT_42p (SEQ ID NO: 120), M9OMT_43p (SEQ ID NO: 121), M9OMT_44p (SEQ ID NO: 122), M9OMT_45p (SEQ ID NO:123), M9OMT_46p (SEQ ID NO:124), M9OMT_47p (SEQ ID NO: 125), M9OMT_48p (SEQ ID NO:126), M9OMT_49p (SEQ ID NO: 127), M9OMT_50p (SEQ ID NO:128), M9OMT_51p (SEQ ID NO:129), M9OMT_52p (SEQ ID NO:130), M9OMT_53p (SEQ ID NO:131), M9OMT_54p (SEQ ID NO:132), M9OMT_55p (SEQ ID NO:133), M9OMT_56p (SEQ ID NO:134), M9OMT_57p (SEQ ID NO: 135), M9OMT_58p (SEQ ID NO: 136), M9OMT_59p (SEQ ID NO:137), M9OMT_60p (SEQ ID NO:138), M9OMT_61p (SEQ ID NO:139), M9OMT_62p (SEQ ID NO:140), M9OMT_63p (SEQ ID NO:141), M9OMT_64p (SEQ ID NO:142), or a sequence having about 90%, 91%, 92%, 93%, 94%, 95%, 96%, 97%, 98%, or 99% sequence identity to any of the M9OMT sequences.

In some embodiments, the method comprises an enol O-methyltransferase (enolMT) that catalyzes the methylation of enol oxygens, leading to the conversion of compounds such as demethylcorynantheidine to produce corynantheidine. In some embodiments, the method comprises an enol-O-methyltransferase (enolMT) gene in the Sequence Listing provided herewith, including cnolMT_1n (SEQ ID NO:271), enolMT_2n (SEQ ID NO:272), enolMT_3n (SEQ ID NO:273), enolMT_4n (SEQ ID NO:274), enolMT_5n (SEQ ID NO:275), cnolMT_6n (SEQ ID NO:276), or enolMT_7n (SEQ ID NO:277), or an enol-O-methyltransferase (enolMT) amino acid sequence in the Sequence Listing provided herewith, including enolMT_Ip (SEQ ID NO: 143), cnolMT_2p (SEQ ID NO:144), enolMT_3p (SEQ ID NO: 145), enolMT_4p (SEQ ID NO: 146), enolMT_5p (SEQ ID NO: 147), enolMT_6p (SEQ ID NO:148), or enolMT_7p (SEQ ID NO: 149), or a sequence having about 90%, 91%, 92%, 93%, 94%, 95%, 96%, 97%, 98%, or 99% sequence identity to any of the enolMT sequences.

In some embodiments, the method comprises an indole ring or aromatic hydroxylase (M9H) that catalyzes the hydroxylation of compounds at the 4′ position of the indole ring in conjunction with the cofactors NAD (P) H, FMN, and FAD+ such as in corynantheidine to produce 9-hydroxycorynantheidine. Other P450 CYPs enzymes can also catalyze the hydroxylation of compounds at the 7′ position of the indole ring such as in mitragynine to produce 7-hydroxymitragynine. In some embodiments, the method comprises an M9H indole ring or aromatic hydroxylase gene of the Sequence Listing provided herewith, including M9H_In (SEQ ID NO:278), M9H_2n (SEQ ID NO:279), M9H_3n (SEQ ID NO:280), M9H_4n (SEQ ID NO:281), M9H_5n (SEQ ID NO:282), M9H_6n (SEQ ID NO:283), M9H_7n (SEQ ID NO: 284), M9H_8n (SEQ ID NO:285), M9H_9n (SEQ ID NO:286), M9H_10n (SEQ ID NO: 287), M9H_1 In (SEQ ID NO:288), M9H_12n (SEQ ID NO:289), M9H_13n (SEQ ID NO: 290), M9H_14n (SEQ ID NO:291), M9H_15n (SEQ ID NO:292), M9H_16n (SEQ ID NO: 293), M9H_17n (SEQ ID NO:294), M9H_18n (SEQ ID NO:295), M9H_19n (SEQ ID NO: 296), M9H_20n (SEQ ID NO:297), M9H_21n (SEQ ID NO:298), M9H_22n (SEQ ID NO: 299), M9H_23n (SEQ ID NO:300), M9H_24n (SEQ ID NO:301), M9H_25n (SEQ ID NO: 302), M9H_26n (SEQ ID NO:303), M9H_27n (SEQ ID NO:304), M9H_28n (SEQ ID NO: 305), M9H_29n (SEQ ID NO:306), M9H_30n (SEQ ID NO:307), M9H_3In (SEQ ID NO: 308), M9H_32n (SEQ ID NO:309), M9H_33n (SEQ ID NO:310, M9H_34n (SEQ ID NO: 311), M9H_35n (SEQ ID NO:312), or M9H_36n (SEQ ID NO:313), or a (M9H) amino acid sequence of the Sequence Listing provided herewith, including M9H_Ip (SEQ ID NO: 150), M9H_2p (SEQ ID NO:151), M9H_3p (SEQ ID NO:152), M9H_4p (SEQ ID NO:153), M9H_5p (SEQ ID NO:154), M9H_6p (SEQ ID NO: 155), M9H_7p (SEQ ID NO:156), M9H_8p (SEQ ID NO: 157), M9H_9p (SEQ ID NO: 158), M9H_10p (SEQ ID NO:159), M9H_11p (SEQ ID NO: 160), M9H_12p (SEQ ID NO:161), M9H_13p (SEQ ID NO:162), M9H_14p (SEQ ID NO: 163), M9H_15p (SEQ ID NO: 164), M9H_16p (SEQ ID NO: 165), M9H_17p (SEQ ID NO: 166), M9H_18p (SEQ ID NO:167), M9H_19p (SEQ ID NO:168), M9H_20p (SEQ ID NO: 169), M9H_21p (SEQ ID NO:170), M9H_22p (SEQ ID NO: 171), M9H_23p (SEQ ID NO: 172), M9H_24p (SEQ ID NO:173), M9H_25p (SEQ ID NO:174), M9H_26p (SEQ ID NO: 175), M9H_27p (SEQ ID NO:176), M9H_28p (SEQ ID NO:177), M9H_29p (SEQ ID NO: 178), M9H_30p (SEQ ID NO:179), M9H_31p (SEQ ID NO:180), M9H_32p (SEQ ID NO: 181), M9H_33p (SEQ ID NO:182), M9H_34p (SEQ ID NO:183), M9H_35p (SEQ ID NO: 184), or M9H_36p (SEQ ID NO:185), or a sequence having about 90%, 91%, 92%, 93%, 94%, 95%, 96%, 97%, 98%, or 99% sequence identity to any of the M9H sequences.

In embodiments, the polynucleotide sequence comprises a CYB5 gene comprising any one or more of the CYB5 sequences disclosed in the Sequence Listing provided herewith, or a sequence having at least about 90%, 91%, 92%, 93%, 94%, 95%, 96%, 97%, 98%, or 99% sequence identity to any one of sequences CYB5_In (SEQ ID NO:315) or CYB5_2n (SEQ ID NO: 316).

In embodiments, the polynucleotide sequence comprises a CPR gene comprising any one or more of the CPR sequences disclosed in the Sequence Listing provided herewith, or a sequence having at least about 90%, 91%, 92%, 93%, 94%, 95%, 96%, 97%, 98%, or 99% sequence identity to any one of sequences CPR_In (SEQ ID NO:317), CPR_2n (SEQ ID NO: 318), CPR_3n (SEQ ID NO:319), or CPR_4n (SEQ ID NO:320).

In embodiments, the polynucleotide sequence comprises a transporter gene comprising any one or more of the sequences disclosed in the Sequence Listing provided herewith, or a sequence having at least 90%, 91%, 92%, 93%, 94%, 95%, 96%, 97%, 98%, or 99% sequence identity to any one of sequences trxporter_In (SEQ ID NO:321), trxporter_2n (SEQ ID NO: 322), trxporter_3n (SEQ ID NO:323), trxporter_4n (SEQ ID NO:324), trxporter_5n (SEQ ID NO: 325), or trxporter_6n (SEQ ID NO:326).

In some alternative embodiments, the polynucleotide sequence comprises a tryptamine hydroxylase which can hydroxylate an indole, tryptamine, or tryptophan molecule, such as PsiH-type and other hydroxylase genes, such as a tryptamine-4-hydroxylase (T4H), disclosed in U.S. patent application Ser. No. 18/000,931 (ENZYMES AND REGULATORY PROTEINS IN TRYPTAMINE METABOLISM) and U.S. Patent Application No.: 11,441,164 (BIOSYNTHETIC PRODUCTION OF PSILOCYBIN AND RELATED INTERMEDIATES IN RECOMBINANT ORGANISMS).

In some alternative embodiments, the polynucleotide sequence comprises an O-methyltransferase which can methylate hydroxyl groups on an indole or tryptamine molecule, such as the indole (or tryptamine)O-methyltransferases (IOMTs) disclosed in U.S. patent application Ser. No. 18/000,931 (ENZYMES AND REGULATORY PROTEINS IN TRYPTAMINE METABOLISM).

In some alternative embodiments, the polynucleotide sequence comprises a kinase which can phosphorylate a hydroxylated indole, tryptamine, or tryptophan such as PsiK-type and other kinases genes disclosed in U.S. patent application Ser. No. 18/000,931 (ENZYMES AND REGULATORY PROTEINS IN TRYPTAMINE METABOLISM) and U.S. Patent Application No.: 11,441,164 (BIOSYNTHETIC PRODUCTION OF PSILOCYBIN AND RELATED INTERMEDIATES IN RECOMBINANT ORGANISMS).

In some alternative embodiments, the polynucleotide sequence comprises a tryptophan or tryptamine halogenase (TrpHalo) which can halogenate indole, tryptamine, and/or tryptophan molecules, such as the halogenases disclosed in U.S. patent application Ser. No. 18/000,931 (ENZYMES AND REGULATORY PROTEINS IN TRYPTAMINE METABOLISM).

In another aspect the disclosure provides for amino acid sequences (e.g., enzymes, transporters, etc.) that are useful in the biosynthesis of a downstream monoterpene indole alkaloid. In some embodiments, amino acid sequence comprises a modified sequence of a polypeptide (e.g., protein, enzyme, etc.) sequence that comprises a NADPH-dependent reductase (DCS); an enol-O-methyltransferase (enolMT); an indole ring hydroxylase (M9H); or an indole ring O-methyltranferase (M9OMT); or a combination of any two or more of such polypeptides.

In some further embodiments the amino acid sequence useful in the biosynthesis comprising a polypeptide having a wide variety of biological activity including, for example, a polypeptide associated with electron transport, a polypeptide associated with metabolic processes, transporter protein (importer protein, efflux protein, etc.), an oxidase, a reductase, a dehydrogenase, a peptide synthetase, a halogenase, a transferase (e.g., glycosyltransferase), a synthetase (e.g., tryptophan synthetase) or other polypeptides having functional utility in the biosynthesis of a monoterpene indole alkaloid. In some embodiments the polypeptide comprises a sequence of a cytochrome P450 (CYP), a cytochrome reductase (CPR), a cytochrome B (CYB), a tryptophan synthetase (TRPS), or combinations thereof. In such further embodiments, one or more of the polypeptides can be used in combination.

In embodiments, the amino acid sequence comprises a DCS sequence disclosed in the Sequence Listing provided herewith, or a sequence having at least about 90%, 91%, 92%, 93%, 94%, 95%, 96%, 97%, 98%, or 99% sequence identity to any one of sequences DCS_Ip-26p.

In embodiments, the amino acid sequence comprises a M9OMT sequence disclosed in the Sequence Listing provided herewith, or a sequence having at least about 90%, 91%, 92%, 93%, 94%, 95%, 96%, 97%, 98%, or 99% sequence identity to any one of sequences M90MT_Ip-64p.

In embodiments, the amino acid sequence comprises a enolMT sequence disclosed in the Sequence Listing provided herewith, or a sequence having at least about 90%, 91%, 92%, 93%, 94%, 95%, 96%, 97%, 98%, or 99% sequence identity to any one of sequences enolMT_1p-7p.

In embodiments, the amino acid sequence comprises a M9H sequence disclosed in the Sequence Listing provided herewith, or a sequence having at least about 90%, 91%, 92%, 93%, 94%, 95%, 96%, 97%, 98%, or 99% sequence identity to any one of sequences M9H_1p-36p.

In embodiments, the amino acid sequence comprises a CYB5 sequence disclosed in the Sequence Listing provided herewith, or a sequence having at least about 90%, 91%, 92%, 93%, 94%, 95%, 96%, 97%, 98%, or 99% sequence identity to any one of sequences CYB5_Ip (SEQ ID NO: 187) or CYB5_2p (SEQ ID NO:188).

In embodiments, the amino acid sequence comprises a CPR sequence disclosed in the Sequence Listing provided herewith, or a sequence having at least about 90%, 91%, 92%, 93%, 94%, 95%, 96%, 97%, 98%, or 99% sequence identity to any one of sequences CPR_Ip (SEQ ID NO: 189), CPR_2p (SEQ ID NO:190), CPR_3p (SEQ ID NO:191), or CPR_4p (SEQ ID NO: 192).

In embodiments, the amino acid sequence comprises a transporter protein comprising any one or more of the sequences disclosed in the Sequence Listing provided herewith, or a sequence having at least about 90%, 91%, 92%, 93%, 94%, 95%, 96%, 97%, 98%, or 99% sequence identity to any one of sequences trxporter_Ip (SEQ ID NO:193), trxporter_2p (SEQ ID NO: 194), trxporter_3p (SEQ ID NO:195), trxporter_4p (SEQ ID NO:196), trxporter_5p (SEQ ID NO: 197), or trxporter_6p (SEQ ID NO:198).

In some alternative embodiments, the amino acid sequence comprises a tryptamine hydroxylase which can hydroxylate an indole, tryptamine, or tryptophan molecule, such as PsiH-type and other hydroxylase genes, such as a tryptamine-4-hydroxylase (T4H), disclosed in U.S. patent application Ser. No. 18/000,931 (ENZYMES AND REGULATORY PROTEINS IN TRYPTAMINE METABOLISM) and U.S. Patent Application No.: 11,441,164 (BIOSYNTHETIC PRODUCTION OF PSILOCYBIN AND RELATED INTERMEDIATES IN RECOMBINANT ORGANISMS).

In some alternative embodiments, the amino acid sequence comprises an O-methyltransferase which can methylate hydroxyl groups on an indole or tryptamine molecule, such as the indole (or tryptamine)O-methyltransferases (IOMTs) disclosed in U.S. patent application Ser. No. 18/000,931 (ENZYMES AND REGULATORY PROTEINS IN TRYPTAMINE METABOLISM).

In some alternative embodiments, the polynucleotide sequence comprises a kinase which can phosphorylate a hydroxylated indole, tryptamine, or tryptophanm such as PsiK-type and other kinases genes disclosed in U.S. patent application Ser. No. 18/000,931 (ENZYMES AND REGULATORY PROTEINS IN TRYPTAMINE METABOLISM) and U.S. Patent Application No.: 11,441,164 (BIOSYNTHETIC PRODUCTION OF PSILOCYBIN AND RELATED INTERMEDIATES IN RECOMBINANT ORGANISMS).

In some alternative embodiments, the amino acid sequence comprises a tryptophan or tryptamine halogenase (TrpHalo) which can halogenate indole, tryptamine, and/or tryptophan molecules, such as the halogenases disclosed in U.S. patent application Ser. No. 18/000,931 (ENZYMES AND REGULATORY PROTEINS IN TRYPTAMINE METABOLISM).

In another aspect the disclosure provides for vectors (e.g., expression cassettes, episomes) that comprise one or more of the polynucleotide sequences, including multiple copies of the same sequence or a sequence encoding the same or similar type of functional amino acid sequence, as described above.

In another aspect the disclosure provides recombinant cells (or “host cells”) that are engineered to produce an upstream monoterpene indole alkaloid, as discussed herein. In some embodiments, the recombinant cell comprises one or a plurality of the polynucleotide sequences of the disclosure. In some embodiments, the recombinant cell comprises one or a plurality of the polypeptide sequences of the disclosure. In some embodiments, the recombinant cell is transformed with a vector, or otherwise genetically manipulated, to express one or a plurality of the polynucleotide sequences of the disclosure. In some embodiments, the recombinant cell comprises a modification to one or more naturally-occurring genes in the cell. In some further embodiments, the genetic modification comprises a deletion of an endogenous gene. In some embodiments, the genetic modification comprises an addition of an exogenous gene. In yet further embodiments, the exogenous gene encodes a polypeptide that is different from the polypeptides of the disclosure. In some embodiments, the genetic modification comprises a combination of exogenous genes comprising a sequence that encodes a polypeptide of the disclosure and a sequence that encodes a polypeptide that is different from the polypeptides of the disclosure.

In any of the above aspects and embodiments relating to a recombinant cell, the cell can be fungal cell or a microbial cell. In some embodiments, a microbial cell can be a bacterial cell (e.g., E. coli, or the like) or a yeast cell (e.g., Saccharomyces or Pichia, or the like). In some embodiments, the recombinant cell comprises a further genetic modification that improves the cells ability to produce a monoterpene indole alkaloid, or a precursor or metabolite thereof.

In another aspect, the disclosure provides a method for the biosynthesis of a downstream monoterpene indole alkaloid, or a precursor or metabolite thereof, the method comprising culturing a recombinant cell in accordance with the aspects and embodiments of the disclosure under conditions that allow for the biosynthesis.

In some embodiments, the method provides for the biosynthesis of one or more compounds including strictosidine, hydroxystrictosidine, methoxystrictosidine, strictosidine aglycone, hydroxystrictosidine aglycone, methoxystrictosidine aglycone, demethylcorynantheidine (20R, 20S), 9-hydroxydemethylcorynantheidine (20R, 20S), 9-methoxydemethylcorynantheidine (20R, 20S), corynantheidine, dihydrocorynantheine, 9-hydroxycorynantheidine, 9-hydroxydihydrocorynantheine, speciogynine, and/or mitragynine, among others. In some embodiments, the method provides for the biosynthesis of mitragynine.

In some embodiments, the method provides for the biosynthesis of demethylcorynantheidine.

In some embodiments, the method provides for the biosynthesis of corynantheidine. In some embodiments, the method provides for the biosynthesis of 9-hydroxycorynantheidine.

In some embodiments, the method provides for the biosynthesis of a downstream monoterpene indole alkaloid that is fed precursors generated by an upstream MIA biosynthetic pathway providing compounds such as loganin, loganic acid, strictosidine, or strictosidine aglycone, etc.

Other aspects and embodiments falling within the scope and spirit of the disclosure will be apparent to those of skill in the art in light of the following description and illustrative examples.

### BRIEF DESCRIPTION OF DRAWINGS

FIG. 1 depicts the chemical structures of example monoterpene indole alkaloid (MIA) compounds, including (A) the orientation of the monoterpene and indole structures in an illustrative MIA compound, strictosidine, (B) mitragynine, (C) the mitragynine stereoisomer, speciogynine, and (D) R-group variable substitutions of mitragynine resulting in analogs and derivatives of mitragynine.

FIG. 2. depicts the biosynthesis pathway to form the 4-ring structure of mitragynine from a monoterpene-derived iridoid glucoside such as secologanin combined with (A) tryptamine to form the strictosidine aglycone precursor for mitragynine, (B) 4-hydroxytryptamine to form the hydroxystrictosidine aglycone precursor for mitragynine, and (C) 4-methoxytryptamine to form the methoxystrictosidine aglycone precursor for mitragynine.

FIG. 3 depicts routes to generate mitragynine-type analogs from substituted indoles and tryptamines, such as from (A) a modified indole that leads to a substituted tryptophan, and (B) enzymatic modifications of tryptamine which can lead to downstream analogs of alkaloids including substituted strictosidine through mitragynine analogs.

FIG. 4 depicts an illustrative diagram of a recombinant host cell expressing combinations of genes for downstream MIA synthesis in accordance with embodiments of the disclosure that can produce MIAs such as (A) a recombinant cell expressing an upstream pathway that leads to precursors and intermediates for mitragynine biosynthesis and (B) a recombinant cell which can express a partial or full pathway to produce mitragynine, upstream mitragynine precursors, and related molecules.

FIG. 5 depicts the bioconversion of MIA precursors into successive later (downstream) target MIAs, such as (A) MIA precursors, such as loganin, from a plant source fed to a cell to generate mitragynine, a (B) a co-culture, mixed lysate, or cell-free conversion of MIA precursor compounds (e.g., strictosidine, etc.) generated by or sourced from an exogenous recombinant cell, and reacted with (i.e., fed to) a recombinant cell or cell lysate from a cell expressing downstream pathway enzymes to generate mitragynine, and/or (C) a media feed supplemented with any source of precursors, such as modified tryptamines and secologanin to be utilized for mitragynine production.

FIG. 6 depicts LC-MS extracted ion counts (EIC) and detection of fermentation-derived demethylcorynantheidine from a recombinant host expressing DCS in accordance with example embodiments of the disclosure.

FIG. 7 depicts LC-MS extracted ion counts (EIC) and detection of fermentation-derived MIA pathway product, corynantheidine, from a recombinant host in accordance with example embodiments of the disclosure.

FIG. 8 depicts high resolution mass (from HRMS) and detection of fermentation-derived MIA pathway product, corynantheidine, from a recombinant host in accordance with example embodiments of the disclosure.

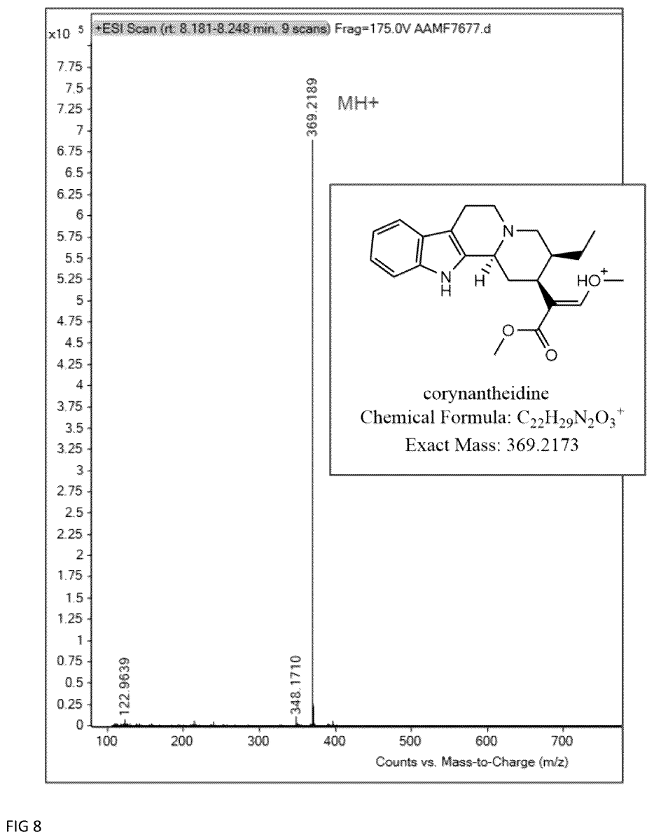

FIG. 9 depicts LC-MS/MS fragmentation of yeast fermentation-derived corynantheidine in accordance with example embodiments of the disclosure.

FIG. 10 depicts the characterization of corynantheidine by comparing retention time, LC-MS, LC-MS/MS fragmentation of yeast fermentation-derived corynantheidine in accordance with example embodiments of the disclosure, corynantheidine from kratom extract, hirsutine commercial standard, and corynantheidine commercial standard

FIG. 11 depicts a phylogenetic analysis of enolMT class in comparison to the SABATH family methyltransferases showing distinct grouping of the enolMT class from IAMT, SAMT, JMT, BAMT, XMT, LAMT, FAMT, M9OMT, C3OMT, and COMT methyltransferase classes.

FIG. 12 provides an illustrative scheme showing 4-hydroxytryptamine feeding to yeast fermentation cultures to biosynthesize 9-hydroxycorynantheidine (normitragynine).

FIG. 13 depicts LC-MS extracted ion counts (EIC) and detection of fermentation-derived MIA pathway products 9-hydroxycorynantheidine and its stereoisomer, 9-hydroxy-dihydrocorynantheidine, from a recombinant host in accordance with example embodiments of the disclosure.

FIG. 14 provides an illustrative scheme showing 4-methoxytryptamine feeding to yeast fermentation cultures to biosynthesize mitragynine in accordance with example embodiments of the disclosure.

FIG. 15 depicts LC-MS extracted ion counts (EIC) and detection of fermentation-derived MIA pathway products mitragynine and speciogynine, from a recombinant host in accordance with example embodiments of the disclosure.

FIG. 16 depicts LC-MS extracted ion counts (EIC) and detection of biosynthetic-derived MIA pathway products mitragynine and speciogynine, from a cell-free reaction in accordance with example embodiments of the disclosure.

### DETAILED DESCRIPTION

In a general sense, the disclosure provides for novel sequences (polynucleotide, proteins, and enzymes) that are useful in the production of recombinant cells and biosynthetic processes for the synthesis of indole alkaloids. In various aspects and embodiments, the disclosure provides a biosynthetic method that is sufficient for the production of at least one monoterpene indole alkaloid (MIA) (e.g., a downstream MIA in the mitragynine pathway), synthetic intermediates, metabolites, or precursor species thereof. In various aspects and embodiments, the disclosure provides sequences, recombinant cells, and methods comprising the same for the biosynthesis of monoterpene indole alkaloids, synthetic intermediates, metabolites, or precursor species thereof. In some aspects and embodiments, the disclosure provides a sequence comprising one or more polynucleotide sequences, including polynucleotide sequences that encode for enzymes, transporter proteins, localization proteins, and/or regulatory protein sequences, associated with the biosynthetic pathway for the production of indole alkaloids including mitragynine, that comprise any one or combination of a NADPH-dependent reductase (DCS); an enol-O-methyltransferase (enolMT); an indole ring hydroxylase (M9H); an indole ring O-methyltranferase (M9OMT), a cytochrome b5 (CYB5), a cytochrome P450 reductase (CPR), a tryptophan importer (TAT2), a SAMe importer (SAM3), a transporter (trxporter), a methionine importer (MUP1), a mRNA stabilizer (IME4), a P450/CYP activity enhancer (ICE2 and/or INO2), a SAMe pathway enhancer (ADK1 and/or SAM2), and/or a tryptamine 4-hydroxylase (PsiH or T4H), a tryptamine O-methyltransferase (IOMT), and/or a halogenase (TrpHalo).

### Abbreviations and Definitions

Unless defined otherwise, all technical and scientific terms used herein have the same meaning as commonly understood by one of ordinary skill in the art to which the disclosure belongs. A number of terms and abbreviations appear throughout the disclosure and, unless otherwise indicated, should be understood to have the definitions that follow.

A “nucleic acid” or “polynucleotide” sequence are used herein to refer to deoxyribonucleotides or ribonucleotides and polymers thereof in either single- or double-stranded form. The terms encompass nucleic acids containing known analogues of natural nucleotides that have similar binding properties as the reference nucleic acid and are metabolized in a manner similar to naturally-occurring nucleotides. Unless otherwise indicated, a particular nucleic acid sequence also implicitly encompasses conservatively modified or degenerate variants thereof (e.g., degenerate codon substitutions) and complementary sequences, as well as the sequence explicitly indicated.

The terms “nucleic acid primer,” “nucleic acid probe,” and “oligonucleotide” are all used herein to refer to a short nucleic acid sequence, which may comprise or consist of a fragment of a longer polynucleotide sequence. Oligonucleotides, nucleic acid primers, and/or nucleic acid probes can be DNA, RNA, or a hybrid thereof, or chemically modified analogs or derivatives thereof and are typically single-stranded. However, they can also be double-stranded having two complementing strands that can be separated (e.g., melted) under denaturating conditions (e.g., stringent, moderately stringent, or highly stringent conditions). In some embodiments an oligonucleotide, primer, and/or probe has a length of from about 8 nucleotides to about 200 nucleotides, or from about 12 nucleotides to about 100 nucleotides, or from about 18 to about 50 nucleotides. In embodiments, oligonucleotides, primers, and/or probes can be labeled with detectable markers or modified in any conventional manners for various molecular biological applications (e.g., to inhibit or prevent degradation).

The terms “amino acid sequence,” “polypeptide,” “protein,” and “peptide” as used herein all refer to a sequence of amino acid residues linked by peptide bonds or modified peptide bonds. The amino acid sequence can be of any length of greater than two amino acids. Polypeptides can include modified forms of the sequence, such as naturally occurring or synthetically generated post-translational modifications, or modifications to the chemical structure of one or more amino acid residues. Non-limiting examples of modified forms include glycosylated sequences, phosphorylated sequences, myristoylated sequences, palmitoylated sequences, ribosylated sequences, acetylated sequences, and the like. Modifications can also include intra- or inter-molecular crosslinking or covalent attachments to moieties such as lipids, flavin, biotin, polyethylene glycol or derivatives thereof, and the like. In addition, modifications may also include protein cyclization, branching of the amino acid chain, and cross-linking of the protein. Further, amino acids other than the naturally-encoded twenty amino acids may also be included in a polypeptide.

The polypeptide sequences or polynucleotide sequences can be isolated and/or purified, both of which refer to a polypeptide (or a polynucleotide) that is substantially separated from other cellular components (i.e., proteins, DNA, RNA, lipids, membranes, cell debris) of the organism in which the sequence is produced (e.g., 30%, 40%, 50%, 60%, 70%, 80%, 90%, 95%, 96%, 97%, 98%, 99%, 100% free of contaminants).

As used herein, a “conservative amino acid substitution” refers to an amino acid substitution in a polypeptide sequence wherein the substituted amino acid(s) has similar characteristics to the amino acid in the native sequence, for example charge, hydrophobicity and/or hydrophilicity profile, polarity, size, and the like. Non-limiting examples of conservative amino acid substitutions are Ser for Ala, Thr, or Cys; Lys for Arg; Gln for Asn, His, or Lys; His for Asn; Glu for Asp or Lys; Asn for His or Gln; Asp for Glu; Pro for Gly; Leu for Ile, Phe, Met, or Val; Val for Ile or Leu; Ile for Leu, Met, or Val; Arg for Lys; Met for Phe; Tyr for Phe or Trp; Thr for Ser; Trp for Tyr; and Phe for Tyr. Non-natural amino acids can also serve as a conservative amino acid substitution for a naturally occurring amino acid.

The term “functional variant” refers to a recombinant biological sequence that is structurally different from a naturally occurring sequence and capable of performing the same function as the naturally occurring sequence. For example, a functional variant of a pathway gene or enzyme comprises a nucleotide and/or amino acid sequence that is altered by one or more nucleotides and/or amino acids compared to the native indole alkaloid pathway gene or enzyme sequences, and is capable of performing the function of the native or parent indole alkaloid pathway gene or enzyme (e.g., an enolMT enzyme capable of generating corynantheidine). In embodiments, a modification to the native or parent sequence may provide for the same or improved functional activity and reaction parameters without altering the underlying function of the native biological sequence. Functional variants may comprise conservative sequence substitutions, sequence additions, and sequence deletions. The sequence modifications can be introduced by standard techniques known in the art, such as site-directed mutagenesis and random PCR-mediated mutagenesis, and may comprise natural as well as non-natural nucleotides and amino acids, and/or analogs thereof. In some embodiments, recombinant biological sequences, including functional variants, comprise amino acid analogs (e.g. amino acids other than the 20 amino acids encoded by DNA or RNA) and/or labeled amino acids and amino acid analogs comprising, for example, fluorescent dyes, radioisotopes, electron dense agents, and the like.

A “recombinant” nucleic acid or amino acid sequence is a nucleic acid or polypeptide produced by recombinant DNA technology, e.g., as described in Green and Sambrook (2012). The terms “recombinant,” “heterologous,” and “exogenous,” can be used interchangeably herein and, when referring to polynucleotides, mean a polynucleotide (e.g., a DNA or RNA sequence or a gene) that originates from a source foreign to the particular host cell or, if from the same source, is modified from its original form. Thus, a heterologous gene in a host cell includes a gene that is endogenous to the particular host cell but has been modified through, for example, the use of site-directed mutagenesis or other recombinant techniques. The terms also include non-naturally occurring multiple copies of a naturally occurring DNA sequence. Thus, the terms refer to a DNA segment that is foreign or heterologous to the cell, or homologous to the cell but in a position or form within the host cell in which the element is not ordinarily found. Similarly, the terms when referring to polypeptides, means a polypeptide or amino acid sequence that originates from a source foreign to the particular host cell or, if from the same source, is modified from its original form. As such, recombinant DNA molecules can be expressed in a host cell to produce a recombinant polypeptide.

The terms “transformed,” “transgenic,” and “recombinant,” when used with reference to host cells typically refer to an isolated cell or a cell in culture, such as a plant, fungal, or microbial (e.g. bacterial or yeast) cell, into which a heterologous polynucleotide has been introduced or a heterologous amino acid sequence is expressed. The polynucleotide can be integrated into the genome of the host cell, or it can be present as an extrachromosomal molecule. Such an extrachromosomal molecule can be auto-replicating, as discussed herein.

Transformed cells, tissues, or subjects are understood to encompass not only the end-product of a transformation process, but also transgenic progeny thereof.

The terms “plasmid,” “vector,” and “cassette” (e.g., transformation cassette, expression vector, or expression cassette) generally refer to an extra-chromosomal element that comprises nucleic acid sequences (e.g., genes, promoters, regulatory elements (inducers, repressors, etc.) and the like) which are not part of the central metabolism of the cell, and can be circular, double-stranded DNA molecules. Such elements may be autonomously replicating sequences, genome integrating sequences, phage or nucleotide sequences (linear or circular) of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. Merely for purposes of clarity, a “transformation cassette” refers to a vector containing a foreign gene and having elements in addition to the foreign gene that facilitate transformation of a particular host cell. An “expression cassette” or “expression vector” refers to a vector containing a foreign gene and having elements in addition to the foreign gene that allow for expression and/or enhanced expression of that gene in a foreign host. Thus, the terms can refer to a nucleic acid molecule capable of transporting another nucleic acid molecule to which it has been linked. A non-limiting example of an expression vector includes a gene encoding an enzyme with a promoter that is functional in yeast, where the promoter and gene are oriented such that the promoter drives expression of the enzyme in the yeast cell. A non-limiting example of a vector capable of extra-chromosomal replication is an episome.

A “linker” refers to a short amino acid sequence that separates multiple domains of a polypeptide. In some embodiments, the linker prohibits energetically or structurally unfavorable interactions between the discrete domains.

A recombinant gene can be “codon optimized” when its nucleotide sequence is modified to accommodate codon bias of the host organism, typically to improve gene expression and increase translational efficiency of the gene.

As used herein a “coding sequence” generally refers to a DNA sequence that encodes for a specific amino acid sequence.

A “regulatory sequence” is generally used to refer to a polynucleotide sequence located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

A “promoter” refers to a DNA sequence capable of controlling the expression of a coding sequence or functional RNA. Commonly, a coding sequence is oriented or located 3′ to a promoter sequence. Promoters may be derived in their entirety from a native gene, or be composed of different elements derived from different natural promoters, or comprise synthetic DNA segments. Different promoters may direct the expression of a gene in different cell types, or at different stages of development or cell growth/cycle, or in response to different environmental conditions. Promoters that cause a gene to be expressed in most cell types at most times are commonly referred to as “constitutive promoters.”

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression” generally refers to the transcription and stable accumulation of sense (mRNA) or antisense RNA derived from a nucleic acid sequence. “Over-expression” refers to the production of a gene product in transgenic or recombinant organisms that exceeds levels of production in normal or non-transformed organisms.

“Transformation” is used according to its ordinary and customary meaning as understood by a person of ordinary skill in the art, and is used without limitation to refer to the transfer of a polynucleotide into a target cell. The transferred polynucleotide can be incorporated into the genome or chromosomal DNA of a target cell, resulting in genetically stable inheritance, or it can replicate independent of the host chromosomal. Host organisms containing the transformed nucleic acid fragments are referred to as “transgenic” or “recombinant” or “transformed” organisms.

Compounds that fall within the scope of the disclosure comprise compounds that can be used in or generated by at least one step or part of the downstream indole alkaloid biosynthetic pathway described herein, including indole alkaloids, synthetic intermediates, metabolites, or precursor species thereof (which may also be referred to as “target” compounds), as generally described herein. Non-limiting examples of such compounds include strictosidine, hydroxystrictosidine, methoxystrictosidine, strictosidine aglycone, hydroxystrictosidine aglycone, methoxystrictosidine aglycone, demethylcorynantheidine (20R, 20S), 9-hydroxydemethylcorynantheidine (20R, 20S), 9-methoxydemethylcorynantheidine (20R, 20S), corynantheidine, dihydrocorynantheine, 9-hydroxycorynantheidine, 9-hydroxydihydrocorynantheine, speciogynine, and/or mitragynine, among others.

Sequences, including polynucleotide and amino acid sequences that fall within the scope of the disclosure include sequences that comprise a protein or enzyme (or sequences encoding the same) involved in at least a portion of the indole alkaloid biosynthetic pathway for mitragynine, including sequences that may facilitate the expression or activity of one or more other sequences of the biosynthetic pathway. Non-limiting examples of such sequences include enzymes and transport and regulatory proteins, as well as polynucleotides that encode for such enzymes and proteins comprising: a NADPH-dependent reductase (DCS); an enol-O-methyltransferase (enolMT); an indole ring hydroxylase (M9H); or an indole ring O-methyltransferase (M9OMT), among others.

Other non-limiting examples of such sequences associated with the biosynthetic pathways include enzymes and regulatory proteins, as well as polynucleotides that encode for such enzymes and regulatory proteins comprising: a cytochrome b5 (CYB5), a cytochrome P450 reductase (CPR), a tryptophan importer (TAT2), a SAMe importer (SAM3), a transporter (trxporter), a methionine importer (MUP1), a mRNA stabilizer (IME4), a P450/CYP activity enhancer (ICE2 and/or INO2), a SAMe pathway enhancer (ADK1 and/or SAM2)), a tryptamine 4-hydroxylase (PsiH or T4H), an indole O-methyltransferase (IOMT), a kinase (PsiK), and/or a halogenase (TrpHalo), among others.

### Polynucleotides and Related Enzymes and Proteins

In various aspects the disclosure provides for polynucleotide sequences that encode amino acid sequences (e.g., enzymes, regulatory proteins, transporters, etc.) that are useful in the biosynthesis of an indole alkaloid. In various aspects the disclosure provides for polynucleotide sequences that encode amino acid sequences (e.g., enzymes, transporters, etc.) that are useful in the biosynthesis of an indole alkaloid such as, for example, a compound in the biosynthetic pathway for mitragynine production. In embodiments of this aspect, the polynucleotide sequence comprises a gene that encodes a polypeptide that is associated with downstream indole alkaloid synthesis (i.e., compounds in a biosynthetic pathway from strictosidine or strictosidine aglycone through to mitragynine, as well as precursors and metabolites thereof).

Thus, in embodiments, the polynucleotide sequence comprises a modified gene sequence that encodes an enzyme or regulatory protein associated with the indole alkaloid biosynthetic pathway (i.e., from strictosidine or strictosidine aglycone to mitragynine), comprising one or more of a NADPH-dependent reductase (DCS); an enol-O-methyltransferase (enolMT); an indole ring hydroxylase (M9H); or an indole ring O-methyltransferase (M90MT), a cytochrome b5 (CYB5), a cytochrome P450 reductase (CPR), a tryptophan importer (TAT2), a SAMe importer (SAM3), a transporter (trxporter), a methionine importer (MUP1), a mRNA stabilizer (IME4), a P450/CYP activity enhancer (ICE2 and/or INO2), a SAMe pathway enhancer (ADK1 and/or SAM2), a tryptamine 4-hydroxylase (PsiH or T4H), an indole O-methyltransferase (IOMT), a kinase (PsiK), and/or a halogenase (TrpHalo).

In various embodiments of the biosynthetic methods and recombinant organisms disclosed herein the disclosure provides a combination of one or more of the polynucleotide sequences each encoding a functional amino acid sequence (e.g., enzyme, regulatory protein, transport protein, etc.), as described herein, that participates in the biosynthetic process.

In embodiments, the sequences comprise a DCS polynucleotide sequence disclosed as DCS_In-26n, or a sequence having at least about 90%, 91%, 92%, 93%, 94%, 95%, 96%, 97%, 98%, or 99% sequence identity to any such DCS polynucleotide sequence. In some embodiments, the DCS polynucleotide encodes a DCS amino acid sequence of DCS_Ip-26p, or a sequence having at least about 90%, 91%, 92%, 93%, 94%, 95%, 96%, 97%, 98%, or 99% sequence identity to any such DCS amino acid sequence.

In embodiments, the sequences comprise a M9OMT polynucleotide sequence disclosed as M9OMT_In-64n, or a sequence having at least about 90%, 91%, 92%, 93%, 94%, 95%, 96%, 97%, 98%, or 99% sequence identity to any such M9OMT polynucleotide sequence. In some embodiments, the M9OMT polynucleotide encodes a M9OMT amino acid sequence of M9OMT_Ip-64p, or a sequence having about 90% sequence identity to any such M90MT amino acid sequence.

In embodiments, the sequences comprise an enolMT polynucleotide sequence disclosed as enolMT_In-7n, or a sequence having at least about 90%, 91%, 92%, 93%, 94%, 95%, 96%, 97%, 98%, or 99% sequence identity to any such enolMT polynucleotide sequence. In some embodiments, the enolMT polynucleotide encodes a enolMT amino acid sequence of enolMT_Ip-7p, or a sequence having about 90% sequence identity to any such enolMT amino acid sequence.

In embodiments, the sequences comprise a M9H polynucleotide sequence disclosed as M9H_In-36n, or a sequence having at least 90% sequence identity to any such M9H polynucleotide sequence. In some embodiments, the M9H polynucleotide encodes a M9H amino acid sequence of M9H_1p-36p, or a sequence having about 90% sequence identity to any such M9H amino acid sequence.

In some embodiments, the polynucleotide comprises a cytochrome P450 reductase (CPR) gene comprising any of the sequences in the Sequence Listing provided herewith, including CPR_In, CPR_2n, CPR_3n, or CPR_4n, or a sequence having at least about 90%, 91%, 92%, 93%, 94%, 95%, 96%, 97%, 98%, or 99% sequence identity to the CPR sequences. In some embodiments, the polynucleotide comprises a cytochrome P450 reductase (CPR) gene sequence that encodes a cytochrome P450 reductase (CPR) amino acid sequence in the Sequence Listing provided herewith, including CPR_Ip, CPR_2p, CPR_3p, or CPR_4p, or a sequence having at least about 90%, 91%, 92%, 93%, 94%, 95%, 96%, 97%, 98%, or 99% sequence identity to any of the CPR amino acid sequences.

In some embodiments, the sequences comprise trxporter genes that are in the Nitrate/Peptide Family (NPF) transporters that uptake exogenous iridoid glucosides including loganic acid and loganin, increasing the intracellular concentrations of these iridoid glucosides. In some embodiments, the polynucleotide comprises a transporter (trxporter) gene comprising any of the sequences in the Sequence Listing provided herewith, including trxporter_In, trxporter_2n, trxporter_3n, trxporter_4n, trxporter_5n, or trxporter_6n, or a sequence having at least about 90%, 91%, 92%, 93%, 94%, 95%, 96%, 97%, 98%, or 99% sequence identity to the trxporter sequences.

In some embodiments, the polynucleotide comprises a transporter (trxporter) gene sequence that encodes a transporter (trxporter) amino acid sequence in the Sequence Listing provided herewith, including trxporter_lp, trxporter_2p, trxporter_3p, trxporter_4p, trxporter_5p, or trxporter_6p, or a sequence having at least about 90%, 91%, 92%, 93%, 94%, 95%, 96%, 97%, 98%, or 99% sequence identity to any of the trxporter amino acid sequences. In some embodiments, the polynucleotide comprises a cytochrome b5 (CYB5) gene, which together with CPR can relay electrons from NADPH to a cytochrome p450 enzyme using FAD and FMN as cofactors. Coexpression of CPR and CYB5 proteins can enhance enzymatic oxidations, such as increasing the activity of the P450 enzymes described herein. In some embodiments, the polynucleotide comprises a CYB5 gene comprising any of the sequences in the Sequence Listing provided herewith, including CYB5_In (SEQ ID NO:315), or CYB_2n (SEQ ID NO:216), or a sequence having at least about 90%, 91%, 92%, 93%, 94%, 95%, 96%, 97%, 98%, or 99% sequence identity to the CYB5 sequences. In some embodiments, the polynucleotide comprises a cytochrome b5 (CYB5) gene sequence that encodes a cytochrome b5 (CYB5) amino acid sequence in the Sequence Listing provided herewith, including CYB5_1p (SEQ ID NO:187) or CYB5_2p (SEQ ID NO:188), or a sequence having at least about 90%, 91%, 92%, 93%, 94%, 95%, 96%, 97%, 98%, or 99% sequence identity to any of the CYB5 amino acid sequences.

In some embodiments, the polynucleotide comprises a tryptophan importer (TAT2) gene comprising any of the sequences in the Sequence Listing provided herewith, including TAT2_In (SEQ ID NO:366) or a sequence having at least about 90%, 91%, 92%, 93%, 94%, 95%, 96%, 97%, 98%, or 99% sequence identity to the TAT2 sequences. In some embodiments, the polynucleotide comprises a tryptophan importer (TAT2) gene sequence that encodes a tryptophan importer (TAT2) amino acid sequence.

In some embodiments, the polynucleotide comprises a SAMe importer (SAM3) gene comprising any of the sequences in the Sequence Listing provided herewith, including SAM3_In (SEQ ID NO:364) or a sequence having at least about 90%, 91%, 92%, 93%, 94%, 95%, 96%, 97%, 98%, or 99% sequence identity to the SAM3 sequences. In some embodiments, the polynucleotide comprises a SAMe importer (SAM3) gene sequence that encodes a SAMe importer (SAM3) amino acid sequence.

In some embodiments, the polynucleotide comprises a methionine importer (MUP1) gene comprising any of the sequences in the Sequence Listing provided herewith, including MUP1_In (SEQ ID NO:365) or a sequence having at least about 90%, 91%, 92%, 93%, 94%, 95%, 96%, 97%, 98%, or 99% sequence identity to the MUP1 sequences. In some embodiments, the polynucleotide comprises a methionine importer (MUP1) gene sequence that encodes a methionine importer (MUP1) amino acid sequence.

In some embodiments, the polynucleotide comprises a mRNA stabilizer (IME4) gene, a P450/CYP activity enhancer (ICE2 and/or INO2) gene and INO2_In (SEQ ID NO:361), a SAMe pathway enhancer (ADK1 and/or SAM2) gene, comprising any of the sequences identified in the Sequence Listing provided herewith, including IME4_In (SEQ ID NO:367), ICE2_In (SEQ ID NO:360), INO2_In (SEQ ID NO:361), ADK1_In (SEQ ID NO:363), and/or SAM2_In (SEQ ID NO:362), SAM3_In (SEQ ID NO:364), or a sequence having about 90% sequence identity to these sequences. In some embodiments, the polynucleotide comprises a mRNA stabilizer (IME4) gene, e.g., IME4_In (SEQ ID NO:367), a P450/CYP activity enhancer (ICE2 and/or INO2) gene, a SAMe pathway enhancer (ADK1 and/or SAM2) gene that encodes the corresponding amino acid sequence, i.e., (IME4), (ICE2), (INO2), (ADK1), a (SAM2) amino acid sequence, a tryptamine 4-hydroxylase (PsiH or T4H), an indole O-methyltransferase (IOMT), a kinase (PsiK), and/or a halogenase (TrpHalo).

In some embodiments, N terminal peptide sequences for membrane localization are fused to the N terminus of P450s such as the M9H and CPR proteins. In some embodiments, membrane localization signals LclTag_Ip-28p (SEQ ID NO:202, 203, 204, 205, 206, 207, 208, 209, 210, 211, 212, 213, 214, 215, 216, 217, 218, 219, 220, 221, 222, 223, 224, 225, 226, 227, 228, and 229, rewpectively), encoded by LclTag_In-28n (SEQ ID NO:330, 331, 332, 333, 334, 335, 336, 337, 338, 339, 340, 341, 342, 343, 344, 345, 346, 347, 348, 349, 350, 351, 352, 353, 354, 355, 356 and 357, respectively), are fused to any of the sequences described herein. Such fusion polypeptides when expressed may lead to increased P450/CYP and CPR activity due to enhanced membrane localization.

Hmx1 is a heme oxygenase involved in the degradation of heme, and can be incorporated in accordance with some aspects and example embodiments of the disclosure. For example, in some embodiments, the HMX1 gene of the host is deleted to reduce heme degradation. Heme depletion is a major source of cellular stress and cells are particularly susceptible to this source of stress when several p450 monooxygenase enzymes are expressed in the cell. In other embodiments, enzymes to increase cellular heme are expressed, including Hem3, Hem2 and Hem12. Iron (II) citrate and 8-aminolevulinic acid can be fed into fermentation media to contribute to heme production in a recombinant host cell.

The tryptophan importer (TAT2) is a high affinity tryptophan and tyrosine permease. Sec, e.g., TAT2_In (SEQ ID NO:366). Heterologous pathway enzymes that are expressed to produce compounds with an indole core such as tryptamine and serotonin use L-tryptophan as a directing molecule. Tryptophan production in cells is normally tightly regulated. Tryptophan accumulation in a recombinant host is increased by overexpressing a recombinant L-tryptophan transporter (TAT2). This allows for exogenous tryptophan to be fed to the cells and transported in the recombinant host. See also, WO/2021/248087.

SAMe importer (SAM3) is a high-affinity S-adenosylmethionine permease. In some embodiments, a recombinant host is modified to increase the accumulation of the methyl donor, SAMe, which is used in the upstream pathway by enzymes such as the recombinant LAMT enzymes to methylate loganic acid. SAMe accumulation in the recombinant host cell can be increased by overexpressing this permease and feeding exogenous SAMe into the media. See also PCT Patent Application Publication WO/2021/248087.

The methionine importer (MUP1) is a high affinity methionine permease. An example is MUP_In (SEQ ID NO:365). SAMe is a robust methyl donor synthesized from methionine and ATP. In some embodiments, to increase the uptake of exogenous L-methionine fed into the SAMe pathway, recombinant Mupl is overexpressed. See also PCT Patent Application Publication WO/2021/248087.

In some embodiments, the nucleic acids described herewith encode a polypeptide or oligopeptide having an amino acid sequence that is naturally occurring. In other embodiments, the nucleic acids encode a polypeptide or oligopeptide having an amino acid sequence that is not naturally occurring. The encoded polypeptides or oligopeptides that are not naturally occurring can vary from a naturally occurring polypeptide or oligopeptide, or portion thereof, by a small amount (e.g., one conservative amino acid substitution or a histidine tag) or extensively (e.g., further comprising a fusion peptide, a substituted or added domain from another protein, a scaffold, etc.).

In some of the above embodiments, the nucleic acid provided herein comprises the sequence of any one of the nucleic acid sequences PCT Patent Application Publication.

Nucleic acid sequences in accordance with the disclosure are synthesized and cloned using techniques known in the art. Gene expression can be controlled by inducible or constitutive promoter systems using the appropriate expression vectors. Genes are transformed into an organism using standard yeast or fungus transformation methods to generate modified host strains (i.e., the recombinant host organism). The modified strains express the genes for biosynthetic pathways that generate indole alkaloid products in the mitragynine biosynthetic pathway, which can be fed with compounds produced in an upstream biosynthetic pathway pathway (e.g., loganin, loganic acid, strictosidine, strictosidine aglycone, etc.). The indole alkaloid pathway genes herein can be integrated into the genome of the cell or maintained as an episomal plasmid. Recombinant host fermentation samples are: (i) prepared and extracted using a combination of fermentation, dissolution, and purification steps; and (ii) analyzed by HPLC for the presence of directing molecules, precursor molecules, intermediate molecules, and target molecules such as those illustrated in the Examples and otherwise disclosed herein.

The polynucleotides can be used in, or used to generate, modified strains of host cells, which produce target compounds including mitragynine alkaloids, precursors, intermediates, or metabolites thereof.

In some embodiments, the nucleic acid sequence (i.e., polynucleotide) encoding a gene or a complementary nucleic acid sequence to such a coding sequence can be codon optimized for production in a selected microorganism. A number of factors can be used in determining a codon-optimized sequence (see, e.g., U.S. Pat. No. 10,435,727). Factors can include, for example, (1) selecting a codon for each amino acid residue in the recombinant polypeptide based on the usage frequency of each codon in the heterologous host cell (e.g., Saccharomyces cerevisiae) genome; (2) removing sequences that provide for restriction sites for enzymes to prevent DNA cleavage; (3) modifying long repeats (e.g., consecutive sequences of 5 or more nucleotide) to prevent low-complexity regions; (4) adding a ribosome binding site to the N-terminus; (5) adding a stop codon; (6) changing nucleotides that encode amino acids susceptible to undesirable post-translational modifications (e.g., changing codons for a surface exposed LYS to an ARG codon to avoid ubiquitination); (7) removing or replacing a localization signal sequence.

In various embodiments, the nucleic acid sequences can further comprise additional sequence encoding amino acids that are not part of the included enzymes or regulatory proteins herein. In some of these embodiments, the additional sequences encode additional amino acids present when the nucleic acid is translated, encoding, for example, a co-folding peptide, as disclosed herein, or an additional protein domain, with or without a linker sequence, creating a fusion protein. Other examples are localization sequences, i.e., signals directing the localization of the folded protein to a specific subcellular compartment or membrane. Additional non-limiting examples are an affibody tag, a localization scaffold, a vacuolar localization tag, a secretion signal, and a histidine tag (e.g., 6×his tag). Additional examples include cleavage sites, such as a TEV protease recognition sequence or 2A-self-cleaving peptides encoded between two or more genes for cleavage of individual proteins post-translation and enabling polycistronic gene expression.

In some embodiments, two or more recombinant polynucleotide sequences can be fused together (i.e., co-expressed) by including a nucleotide linker sequence between a polynucleotide sequence and an additional polynucleotide sequence or polynucleotide sequences, such as the sequences disclosed herein. The resulting chimeric genes encode for fusions (i.e., a polypeptide fused to another polypeptide or polypeptides), such as the recombinant amino acid sequences disclosed herein. In embodiments, the linker region(s) can be the same or different, and can comprise from 3 to 50 amino acids. In such embodiments the fused polynucleotide sequences encoding fused polypeptides, such as fusions between the sequences disclosed herein (DCS with SGD, DCS with enolMT, among other combinations), can lead to enhanced yields by increasing the local concentration of substrate and active enzyme due to the increased co-localization of the fused enzymes. These embodiments relating to fusions of one or more sequences in accordance with the disclosure also confer a benefit that can allow for a single promoter to control expression of multiple recombinant proteins, which can decrease the size of expression cassettes.

In some embodiments, the nucleic acid comprises additional nucleotide sequences that are not translated. Non-limiting examples include promoters, terminators, barcodes, Kozak sequences, targeting sequences, and enhancer elements. In some particular embodiments the polynucleotide sequences comprise a promoter that is functional in yeast, fungi, and bacteria. In embodiments, expression of a gene encoding an enzyme or regulatory protein is controlled by the promoter operably linked to the gene sequence. For a gene to be expressed, a promoter must be present within 1,000 nucleotides upstream of the gene. A gene is generally cloned under the control of a desired promoter. The promoter is placed upstream of the gene in the genome or on an episomal plasmid. The promoter regulates the amount of enzyme expressed in the cell and the timing of expression, or expression in response to external factors such as carbon source.

Any promoter can be utilized to drive the expression of the enzymes and regulatory proteins described herein. Listings of various promoters in organisms such as yeast are readily available (See, e.g., the registry of Standard Biological Parts for yeast at the website: parts.igem.org/Yeast). Several of the exemplary promoters listed in Table 1 below drive strong expression, constant gene expression, medium or weak gene expression, or provide inducible gene expression. Inducible or repressible gene expression is dependent on the presence or absence of a certain molecule (inducer/repressor). For example, the GALI, GAL7, and GALIO promoters are activated by the presence of galactose and are repressed by the presence of glucose. The HO promoter is active and drives gene expression only in the presence of the alpha factor peptide. The HXTI promoter is activated by the presence of glucose while the ADH2 promoter is repressed by the presence of glucose.

TABLE 1
Exemplary promoters

| Constitutive | | Inducible/ |
|---|---|---|
| promoters | Constitutive promoters | repressible |
| (Strong) | (Medium and weak) | promoters |
| TEF1 | STE2 | GAL1 |
| PGK1 | TPI1 | GAL7 |
| PGI1 | PYK1 | GALI0 |
| TDH3 | CYC1 | HO |
| T7 | ADH1 | HXT1 |
| | | ADH2 |
| | | AOX1 |
| | | T5-lac |

As discussed herein, various embodiments of the disclosure provide the nucleic acid sequence as an expression cassette, e.g., a yeast expression cassette. Any yeast expression cassette capable of expressing the enzyme in a yeast cell can be utilized. Additional regulatory elements can also be present in the expression cassette, including restriction enzyme cleavage sites, antibiotic resistance genes, integration sites, auxotrophic selection markers, origins of replication, and degrons.

The expression cassette can be present in a vector that, when transformed into a host cell, either integrates into chromosomal DNA or remains episomal in the host cell. Such vectors are well-known in the art. One non-limiting example of a yeast vector is a yeast episomal plasmid (YEp) that contains the pBluescript II SK (+) phagemid backbone, an auxotrophic selectable marker, yeast and bacterial origins of replication and multiple cloning sites enabling gene cloning under a suitable promoter (see Table 1). Other non-limiting vectors include pRS series plasmids.

Mutations introduced into the DNA can provide enzyme variations that can prevent or promote post-translational modifications of the protein. Non-limiting examples of post-translational modifications include phosphorylation, acetylation, methylation, SUMOylation, ubiquitination, proteolytic cleavage, lipidation, prenylation such as farnesylation or myristoylation, glycosylation, nitrosylation and biotinylation.

The nucleic acid sequences can be modified from a gene from any source, e.g., any microorganism, protist, virus, plant, or animal. In some embodiments, the gene encoding an enzyme or regulatory protein is derived from a bacterium. For example, the bacterium can be from phylum Abditibacteriota, including class Abditibacteria, including order Abditibacteriales; phylum Abyssubacteria or Acidobacteria, including class Acidobacteriia, Blastocatellia, Holophagae, Thermoanaerobaculia, or Vicinamibacteria, including order Acidobacteriales, Bryobacterales, Blastocatellales, Acanthopleuribacterales, Holophagales, Thermotomaculales, Thermoanaerobaculales, or Vicinamibacteraceae; phylum Actinobacteria, including class Acidimicrobiia, Actinobacteria, Actinomarinidae, Coriobacteriia, Nitriliruptoria, Rubrobacteria, or Thermoleophilia, including orders Acidimicrobiales, Acidothermales, Actinomycetales, Actinopolysporales, Bifidobacteriales, Nanopelagicales, Catenulisporales, Corunebacteriales, Cryptosporangiales, Frankiales, Geodermatophilales, Glycomycetales, Jiangellales, Micrococcales, Micromonosporales, Nakamurellales, Propionibacteriales, Pseudonocardiales, Sporichthyales, Streptomycetales, Streptosporangiales, Actinomarinales, Coriobacteriales, Eggerthellales, Egibacterales, Egicoccales, Euzebyales, Nitriliruptorales, Gaiellales, Rubrobacterales, Solirubrobacterales, or Thermoleophilales; phylum Aquificae, including class Aquificae, including order Aquificales or Desulfurobacteriales; phylum Armatimonadetes, including class Armatimonadia, including order Armatimonadales, Capsulimonadales, Chthonomonadetes, Chthonomonadales, Fimbriimonadia, or Fimbriimonadales; phylum Aureabacteria or Bacteroidetes, including class Armatimonadia, Bacteroidia, Chitinophagia, Cytophagia, Flavobacteria, Saprospiria or Sphingobacteriia, including order Bacteroidales, Marinilabiliales, Chitinophagales, Cytophagales, Flavobacteriales, Saprospirales, or Sphingopacteriales; phylum Balneolaeota, Caldiserica, Calditrichaeota, or Chlamydiae, including class Balneolia, Caldisericia, Calditrichae, or Chlamydia, including order Balneolales, Caldisericales, Calditrichales, Anoxychlamydiales, Chlamydiales, or Parachlamydiales; phylum Chlorobi or Chloroflexi, including class Chlorobia, Anaerolineae, Ardenticatenia, Caldilineae, Thermofonsia, Chloroflexia, Dehalococcoidia, Ktedonobacteria, Tepidiformia, Thermoflexia, Thermomicrobia, or Sphaerobacteridae, including order Chlorobiales, Anaerolineales, Ardenticatenales, Caldilineales, Chloroflexales, Herpetosiphonales, Kallotenuales, Dehalococcoidales, Dehalogenimonas, Ktedonobacterales, Thermogemmatisporales, Tepidiformales, Thermoflexales, Thermomicrobiales, or Sphaerobacterales; phylum Chrysiogenetes, Cloacimonetes, Coprothermobacterota, Cryosericota, or Cyanobacteria, including class Chrysiogenetes, Coprothermobacteria, Gloeobacteria, or Oscillatoriophycideae, including order Chrysiogenales, Coprothermobacterales, Chroococcidiopsidales, Gloeoemargaritales, Nostocales, Pleurocapsales, Spirulinales, Synechococcales, Gloeobacterales, Chroococcales, or Oscillatoriales; phyla: Eferribacteres, Deinococcus-thermus, Dictyoglomi, Dormibacteraeota, Elusimicrobia, Eremiobacteraeota, Fermentibacteria, or Fibrobacteres, including class Deferribacteres, Deinococci, Dictyoglomia, Elusimicrobia, Endomicrobia, Chitinispirillia, Chitinivibrionia, or Fibrobacteria, including order Deferribacterales, Deinococcales, Thermales, Dictyoglomales, Elusimicrobiales, Endomicrobiales, Chitinspirillales, Chitinvibrionales, Fibrobacterales, or Fibromonadales;

phylum Firmicutes, Fusobacteria, Gemmatimonadetes, or Hydrogenedentes, including class Bacilli, Clostridia, Erysipelotrichia, Limnochordia, Negativicutes, Thermolithobacteria, Tissierellia, Fusobacteriia, Gemmatimonadetes, Longimicrobia, including order Bacillales, Lactobacillus, Borkfalkiales, Clostridiales, Halanaerobiales, Natranaerobiales, Thermoanaerobacterales, Erysipelotrichales, Limnochordales, Acidaminococcales, Selenomonadales, Veillonellales, Thermolithobacterales, Tissierellales, Fusobacteriales, Gemmatimonadales, or Longimicrobia; phylum Hydrogenedentes, Ignavibacteriae, Kapabacteria, Kiritimatiellaeota, Krumholzibacteriota, Kryptonia, Latescibacteria, LCP-89, Lentisphaerae, Margulisbacteria, Marinimicrobia, Melainabacteria, Nitrospinae, or Omnitrophica, including class Ignavibacteria, Kiritimatiellae, Krumholzibacteria, Lentisphaeria, Oligosphaeria, or Nitrospinae, including order Ignavibacteriales, Kiritimatiellales, Krumholzibacteriales, Lentisphaerales, Victivallales, Oligosphaerales, or Nitrospinia; phylum Omnitrophica or Planctomycetes, including class Brocadiae, Phycisphaerae, Planctomycetia, or Phycisphaerales, including order Sedimentisphaerales, Tepidisphaerales, Gemmatales, Isosphaerales, Pirellulales, or Planctomycetales; phylum Proteobacteria including class Acidithiobacillia, Alphaproteobacteria, Betaproteobacteria, Lambdaproteobacteria, Muproteobacteria, Deltaproteobacteria, Epsilonproteobacteria, Gammaproteobacteria, Hydrogenophilalia, Oligoflexia, or Zetaproteobacteria, including order Acidithiobacillales, Caulobacterales, Emcibacterales, Holosporales, lodidimonadales, Kiloniellales, Kopriimonadales, Kordiimonadales, Magnetococcales, Micropepsales, Minwuiales, Parvularculales, Pelagibacterales, Rhizobiales, Rhodobacterales, Rhodospirillales, Rhodothalassiales, Rickettsiales, Sneathiellales, Sphingomonadales, Burkholderiales, Ferritrophicales, Ferrovales, Neisseriales, Nitrosomonadales, Procabacteriales, Rhodocyclales, Bradymonadales, Acidulodesulfobacterales, Desulfarculales, Desulfobacterales, Desulfovibrionales, Desulfurellales, Desulfuromonadales, Myxococcales, Syntrophobacterales, Campylobacterales, Nautiliales, Acidiferrobacterales, Aeromonadales, Alteromonadales, Arenicellales, Cardiobacteriales, Cellvibrionales, Chromatiales, Enterobacterales, Immundisolibacterales, Legionellales, Methylococcales, Nevskiales, Oceanospirillales, Orbales, Pasteurellales Pseudomonadales, Salinisphaerales, Thiotrichales, Vibrionales, Xanthomonadales, Hydrogenophilales, Bacteriovoracales, Bdellovibrionales, Oligoflexales, Silvanigrellales, or Mariprofundales; phylum Rhodothermaeota, Saganbacteria, Sericytochromatia, Spirochaetes, Synergistetes, Tectomicrobia, or Tenericutes, including class Rhodothermia, Spirochaetia, Synergistia, Izimaplasma, or Mollicutes, including order Rhodothermales, Brachyspirales, Brevinematales, Leptospirales, Spirochaetales, Synergistales, Acholeplasmatales, Anaeroplasmatales, Entomoplasmatales, or Mycoplasmatales; phylum Thermodesulfobacteria, Thermotogae, Verrucomicrobia, or Zixibacteria, including class Thermodesulfobacteria, Thermotogae, Methylacidiphilae, Opitutae, Spartobacteria, or Verrucomicrobiae, including order Thermodesulfobacteriales, Kosmotogales, Mesoaciditogales, Petrotogales, Thermotogales, Methylacidiphilales, Opitutales, Puniceicoccales, Xiphinematobacter, Chthoniobacterales, Terrimicrobium, or Verrucomicrobiales.

In other embodiments, the gene encoding the enzyme or regulatory protein is modified from an archacon. For example, the archacon can be from: phylum Euryarchaeota, including class Archaeoglobi, Hadesarchaea, Halobacteria, Methanobacteria, Methanococci, Methanofastidiosa, Methanomicrobia, Methanopyri, Nanohaloarchaea, Theionarchaea, Thermococci, or Thermoplasmata, including order Archaeoglobales, Hadesarchaeales, Halobacteriales, Methanobacteriales, Methanococcales, Methanocellales, Methanomicrobiales, Methanophagales, Methanosarcinales, Methanopyrales, Thermococcales, Methanomassiliicoccales, Thermoplasmatales, or Nanoarchaeales; DPANN superphylum, including subphyla Aenigmarcheota, Altiarchaeota, Diapherotrites, Micrarchaeota, Nanoarchaeota, Pacearchaeota, Parvarchaeota, or Woesearchaeota; TACK superphylum, including subphylum Korarchaeota, Crenarchaeota, Aigarchaeota, Geoarchaeota, Thaumarchaeota, or Bathyarchaeota; Asgard superphylum including subphylum Odinarchaeota, Thorarchaeota, Lokiarchaeota, Helarchaeota, or Heimdallarchaeota.

In additional embodiments, the gene encoding the enzyme or regulatory protein is modified from a fungus. For example, the fungus can be from: phyla Chytridiomycota, Basidiomycota, Ascomycota, Blastocladiomycota, Ascomycota, Microsporidia, Basidiomycota, Glomeromycota, Symbiomycota, and Neocallimastigomycota; phylum Ascomycota, including classes and orders Pezizomycotina, Arthoniomycetes, Coniocybomycetes, Dothideomycetes, Eurotiomycetes, Geoglossomycetes, Laboulbeniomycetes, Lecanoromycetes, Leotiomycetes, Lichinomycetes, Orbiliomycetes, Pezizomycetes, Sordariomycetes, Xylonomycetes, Lahmiales, Itchiclahmadion, Triblidiales, Saccharomycotina, Saccharomyces, Taphrinomycotina, Archaeorhizomyces, Neolectomycetes, Pneumocystidomycetes, Schizosaccharomyces, Taphrinomycetes; phylum Basidiomycota including subphyla or classes Pucciniomycotina, Ustilaginomycotina, Wallemiomycetes, and Entorrhizomycetes; subphylum Agaricomycotina including classes Tremellomycetes, Dacrymycetes, and Agaricomycetes; phylum Symbiomycota, including class Entorrhizomycota; subphylum Ustilaginomycotina including classes Ustilaginomycetes and Exobasidiomycetes; phylum Glomeromycota including classes Archaeosporomycetes, Glomeromycetes, and Paraglomeromycetes; subphylum Pucciniomycotina including orders and classes: Pucciniomycotina, Cystobasidiomycetes, Agaricostilbomycetes, Microbotryomycetes, Atractiellomycetes, Classiculomycetes, Mixiomycetes, and Cryptomycocolacomycetes; subphylum incertae sedis Mucoromyceta including orders Calcarisporiellomycota and Mucoromycota; phylum Mortierellomyceta including class Mortierellomycota; subphylum incertae sedis Entomophthoromycotina including order Entomophthorales; phylum Zoopagomyceta including classes Basidiobolomycota, Entomophthoromycota, Kickxellomycota, and Zoopagomycotina; subphylum incertae sedis Mucoromycotina including orders Mucorales, Endogonales, and Mortierellales; phylum Neocallimastigomycota including class Neocallimastigomycetes; phylum Blastocladiomycota including classes Physodermatomycetes and Blastocladiomycetes; phylum Rozellomyceta including classes Rozellomycota and Microsporidia; phylum Aphelidiomyceta including class Aphelidiomycota; phylum Chytridiomyceta including classes Chytridiomycetes and Monoblepharidomycetes; phylum Oomycota including classes or orders Leptomitales, Myzocytiopsidales, Olpidiopsidales, Peronosporales, Pythiales, Rhipidiales, Salilagenidiales, Saprolegniales, Sclerosporales, Anisolpidiales, Lagenismatales, Rozellopsidales, and Haptoglossales.

In additional embodiments, the gene for the enzyme or regulatory protein is derived from the organism below. This includes but is not limited to: Acanthurus tractus, Aplysina aerophoba, Stevia rebaudiana, Bos Taurus, Bufo bufo, Bufotes viridis, Chrysochloris asiatica, Fukomys damarensis, Streptomyces reticuliscabie, Homo sapiens, Rattus norvegicus, Rhinella marina, Rhinella spinulosa, Schistosoma mansoni, Xenopus laevis, Xenopus tropicalis, Acacia koa, Arabidopsis thaliana, Psilocybe cubensis, Brassica oleracea, Citrus sinensis, Hordeum vulgare, Juglans cinereal, Juglans regia, Lophophora williamsii, Nymphaea colorata, Oryza sativa, Ipomoea violaceae, Rivea corymbosa, Argyreia nervosa, Gentiana rigescens, Nyssa sinensis, Camptotheca acuminata, Merremia tuberose, Mitragyna speciosa, Tabernanthe iboga, Tabernaemontana elegans, Voacanga africana, Tabernaemontana undulata, Ricinus communis, Solanum lycopersicum, Sorghum bicolor, Theobroma cacao, and Triticum aestivum, Catharanthus roseus, Actinidia polygama, Corydalis cheilanthifolia, Lonicera japonica, Olea europaea, Antirrhinum majus, Salix suchowensis, Sesamum indicum, Salvia splendens, Nepeta racemosa, Nothapodytes nimmonian, Vinca minor, Rhazya stricta, Corydalis yanhusuo, Linum usitatissimum, Rauvolfia serpentina, Ophiorrhiza pumila, Kalopanax septemlobus, Amsonia hubrichtii, Phaedon cochleariae, Aphis glycines, Pogonomyrmex barbatus, Thermobifida fusca YX, Nocardiopsis kunsanensis, Actinorugispora endophytica, Swertia mussotii, Aedes aegypti, Arabidopsis thaliana, Helianthus tuberosus, Phratora vitellinae, Macadamia integrifolia, Mentha canadensis, Coptis chinensis, Durio zibethinus, Hibiscus syriacus, Populus alba, Capsicum pubescens, Uncaria rhynchophylla, Coffea canephora and Coffea eugenioides.

### Enzymes and Amino Acid Sequences

The disclosure provides for non-naturally occurring amino acid sequences (i.e., enzymes or regulatory proteins) comprising a sequence encoded by any of the nucleic acids described above. In some embodiments, the amino acid sequence is 85%, 90%, 95%, 98%, or 100% identical to any one of the sequences in the amino acid sequences disclosed herein (e.g., listed in the Sequence Listing provided herewith). In these embodiments, the enzyme or regulatory protein can be isolated in vitro and used in vitro to provide enzyme activity. Alternatively, the enzyme can be expressed in a recombinant organism as described herein. In some embodiments, the recombinant microorganism for the recombinant production of an amino acid sequence is a bacterium, for example an E. coli. In other embodiments, the recombinant microorganism is a yeast or fungal cell, e.g., a species of Saccharomyces (for example S. cerevisiae), Candida, Pichia, Schizosaccharomyces, Scheffersomyces, Blakeslea, Rhodotorula, Aspergillus or Yarrowia.

In some embodiments relating to production and purification of the recombinant amino acid sequences, the gene encoding the enzyme and/or regulatory protein is cloned into an expression vector such as the pET expression vectors from Novagen, transformed into a protease deficient strain of E. coli such as BL21 and expressed by induction with IPTG. The protein of interest may be tagged with an affinity tag to facilitate purification, e.g. hexahistidine, GST, calmodulin, TAP, AP, CAT, HA, FLAG, MBP, etc. Coexpression of a bacterial chaperone such as dnak, GroES/GroEL or SecY may help facilitate protein folding. See Green and Sambrook (2012).

In some aspects relating to sequences comprising amino acids (e.g., enzymes) and nucleotides (e.g., polynucleotides/genes), the disclosure provides sequence variants of the sequences disclosed herein. With regard to amino acid sequences, variants (e.g., substitution, deletion, addition) can be considered as “similar” to the original polypeptide or to have a certain “percent identity” to the original polypeptide, and include those polypeptides wherein one or more amino acid residues of a polypeptide are removed and replaced with alternative residues. In embodiments, the substitutions in amino acid sequences are conservative in nature, however, the disclosure embraces substitutions that are also non-conservative.

For amino acid sequences, sequence identity and/or similarity can be determined by using standard techniques known in the art such as, for example, the local sequence identity algorithm of Smith and Waterman, 1981, Adv. Appl. Math. 2:482, the sequence identity alignment algorithm of Needleman and Wunsch, 1970, J. Mol. Biol. 48:443, the search for similarity method of Pearson and Lipman, 1988, Proc. Nat. Acad. Sci. U.S.A. 85:2444, computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., 1984, Nucl. Acid Res. 12:387-395, using the default settings, or by inspection. Various alignment parameters can be set according to known methods (e.g., “Current Methods in Sequence Comparison and Analysis,” Macromolecule Sequencing and Synthesis, Selected Methods and Applications, pp 127-149 (1988), Alan R. Liss, Inc.). Additional useful algorithms include PILEUP, which can align multiple sequences from a group of related sequences using progressive, pairwise alignments; BLAST, including gapped BLAST, WU-BLAST-2 (see, e.g., Altschul et al., 1990, J. Mol. Biol. 215:403-410; Altschul et al., 1993, Nucl. Acids Res. 25:3389-3402; Altschul et al., 1997, Nucleic Acids Res. 25:3389-3402; Altschul et al., 1996, Methods in Enzymology 266:460-480; and Karin et al., 1993, Proc. Natl. Acad. Sci. U.S.A. 90:5873-5787).

Generally, the amino acid homology, similarity, or identity between sequences are at least 80%, including at least 85%, 90%, 91%, 92%, 93%, 94%, 95%, 96%, 97%, 98%, 99%, and from 99% to almost 100% identity. Similarly, the “percent (%) nucleic acid sequence identity” with respect to the nucleic acid sequences described herein refers to the percentage of nucleotide residues in a candidate sequence that are identical with the nucleotides disclosed herein, in the gene or coding sequence of the related polypeptide. Specific methods can include the default parameters of algorithms such as, for example, BLASTN (WU-BLAST-2).

### Recombinant Host Cells

In an aspect the disclosure provides recombinant cells that comprise the polynucleotides and polypeptides described herein. In embodiments, the host cells can comprise any type of cell that is adaptable to genetic manipulation and/or expression of foreign genes and proteins. In some embodiments, host cells can include any species of filamentous fungus, including but not limited to any species of Aspergillus, which may be optionally genetically altered to accumulate and/or produce target, precursor, or intermediate indole alkaloid molecules. In embodiments, host cells may also be any species of bacteria, including but not limited to Escherichia, Corynebacterium, Caulobacter, Pseudomonas, Streptomyces, Bacillus, or Lactobacillus. In some embodiments, the host cell is a yeast cell capable of being genetically engineered and can be utilized in these embodiments. Non-limiting examples of such yeast cells include species of Saccharomyces, Candida, Pichia, Schizosaccharomyces, Scheffersomyces, Blakeslea, Rhodotorula, or Yarrowia.

These cells can achieve gene expression controlled by inducible promoter systems, natural or induced mutagenesis, recombination, and/or shuffling of genes, pathways, and whole cells performed sequentially or in cycles; overexpression and/or deletion of single or multiple genes and reducing or eliminating parasitic side pathways that reduce target compounds, intermediate or precursor concentrations.

The host cells of the recombinant organism may also be engineered to produce any or all precursor molecules necessary for the biosynthesis of the target, precursor, or intermediate indole alkaloid compounds and can comprise any of the disclosed polynucleotide sequences, vectors or expression cassettes that are capable of expressing the recombinant enzyme encoded therein.

Construction of modified host cells such as Saccharomyces cerevisiae strains expressing the enzymes and regulatory proteins provided herein is carried out via expression of a gene which encodes for the enzyme. The gene encoding the enzyme can be cloned into vectors with the proper regulatory elements for gene expression (e.g. promoter, terminator) and the derived plasmid can be confirmed by DNA sequencing. As an alternative to expression from an episomal plasmid, the gene encoding the enzyme may be inserted into the recombinant host genome. Integration may be achieved by a single or double cross-over insertion event of a plasmid, or by nuclease-based genome editing methods, as are known in the art e.g. CRISPR, TALEN and ZFR. Strains with the integrated gene can be screened by rescue of auxotrophy and genome sequencing. See, e.g., Green and Sambrook (2012).

As described herein, the recombinant cell may be any species of yeast, including but not limited to any species of Saccharomyces, Candida, Schizosaccharomyces, Yarrowia, etc., which have been genetically altered to produce monoterpene indole alkaloid (MIA) molecules, including precursors and intermediates. Additionally, genetically engineered host cells may be any species of filamentous fungus, including but not limited to any species of Aspergillus, which have been genetically altered to produce precursor molecules such as GPP, geraniol, tryptamine, serotonin, 4-hydroxytryptamine, 5-methoxytryptamine, 4-methoxytryptamine, and strictosidine for use as precurors for the downstream MIA compound production in accordance with the present disclosure. Some of the species of yeast include but are not limited to: Schizosaccharomyces cerevisiae, Schizosaccharomyces japonicus, Schizosaccharomyces pombe, Schizosaccharomyces cryophilus, Saccharomyces cerevisiae, Kluyveromyces lactis, Kluyveromyces dobzhanskii, and Yarrowia lipolytica.

In various embodiments, the recombinant cell can comprise combinations of endogenously upregulated and/or codon optimized genes encoding for proteins which can improve: protein production, correct protein folding, protein secretion, proper protein intracellular localization, enzyme activity, correct post-translational modifications for protein features, mRNA stability, cell tolerance to stress, cellular metabolic activity, availability of cofactors for enzyme activity, glycolysis, fatty acid metabolism, feedstock conversion, amino acid biosynthesis, mevalonate pathway flux, coenzyme A (CoA) flux, acetyl-CoA production, tolerance to oxidative stress (e.g. H₂O₂) from increased protein production, tolerance to oxidative stress from monoterpene indole alkaloid (MIA) and related precursor enzymatic pathway steps, titer of alkaloids and alkaloid pathway precursors, intermediates, and compounds.

As discussed herein, the recombinant cells and modified strains express the genes for biosynthetic pathways that generate monoterpene indole alkaloid pathway products, such as demethylcorynantheidine, corynantheidine, 9-hydroxycorynantheidine, mitragynine, etc., and/or all intermediate monoterpene indole alkaloids compounds and precursors described herein. The monoterpene indole alkaloid pathway genes herein can be integrated into the genome of the cell or maintained as an episomal plasmid. Recombinant host fermentation samples are: (i) prepared and extracted using a combination of fermentation, dissolution, and purification steps; and (ii) analyzed by HPLC for the presence of target molecules, precursor molecules, and/or intermediate molecules. In some embodiments, the product MIA pathway products are generated from an upstream pathway feed that comprises compounds such as geraniol, nepetalactol, loganic acid, loganin, secologanin, tryptamine, serotonin, 5-methoxytryptamine, 4-methoxytryptamine, 4-hydroxytryptamine, strictosidine, any of which can also be detected.

In some related embodiments, the genes and proteins that may be expressed by the recombinant cell (with reference to exemplary Genbank protein accession numbers) can include one or more of:
rRNA processing proteins for ribosome biogenesis, such as BFR2 (QHB07760.1)
14-3-3 proteins which regulate vesicle and protein transport, such as BMH2 (CAA59275.1)
Transcription factors which upregulate the unfolded protein response, such as HACI (QHB08305.1).
Karyopherin overexpression to help the transport and translocation of hydrophobic and/or chimeric proteins, such as PSEI (QHB11037.1)
Ribosomal proteins for maintaining translational productivity through cell stress, such as RPPO (QHB10483.1),
Oxidative stress protection, such as CCS1 (QHB 10774.1), SOD1, (CAA89634.1), and/or SSA4, (KZV11871.1)
ER stress sensors which can upregulate protein folding chaperones and heat shock responses, such as IREI (QHB09072.1)
Proteins which can increase cellular glycosylation status, such as PSA1, (CAA98617.1)
Peroxide consuming proteins including catalase to protect from oxidative damage, such as CTT1, (CAA97090.1)
Protein folding chaperones and chaperonins, such as SSE1, (KZV07411.1), JEM1, (QHB09554.1), KAR2, (CAA89325.1), LHS1, (CAA81910.1), SCJ1, (CAA41529.1), SIL1, (QHB11576.1), GroEL (QEP09777.1), GeoES (QEP09776.1), and/or SSS1, (CAA98906.1)
Post-translational support, such as UBI4, (CAA97489.1)
Isomerases for proper protein folding, such as CPR5 (KZV12543.1) and/or CPR2 (KZV10787.1).
Heat shock response ER membrane proteins, including calnexin, such CNE1, (QHB06590.1)
Endoplasmic reticulum redox balance for proper protein folding including disulfide bond formation, such as ERO1, (QHB10609.1), PDI1, (KZV12810.1), and/or EUG1, (KZV12759.1).
Protein translocation and protein folding enhancers, such as SBH1, (KZV11852.1), and/or SEC61 (CAA44215.1)
Enhancers of protein trafficking, vesicle formation, tethering, and fusion between organelles and membranes, SEC31 (CAA98772.1), SLY1, (CAA38221.1), BOS1, (CAA97636.1), BET1, (CAA86247.1), SEC22, (QHB10420.1), SED5, (CAA97549.1), COG7, (QHB08640.1), COY1, (QHB09825.1), IMH1, (QHB10458.1), SEC1, (QHB07623.1), SEC4, (QHB08330.1), SNC1, (KZV13437.1), SNC2, (CAA89974.1), SSO1, (CAA97949.1), and/or SSO2, (KZV09033.1)
Secretory pathway processing enzymes, including glycosyltransferase, KEX2, (CAA96143.1), MNN1, (AAA53676.1), MNN2, (QHB06786.1), MNN9, (KZV07467.1), MNN10 (QHB07704.1), MNN11, (QHB09454.1), and/or OCH1, (CAA96740.1)
Cell wall related proteins for stress tolerance and cell wall stability, such as CCW12 (KZV09357.1), CWP2, (CAA81937.1), YPS1, (KZV09366.1), MKC7, (KZV12382.1), and/or SED1 (BAI99734.1)
Flavin cofactor synthesis and recycling proteins for FAD+/FADH use by enzymes expressed in a modified host, such as FADI (CAA98604.1) or FMN1 (NM_001180544.1).
Nicotinamide cofactor synthesis and recycling proteins for NAD (P)+/NAD (P) H use by enzymes expressed in a modified host, such as UTRI (CAA89577.1), POS5 (CAA97900.1),
Upregulation of the pentose phosphate pathway (PPP) such as increased expression of pentose phosphate genes encoding for enzymes in the PPP, such as GND1 (KZV10926.1) and/or ZWF1 (CAA96146.1)
Enzymes to increase acetyl-CoA pool including alcohol and aldehyde dehydrogenases and acetyl-CoA synthetases such as ALD6 (QEP09776.1), ADH2 (QEP09776.1), and/or ACS (AAL23099.1)
Enzymes to upregulate heme biosynthesis, such as delta-aminolevulinic acid dehydratase (Hem2) (CAA96742.1), porphobilinogen deaminase (Hem3) (CAA98783.1), uroporphyrinogen decarboxylase (Hem12) (QHB07515.1), oxygen-dependent coproporphyrinogen III oxidase (Hem13) (QHB07512.1), and/or ferrochelatase (Hem15) (CAA99385.1).
Transporters to increase metal ion import, such as FET3 (QHB10789.1), FTR1 (QHB08222.1), and/or FET4 (QHB11048.1)
Metal ion oxidoreductases to convert metals into usable forms, such as the ferric and copper reductases, FREI (QHB10365.1) and/or FRE3 (KZV08274.1)

In various embodiments, the recombinant cell comprising endogenous genes encoding proteins for an improved effect(s) for the expression of a target compound (monoterpene indole alkaloid (MIA), intermediate, precursor, or metabolite thereof) may be adapted or manipulated (i.e., genetically modified) in a way that modifies endogenous gene expression. Non-limiting examples include swapping an endogenous promoter for the gene of interest with a stronger promoter (constitutive or inducible, such as a glycolytic TEFI promoter or a galactose inducible GALI promoter); codon optimized gene sequence encoding proteins (e.g., genetic integration, episomal plasmids, or artificial chromosomes of such genes in an expression cassette (e.g. promoter, coding region, terminator)) any of which can be accomplished using methods known in the art.

The recombinant cell can contain combinations of modifications to host genes where genes or combinations thereof have downregulated or no (i.e. knocked out) expression, and/or silenced message, which can improve: protein production, correct protein folding, protein secretion, proper protein intracellular localization, enzyme activity, correct post-translational modifications for protein features, mRNA stability, cell tolerance to stress, cellular metabolic activity, availability of cofactors for enzyme activity, glycolysis, fatty acid metabolism, feedstock conversion, amino acid biosynthesis, mevalonate pathway flux, coenzyme A (CoA) flux, acetyl-CoA production, tolerance to oxidative stress (e.g. H₂O₂) from increased protein production, tolerance to oxidative stress from monoterpene indole alkaloid (MIA) and related precursor enzymatic pathway steps, titer of alkaloids and alkaloid pathway precursors, intermediates, and compounds.

Such genes (with examples of Genbank nucleotide accession numbers) include combinations thereof:
Transcription factors such as ROX1 (NM_001184162.1)
Heme depletors, including oxygensases, such as HMX1 (NM_001182092)
Proteases, such as PEP4 (NM_001183968.1), PRC1 (NM_001182806.1), PRB1 (NM_001178875.1)
Ubiquitin ligases, such as BUL1 (NM_001182782.1) and/or BUL2 (NM_001182473.1)
O-acetyltransferases, such as ATF1 (NM_001183797) and/or ATF2 (NM_001181306.1).
Dehydrogenases and reductases, such as ARII (NM_001181022), ADH6 (NM_001182831.3), OYE2 (NM_001179310), and/or OYE3 (NM_001183985.1).
Genes encoding for proteins involved in amino acid metabolism, such as BNA2 (NM_001181736.3), ARO10 (KU050081.1), PDC6 (NM_001181216.3)
Genes that promote cell death in response to toxicity of high MIA precursor concentrations, such as YCA1 (AY692832.1).

### Biosynthetic Methods

In some aspects, the disclosure provide a biosynthetic method for producing one or more target compounds (e.g., an iboga compound, an indole alkaloid, or an intermediate, precursor, or metabolite thereof), comprising: (i) generating a recombinant host cell; (ii) growing the recombinant host cell under conditions effective to produce the target compound(s), or precursor(s) thereof; and (iii) isolating the target compound(s) from the recombinant host cell. In some embodiments of the method, the method comprises growing the recombinant host cell under conditions effective to express the genes encoding the enzymes and/or proteins, and fermenting the recombinant host cell to produce the target compound(s). Endogenous pathways of the recombinant host can be modified by the systems and methods herein to produce high purity target compounds.

In various embodiments of the biosynthetic methods to produce the desired monoterpene indole alkaloids and related intermediates and precursors, the nucleic acid encoding the enzyme(s) and/or regulatory protein(s) are introduced into a host cell using standard cell (e.g., yeast) transformation techniques (Green and Sambrook, 2012). Cells are subjected to fermentation under conditions that activate the promoter controlling the synthesis of the enzyme and/or regulatory protein. The broth may be subsequently subjected to HPLC analysis to determine the presence or yield of the desired monoterpene indole alkaloid and/or related intermediate and/or precursor products.

In various embodiments, the host cells are provided with various feedstocks to drive production of the desired monoterpene indole alkaloids and the related precursors or pathway intermediates, (e.g., feedstocks comprising glucose, fructose, sucrose, galactose, raffinose, maltose, ethanol, xylose, fatty acids, glycerol, acetate, molasses, malt syrup, corn steep liquor, dairy, flour, protein powder, olive mill waste, fish waste, etc., as is known in the art.

In some embodiments, the biosynthesis of MIAs, intermediates, and analogs, is carried out by cell-free lysate, crude or purified, or via purified enzymes from a recombinant host, or a combination of modified hosts, which has been modified to express MIA biosynthetic pathway enzymes. As described above, the modified strains express the genes for biosynthetic pathways that generate downstream biosynthetic pathways to generate, e.g., demethylcorynantheidine, corynantheidine, 9-hydroxycorynantheidine, or mitragynine, and/or other intermediate monoterpene indole alkaloids compounds and precursors.

Recombinant host cells expressing the MIA pathway to generate precursors, intermediates, and end products of the MIA pathway can be grown, fermented, and produce products on various carbon and nitrogen sources as described herein. In some embodiments, additions such as vitamin mixes and trace minerals are added to a fermentation media, which can include choline chloride, niacin, pyridoxine hydrochloride, riboflavin, calcium pantothenate, para-aminobenzoic acid (PABA), thiamine HCl, biotin, cyanocobalamin, and/or folic acid, and mineral mixes, which can include calcium chloride dihydrate, ferrous sulfate heptahydrate, manganese (II) sulfate monohydrate, copper sulfate pentahydrate, zinc sulfate heptahydrate, magnesium chloride, and solutes, such as glycerol.

In some applications, MIA precursors and products generated from a recombinant host are captured using in situ product removal (ISPR). In some embodiments, 1-30% volume to volume of fermentation broth to ISPR reagent is used. Such ISPR reagents can include, but are not limited to, isopropyl myristate, vegetable oils such as corn oil or olive oil, oleic acid, MTBE, 1-decanol, oleyl alcohol, bis(2-ethylhexyl) phthalate, alkanes such as octane and dodecane, styrene, silicone oil, non-polar macroporous resin such as C8 or C18 resin, ion-exchange resin, trioctylamine, activated carbon, ethyl oleate, lauryl acetate, farnesane, TWEEN-80, TX-100, squalene, glycerin, and/or propylene glycol. For example, ISPR and analysis of MIA products or precursors can be carried out by an ISPR process using dodecane. In this case, a 20% volume to volume dodecane layer is added to a recombinant host fermentation. After 72h of fermentation, the dodecane is removed, diluted 1:10 with ethanol, and filtered for volatile compound detection via HPLC. The broth extraction and subsequent HPLC detection can be carried out as described herein and/or as in WO/2021/248087 (PCT/US21/36031). The use of these reagents also contributes to increased MIA yield by reducing the amount of pathway intermediate lost to evaporation.

Isolation and detection of MIA products and precursors from recombinant cell, lysate, or co-culture fermentation broth can be performed by solvent extraction, filtration, and analytical methods including high performance liquid chromatography (HPLC), liquid chromatography mass spectrometry (LC-MS), high resolution mass spectrometry (HRMS), gas chromatography (GC), gas chromatography mass spectrometry (GC-MS), and/or nuclear magnetic resonance (NMR) methods, as known to those skilled in the art. For example, recombinant host strains expressing the MIA biosynthesis pathway can be mixed with a solvent such as acetonitrile, ethanol, isopropanol, methanol, ethyl acetate, acetone, water, and mixtures thereof, which can include additives in amounts of 0.01-20% volume to volume formic acid (FA), ammonium carbonate, or trifluoroacetic acid (TFA). Such solvent mixtures can be applied to whole broth, cell pellets, or fermentation supernatant in combination with centrifugation, vortexing, bead-beating, homogenization, filtration, such as tangential flow filtration (TFF), and/or shaking and incubation, at varying temperatures from 4-100 degrees Celsius from 1-120 minutes. Such extractions can then be filtered for running on analytical equipment, such as dead-end filtration through a 0.1-0.22 micron filter of polytetrafluoroethylene (PTFE), polyvinylidene fluoride (PVDF), nylon, cellulose, or polyethersulfone (PES) to remove unwanted debris from sensitive analytical procedures.

### Generating Downstream Mitragynine Alkaloids

As discussed throughout the disclosure, methods are provided for the biosynthetic production of molecules that are part of the downstream alkaloid biosynthetic pathway related to mitragynine, its precursors, and analogs (e.g., including isotopic analogs (e.g., deuterium analogs), substituted analogs (e.g., oxa- or thia-tryptamines; halogenated compounds), and stereoisomers).

In some embodiments, the methods for generating a downstream alkaloid (mitragynine) compound comprises one or a combination of polynucleotides, enzymes, proteins, or recombinant cells as described herein that are maintained under conditions that allow for the expression and/or activity of the polynucleotide(s) and/or enzymes or proteins and which are sufficient for the synthesis and production for the downstream alkaloid compound.

In some embodiments of the methods, the disclosure provides for an increase in the production yield and/or titer of one or more of the target compound(s) that are generated. In further embodiments, the increase in the production yield and/or titer of the target compound(s) is relative to the amount or titer of the target compound(s) that is present in a naturally-occurring source. In further embodiments, the increase in the production yield and/or titer of the target compound(s) is relative to the amount or titer of the target compound(s) that can be generated in alternative chemical synthetic methods. In further embodiments, the increase in the production yield and/or titer of the target compound(s) is relative to the amount or titer of the target compound(s) that can be produced from alternative recombinant host cells.

In some embodiments, the method comprises a DCS polynucleotide sequence including any one or more of the sequences DCS_In-26n as disclosed herein, or a sequence having at least 90% sequence identity to any such DCS polynucleotide sequence. In some embodiments, the method comprises a DCS amino acid sequence including any one or more of the sequences DCS_Ip-26p as disclosed herein, or a sequence having about 90% sequence identity to any such DCS amino acid sequence.

In some embodiments, the method comprises a M9OMT polynucleotide sequence including any one or more of the sequences M9OMT_In-64n as disclosed herein, or a sequence having at least 90% sequence identity to any such M9OMT polynucleotide sequence. In some embodiments, the method comprises a M9OMT amino acid sequence including any one or more of the sequences M9OMT_Ip-64p as disclosed herein, or a sequence having about 90% sequence identity to any such M9OMT amino acid sequence.

In some embodiments, the method comprises an enolMT polynucleotide sequence including any one or more of the sequences enolMT_In-7n as disclosed herein, or a sequence having at least 90% sequence identity to any such enolMT polynucleotide sequence. In some embodiments, the method comprises an enolMT amino acid sequence including any one or more of the sequences enolMT_Ip-7p as disclosed herein, or a sequence having about 90% sequence identity to any such enolMT amino acid sequence.

In some embodiments, the method comprises a M9H polynucleotide sequence including any one or more of the sequences M9H_In-36n as disclosed herein, or a sequence having at least 90% sequence identity to any such M9H polynucleotide sequence. In some embodiments, the method comprises a M9H amino acid sequence including any one or more of the sequences M9H_1p-36p as disclosed herein, or a sequence having about 90% sequence identity to any such M9H amino acid sequence.

In some embodiments, the method comprises a cytochrome P450 reductase (CPR) gene of the Sequence Listing provided herewith, including any one or more of sequences CPR_In-CPR_4n, or a cytochrome P450 reductase (CPR) amino acid sequence of the Sequence Listing provided herewith, including any one or more of sequences CPR_Ip-CPR_4p, or a sequence having at least 90% sequence identity to any of the CPR sequences.

In some embodiments, the method comprises a cytochrome b5 (CYB5) gene, which together with CPR can participate in electron transfer reactions. Coexpression of CPR and CYB5 proteins can enhance enzymatic oxidations, such as increasing the activity of the P450 enzymes described herein. In some embodiments, the method comprises a CYB5 gene of the Sequence Listing provided herewith, including CYB5_In, or CYB_2n, or a cytochrome b5 (CYB5) amino acid sequence of the Sequence Listing provided herewith, including CYB5_Ip, or CYB5_2p, or a sequence having at least 90% sequence identity to any of the CYB5 sequences.

In some embodiments, the method comprises a transporter (trxporter) gene of the Sequence Listing provided herewith, including any one or more of sequences trxporter_In-trxporter_6n, or a transporter (trxporter) amino acid sequence of the Sequence Listing provided herewith, including any one or more of sequences trxporter_Ip-trxporter_6p, or a sequence having at least 90% sequence identity to any of the trxporter sequences.

In some embodiments, the method comprises a tryptophan importer (TAT2) gene of the Sequence Listing provided herewith, including TAT2_In or a tryptophan importer (TAT2) amino acid sequence, or a sequence having at least 90% sequence identity to any of the TAT2 sequences.

In some embodiments, the method comprises a SAMe importer (SAM3) gene of the Sequence Listing provided herewith, including SAM3_In or a SAMe importer (SAM3) amino acid sequence, or a sequence having at least 90% sequence identity to any of the SAM3 sequences.

In some embodiments, the method comprises a methionine importer (MUP1) gene of the Sequence Listing provided herewith, including MUP1_In or a methionine importer (MUP1) amino acid sequence, or a sequence having at least 90% sequence identity to any of the MUP1 sequences.

In some further alternative embodiments, the method comprises a tryptamine hydroxylase which can hydroxylate an indole, tryptamine, or tryptophan molecule, such as PsiH-type and other hydroxylase genes, such as a tryptamine-4-hydroxylase (T4H), disclosed in U.S. patent application Ser. No. 18/000,931 (ENZYMES AND REGULATORY PROTEINS IN TRYPTAMINE METABOLISM) and U.S. Pat. No. 11,441,164 (BIOSYNTHETIC PRODUCTION OF PSILOCYBIN AND RELATED INTERMEDIATES IN RECOMBINANT ORGANISMS).

In some further alternative embodiments, the method comprises an O-methyltransferase which can methylate hydroxyl groups on an indole or tryptamine molecule, such as the indole (or tryptamine)O-methyltransferases (IOMTs) disclosed in U.S. patent application Ser. No. 18/000,931 (ENZYMES AND REGULATORY PROTEINS IN TRYPTAMINE METABOLISM).

In some further alternative embodiments, the method comprises a kinase which can phosphorylate a hydroxylated indole, tryptamine, or tryptophanm such as PsiK-type and other kinases genes disclosed in U.S. patent application Ser. No. 18/000,931 (ENZYMES AND REGULATORY PROTEINS IN TRYPTAMINE METABOLISM) and U.S. Pat. No. 11,441,164 (BIOSYNTHETIC PRODUCTION OF PSILOCYBIN AND RELATED INTERMEDIATES IN RECOMBINANT ORGANISMS).

Other useful sequences include cofolding tags CoFoldtag_1 (SEQ ID NO:358) and CoFoldtag_2 (SEQ ID NO:359); copper reductase oxidase gene CRO_In (SEQ ID NO:368); horseradish peroxidase gene HRP_In (SEQ ID NO:369); and histidine tag HHHHHH (SEQ ID NO: 232).

In some further alternative embodiments, the method comprises a tryptophan or tryptamine halogenase (TrpHalo) which can halogenate indole, typtamine, and/or tryptophan molecules, such as the halogenases disclosed in U.S. patent application Ser. No. 18/000,931 (ENZYMES AND REGULATORY PROTEINS IN TRYPTAMINE METABOLISM).

In some embodiments, the method can include production of one or more labile intermediate compounds that can form spontaneously and which can feed into a separate portion of the pathway for production of indole alkaloids including mitragyninc.

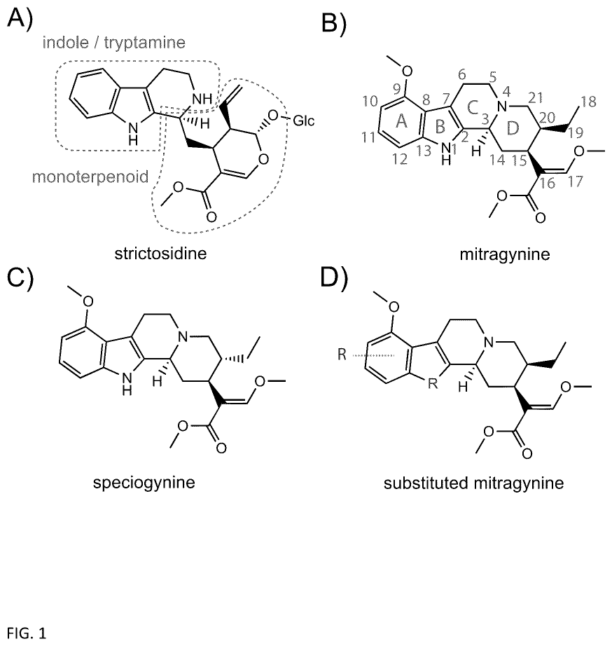

For purposes of illustration and clarity, FIG. 1 depicts the tryptamine and alkaloid skeletons. The portion of the molecule from the tryptophan indole is highlighted. The portion of the molecule from the monoterpenoid is highlighted. In embodiments, engineering of the native yeast tryptophan pathway to enable high flux toward tryptophan and tryptamine production is used in the systems herein, and is described in, e.g., WO 2021/248087 (PCT/US2021/036031).

The terpene moiety of strictosodine, secologanin, can be fermentation-derived by the enzyme-engineered host as described herein or fed into the media. Secologanin exogenously provided to a fermentation media can be crude extract material from plants which contain MIA precursors. For example, extracts from plants from the Symphoricarpos genus, such as snowberry (Symphoricarpos albus), and other plants such as Silvervine (Actinidia polygama), Lonicera japonica and Honeysuckle (Lonicera caerulea) can be a source of exogenous MIA precursors such as secologanin. In other embodiments, material and extracts thereof from plants of the Gentianales order, which can include Mitragyna speciosa (kratom), Uncaria tomentosa, Uncaria rhynchophylla, Voacanga africana, Voacanga thouarsii, Tabernanthe iboga, Tabernanthe mannii, Picralima nitida, Tabernaemontana elegans, and/or Catharanthus roseus. In some embodiments the secologanin is provided by the plant and/or berry extract for the host cell to uptake as a starting substrate to feed into the biosynthetic pathway for MIA production. Likewise, other iridoid precursors for MIA production can be sourced and extracted, whether to high purity or remaining as a crude extract, from the plants listed herein. For example, snowberry (Symphoricarpos albus) extract and Honeysuckle (Lonicera caerulea) extract contain loganic acid and loganin. In some embodiments the loganic acid and/or loganin is provided by plant and/or berry extract for the host to uptake as starting substrate to feed into the biosynthetic pathway for indole alkaloid production.

In embodiments wherein plant material is incorporated as a fermentation media supplement for recombinant hosts to produce MIAs, the plant material comprising MIA precursors is processed by methods known by those skilled in the art. For example, in some embodiments, the plant material, such as Honeysuckle, is crushed up into powder using a mortar and pestle, a homogenizer, vortexing, or a blender, and can be extracted using a variety of organic solvents ranging in polarity, including but not limited to methanol, ethanol, butanol, ethyl acetate, acetonitrile, methyl tertiary-butyl ether (MTBE), and/or acetone. To lyse plant cells for efficient extraction of indole alkaloid pathway compounds, the powder is dissolved in water (e.g., ddH₂O) and subjected to either bead beating for 3-6 min. repeated one to five times, homogenization, or freeze-thaw cycles (e.g., alternating boiling water bath and frozen ethanol bath or liquid nitrogen) for three to six times. Organic solvent listed above is then added to the lysate and incubated with the lysate for 1-24 hrs at 20-50° C. The organic layer is then collected, dried and resuspended in acetonitrile for analytical detection and quantification. The leftover aqueous layer is also extracted with acetonitrile and subjected to analysis by HPLC, LC-MS, HRMS, and LC-MS/MS. The dried plant extract containing MIA precursors can be used as a fermentation media supplement for MIA production by recombinant host cells.

For selective detection and characterization of MIA compounds and precursors including ketones and aldehydes produced by recombinant hosts or present in plant extracts, derivatization techniques known to those skilled in the art can be used. Reagents (e.g., modifying or derivatizing agents) that can be used for detection and characterization of MIA compounds can include, but not be limited to, 2-amino benzamidoxine (ABAO), para-methoxy-2-amino benzamidoxime (PMA), 4-bromo-N-methylbenzylamine, 9-fluoromethylchloroformate (FMOC), 3-methyl-2-benzothiazolinone hydrazone (MBTH), 1-(5-fluoro-2,4-dinitrophenyl)-4-methylpiperazine (PPZ), 2,4-dinitrophenylhydrazine (DNPH) and 4-bromo-N-methylbenzylamine (4-BNMA). For example, in some embodiments, fermentation broth or plant material extracts are derivatized using 2-amino benzamidoxime (ABAO). In such embodiments, fermentation broth or plant material extracts (e.g., in acetonitrile) are subjected to reacting with 0.1-1 mM ABAO at pH 4-7. The reaction can be performed at 30-37° C. and incubated for at least 15 min or allowed to incubate for hours (i.e., overnight). In embodiments comprising an ABAO derivatization of a MIA precursor and/or product, the derivatized product formation is monitored with fluorescence excitation at 360 nm and emission at 528 nm. Alternatively, both the ABAO reagent and derivatized adducts can be monitored with a UV absorbance at 405 nm. All derivatized products can be further analyzed with LC-UV-MS.

In some embodiments, sugars and nitrogen from plant and berry extracts also provide the nutrients necessary for the growth and fermentation of recombinant host cells in addition to providing iridoid precursors for MIA production, minimizing or eliminating any other additional media additives for the recombinant host.

In some embodiments, these precursors are converted to MIAs in a cell-free system, such as via crude or purified lysate, microsomes, or purified enzymes from a recombinant host or combinations of recombinant hosts which contain the biosynthetic enzymes for MIA production.

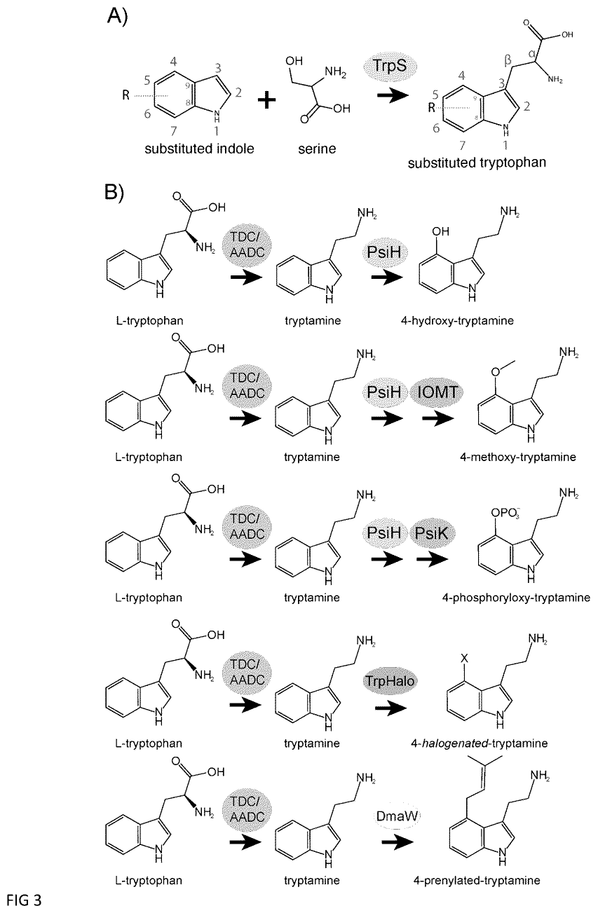

In some further downstream biosynthetic pathway embodiments, FIG. 3 shows example enzyme conversion of substituted indoles and tryptamines, such as from (A) a modified indole that leads to a substituted tryptophan, and (B) enzymatic modifications of tryptamine which can lead to downstream analogs of alkaloids including strictosidine-type analogs through mitragynine analogs. Such a pathway can be carried out in a recombinant host expressing combinations of sequences described here in, including genes encoding enzymes for enolMT, DCS, M9OMT, and/or M9H, in addition to other genes described herein (e.g., PsiH or T4H), IOMT, PsiK, TDC/AADC, and TrpHalo) and genes otherwise known in the art to generate MIA pathway precursors and intermediates.

FIG. 1C-1D depicts a stereoisomer (1C) and substituted analogs with a modified or substituted indole ring (1D). Sources of modified indole rings can stem from substituted tryptophan or tryptamine-like molecules can be found in WO/2021/248087. R substitutions on the indole ring positions include single or multiple independent substitutions of H, deuterium, OR2, NR2 R2, N+R2, SR2, S+R2, P+R2, P (O) R2, P (O) R2, S (O) R2, SO2R2, SO3R2, CO2R2, CN, oxo, a halide, a haloalkyl and substituted or unsubstituted alkyl, alkenyl, alkynyl, alkoxy, alkoxyalkyl, alkylamino, dialkylamino and alkylthio, wherein R2 independently comprises H, deuterium, alkyl, alkenyl, alkynyl, oxoalkyl, alkenyl, alkynyl, alkoxy, alkoxyalkyl, alkylamino, a halide, a haloalkyl, dialkylamino and alkylthio. Additional variants of the indole ring nitrogen (exemplified as “R” in the indole ring in FIG. 1D) can also be incorporated including, for example, the substitutions of the indole ring nitrogen with an oxygen atom (i.e. benzofurans), a sulfur atom, a carbon atom (optionally substituted), or a substituted ring nitrogen (such as with R2 as defined above (e.g., methylated, deuterated, or a halogenated)) as indicated. Such substitutions of the nitrogen of the indole ring can lead to substituted strictosidines, as well as derivates to downstream indole alkaloids such as, for example corynantheidines and mitragynincs. A nonlimiting example of these substituted MIAs where the indole nitrogen is replaced, for instance with an oxygen (e.g. benzofuran derivatives) can lead to ‘oxa’ versions of MIAs, including oxa-strictosodine and downstream alkaloid oxa-derivatives thereof (see FIG. 1D) and many other corynanthe-type MIAs, where the benzofuran moiety can originate from a benzofuran precursor added to a fermentation or cell free reaction. Another nonlimiting example can stem from a 1-(or N-) methylated substituted corynanthe molecule (see FIG. 1D) where the N-methylated indole moiety can originate from a 1-methylated tryptophan or tryptamine precursor added to a fermentation or cell free reaction.

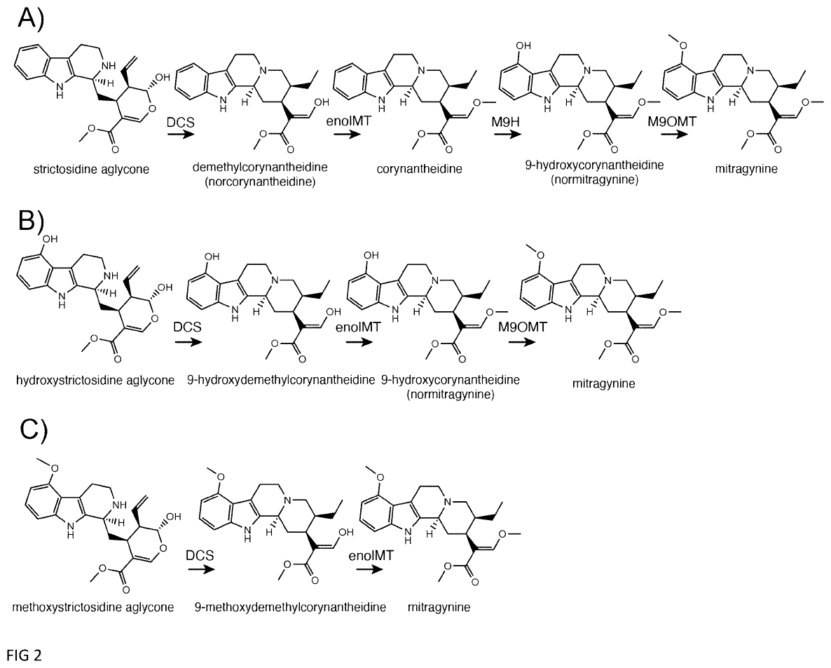

FIG. 2 depicts the biosynthetic pathway to mitragynine from various starting strictosidine aglycone-type analogs. From strictosidine and strictosidine aglycone, as in FIG. 2A, the DCS, enolMT, M9H, and M9OMT yield mitragynine. From hydroxystrictosidine and hydroxystrictosidine aglycone, as in FIG. 2B, the DCS, enolMT, and M9OMT yield mitragynine. From methoxystrictosidine and methoxystrictosidine aglycone, as in FIG. 2C, the DCS, and enolMT yield mitragynine.

FIG. 3 depicts the routes leading to substituted tryptophan and modified tryptamines, which can go on to react with secologanin via the STR enzyme to create strictosidine analogs which can enter a pathway withSGD, DCS, enolMT, M9H, and/or M9OMT enzymes to yield analogs of mitragynine intermediates and analogs of mitragynine. In FIG. 3A. the modification originates from an indole, that is then condensed into a modified tryptophan, which can then enter the MIA pathway. In FIG. 3B, upstream enzymatic modifications to tryptamine yield modified tryptamines which can then enter the MIA pathway.

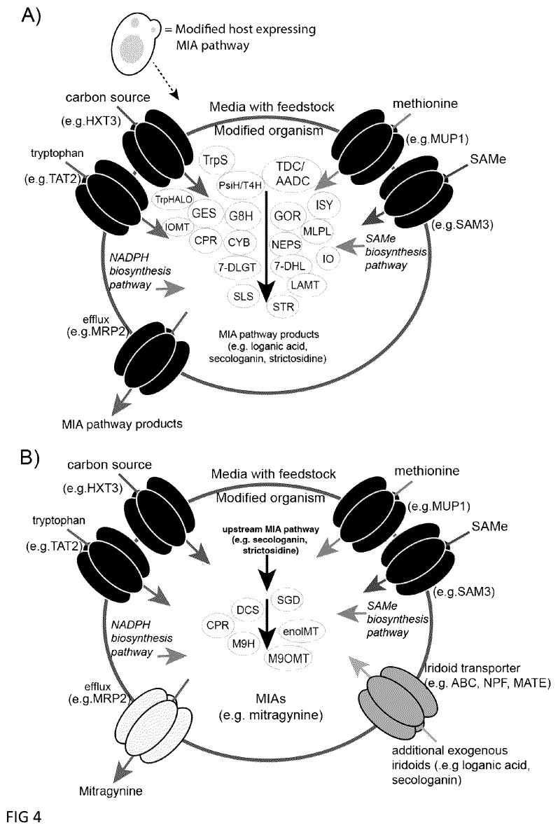

FIG. 4 depicts example embodiments comprising modified cell hosts expressing gene combinations for generating MIA pathway products, in accordance with the disclosure, and embodiments that utilize the compounds generated by the disclosed methods to feed into various downstream MIA biosynthetic pathways. FIG. 4A depicts a modified host cell which generates MIA intermediates, such as strictosidine, hydroxystrictosidine, methoxystrictosidine, among others, which can feed into or be utilized by downstream MIA pathway enzymes or by a recombinant host expressing the downstream MIA pathway leading to mitragynine and mitragynine analogs. FIG. 4B depicts a modified host cell which expresses both an upstream (as in FIG. 4A) and a downstream MIA biosynthetic pathway to generate mitragynine and mitragynine analogs.

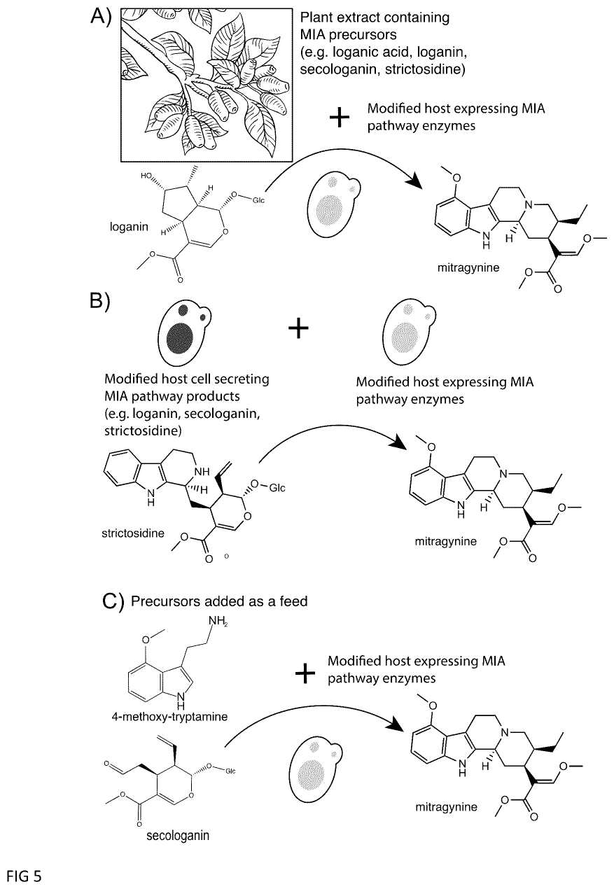

To illustrate some additional embodiments, FIG. 5A depicts an example of bioconversion by a modified cell host of an MIA precursor into a MIA target compound. Exogenous pure precursor or crude extract of precursors, e.g., containing iridoids, loganic acid, loganin, secologanin, strictosidine, among others, can be fed to recombinant cells expressing the MIA biosynthesis pathway. FIG. 5B shows an example of bioconversion using multiple organisms via a modified cell host expressing a portion of the MIA pathway, for example producing up to strictosidine, while another modified host cell expressing a downstream MIA pathway uses the products from the first host cell to produce MIAs such as mitragynine. Precursors made by a modified cell host can be excreted or derived from crude or purified lysate and fed to cells expressing downstream pathway enzymes. FIG. 5C depicts an example of bioconversion by a modified cell host of an MIA precursor which includes modified tryptamines into a MIA target compound.

In embodiments, the de novo biosynthesis pathway of L-tryptophan and SAM/SAMe are utilized as precursor molecules and cofactors in the systems and methods herein. The precursor molecules lead to target molecules of the tryptamine pathways, when incorporated on-pathway. In some embodiments of the systems (e.g., organisms) and methods herein, glycolysis leads to chorismate (e.g., comprising chorismate synthase) via the shikimate pathway; glutamate biosynthesis pathway leads to L-glutamine via L-glutamate; and the L-serine biosynthesis pathway leads to L-serine via 3-phospho-L-serine (i.e., dephosphorylation). Chorismate, glutamine, and L-serine can be combined to form L-tryptophan as a precursor molecule, and which can be incorporated into the biosynthetic pathway that can yield tryptamine. In some embodiments of the systems (e.g., organisms) and methods herein, L-methionine is a direct precursor that can produce SAMe, when combined with ATP in the presence of Sam2 and Adkl enzymes. Methods described or referenced herein to increase flux through the tryptophan and SAMe biosynthesis pathways, such as overexpressing genes involved in the shikimate pathway, chorismate pathway, and methionine pathway, can lead to increased MIA product yield.

Describing this further, in some embodiments, tryptophan accumulation in a recombinant host cell can be increased by one or more of: (a) overexpressing feedback-resistant versions of tryptophan-synthesizing enzymes; (b) knocking out and/or inhibiting expression of off-pathway tryptophan-consuming genes and enzymes; and/or (c) overexpressing a recombinant L-tryptophan transporter for exogenous tryptophan import (see also U.S. PGPub 2021/0147888 and PCT Patent Application Publication WO 2021/248087). In some embodiments the biosynthesis can comprise (over) expression of enzymes (e.g., TRP1, TRP2, TRP3, TRP4 and/or TRP5) which can increase intracellular tryptophan and maintain or direct tryptophan flux through the pathway. In some embodiments the biosynthesis can comprise (over) expression of one or more genes useful for tryptophan biosynthesis, such as ARO1, ARO4, AROL, SER1, SER3, SER33, and/or SER2, and including feedback resistance mutant versions thereof, which increase shikimate pathway flux and/or supply serine.

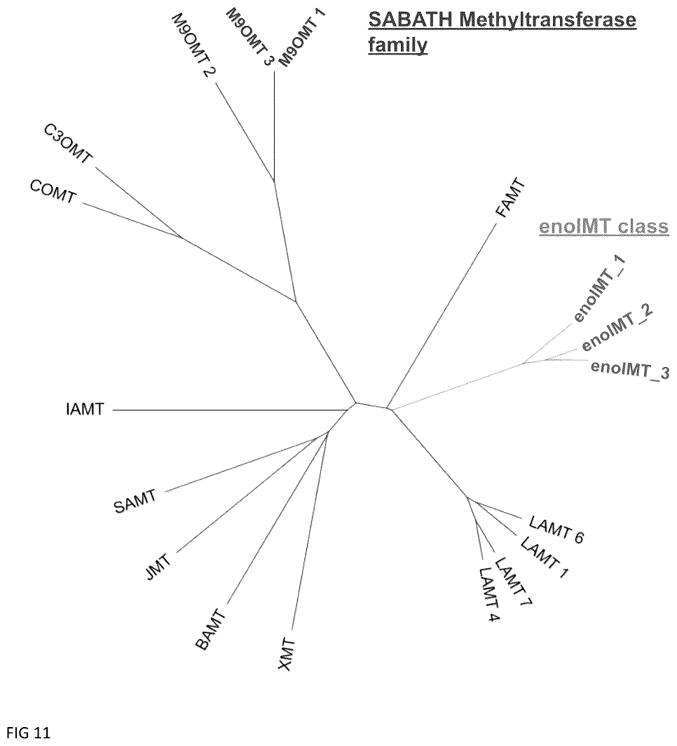

FIG. 11 illustrates the novelty of this newly discovered enolMT class in relation to other methyltransferases in the SABATH (SAMT, BAMT, and Theobromine synthase) family, which is a common family of small molecule methyltransferases in plants. FIG. 11 depicts a phylogenetic tree of SABATH family methyltransferases in comparison with enol methyltransferases (enolMTs). Abbreviations: IAMT for indole-3-acetic acid OMT, SAMT for salicylic acid OMT, JMT for jasmonic acid OMT, BAMT for benzoic acid OMT, XMT for xanthosine NMT, LAMT for loganic acid OMT, enolMT for enol OMT, FAMT for farnesoic acid OMT, M9OMT for mitragynine indole ring OMT, C3OMT for caffeic acid 3-OMT, COMT for catechol OMT. The closest relatives, loganic acid methyltransferases (LAMTs) only show ˜40% sequence similarity to enolMTs whereas farnesoic acid methyltransferases (FAMT) show ˜30% sequence similarity to enolMTs.

The following Examples illustrate several aspects and embodiments in accordance with the disclosure and in no way serve to limit the scope or spirit of the claims.

### EXAMPLES

### Example 1. Method of Cell Growth

Modified host cells that yield MIA precursors and compounds, such as the downstream alkaloid-producing strains described herein, express engineered genes and enzymes. More specifically, the downstream alkaloid-producing strains herein are grown in a minimal, complete culture media containing yeast nitrogen base, amino acids, vitamins, ammonium sulfate, and a carbon source of glucose and galactose. The recombinant host cells are grown in 24-well plates or shake flasks in a volume range of 2 mL to 100 mL of media starting from an inoculation density of OD₆₀₀ₙₘ=1. Exogenous compounds (e.g., upstream precursor compounds, such as strictosidine, strictosidine aglycone, or coronaridine) up to 1% can be added to media to supplement the precursor pool for downstream alkaloid-production. In addition or alternatively, early (upstream) pathway precursors can be imported into host cells. Exogenous L-tryptophan can be taken up by strains expressing the TAT2 L-tryptophan importer protein and exogenous L-methionine can be taken up by strains expressing the MUP1 L-methionine permease protein. The strains herein can be harvested during a fermentation period ranging from 12 hours onward from the start of pathway enzyme induction.

Alternatively, strains such as Saccharomyces cerevisiae that are modified to produce downstream alkaloids can be grown and fermented in media containing in the juice of Snowberry (Symphoricarpos albus) or Honeysuckle (Lonicera caerulea) to provide the secologanin precursor. Platform strains are grown in berry juice supplemented with compounds essential for growth (20 mg/L adenine, 20 mg/L L-histidine, 30 mg/L lysine, 20 mg/L L-leucine, and 20 mg/L tryptophan, pH 5.8). Carbon source, such as sucrose, glucose, galactose, xylose, lactose, and/or glycerol at 10-60 g/L is added to the media. Alternatively, secologanin can be provided as a feed-in to the media at 2 mM final concentration.

Some embodiments can include, during the fermentation, addition of substituted substrate molecules (e.g., substituted tryptophan or tryptamine) that allow for the synthesis and production of substituted downstream MIAs. For example, oxa-tryptamine (i.e. a benzofuran) can be added to a fermentation at a concentration of 0.1-0.5 g/L, and maintained at such concentrations for 12-48 hrs by bolus addition of a concentrated oxa-tryptamine stock solution or fed-in at a rate to maintain the concentration of the substrate in the fermentation, to generate ‘oxa’ analogs of downstream MIAs. Similarly, 1-methyl-tryptamine (i.e., N-methylated at the indole ring N) or 4-methoxytryptamine can be added to a fermentation at a concentration of 0.1-0.5 g/L, and maintained at such concentrations for 12-48 hrs by bolus addition of a concentrated stock solutions or fed-in at a rate to maintain the concentration of the substrate in the fermentation to generate N-methyl or substitution analogs of downstream MIAs. In addition the non-limiting examples mentioned above, the methods can incorporate deuterated molecules (e.g., as feedstocks, solvents, precursors, etc. comprising deuterium), in order to generate and synthesize deuterated forms of the alkaloid compounds generated by the methods described herein. Similarly halogenated molecules can be incorporated in a similar fashion in order to generate and synthesize halogenated forms of the alkaloid compounds generated in accordance with the aspects and embodiments of the disclosure.

After fermentation, the broth can be extracted as described herein, for analysis of MIA pathway products.

### Example 2—Detection of Isolated Product

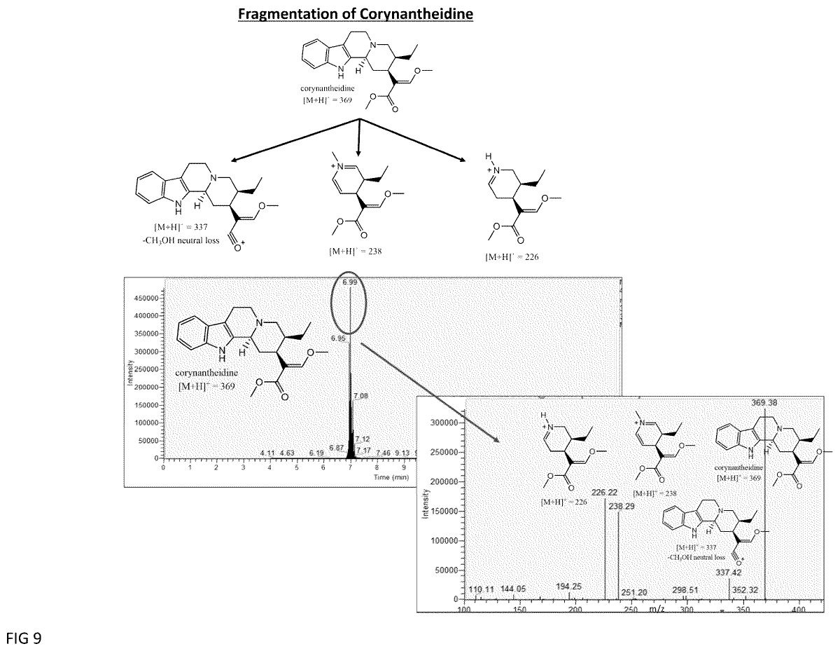

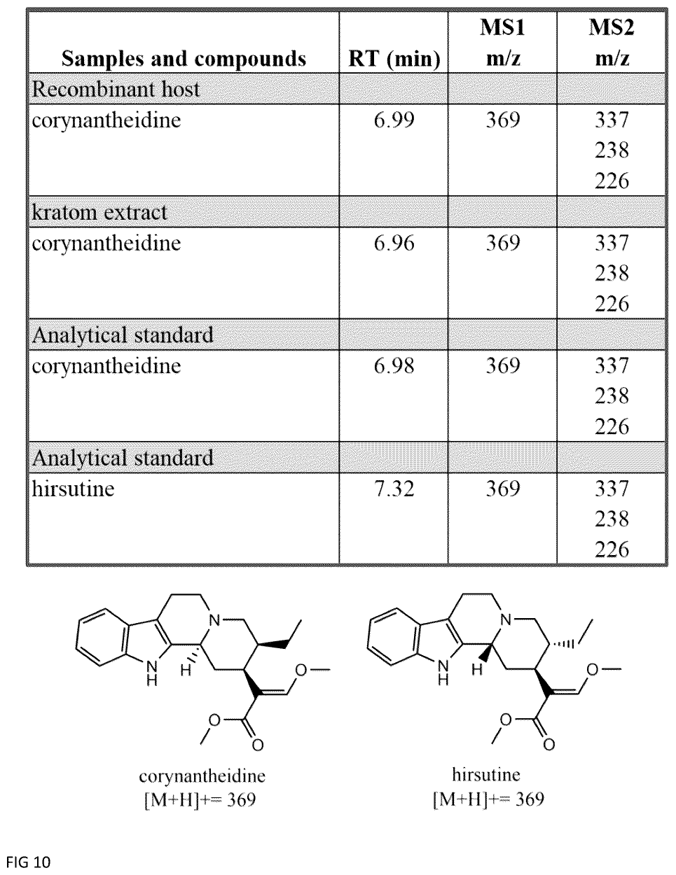

To identify fermentation-derived monoterpene indole alkaloids (MIAs), their precursors, and all other products of a recombinant host expressing an engineered biosynthetic pathway for alkaloids, an Agilent 1100 series liquid chromatography (LC) system and Agilent 6125 coupled to an Agilent 1260 LC stack, equipped with a reverse phase C18 column (Agilent Eclipse Plus C18, Santa Clara, CA, USA) is used. For high accuracy small molecule analysis and high resolution mass spectrometry (HRMS), an Agilent 6230 LC system coupled with Time-of-Flight (TOF) is used with a reverse phase C18 column (Agilent Eclipse Plus C18, Santa Clara, CA, USA). A gradient is composed of mobile phase A (ultraviolet (UV) grade H₂O+0.1% formic acid) and mobile phase B (UV grade acetonitrile+0.1% formic acid). Column temperature is set at 30° C. Compound absorbance is measured at 210 nm, 235 nm, 270 nm, and 305 nm using a diode array detector (DAD) and spectral analysis from 200 nm to 400 nm wavelengths. A mass range of m/z 10-2000 is enabled for mass spectrometry analysis of small molecules. A 0.1 mg/mL analytical standard is made from certified reference material for each compound (Cayman Chemical Company, USA). Each sample is prepared by diluting fermentation biomass from a recombinant host expressing the engineered biosynthesis pathway 1:3 or 1:20 in 100% acetonitrile and filtered in 0.2 um nanofilter vials. The retention time and UV-visible absorption spectrum (i.e., spectral fingerprint) of the samples are compared to the analytical standard retention time and UV-visible spectra (i.e. spectral fingerprint) when identifying the monoterpene indole alkaloids, their precursors, and all other related compounds. In addition or alternatively, mass fragmentation of analytes can be performed for additional compound identification data (e.g., LC-MS and LC-MS/MS). LC-MS/MS as depicted in FIG. 9, and FIG. 10, was carried out using an Agilent Eclipse C18, 4.6×100 mm, 2.5 μm particles column, at 40 degrees celsius, a flow rate of 1 ml/min, with mixtures of solvents A (H₂O with 0.1% Formic acid) and solvents B (Acctonitrile with 0.1% Formic acid), with UV-vis detection and with a Thermo LTQ Orbitrap. FIG. 9 and FIG. 10 depicts LC-MS/MS fragmentation pattern data identifying corynantheidine produced by recombinant Saccharomyces cerevisiae cells expressing the MIA pathway with an enolMT gene (enolMT_In) encoding an enolMT enzyme (cnolMT_1p).

### Example 3—Construction of Saccharomyces cerevisiae Platform Strains with Accumulation of MIA Pathway Intermediates and Products

Construction of the Saccharomyces cerevisiae platform strain with accumulated intermediates in the mitragynine biosynthetic pathways is carried out via knocking out transporter genes that can be implicated in the export of aforementioned intermediates. MIA upstream biosynthetic pathway intermediates and products such as nepetalactol, loganic acid, loganin, secologanin, strictosidine, and strictosidine aglycone, as well as downstream mitragynine biosynthetic pathway compounds such as, corynantheidine, demethyl corynantheidine, 9-hydroxycorynantheidinc, mitragyninc, and/or other MIA products intermediates, can exit cells via transporters, permeases, and exporters. The loss of MIA pathway intermediates to the extracellular space decreases the availability of these pathway intermediates inside the cells to act as substrates for the MIA biosynthetic enzymes and lowers the flux of the pathway towards mitragynine MIAs. To accumulate these key intermediates in the cells, the endogenous Saccharomyces cerevisiae genes, including but not limited to ERCI, TPO1, FLR1, SNQ2, STE6, YORI, AUSI, MDL1, MDL2, PTR2, NPF, PDR5, PDR10, PDR12 and PDR15, are deleted as single or combinatorial knockouts. Knockout of these transporter genes is performed by placement of an early stop codon in the coding region of the corresponding gene sequences using techniques known in the art. Such insertions can proceed via homologous recombination using the host machinery and make use of a synthesized DNA framing (oligonucleotide) with 40-800 nucleotides on either or both sides of a STOP codon that are complementary to the gene locus to be knocked out. Further verification of the modification in said strain can be carried out by genome sequencing and analyzed by the techniques disclosed in U.S. Pat. No. 10,671,632. Once the Saccharomyces cerevisiae platform strains with single and/or multiple transporter knockouts are completed, combinatorial episomal expression of MIA pathway enzymes, such as those encoded by, for example, M9H, enolMT, and M9OMT genes can consume the intracellular trapped MIA precursors to generate additional amounts of downstream MIA products, including mitragyninc.

### Example 4—Cell-Free Production of MIA Compounds Using a Recombinant MIA Pathway-Expressing Saccharomyces cerevisiae Strain

Cells expressing any membrane-associated/tethered enzymes and cytosolic biosynthetic enzymes encoded by the genes in accordance with certain embodiments of the disclosure are inoculated in 25-50 mL of appropriately selective media and cultured at 30° C. at 250 rpm for 40-56 hours, where the media contains 40 g/L glucose, 15 g/L ammonium sulfate, 2 g/L yeast nitrogen base (YNB), 1 g/L complete minimal dropout media (CSM) supplement, buffered with 60 mM KH2PO4 and 10 mM K2HPO4. An inducer such as 0.2-10% v/v galactose is added to the culture media and induction proceeds under the same conditions as growth for a further 18-48 hrs. Cell growth via optical density (OD) is measured and cells are harvested by centrifugation 11,000 rcf for 10 min. Cell pellets are normalized by OD. Cell pellets are washed once with 25 mL cold water. Pellets are resuspended in water and aliquoted at 30 OD. Cells are pelleted once again at 1 min at 8000 ref and then the supernatant removed. Remaining cells can be frozen and stored at −80° C. for MIA producing reactions as needed, or used immediately. Cells are then washed and lysed by adding 250 uL lysis buffer per pellet and bead beated for 1-3 min over 1-3 intervals. Lysis buffer included 100 mM potassium phosphate buffer pH7, 200 mM sodium chloride, 5% glycerol, protease inhibitor cocktail tablet, and ImM DTT. If pellets were previously frozen, pellets are first thawed on ice. Resuspended pellets are added to a tube with glass beads and cells are disrupted in a bead beater 3×3 min at 2400 rpm with 1 min on ice in between. Once the lysate is prepared, an end-point activity assay is performed by incubating the lysate with 0.01-1 mg per ml of substrate and corresponding cofactors at 30° C. for 2 hr. Substrates can include 4-methoxytryptamine, corynantheidine, secologanin, strictosidine, methoxystrictosidine, and/or 9-hydroxy-corynantheidineSamples are extracted, isolated, and analyzed via methods described herein (Scc Example 2).

### Example 5—Expression of a NADPH-Dependent Reductase Dihydrocorynantheine Aldehyde Synthase (DCS) in a Modified Host Organism to Produce Demethylcorynantheidine and Related Pathway Intermediates

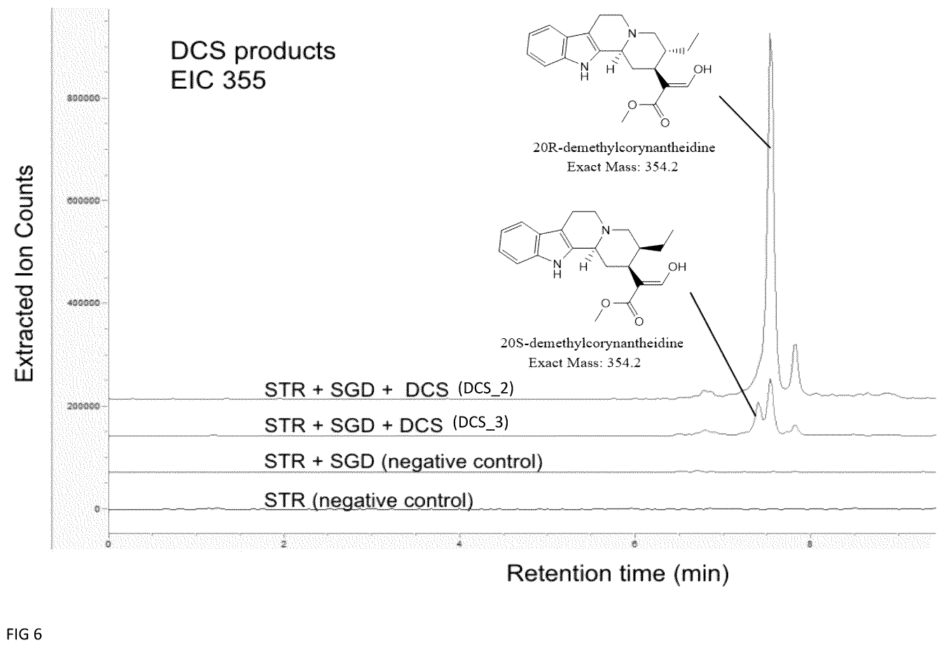

The removal of the glycosyl moiety from strictosidine triggers a cascade of chemically labile intermediates starting with strictosidine aglycone to spontaneously form unstable 4-ring intermediates such as dehydrogeissoschizine and more stable shunt products such as 5-ring cathenamine and related alkaloids. The DCS enzyme is believed to function as an NADPH-dependent reductase that iteratively reduces dehydrogeissoschizine from its iminium schiff base form to a tertiary amine to form demethylcorynantheidine, stereoisomers, and related side products. This reduction by DCS enzyme can stabilize the otherwise unstable and difficult-to-isolate dehydrogeissoschizine. Construction of Saccharomyces cerevisiae production of demethylcorynantheidine can be carried out via the expression of optimized STR, SGD and DCS genes that are designed and synthesized in techniques as described above. Alternatively, the set of genes can be integrated into the recombinant host genome by a single cross-over insertion event of the plasmid. The resultant strains can be verified by genome sequencing. Culture experiments where recombinant hosts were grown as described above and expressed DCS enzymes were performed by feeding secologanin and tryptamine as substrates (0.1-10 mM) during the fermentation and by monitoring the formation of demethylcorynantheidine and related intermediates that transiently form in the late-stage MIA pathway via LC-MS, HRMS, and/or LC-MS/MS. In FIG. 6, DCS activity is detected only in recombinant hosts expressing DCS and not in negative control strains. The DCS depicted are DCS_2n and DCS_3n genes encoding the DCS_2p (first from top in FIG. 6) andDCS_3p (second from top in FIG. 6) enzymes.

### Example 6-Biosynthesis of Demethylcorynantheidine, Corynantheidine, 9-Hydroxycorynantheidine, and Mitragynine

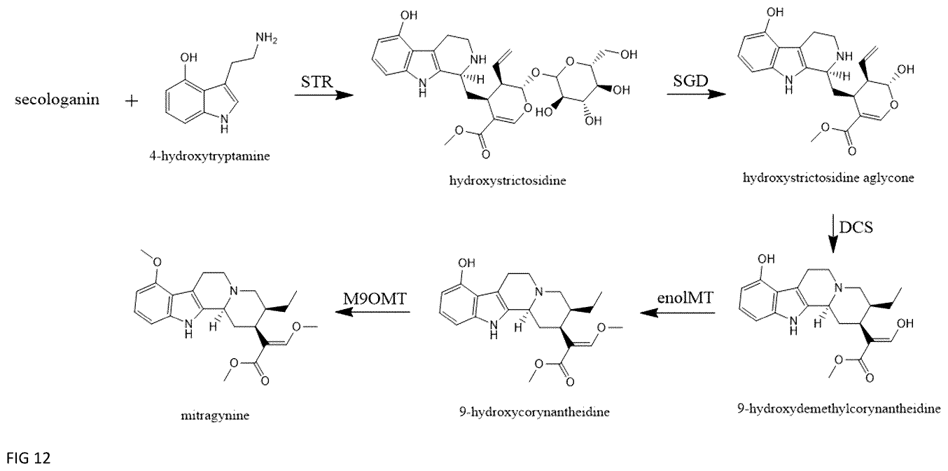

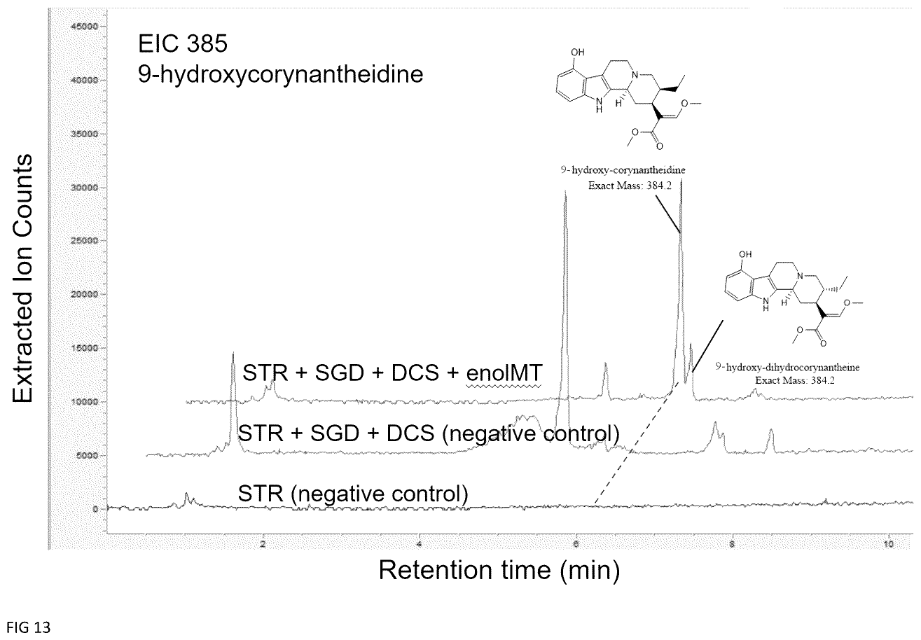

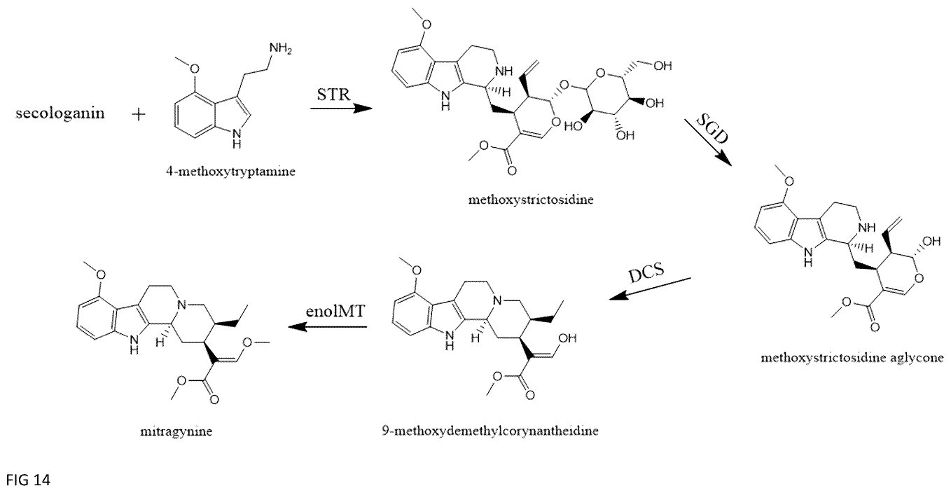

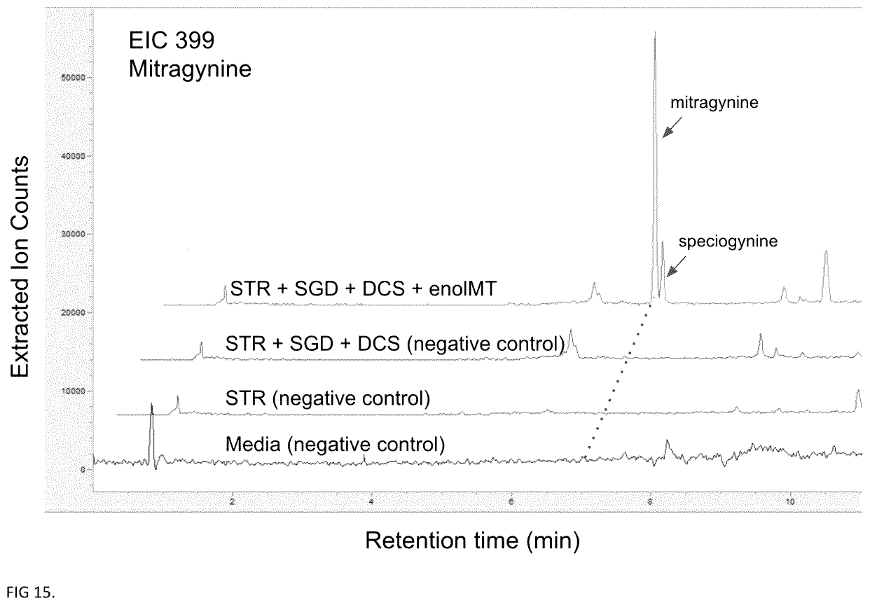

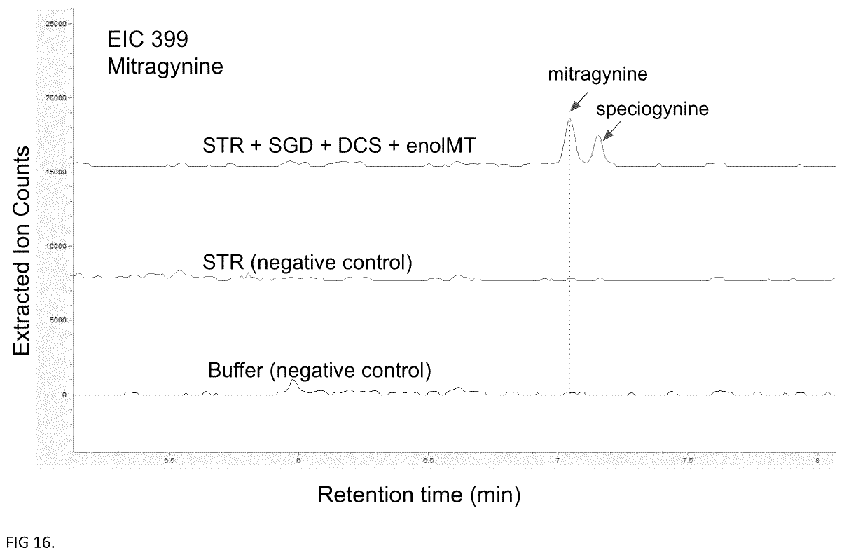

Biosynthesis of demethylcorynantheidine. The removal of the glycosyl moiety from strictosidine triggers a cascade of chemically labile intermediates starting with strictosidine aglycone to spontaneously form unstable 4-ring intermediates such as dehydrogeissoschizine and more stable shunt products such as 5-ring cathenamine and related alkaloids The DCS enzyme is incorporated into the recombinant cell to act as a NADPH-dependent reductase that iteratively reduces dehydrogeissoschizine from its iminium schiff base form to a tertiary amine to form demethylcorynantheidine and related side products. This reduction by DCS enzyme can stabilize the otherwise unstable and difficult-to-isolate dehydrogeissoschizine. Construction of a Saccharomyces cerevisiae strain for production of demethylcorynantheidine can be carried out via the expression of optimized STR, SGD and DCS genes that are designed and synthesized in techniques as described above, filling in the pathway depicted in FIG. 2. Alternatively, the set of genes can be integrated into the recombinant host genome by a single cross-over insertion event of the plasmid. The resultant strains can be verified by genome sequencing. Culture experiments where recombinant hosts were grown as described above and expressed DCS enzymes were performed by feeding secologanin and tryptamine (or tryptamine derivatives) as substrates (0.05-0.2 mg/ml) during the fermentation and by monitoring the formation of demethylcorynantheidine and related intermediates that transiently form in the late stage mitragynine MIA pathway via LC-MS. An example pathway using hydroxytryptamine is shown in FIG. 12. Example product formation from such a hydroxytryptamine substrate can be observed in FIG. 13. An example pathway using methoxytryptamine is shown in FIG. 14. Example product formation from a methoxytryptamine substrate can be observed in FIGS. 15 and 16.

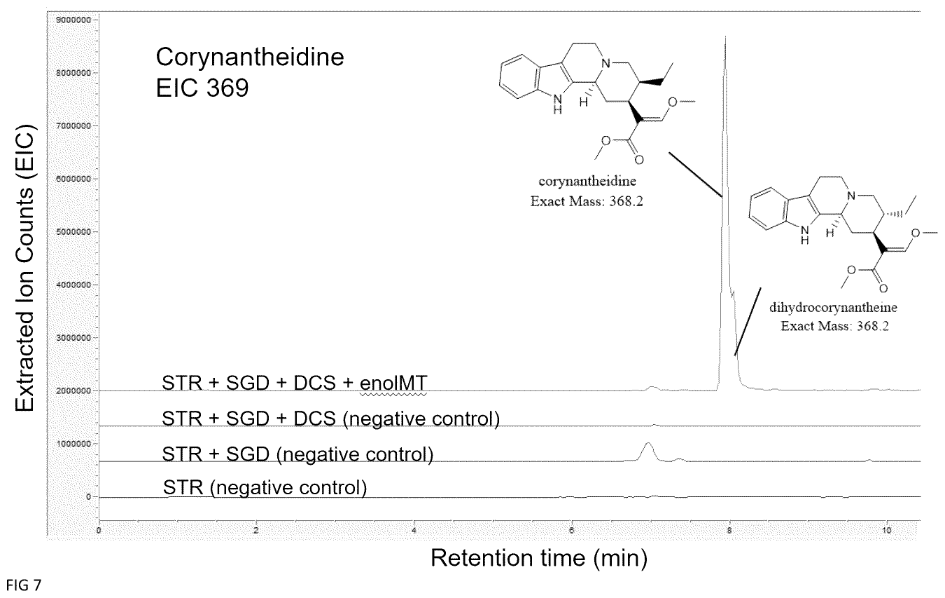

Biosynthesis of corynantheidine from demethylcorynantheidine. Generation of corynantheidine is achieved by incorporating an enol methyltransferase (enolMT) enzyme (e.g., either by integration into the host cell genome, or episomally incorporating it into the cell as described above) to methylate demethylcorynantheidine and form corynantheidine. Such biosynethtic reactions using a recombinant host are carried out as above. In FIG. 7, enolMT activity is detected only in recombinant hosts expressing enolMT and not in negative control strains. The enolMT depicted is enolMT_In genes encoding enolMT_Ip enzymes.

Biosynthesis of 9-hydroxycorynantheidine from corynantheidine. Generation of 9-hydroxycorynantheidine from corynantheidine is achieved by incorporating an indole ring hydroxylase (M9H) enzyme (e.g., either by integration into the host cell genome, or by otherwise incorporating it into the cell as described above) and optionally in the presence of an oxidase to hydroxylate corynantheidine and form 9-hydroxycorynantheidine. The generation of 9-hydroxycorynantheidine can also emerge from a previously hydroxylated product, which can result from feeding in or generating 4-hydroxytryptamine, whereby DCS and enolMT enzymatic activities yield 9-hydroxycorynantheidine and its stereoisomer from the hydroxylated precursors, as shown in FIG. 13.

Biosynthesis of mitragynine from 9-hydroxycorynantheidine. Generation of mitragynine from 9-hydroxycorynantheidine is achieved by incorporating an indole ring O-methyltransferase (M9OMT) enzyme (e.g., either by integration into the host cell genome, or by otherwise incorporating it into the cell as described above) to methylate the hydroxyl group at the 9-position of the indole ring of 9-hydroxycorynantheidine. The generation of mitragynine can also emerge from a previously methoxylated product, which can results from feeding in or generating 4-methoxytryptamine, whereby DCS and enolMT enzymatic activities generate mitragynine from the methoxylated precursors, as shown in FIG. 15 and FIG. 16.

In view of the above, it will be seen that several objectives of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification, including but not limited to patent publications and non-patent literature, and references cited therein, are hereby incorporated by reference. The discussion of the references herein is intended merely to summarize the assertions made by the authors and no admission is made that any reference constitutes prior art. Applicants reserve the right to challenge the accuracy and pertinence of the cited references.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The indefinite articles “a” and “an.” as used herein in the specification and in the embodiments. unless clearly indicated to the contrary, should be understood to mean “at least one.”

The phrase “and/or.” as used herein in the specification and in the embodiments, should be understood to mean “either or both” of the elements so conjoined. i.e., elements that are conjunctively present in some cases and disjunctively present in other cases. Multiple elements listed with “and/or” should be construed in the same fashion. i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”. when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments. “or” should be understood to have the same meaning as “and/or” as defined above. For example, when separating items in a list. “or” or “and/or” shall be interpreted as being inclusive, i.e., the inclusion of at least one, but also including more than one, of a number or list of elements, and, optionally, additional unlisted items. Only terms clearly indicated to the contrary, such as “only one of′ or “exactly one of.” or, when used in the embodiments. “consisting of.” will refer to the inclusion of exactly one element of a number or list of elements. In general, the term “or” as used herein shall only be interpreted as indicating exclusive alternatives (i.e. “one or the other but not both”) when preceded by terms of exclusivity. such as “either.” “one of.” “only one of.” or “exactly one of.” “Consisting essentially of.” when used in the embodiments. shall have its ordinary meaning as used in the field of patent law.

As used herein in the specification and in the embodiments, the phrase “at least one.” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example. “at least one of A and B” (or. equivalently. “at least one of A or B.” or. equivalently “at least one of A and/or B”) can refer, in one embodiment, to at least one, optionally including more than one, A, with no B present (and optionally including elements other than B); in another embodiment, to at least one, optionally including more than one, B. with no A present (and optionally including elements other than A); in yet another embodiment, to at least one, optionally including more than one, A, and at least one, optionally including more than one, B (and optionally including other elements); etc.

1. A polynucleotide comprising a sequence as disclosed in a Sequence Listing filed herewith, or a sequence having greater or equal to about 90% sequence identity to any one of said sequences.

2. The polynucleotide according to claim 1, having greater or equal to about 95% sequence identity to any of said sequences.

3. An expression vector comprising one or more polynucleotide sequences according to claim 1 and an operatively linked promoter sequence that allows for expression of the one or more polynucleotide sequences in a cell.

4. A recombinant cell comprising a polynucleotide according to claim 1.

5. The recombinant cell according to claim 4, wherein the cell comprises a bacterial cell, a fungal cell, or a yeast cell.

6. The recombinant cell according to claim 5, wherein the cell is a species of yeast that includes Saccharomyces, Candida, Pichia, Schizosaccharomyces, Scheffersomyces, Blakeslea, Rhodotorula, or Yarrowia.

7. The recombinant cell according to claim 5, wherein the cell is a species of a filamentous fungus that includes Aspergillus or Penicillium.

8. The recombinant cell according to claim 3, wherein the cell is a species of bacteria that includes Escherichia, Corynebacterium, Caulobacter, Pseudomonas, Streptomyces, Bacillus, or Lactobacillus.

9. The recombinant cell according to claim 4, wherein at least one copy of the polynucleotide sequence or polynucleotide sequences is located on at least one plasmid.

10. The recombinant cell according to claim 4, wherein at least one copy of the polynucleotide sequence or polynucleotide sequences is stably integrated into the genome of the cell.

11. The recombinant cell according to claim 9, wherein a plurality of copies of the polynucleotide sequence or polynucleotide sequences are stably integrated a plasmid or into the genome of the cell.

12. The recombinant cell according to claim 4, wherein the polynucleotide sequence or polynucleotide sequences are the same sequence.

13. The recombinant cell according to claim 4, wherein the polynucleotide sequence or polynucleotide sequences are different sequences.

14. The recombinant cell according to claim 4, wherein the polynucleotide sequence or polynucleotide sequences encode the same class of enzyme or amino acid sequence.

15. The recombinant cell according to claim 4, wherein the polynucleotide sequence or polynucleotide sequences encode a different class of enzyme or amino acid sequence.

16. A method for the biosynthesis of a downstream monoterpene indole alkaloid compound, or a precursor or metabolite thereof, the method comprising culturing a recombinant cell of claim 4 under conditions that allow for the biosynthesis of the compound.

17. The method according to claim 16, wherein the method further comprises contacting the recombinant cell with a feed comprising one or more exogenous substrate compounds, wherein the one or more exogenous substrate compounds comprise strictosidine, a strictosidine
analog, corynantheidine, or a corynantheidine analog, or the feed comprises a plant extract that includes the one or more of the exogenous substrate compounds.

18. The method according to claim 16, wherein the one or more exogenous substrate compounds are produced by a recombinant cell.

19. The method according to claim 16, wherein the method generates one or more strictosidine, hydroxystrictosidine, methoxystrictosidine, strictosidine aglycone, hydroxystrictosidine aglycone, methoxystrictosidine aglycone, demethylcorynantheidine (20R, 20S), 9-hydroxydemethylcorynantheidine, 9-methoxydemethylcorynantheidine, corynantheidine, dihydrocorynantheine, 9-hydroxycorynantheidine, hydroxydihydrocorynantheine, speciogynine, and/or mitragynine, among others.


SEQUENCE LISTING (369 sequences)

SEQ ID NO: 1 - DNA, 1101 bp, synthetic construct
  source 1..1101: mol_type = other DNA
atggcagccggtaagagccccgaggaggaacatcccgttaaggcttatggttgggcagtgaaagacagaaccaccggtatcctgagccctttcaagtttagcaggagggctactggggataacgatatccgtattaaaatattatattgcgggatatgccatacagacctaacgagtgttaaaaatgagtatgagttcctatcttatcccttggtgccaggtatggagattgtcggcattgccacagaggtcggatctaaagttaccaagattaaagtcggtgaaaaagtcgcagttgcagcgtatttgggaacatgtggtaaatgttataactgtgtcaatgacctagagaactattgtcccgaagtcataatagggtatggtacgccctaccacgatggtacaataaattatggtggactgagtaacgaaactgttgtgaatgaaaggttcgtattacgtttccctgagaagttgtcccccgctggcggagcgccgctgttgagcgcaggtattacagcgtatagcgcaatgaggaatcatggactggataaaccgggcattcaattgggcgtggtcggccttgggggtctaggacatttagccgttaagtttgcaaaagcctttggggtgagggttacggttatttcaacaacaccatcaaagaaggacgaggcaatcaacaatcttggggcggacgcttttctattttctcgtgacgacaaacagatgagagctgccataggaacatttgacgctataattgacacgttagcggtggtccatcctatcgctccgcttttggaccttttgagatcccacggaaaacttgttctggtaggggcccccagcaaaccactggaactgcctaccatcccattgttatcaggcggtaagtctttaataggaagtgctgctggaaatgtgaagcagacgcaagaaatgctggacttcgccgcggaacatgatataactgctaatatagaagttatacccatagactatataaatacggcgatggagaggctagataaaggggatatcagatttaggtttgtagtagatattgaaaacaccttgacgccgccgccggaaccttaa

SEQ ID NO: 2 - DNA, 1065 bp, synthetic construct
  source 1..1065: mol_type = other DNA
atggcagctggaaaaagtcaagaagacggtcagacggttaaggctcttggatgggcagcaagagaggtgtctggggctatatcacctttcgacttctcacgtagagcacctggggaaagagacgttcaagttaagatactttattgtgggatttgttctttcgacaccgaaatgataaacaacaagttcgggtttacacgttatccgttcgtcctaggacatgagatcgtcggcgtcgtatccgaagtgggaaggaaggtacagaaatttaaaataggagataaagtgggagtaggcacaatgataggttcctgccgtacgtgttacagttgtacacacaacttagaaaactattgtccgaaggtgactcttaccgaggctactagcggcggatgctctaatctggttattgcagacgaggactttgtgttccactggccggtcaacctgccattagacctgggcgcccctctactgtgcgccgggattactgtttactcacctttgaaaaattttgaattagataaaccaggacttcgtattggggtcgttggcttaggcgggataggtcacatagcagttaaattcgcaaaggcatttggcgcaaaagtaaccgtaatctcctcctcagagagcaagaaagtagaggccattgagaaatacggtgcggactcattcttggtctcttccgatcctggtcagatgctggccgccgcagggacgctagacggagtaatcgacacagtccccgctccacattcaattttaccctttttagatcttcttttaccgagaggaaagttaatcatcttgggggcgccgatggaacccttcgtcctaccgatctaccccctactgcaaggcggacgtgtagtcgcaggatctgccactggcggtttaaagcagatacaggaaatgttacatttcgcggcggagcataacattgtggccgacggagaagttattccgattgatgacatcaataccgcaattaagaggattgaaaaaggagatgttaaataccgttttgtcgttgacataggaaatacactgaaatctgcgtaa

SEQ ID NO: 3 - DNA, 1065 bp, synthetic construct
  source 1..1065: mol_type = other DNA
atggcagcggggaagtgtgctcaagaggagcacacggtcaaggctttcggttgggcagcgcgtgaggcaagcggtgcacttagtccgtatggtttttccagaagggctacaggggaaagagatgtcagagtgaagatattgtactgcggaatttgtaggacggatgccgaaatgatctcagacaaattctgtttcacaaaatatccccatgtccctgggcatgagatagtaggagttgtgtcagaagtgggaaataaggtgcagaagttcaaggtcggtgctaaggtgggggtaactggaatcatcgggtgctgtcgtacatgttatagttgtactaacgggttggaaagttattgtccaaacgtggcattgactgaggcaggggaagggggctgctcaaacttcatagtcctggatgaagactttgtctttaggtggcccgaaaaattaccactggatctgggggctcctcttctttgtgcaggagcggcatcttatagtccgttgaagaacttcgggcttgacaaacctggtcttcatataggtatagctggccttggcggaatgggccacgtggcagttaaatttgcaaaggcttttggtgccaaagtaacagtaatttccacatccgacaataagaaagaggaagcgatcaagaaatatggtgcggatgcctttttaaacagttccaaccctgaacaaatgcgtgctgctgccggtaccttggctgccatcgtagacactataccgtcaccccatagtcttgtgccgctactggatttactactgccgcacggcaaagtgatcgttttgggggctccgagtgaaccatttgtactgcctgttatgccgttattacaaggaggcagggtggtagctggaagttctggagcttcgttaaagcagattcaagagatgctagatttcgcagccgaacacaacattgttgcagacgccgaagtcatcccaatagattacatcaacactgccatcaagaggattgaaaagggggacattaagtaccgtttcgttgtggatataggtaataccctgaaaagtgcataa

SEQ ID NO: 4 - DNA, 1467 bp, synthetic construct
  source 1..1467: mol_type = other DNA
atggcatatacacctgagaatagtagttactcatccatccttaacacctatatacaaaactcaaggttcatttctccggaagcccctaaacccaaggtaattctgactccggtacacgagtcccaggtacagacggccatctattgcagtaaaagacacggaatgcaaatacgtattaggtccggtggacacgactttgagggcagctcatacatgtccaacgtccccttcttagtcttcgatatgtttaacttcaggtctatttcagtagacctgagatcaaaaacggcttgggtaggagccggtgctactctgggagagacttattataatatttatcagaaaaattccacgctaacatattccgctggatactggcccactgtaggcataggaggccatgtgtctggtggtggatgcgggccgttggtcagagaatacggcttggcggcagataatgttctggatgcgcgtctaattgacgtcaaaggcaggatacttgatcgtaaatcaatgggtgaggatttattttgggcaattagaggaggtatgggtgctaattttggtgtgattttagcttacaagataaaacttgtggaagtgccagagaaggtagtagcattcaaggtaaggagaacattagtgcagaatgcaacaaaattagttcataaatggcagttcgtcgctccgaagctgccagtgaggttgataatatcccttcagatcgcctccattaatagcgaccagacgggaggaagaacaattcaggcgtcctttgcctctgtgtttcgtggggaggtggacgagcttctacagatcatggaggagcacttcccagaattgggccttaaaaaggaagacatcaaggagatgagctggatacagtactttagtttctatggtgatgagccaatagatgatgtagcccacttcttaacaaatagggtctcaacaacaaagccttatttcaaagcaaagagcgattttgttcaaaagccgatcccagaggcaggcatcgaaaagatttggaaaaagctgttcgagaaagatcctttggcagctcagttgcagtggaccccgtttggaggaaaaatggatcaaatatcagactccgagaccccattcccacacagggcaggcaactcattcatcatgtttgaggtgatcttctggaacgggaccgacaaggtactggcgcagaagcacataaactggatcagggaactttacgatgttataggtcagtatgttgacaacaatcccaggggagcatatgccgactacagagacctagaccttggcgtgaataacattgatgagacctcagctgaacaagcaagggtgtggggtaaatcttacttcaaagataattttgatagactggtaaatgtgaaaaccagggtggacccgaataatttctttaagaatgaacagagcattccaccaagactagcccatagcagctca

SEQ ID NO: 5 - DNA, 1611 bp, synthetic construct
  source 1..1611: mol_type = other DNA
atggcagccatgcttagaacaaggatgctacagttcgccttcgttttgttattcagcttctgtggggtaagttcatcaagtcagccagaaaccccgtttctgcaatgtttattccaccacagcgatgactccaccattggttatattatttacacaccggaaaactccagttatagctcaatcctgaactcatgcatccagaactcccgtttcatagttccagagacgccaaaaccgaaagtcattctaactcccgtcagcgagagccaggtacaagctgcaatttactgttccaagaagcatggaatgcaaattagaatccgtagtggcggacacgatttcgaaggatcatcatacacatctaacgtgccgtttcttgtccttgacatgttcaactttagatctatttccgttgacttacaaagcaaaactgcatgggttggcgcaggagccacgcttggagagacatattaccatatctaccaaaaaaactctaccttaacgtactctgcgggttactggccaactgtagggataggtggacacgtctctggtggtggttgtggaccccttgtacgtgagtatgggctggcagcggacaacgtcttggatgcgaggttagtcgatgtgaagggcaggatcttagatcgtaagagcatgggcgatgacctgttttgggctatacgtggtggtatgggagcgtcctttggcgttatacttgcctacaaaataaagttggttgaagtacctgaaaaagtcgtggcttttaaggttcgtaggacacttgaacagaacgcaaccaagctagttcacaagtggcaatttgtggctccgaaactaccagttcgtcttattatcagcctgcagattgcatccataaactcagatcaaactggaggaagaaccatacaggcctccttcgcttccgtctttcgtggaggagtcgatgagcttttccagataatggaggaacactttcctgaactgggattaaagaaagaggatgtaaaggaaatgagttggatacaatatttcagtttctatgccgatgaacccatagatgacgtagcgcatttcctaactaatagagtcagcaccaccaaaccatacttcaaagccaaatccgattttgtccagaaacccattcctgaggctggcatagaaaaaatatggaagaaactatttgaaaaagatcccttggctgcacagctgcagtggactcctttcggaggtaaaatggaccagatctccgagtcagaaactcccttccctcacagagcgggtaactccttcattatgttcgaagtcatattctggaatggcactgataaggtcttagcgcagaagcacataaactggattagaaaattatatggtgtaatcgggcagtatgtggacaacaaccccaggggtgcctacgcggactacagagatttggacttgggtgtcaataacattgatgagacaagtgtggaacaggcaagggtatggggtaagagttacttcaaaaataattttgatcgtctagttaatgtgaaaaccagggtagaccccgataacttcttcaaaaacgagcaaagcattccctctagattggcgcatagttcattt

SEQ ID NO: 6 - DNA, 1611 bp, synthetic construct
  source 1..1611: mol_type = other DNA
atggcaactatgcttcgtaccaggatgctacagttcgcgtttgtcttattgatttctttctgctgcgtctccagtagtagtcagccggagatcccgttcctgcagtgccttttccaccactcagacgacagcaccatcgggaatgtaatgtataccccagaaaatagttcatacagtagcatacttaacacttacattcaaaattcccgtttcataagtccagaagctccaaaacccaaggtgattttaacacccgttagggaatcccaagtccaaacggccatatactgtagcaaaaagcacgggatgcagattaggatacgttcaggaggccacgacttcgaaggttcatcctatatgtcaaacgtaccttttctagtattggacatgttcaattttagaagcatctccgtcgatctgaggagtaaaactgcatgggtcggtgcaggagctaccttaggcgaaacatattataacatatatcagaaaaactccaccctaacgtactcagccgggtattggccgaccgtaggaatcggcggtcatgtgtctggaggtgggtgcggaccccttgttcgtgagtacgggttagcagccgacaatgtcttggatgcgagattaatcgacgtaaaggggaaaatcctagaccgtaaatctatgggagaggatttattctgggctatacgtggtggaatgggtgctaacttcggagtaattttggcgtataagattaagctggtggaagttccagaaaaagtggtcgcttttaaagtaaggaggactcttgtgcagaatgccacgaaattggtacataaatggcaatttgtggctccgaaacttcccgttaggctgatcatttcactacaaatagccagtattaactctgatcaaacaggtggacgtaccatccaagcgtcatttgcctctgtctttagaggtgaggttgacgaattgctgcagattatggaagagcatttcccggaattgggtctaaagaaggaagacatcaaagagatgtcctggatacaatacttttcattctatggagatgaaccaattgatgatgtcgcccattttttgacaaacagggtgagtacaacaaaaccgtatttcaaagctaaatctgactttgttcaaaagccaataccggaggctggaatagagaaaatatggaaaaagctgtttgaaaaagacccactggcggcacagttacagtggactccgtttggcggaaaaatggatcaaattagcgacagtgaaactccctttcctcacagggccggtaattcttttataatgtttgaagtaatcttctggaatggaaccgacaaagtgttggcacaaaagcacattaattggattcgtgaactatatgacgttattgggcaatacgttgacaataatcctaggggagcatatgcagactaccgtgacctagacttgggagtcaataacattgacgagacgtctgctgagcaagcgagagtctggggcaaaagctacttcaagaataacttcgacaggttggttaatgtgaagaccagagtagatccgaataatttctttaagaatgaacagtcaatacctcctaggttggctcactcttcaagt

SEQ ID NO: 7 - DNA, 1059 bp, synthetic construct
  source 1..1059: mol_type = other DNA
atggcagctgggaaatgcccgcaagaggagcaaactgtgaaggcattcggatgggctgcacgtgaagtatcaggagcactgtcaccttacgggttctcccgtcgtgccacgggtgaacgtgacgtaaaaattaatgtactttactctggcatgtgtaatttcgatactgagatgataaataataagtttgggttcacaaagtatccatacgtgttgggacatgagactgtcggagtggtgtccgaagttgggtccaaggtccgtaagttcaaagttggcgataaggttggcgttggcggtctggtgggctcatgccgtaagtgctacagctgcactaacgaccttgagaattattgtactaaggccgaattgaccgaggcaggagccggtggatgtagtaacttcgtagtaacggatgaggatttcgtattccattggcctgaaaagctgcccttagaccttggagcacctctactttgcgcaggagttacaacattctccccactaaagactcacggtctggacaaacccggaatgcacgtcggagtggcaggattaggcggtctaggccatgttgcagttaaatttttaaaggcattcggagcaaaggtaaccgttattagtacctctgacaacaagaagagggaagcgatagaaaagtacggagccgacgccttcttagttagcagcaacccggaacaaatgcaaaccgctgctgggacactgacaggaattatagacaccataccgtctgcgcactcccttgttccccttttaaacctgctgatgccgcaagggaaattgattgtcctaggggtccctaccgaaccgtttgtgctgcctgtagtgccaatgatccaaggaggtaaagtcgtggctggatcttcctttggcggtttaaaactaatacaggaaatgcttgatttcgcagcagaacataatatagttgctgacgctgaagtaatacctgtggattacatcaacacagctattaaaagaatagagaccggagacatcaaatatagattcgtcatcgatgtgggcaacacgctaaaaagt

SEQ ID NO: 8 - DNA, 1080 bp, synthetic construct
  source 1..1080: mol_type = other DNA
atggcagccgagaaatctccagaggaagaacaccctgtaaaggcgtttggccttgctgccaaagacagttccgggatacttagtccgtttaacttctccaggagggctactggtgatcatgatgtccagttaagagtgctttactgcggattgtgttattacgatacgaagatgataaagaataagaggggtgtcacgcgttatcctttcgttttcggccacgagattgtgggagaagtgaccgagatcggcagagaggtacagaaattcaaagtgggcgataaggtaggggtaggttgtatggtagcaagctgtaggtcctgcgaatcatgtgcaaataactgtgagaactactgccccaatgtgtctgtgacggatggggccttcttctttaaaacaggggaggttctatacggaggctgttctgacatcatggtagccgatgagaattttgtgatcaggtggccagaaaattttccactagacgccggtgctccgttgttatgcgctggtatcacaacatactctcctctaaggaattttgggctggacaagcctggaatccacgtaggcatttacggattgggtggcttgggccacgtagccgttcaatttgcaaaagcattcggtgcgaaggtgacggtaataagcagtagtgacaggaagaggatggaagcgattgaaaaattaggcgcagacagtttcctggtaaatagtaatctggaagaaatgcaggcggctatgggcaccatgcacggcattattgatactgtgccagctaatcattctctagtccccttattggatcttctgaaaccacaagggaaattgatcgtcgtaggtggacccgagaagccgttcgagcttcctgtgttccctctattgcagggtggcagattggtagcgggaagcgcgacgggtggaattaagcaaacacaagaaatgatcgacttcgctgccgagcataacattcttccacacgtagaagtcgtaagtgtcgattatgtgaacaccgcgattgaaaggaccgagagaggcgatgttaagtatcgtttcgtgatagatattggaaatacactt

SEQ ID NO: 9 - DNA, 951 bp, synthetic construct
  source 1..951: mol_type = other DNA
atggcagccgaaaagtccccagaggaggagcagacggtcaaggccataggctgggcagtacttgaggctgcgggtgtcctgtctccatttgagttcagcagaagagcaactggagaacgtgatgtacaggttaaggtcctttactgcgggatctgcaacttcgatactgagatgattaacaacaaattcggctttactcgttatccctttgtcttgggctgcaccaataacctggagaactactgtcccaaagcagtacttattgaagctaacaatggaggttgttctaacttagtggtggcagacgaagatttcgtatttcattggccagaaaagctgcccttggatctaggcgctcccttactttgtgcaggcattacgacatattccccactaaagaattttggattggataagcccggactacacgtgggggtagcaggcttgggtggcttgggtcatgtagccataaagttcgcaaaggccttcggagcgaaggtgacggtcatctcatcatctgacaagaagaagaaagaggctatagaaaagtacggggctgatgagtttctagtgagtagtaatcccgagcaaatgcaagctacggcaggaacactaaatggtataatcgacacggttccgtccccgcactccttagtgccgttacttaatctgctgcttccccagggaaagctaatagttctaggtgctcccatcgaaccttttgtgttaccgatctaccccttgttacagggtggtagggtcgtggctggttccgccacgggaggtctaaagcaaattcaagaaatgcttcatttcgcggcagagcattccatagctgctgatgcagaggttatacctattgactacatcaatacagctgtcgagcgtatagaaaagggagacattaagtacaggtttgttgttgatataggcaatacactaaagtcagca

SEQ ID NO: 10 - DNA, 1080 bp, synthetic construct
  source 1..1080: mol_type = other DNA
atggcagctgagcaatccccacaggaccagcaccaggttaaagttctaggttgggctgctagggatagttccggtatcctatccccgttcaaatttagcaggagagcaaccggcgagcatgatgtgcagctaaaggttatctattgcggaatttgcacttatgacgtcgagatgatcaagaacaagtgggggatgacaaattacccagtagtttttggacatgaagccgtgggtgtggtgacggaaatcggctccaaggttcagaaattcaaggtgggggatacagtcggtgtaggaactcaagtcggtagctgtggggaatgcgactgttgcaccaacgacttaaatcagtattgtcctgaaatgattgcaacggatggcttcggttctaatgatgtgagttgcggcgcatgctgtgacataatggtagttgatgaaaatttcgttatcgtatggcctcagaaccttccaatggatgctggagcacctcttctgtgtgcgggtattaccacttacagtccccttcgtaactttggtctggacaagccgagaatacatgtggggatcgttggacttgggggtctaggtcatgtggcagtgagatttgccaaagcgttcggagccaaagttaccgttatatctacatccatgaataagaaacaagaggctttgcataaatatggcgctgacagctttgtagtcagttccgaacaagaacaaatgcaagccgcggtctcaacaatgaacgggatcatcgacacgtcacctgtgcctcatgccctagtgccccttttgaagctattgaaaccgcagggtaagttgatattgattggagcaccggacaaaccgtatgagttgcctgtgttccctctaatagaaggtggccgtatcgtggcaggtagcgctaccgggtcattcagacagacacaagaaatgatagatttcgcagctaaacacaatattgtagcagatgtcgaggtaatccctaccaattatctgaatactgcggtagaacgtattgagaagggtgatgttaaatacagatttgtcatcgacttgggaaacggacttaaactagca

SEQ ID NO: 11 - DNA, 1062 bp, synthetic construct
  source 1..1062: mol_type = other DNA
atggcagctgggaaatgtgcacaggaagagcacacagtaaaagccttcggttgggcagcaagagaagccagcggtgcacttagcccttatggattttcccgtagagcgacaggagaacgtgacgtaagggttaagattctgtattgcgggatctgcaggacagacgccgagatgattaacaacaaatttggctttaccacctacccgtttgttcttgggcatgagatagtgggcgtcgtatccgaggtcggatcaaaggtgaagaaatttcagttcggtgataaagttggagtggggacgatgattggagcttgcagaacctgttacagctgcactaataatttagaaaattattgtccaaaggcagtcctaatagaggccaacaatggaggatgtagcaatttggtcgtcgctgacgaagatttcgtatttaggtggcctgagaagctgccattagatcttggtgctccgctgctatgtgctggtatcacaacctactccccgttgaaaaacttcggattggacaaacctggactgcatgttggcgtggctggtttgggaggtttaggtcatatagcggtcaagtttcttaaagcgtttggtgcaaaggtgactgttatctcaagctctgataagaagaaaaaggaagctatcgaaaaatactcagccgatgagtttttggttagttccaatcccgagcagctgcaggcggctgctgggactctaaatggtattatagatacagtcccctcaccgcattccttggtgcccttcttgaacttgctgcttcctcagggaaaattaatagttttgggtgccccaattgaaccattcgttttgccaatcttcccattgctacagggaggcagagttgtcgcgggcagcgcaacggggggcctaaaacaaatccaagaaatgttattgtttgcggcggaacacaacatagcagctgacgcagaagtgatccccatagattacattaacactgcggtagaacgtatagagaagggggacatcaagtataggttcgtggttgacatacgtaatacattaaaaagtgca

SEQ ID NO: 12 - DNA, 1086 bp, synthetic construct
  source 1..1086: mol_type = other DNA
atggcagctggcaagactccagagactgttcacccggtaaaggccttcggttgggctgctcgtgatactagcggggaactgagtcctttcaagttctcaagaagagcaacgggtgaacacgacgtacaattcaaggtcctgtactgcggaatatgccactcagaccttcatatgatcaagaatgagtggggaatgacagtatatcccataatccctggtcacgaaattgttggtgttgtcaccgaagtgggtaacaaagtggaaaaggtaaaagtgggagataaggtcggcgttggatgtctggtaggttcctgcggcaactgtaatatgtgcagtcaggacctagaaaactactgcccgaaacagatcctgacttactccgcgatcgacacagatggtagcataacgtacggaggctattccgacgtgatggttgccaatgagcactttataatcagatggccggaaaacttacctctagacgcgggtgctccactactatgcgctggcattacaacatactcacccctacgttacttcgggcttgataaaccaggcatgcacgttggtatcgtagggctgggtgggctagggcacgtcgctgttaagttcgcaaaagcattcggttctaaggttacagtgatctcaacaagtgagaataagaagaaggaggcgatagaaaaacttggcgctgacgctttcttaaacagcagagatccacaacaaatgcaggcagcggtcggtacattcgatgggatcttggacactgtctctgctgtgcacccgatcgtaccactgatcaacctagttaaatctcaggggaaagtaatcatggtcggggctcctgaaaagccattagaattggcgacatttccgttactagtcggaagaaagacgctaagcggcagtgcaataggtggactgaaagaaacacaagagatgatagatttcgctgccaagcacaacatcttggccgatgtggaagtaatacctatgaattacgtgaacaccgcgatggagcgtttgttgaaggcggatgttaagtaccgtttcgttattgatataggtaacagtcttaaatcagct

SEQ ID NO: 13 - DNA, 1086 bp, synthetic construct
  source 1..1086: mol_type = other DNA
atggcagcaggtaaatgcgcacaagaagagcacccagtcaagagctttgggtgggctgcacgtgatacgagcggcatcctgagcccttttaagttctctaggcgtgccactgatgagcacgatgtccgtttgaaagttctttattgtggaatatgtcactcagacttacacaccatcaaagacgagtggggaattacggagtatccggttgtgccaggccatgagatagctggcgtagtcactgaggtaggtagcaaggttcagaagttcaaggtcggcgacaaagtcggagttgggtgttttgtaggctcttgtcgtaattgcaacatgtgtgataagaatttggaaaattactgcccagcgcagatatttacatatggtagcccgtacacggatggcactccaacatatggaggctattctgatatgatggtagtagacgaacactttgttattagatggcctgaaaattttccgctggacgcaggagcgcctttgctatgcgcaggcattaccacgtactccccacttagatacttcgagctggataagcctggcatccatataggcattgtgggacttggtggcttaggtcatgtagctgtgaaatttgctaaggctttaggggctaaggttaccgttattagcacatctgccagtaaaagacaagaggcatttgaaaagctcggagcagacagctttttactgagtagcgaccctgagcagatacaagcggctgttggaaccatggatggtataatagatactgtgtcagcggtgcatccgatagtgcccttgattgatctgttggctcctcatgggaaactaatagtagtgggttctccagccaaacccttggagctaccgatttataatctgagcataggccgtaagatcgtaggagcgtcctccgttggagggcttaaagagacccaagagatgatcgactttgctgcaaagcataacatacttccggacgtagaaataattcccatcgactatgtcaatacagctatggagagattagctcgtggcgatgtgaaatataggttcgtgattgatgtaggtaataccctaaaatcaacc

SEQ ID NO: 14 - DNA, 1086 bp, synthetic construct
  source 1..1086: mol_type = other DNA
atgtcatccgcagagaaaagccccgaagaagagcacccggtcaaggcatttgggctggctgccaaagattcctctggaatattgagtccattcaacttttctagaagggcgaccggggaccatgacgtccaattgagggttctttactgcggtctgtgctactatgatacaaaaatgattaagaacaagaggggagtaacgcgttatccattcgtattcggacatgaaattgttggagaggtcacggaaatcgggcgtgaagtccagaaatttaaggtaggggacaaagtgggtgtgggatgcatggtagcctcatgccgttcctgtgaatcttgtgcgaacaactgcgaaaactattgtcccaatgttagcgttacggatggtgcattcttttttaaaacgggggaagttctgtacggaggttgcagcgatattatggtagcggatgaaaacttcgtaatacgttggccggagaactttcctcttgatgccggtgctccgctgctttgcgccggcattactacatactccccgctgcgtaattttggtttagataaacctgggattcacgtcggcatttatgggttaggcggattaggtcatgtggcagtccaatttgcgaaagctttcggcgctaaggtaacagtcataagttccagcgaccgtaagagaatggaggcaatcgaaaagttaggtgccgacagttttcttgtaaactccaatttggaggaaatgcaagcggccatgggaaccatgcacggtataatagacactgttcccgctaatcattctctagtccctctgctggaccttttgaagccgcagggcaaactaattgttgttggcggccccgaaaagccttttgaattacctgtatttcctctattgcagggcgggaggcttgtggcgggtagcgccactgggggcatcaaacaaactcaagaaatgatagacttcgcggctgaacacaacatattgccccacgtagaagtggtaagtgtagattatgtgaacacggccatagagcgtacagaaagaggcgatgtgaagtacagattcgtgatagacattggtaacacgttgtat

SEQ ID NO: 15 - DNA, 1086 bp, synthetic construct
  source 1..1086: mol_type = other DNA
atgtcatccgccaagacgccggagatggtgcatccagtcagagctttcggatgggcagccagagatacttctggagaattatcccccttcaagttctcaagacgtgcgactggggaacacgacgttcaatttaaggtcctatactgcggcatttgtcattctgatctacacatgataaaaaacgaatggggcatgacagtatatcctattgtacctgggcacgaaatcgtgggggtagtgacggaggtggggtcaaaggtcgaaaaggtcaaagtaggagataaggttggtgtcggatgtcttgtaggatcatgtgggaactgcgatatgtgcagtcaggacttggaaaattattgtcctaaacaaattttgacttacagcgccatcgacgaggatggatcaattacatatggcgggtatagcgacgttatggtagccaacgagcacttcatcatcagatggccagaaaatttgcctttggatgcgggcgctcccttgttatgcgcaggcatcaccacatattccccattaaggtactttggactggacaaaccagggatgcatgtcggtatcgttggacttggtggtttaggtcacgtcgctgtgaagtttgcgaaagcattcggcgcgaaagtgaccgtgatctctacttccgaaaacaagaaaaaagaagcgatcgaaaagctgggagcggatgcgtttttaaattccagggatccacaacaaatgcaagcggcggtaggcacttttgatggcatcctggacactgtatcagcagttcacccgattgtcccgctgattaacctagttaagtcacaaggtaaagtaataatggtcggggcacccgagaagcctttagaactggcaactttcccattattggtcggcagaaaaacattggcggggtcagcgataggcggacttaaagaaacccaggagatgatcgattttgcagctaagcacaatattttggcagaggttgaagtgatccctatgaattatgttaatacagcgatggagcgtctactaaaagccgatgtcaagtatagattcgtgattgacataggaaactctttaaagagtgct

SEQ ID NO: 16 - DNA, 1086 bp, synthetic construct
  source 1..1086: mol_type = other DNA
atgtcatccgcgaaaacacctgggactgagcattcagttaaagcattcgggtgggccgcgagggatacaagtggtgagttaagcccgtttaaattctccaggcgtgaaactggcaaacacgatgtgcaatttaaggttctgtattgcggaatatgtcactcagacttacatatggtaaagaatgagtgggatatgacccgttaccctattgtacctggccatgagatagtgggagttgtgacggaagtgggcagtaaagttgaaaaggtaaaggtgggggacaaagtaggggtcggctgcctagtcggcagctgcagaaactgcgagatgtgttctcaagacctggagaactattgtgcaggccagatccttacttactccgcgatatacaccgacggcactataacatatggtgggtattctgatctaatggtcgcggacgaacactttgttatacgttggcctgagaacttccccttggacgcgggtgcgccccttttatgcgcaggcataacaacctactcacctttgagatattttgggctagataaacccggtacccacgtgggcgtggtaggacttgggggtttaggccacgtagcagtgaagtttgcgaaggcgttcggggcgaaggtcaccgtcatttctacgagcgaaaacaaaaagaaagaggccatcgagaaattgggtgctgacgctttcctggttagtcgtgatccggaacagatgcaggcggcaatcggtacaatggatggaataattgacaccgtgagcgccgtgcacccggtagtaccgttgattaacctagttaaaagccagggtaaaattgtgatggttggtgctccggagaaaccccttgaattcgctacttttccgttaatagttggaagaaaaactctatcaggtagtgctataggaggcctaaaggaaacgcaggagatgatcgattttgcagcgaaacataatatattagctgatgtagaagttattccaatcgattatgtgaacactgcaatggatagactattgaaggcagatgtgaagtatcgttttgtaattgacataggtaactccttgaaaagcgca

SEQ ID NO: 17 - DNA, 1089 bp, synthetic construct
  source 1..1089: mol_type = other DNA
atgtcatccgctaagagttttgaagaagaacaccctgttaaggcctttggttgggctgcccgtgacagcagcggaatcctgagcccgtttaaattctccaggagggcgacgggtaagaaggatattagattcaaagtgctgtacactgggatctgtcacagcgacctacaccacctgaagaacgattggggctcaagtaagtaccctcttgtaccgggacatgaaatagcaggtatggtaactgaagtggggagtgaggtgaagagctttaaagttggagataagatcggggtaggctgcttgattggttcatgtgggacctgcgaggactgtgccgatggactagagaactattgccctaagaagatcctaacatacgatggtgccaaatactacgacggcacaatgactttcggcggctattccgatcacatgatcgtggacgagcattttgcaatccattggccagaaaacatgcccctagacgggggtgcacccttgctttgcgccggaatcaccacttattctccgatgaagtacttcggcttggacaaaccagggttacatataggagtcgtgggtttaggcggcctgggccatgtagcggtgaaattcgcaaaggcctttggtgctaaggttactgtaatctcctcaactccaggcaagaaggaggaagcgattaaaaactttaaggctgattcatttctggtaagccgtgacacagcagctatgcaagccgcagccggcacattacatgggatacttgacacagtcagcgccgttcatcccatcctaccattactgagccttttaaagaatcatagtaaatacattatgcttggcgcaccggagaaaccattagagctaccagtctttccaatgcttatgggcaggaagatcgtcgcagggagcaacatcgggggcgtgaaagaaactgaggaaatgcttaacttcgccgcagagcatggaataacagctgatgttgaggtaatcccaatcgactatgtcaacactgcggtcgaacgtctgggtaagaatgatgtgagatataggttcgtaattgacgttggtaatacactgaaagcgact

SEQ ID NO: 18 - DNA, 1065 bp, synthetic construct
  source 1..1065: mol_type = other DNA
atgtcatccgccggaaagtgcccacaagaagaacagactgttaaagcctttggctgggctgcgagagaagtgagtggagcattgtccccttacgggttttctaggcgtgcgacgggggagagagatgtcaaaattaatgtactgtatagtgggatgtgcaactttgataccgaaatgatcaacaacaagttcggttttaccaaatatccttacgtccttgggcacgagacggttggcgtagtatccgaggtgggctcaaaagtccgtaaatttaaggtgggggataaagtaggcgtggggggccttgtcggttcttgtcgtaagtgttactcctgcacgaatgatctggaaaactattgcactaaagctgaattaactgaggcgggcgcagggggttgttccaattttgttgtaaccgatgaagactttgttttccattggcctgagaaattgccgttagacctgggagcaccacttttgtgcgctggtgtgacgacattttctcctttgaagacgcatggtctagacaagccgggaatgcatgttggtgtcgcagggctgggaggccttggccacgtggccgttaaatttctaaaagcattcggcgctaaggtaactgtgatttcaacaagtgataacaagaagcgtgaagcgatagaaaaatatggagccgatgcgtttctagtgtcatctaatcccgaacaaatgcagacagccgctgggaccctgacaggtattattgataccatacctagtgctcatagccttgttcccttactaaatttattaatgccacaagggaagcttatagtgcttggtgtcccaaccgaaccattcgttctgccggtggtaccgatgattcaaggtggaaaagtcgttgctgggtcctccttcggtggcttaaagttaatacaggagatgcttgactttgccgctgaacataacattgtagccgatgcggaagtcatacctgttgattatatcaataccgctattaagcgtatcgaaaccggagacattaagtacaggtttgtaattgatgttggaaacacgctgaagtcaccc

SEQ ID NO: 19 - DNA, 1065 bp, synthetic construct
  source 1..1065: mol_type = other DNA
atgtcatccgccgggaagtgtgctcaagaagaacacaccgtcaaggcgtttggctgggccgcgagggaagcaagtggggctttaagcccctatggcttctccagaagggccacaggtgagagggatgtgagagtgaagattctttactgtgggatatgtaggacggatgccgagatgatctccgacaaattctgcttcactaaatatcctcatgtacccgggcatgagatagttggagtggtaagtgaggtcgggaacaaagtgcagaagttcaaggtcggtgccaaggtgggagtaactggcatgattggatcttgtcgtacttgttatagctgcacaaataatcttgaaaactactgccaaaaagccgtattaatagaagccaataacgggggttgttcaaatcttgtcgttgcagacgaagatttcgtattcaggtggccagagaagctaccgcttgatctgggcgcacccttgctatgcgctgggatcactacgtattctccgctaaaaaacttcggtttagataaaccaggcctgcatgtcggggtagctgggttaggaggactaggtcacattgcggtgaaatttctgaaagcttttggagctaaagtcaccgtcataagttcctcagataagaaaagaaaagaagctatagaaaaatacggcgcagatgagttccttgtttctagcaatcccgagcagatgcaagccgcagccggcacgcttaacggtatcatcgacaccgtaccgtctccacatagtcttgtaccctttctgaaccttttactaccccagggcaaacttatagttcttggggctcccatagagccctttgtactaccgatttaccctcttttacagggggggaaagtagtagcagggtctgccactggaggattgaagcaaattcaagaaatgttgcacttcgcggctgaacataatatcgccgccgatgcggaggttataccgattgactatattaataccgctgtagaacgtatagagaagggggatattaaatatcgtttcgttgttgatattggtaacaccctgaagagtgcg

SEQ ID NO: 20 - DNA, 1056 bp, synthetic construct
  source 1..1056: mol_type = other DNA
atgtcatccgcgggcaagagccccgaagaagaacagactgttaaagcgataggttgggcggcttccgaggctgcggaggttctatccccctgcgagttttctaggcgtacgacgggagaacatgacgtacaggtgaaagtcctatactgcgggatatgcaacttcgataccgaaatgataaacaataaattcggcttcaccagatacccattcgtactgggacacgaaatcgttggtgtggtatccgaggtggggtcaaaggtcaaaaagtttcaggtgggcgacaaggcgggggtgggtaccatgatcggcacgtgtaggacctgttacagttgcactaataacttagagaattactgtccaaaagcggtactgattgaggctaacaacggtgggtgtagcaatctagtagttgctgacgaggactttgtctttaggtggccggagaaattgccgttggacttgggagcacctcttctatgtgcagggattacgacgtattcaccgctaaagaacttcggtctagataagcctggattacacgtgggagtcgcgggtcttggcgggctgggtcatgtcgccgtcaaattcgctaaggcatttggtgcgaaagtgacagtgatctctagctcagacaataaaaagaaggaggcaattgagaaatacggagctgatgaatttttagtgtcatcaaacatcgagcagatgcaagctgccgcaggaaccctaaacggcattattgatacggtgccttccccgcattctctagtaccgctgcttaatctattgctgccccagggtaaactaatcgtccttggcgcgcctaccgaaccctttgttctgccaatatatccgcttcttcaggggaagtcaattatgccatctttctgctttgcaatgatttgtataggtcaaaaggtgattaacaatgtgcattatgtgcttattgttgtttattacagaaggaaaaactgtagatggaagtgccactggcgttttgaggcgaactccagaaacgcaccgttttgctccaggactcagcattccagcaggtgc

SEQ ID NO: 21 - DNA, 1086 bp, synthetic construct
  source 1..1086: mol_type = other DNA
atgtcatccgctaagtcactggaggagcaacacccgataaaagccttcggatgggctgctcgtgatgttagtggggtacttagcccctttagtttctccagacgtacaaccggagagaaagacatccagattaaaattctttattgcggaatatgccactcagatttacattgcgtcaagaacgagtggggttttagcaactatcccattgtcccaggtcatgagattgtcggtgttgtcacggaggtgggaagcaatattactaaattcaaagtgggggacaaggtaggggtgggctgcatggtaggaagttgtttatcctgcgagaactgctcaaacaacctggagaattactgtaaaaaggtcattccaacttacaattcaacatactatgacaagacccacacatatggtggatactccgatatattggtagtaaatgagcacttcgtagtcaggataccggaaaatatgcccctagatgctggagcgccgttactttgtgctggcatcacgacatacagtcccctgcgttattttggattagacaagcctggcatgcatatcggagtggtgggccttggaggactgggccatgtcgcggttaagtttgcgaaggctttcggcgttaaagtaacggtcattagcacatccctacataaaaagaaaaaggccttggaacacttgggagccgatagttttttagtctccaccgataaggaccaaatgcaggcggcaatggaaagtatggacggaattttagatacagtgagcgccgtacacccccttttacccctgatcaacctgcttaaaagccatgggaaatttgttatggtgggtgctccaaaccgtcctttggaattaccggtgtttcccatgttagctggtagaaaaattttggcaggttcaggtattggtggtatgaaggaaacacaagagatgcttgacttcgctgctgaacacaacatcgttgctgacgtggaggtaattagtatggattacgtgaacaccgcgatggaaagactggcccgtgccgatgtaaagcacaggttcgttattgacatagggaatacactgaaaactgct

SEQ ID NO: 22 - DNA, 1089 bp, synthetic construct
  source 1..1089: mol_type = other DNA
atgtcatccgccaaatcattcgaagaggaacatccagttactgcatttgggtgggcagcaagggatacctccggtttcctaagccctttcaaattctcaagaagggttacaggggagacagacgtgcgttttaaggttctattcactggaatttgtcactcagacttgcaccacctaaaaaacgactggggtgcttccaagtatcccttggttccgggtcatgagatcgtaggtgtggttacagaggtggggtcaaaggttcagaaattcaaagcaggcgacaaagttggggtaggatgtttgatagggagctgtaggacatgcgaggactgtgcggaagatcttgaatcttactgccctaaaaagataactacgtacgatggacccaggtactatgacggcagcctgacgttcggcgggtactccgatcacatggtcgtcgatgagcatttcgttatacactggccagagaagttgcccttggacgggggtgctcctttgctatgtgcgggtattactacgtacagccccatgaagtattttggtcttgataagcccggcctgcacatcggggtggtgggtctgggcggcttaggacacgtcgcggtgaagtttgcgaaagcctttggggccaaagttaccgtcatctcttcctcccccagtaaaaaggaggaggcaattaaaaattttggtgcagattcttttttagttagcagagacacagaggcgatgcaagcggcggcaggcacgcttcatgggatcttggatacagtcagcgcggtacatcccctgcttcctcttctgtccttgcttaaaaatcactctaagtatataatgttgggagcgcctgaaaagccattagagctgccggttttcccaatgttgatgggaagaaaaatagtcgcgggcagtaatatagggggtatcaaagagacggaggagatgcttaactttgcagccgaacatggtataactgcggatgtggaggtaataccaatggaatacgtcaataccgcgattgaaagactgggcaagaatgatgttcgttatcgtttcgtgatcgacgttgcgaataccctaaaagcggcc

SEQ ID NO: 23 - DNA, 1068 bp, synthetic construct
  source 1..1068: mol_type = other DNA
atgtcatccgccggtaagagcccagagaaggagcagacggtaaaagccactggttgggctgcgtctgaggtttcaggcattttaagtccgatcgaattctctagacgtgctacgggtgacagcgatgtacaaattaaagtattgtactgtggaatttgcagcttcgataccgagatgataaacaacaagttcgggttcactcgttatccttatgttttgggacatgaaaccgtaggggtggttagcgaggtcggctccaaagtaaggaagtttaaagtaggcgacaaagtgggcgtcggaggacttgtaggtagctgtggtacatgctatggctgtactaatggtttggagaattattgtacaaaagagcaaataactgaggcaggagcgggcggttgttccaactttatggtcatgggagaagatttcgtttttcactggcctgaaaagctaccgttagacctgggggctccacttctgtgtgccggtgttaccacctattcccccctgaagacccacgggttggataagcctggtcttcacgtcgggatagcagggcttggtggcatgggtcatgtagccgtgaagttcgccaaagcgttcggcgccaaggttacggttataagtacatcagacaataaaaaagaagaggcgatcaagaagtacggggcggacgcctttctgaactcctccaacccagaacagatgcgtgctgctgccggcactttagctgcaatagtggataccatcccctctccacacagccttgtgcccttgctagacttgctattgccgcatggcaaggtcatcgtccttggcgctccaagtgagcccttcgttctgcccgtcatgccgctactgcaaggaggtagagttgttgccgggtcctccggtgcatctcttaagcaaatacaagaaatgctggattttgctgctgaacacaacattgtagcagatgcagaggtcataccaatagattacatcaatacggctataaaaaggatagagaagggggacatcaagtacagatttgttgttgatattggtaacaccttgaagagcgcctga

SEQ ID NO: 24 - DNA, 1065 bp, synthetic construct
  source 1..1065: mol_type = other DNA
atgtcatccgcaggaaagtcccccgaaaaagaacagacagtcaaagccacggggtgggcagcttcagaggttagcgggatcttatccccaatagagttctctaggagggcgacaggagattctgatgttcagattaaggtcctgtactgtggaatatgctcctttgacaccgagatgataaacaataaatttggattcactagatacccgtacgtccttggccatgagacagtcggggtggtaagcgaggttggctctaaggttcgtaagttcaaggtgggggataaagttggggttggcgggttggttgggtcctgtgggacttgttatggctgtacgaacgggcttgaaaactactgtaccaaggaacaaatcacggaggcgggtgctgggggatgttccaacttcatggtaatgggagaagacttcgtatttcattggccagagaagctaccattagacctgggtgccccattgctatgcgctggggttacaacttattccccgttaaagacgcatggtttggacaaacccgggctacacgtcggggtagccggcctgggaggacttgggcacgtagcagtgaaattcctaaaagcttttggcgctaaggtaaccgttataagttcttctgacaataagaaaaaagaagcaatagagaaatacggtgctaatgcttttctggtttcttctaaccccgagcaaatgcaagctgcggcgggcaccctttcaggcatcatagatacgatcccctcaccgcatagcttactaccgttactagatttgttacttccgcagggaaaattaattgtattaggagcccctatagaaccctttgtcctaccaatttatcctctgctacaagggggtcgtgtcgtggcaggatccgcaacgggcggtcttaaacagatccaagaaatgttggactttgcggcggaacacaacgtcgcggcggatggtgaggtgataccaattgatcatgttaatacggctgtcaaaaggatcgaaacaggaaatatccgtcataggttcgttattgacatcggaaatacactaaagtccgct

SEQ ID NO: 25 - DNA, 1065 bp, synthetic construct
  source 1..1065: mol_type = other DNA
atgtcatccgcgggaaaatcccccgaggaagagcaaacggtcaaagcgatagggtgggccgcgtccgaggtttccggtgtgctttcaccctttgaagtcagtaggcgtgccacgggagaaagggacgtccaagtaaaggtcctatattgcggtatctgttcctttgacacagaaatgattaacaacaaatttgggttcacgaaatacccttttgtgcttggacatgaaattgtcggagtagcgtcagaagtcggcagtaaggtgcaaaagtttcaagtcggtgacaaggttggtgtcggtgctttgatcgggtcctgtagaacttgctacagttgcacgaacgatctagagaactattgtccgaaggccgttttgaccgaagccaactcaggagggtgttcaaacttcgtagttgcggatgaagactttgtattcaggtggcccgagaaattgccacttgatttaggtgcccctctgctgtgcgccggaattaccacctactcccctctgaagaattttgggctggataagccgggactacacgtaggggtggcaggtttaggcgggttaggtcatgtcgcggtaaagtttttgaaagctttcggtgcaaaggttaccgttatctccagcagtgacaataagaagaaagaggcaatcgaaaaatatggcgcgaacgcgtttttggtctcatctaatccggaacagatgcaagctgccgccgggacattatctggcataatcgacactataccgagtcctcacagtcttctacctttgttagatttgctattaccgcaagggaagctgattgtgctgggggctcccattgagccctttgtcctgcctatttaccctttattgcaaggagggagagttgtggcaggtagcgcgacgggtggacttaaacagatccaggagatgttggatttcgcggctgaacacaatgttgcggcggacggcgaagtaataccaatcgaccacgtcaacacggcagtgaagcgtatcgagaccggtaatataagacacagattcgttattgatattggtaatacgttgaaatcagct

SEQ ID NO: 26 - DNA, 1086 bp, synthetic construct
  source 1..1086: mol_type = other DNA
atgtcatccgcggagcagtccccagaggaacaacaccccgtgaaggtcctgggttgggccgcacgtgactcatcaggaatcttatcaccgttcaagttttctaggcgtgccacgggagaacatgatgttcagattaaagtgatatattgcggtatatgtacgtatgatgtggagatgataaaaaataagtggggcatgacaaactacccggtagttttcggccatgaagccgttggtatggtcacagaaatagggtcaaaggttcagaagttcaaggtcggggatacagtcggagtgggtacgcaagtgggttcttgtggcaagtgcgagtgctgcaccaacgatctagaccaatactgtcccgaaatgatagctacggacggttttggtagtaatgatgtgagctatggcgcttgttgcgacataatggttgttgacgaaaacttcgtaatagtttggccccagaacctaccaatggatgcaggcgcacccttactatgcgccgggatcacgacctatagcccgctgaggaactttggtcttgacagaccgggaatccatgtcggagttgtaggactggggggcttgggtcatgtggccgtgaggttcgcgaaggcgttcggggcgaaggttacggtgatcagtacatcaatgaataaaaaacaagaagccgttcataagtatggagcggactccttccttgtctctactgatcaagaacagatgcaggtaaatacggcctgggtcgctgccatagttgataccataccatcaccgcattcacttgtaccgctgctagatttgctactaccacacggaaaggtaatagtgcttggagctccgtccgagccatttgttctacccgttatgccactgttacagggtgggagagtagtggccgggtcctccggggcgagtttaaaacagattcaggagatgctggatttcgcggcagagcacaatatagtggcggatgcagaggtcatacccattgattacatcaatacagctattaaaagaatcgagaagggggatataaaatacaggtttgttgtggacattggaaatacccttaaaagtgcttag

SEQ ID NO: 27 - DNA, 1032 bp, synthetic construct
  source 1..1032: mol_type = other DNA
atgtcatccgaaagagttgaggacctgacggagctattagcagcccagcaccatgtcggaaatcaaatgctgaattttcgtaagtcagcctccctaaaatgcgccattgaattagggataccagacgccattaaccaacatggaaagccgattactttatgcgagctagtttcagctcttccaattaatccatccaaagctaaccacatatacaggcttatgaggtttctatcaaatgccggattcttcatactacaagatcagggttatgctcttactgctgccgggaggctacttttaaaagaagagccttttaacctaagggcttttattttctatatgtcagacccagcggtagtcaaaccttggaactccctgacagaatggtttagtaacgacgaccctttcccgttcgatacggcccacggcaaaaatttctgggcctacgctgcggaggagcctaattttgcgaatctctttaatgaagctatggcaaatgattccactctaatcgtccaggtaatgatgacccaatgcaaatttgtattcgacggattgacttctcttgcagatgtgggtggtggtaccggagcggtagctcgtgctatagctcaaaactttccaaacctaaaatgtgtagtctgcgacctaccgcatgtcatcgctgaccaggaggggacagaaaatctggacttcgtggcaggagatatgctagagaaggttccggcagctgacgccattcttttaaagtggattctacatgattggtcagacgaggattgtgtaaagatccttaagaactgtaaggaagcgattccaggaagagataagggtgggaaggtgataattatagacatgattatggaaagtcaaatgaaggatgacgaaagcgtcgaaactcaagttggggtgaacatgcagatgttgatggggtacggagcgaaagagaggtcagaaaaagaatgggccaaattgtttcaagacgccgggttttccgattacaaggtgcttcctcttctttgcgtgtctcat

SEQ ID NO: 28 - DNA, 1080 bp, synthetic construct
  source 1..1080: mol_type = other DNA
atgtcatccgatgccatgaagtcagcggaactattcaaggcacaagcacatatctttaaacaagtcttttgctttactaacggagcctcacttaaatgcgcggtgcaactaggaatccctgatgccatcgacaaccacgggaaggcaatgaccctaagtgagcttacggacgcgcttcccattaacccctccaaagcaccacacatacatagattgatgagaatccttgtaacggctggtttcttcgtagaagagaggttagggaatgggaaggaggagaaagcaaacgggtatgctttaacaccttcttcaaggctactattgaagaataaacccctatccttacgtgcatcagcgttgactatgttagaccctgtcacagtaaaaacatggaacgccctgagtgagtggtttcagaatgaagatcaaactgcctttgaaactgcgcacggaaagaatatgtgggatttctttgctgaagatccgggtctatccaagaagttcaacgaaagtatggcctccgacagccagttggttactgaagttcttgtaactaaatgtaagttcgtcttcgagggtcttacatctatggtggacgtgggtggaggaacagggactgtggcaggtgcaattgcgaaaactttcccgtctctaaggtgtaccgtctttgatctaccgcatgtggttgcgaacttggaacctacggagaacttagacttcgttgcaggcgatatgtttggcaaaattcctccagcaaatgcaattttcttaaagtgggtgttgcacgactggaacgatgaagactgtgtgaagatcctgaagaattgtaagcgtgctattccagggaaggagaagggaggcaaagttattatagttgatatcatcatggagactgagaagcacgatatcgatgagtttgactacgcaaagatgtgcatggacatggaaatgctggtattgtgcaattccaaggaacgtaccgaaaaggagcttgccatgttagtctctgaggccggtttcagcggctacaaaattttccctgtattgggcattaggagtcttattgaggtatatccgtaa

SEQ ID NO: 29 - DNA, 1080 bp, synthetic construct
  source 1..1080: mol_type = other DNA
atgtcatccgggagcgctgaggaagaagcaagcctgttcgccatgcagctagtgacatccgcatccctaccaatggtgctatacaccgcagtcaagctaaatctgtttgaaatcatagctaaagctggtcctggtgcaaagttatcaccctcagaaattgcttgtaggctgcctacaacgaagaaccctgacgctgcatcaatgctggacaggatgctaagattgctaagttctcactcactattgacatatgatgtggtcgaagttgcaggtggagccgaaaccgacataggatatgagcgtgtgtacggcctgagtccggtgggtgagtaccttataccagatgaagaaggaaattctgtggctcctttagtcgagttgctgcttgacaaagtcctgattaatacatggtacgaactagggaacgctgtcctagaaggaggcattccatttaatcgtgttcacgggacccatgcatttgactacccatcaaggaatcccagatacaacgagctgtttaacaatggaatggtgggaccgactgccatcaccatgaaggaacttctacaacagtataaagggttcgagctacttcagactcttgttgacgttggtggtggtttgggtataacgcttcataagattattgctaaataccccagcatccgtggaattaatttcgatctgccacacgttattgaggatgctccgccgtatcctggcatggagcatataggaggtgacatgttcgagtctgttccaggtggcgatgcaattttcatgaagatgattctacacgattggtctgatgatcattgcttaaaactattaaagaactgctatagggcactgccagaccatggtaaagtaatagtagtggacctgatcttgcctgtaaagccggatacgaatatcttcgtcaagggtatatttcaggcagatgtcattatgatgacgcagaatcctggtggcaaggagagatcagaagcagatgtgcgtgcgctggcgatcggagctggatttaaaggtatcaaacttgaatgctgtgctggaaacgtagggatattagaattgtacaaa

SEQ ID NO: 30 - DNA, 1044 bp, synthetic construct
  source 1..1044: mol_type = other DNA
atgtcatccgacgttcaatcagctgagctactgagggctcaagcccacgtgtggtctcaaacttttaacttcaagaactcagctagtttgaaatgcgctgtccaactaggaatcgctgatgcgatccaaaagcatggaaaacctatcacgttaagtgaattggctgcagcgctaccgatcaatccttccaaagctaattatatttacaggctaatgcgtatcttagtcaacgctggcttctttgctcaagagaaggagggctactcccttaccgcagctggaagactattattgagtgatgaaccggtcaatgcaagggcctttatcctagttgttctggaccctgacactatgaagccttggaatttcttaagtgactggttccagaacgatgacccatcaccctttgacaccgcacatgggaagaatttctgggacttcactgcggaagaacccaaattcagtaagctgttcaacgagggcatggtaagcgattcacaccttataacgatggtcttggtcaccgaatgtaagtacgttttcgagggacttacctcattagtggacgttggaggtgggacgggaaccgtcgctaggtcaatcgctgaggcttttccaaacctgaagtgtactgtatttgaccttccacacgttgtcgctaaccaagaaggaacattaaatttagacttcgttgctggtaacatgctggaaaaggtacctcccgcaaatgcaatcttacttaaatggatattgcatgactggtcagacgaagattgcgtaaagatattgaagaattgtaagaaggcaatcccgggtagggacataggcggtaaggtaattataattgatatggtgatggaaagtcaacttgagggtgatgagagcacagaagcacaaatctgtttggacatgcagatgctggttttgtaccgtgcaaaggagagatcagaaaaggagtgggcgaaattattctctgatgctggcttcccatcatataaagtattccctgcactgggaacgcgtgctataatagaggtctatccc

SEQ ID NO: 31 - DNA, 1044 bp, synthetic construct
  source 1..1044: mol_type = other DNA
atgtcatccgatgtccaatcagctgaattactgagagcgcaagcgcacgtgtggtctcagacgtttaatttcaaaaattctgcctcattaaagtgtgcggtacagttgggaattgccgatgttatccagaaacatgggaaacccattaccctatctaagttggcagctgcgctaccaataaatcccagtaaggcaaactatatttaccgtttaatgagaatccttgttaatgctggattcttcgcccaagagaaggagggatactcattaactagcgctggtcgtctattgttaagcgacgagccagtgaacgctcgtgcctttatattagttgtgttggaccctgacacaattaaaccttggaatttcttatccgactggtttcaaaatgatgaccctagtccgttcgacacggctcatggaaagaacttctgggatttcaccgcagaggaaccaaaatttagtaaattgttcaacgagggaatggtttctgattcacacttaatcactatggtgctagtgactgagtgcaaatatgttttcgagggattgacaagcctagtggatgtaggaggtgggacgggaactgtcgccaggagcatcgccgaggcattccctaacttaaaatgcacagtttttgacctgcctcatgtggtagcaaaccaggaaggaacattgaacctagattttgtcgctggaaatatgttggaaaaggtcccacctgctaacgctattctacttaaatggattcttcatgactggtctgatgaagcatgcgtgaagatcttgaagaactgtaagaaagcaatcccagggcgtgacatcggaggtaaggttatcataatagacatggtcatggaatcacaattggaggacgacgagtctacagaagcccagatctgcttagacatgcaaatgcttgtgttatacagagccaaagagagatcagaaaaggagtgggcgaaattattttctgatgcaggtttcccatcctataaagtctttccagcccttggtaccagggccatcatagaggtttacccc

SEQ ID NO: 32 - DNA, 1074 bp, synthetic construct
  source 1..1074: mol_type = other DNA
atgtcatccgacgtacaggcaggcgaactgttacaagcacaggctcacatttggaaccacatattcaatttcatcaactctatgtcccttaaatgcgccgtgcagttagatatacctgacatcatacataagcacgggcaaccaatgactctagcacagctaatacatgcattgccaatcagtagcgctaaggctcctttcgtccaccgtctaatgagaatactgattcactcaggattcttcgtgaaggcaaagattccaggtaatgagctggaagagggctacgagcttacatctgcaagcaagttgctgcttaagtctaatcccctatccgtgaccccattcctgttagccatgctagatccgatcttaactgatccctggcaccacttttctcaatggttccaatgcgacgacgaaacgccgtttgatacttgccacggaaaggcgttctggaaactagcaggtgagcagccaaggctgaatcaattctttaatgagggcatggcctctgacgcgagacttatttcgagcttggttataaagcactgcaaacatgtcttcgagggacttaatagtttagtagatgtcggtggagggactgggacattggcgatggctatagcggatgcgttccccgacctgaaatgcacagtcctagacttgccgcatgtggtggcgggccttgatggcagtagcaaaaatcttgcatacgttggcggagacatgttcgaggcgattccgcctacagacgctgtcttactgaagtggatcttgcacgactggtctgacgaagaatgcgtccagattctgaagagatgcaaagaagccattccttctaagggtaaaggaggtaaggttataattattgatatacttgtcaaggatcagcacagaggtgatgacaatgatgaagctatcgagactcagctattctttgatatgctgatgatggtcctggttaaaggtagggagaggaatcagaaagattgggccaaacttttcttcgaggctgggttcaacgattataagatcacaccagtcttgggtctgcgttccttaatcgaagtatac

SEQ ID NO: 33 - DNA, 1071 bp, synthetic construct
  source 1..1071: mol_type = other DNA
atgtcatccgatgtgcatgcgggtgagttgctacaagcacaggcgcacatctggaatcatatattcaacttcattaattcaatgtctctaaaatgtgccattcagcttgggatacccgacataatccaaaagcacggtcagcccatgaccttggcacaactaatccatgccttgcctatatctaacgcgaaagcgccattcgtttatagactgatgagaatactgatccactcaggtttcttcataaaagccaagatcccaggtaatgaattggaggaaggttacgagctgactcctgcctcaaaactacttcttaagtcaaacccctatagcgtcactcctttcttattagctatgttggacccgatcctgacggacccatggcacaatttctcacagtggttccaatctaacgacgagacccctttctatacatgccacgggagagaactatgggaattagctggcgagaagcctaggctaaatcagttctttaacgagggcatggcctctgacgctcgtctaatctcctccctagtcataaaacactgcaagcatgtgtttgagggtcttaacagcctagtcgatataggaggtgggactgggacgcttgctaacgccatagcggacgcttttccagatttgaagtgtacagtgttggacttgccacacgtggtagcggggttagatggatcatccaagaatctagcctatgtcggcggcgatatgttcgaggccatacctcctaccgatgctgttctgttaaaatggattcttcacgattggtcagacgaggagtgtgtacaaatactaaaaaagtgtaaggaagcaattccgtcaaaggagaaaggtggtaaagtcataattattgacatgttaatgaagaaccaacagaaaggtgatgatgaagacaaagctatagagacacaacttttctttgacatgcttatgatggtgctagttaagggtaaagagcgtaaccaaaaggactgggtcaaacttttcttcgaagcaggcttcaacgactataaaataactcctgtgttgggactacgttcacttattgaaatc

SEQ ID NO: 34 - DNA, 1080 bp, synthetic construct
  source 1..1080: mol_type = other DNA
atgtcatccgacgttcaggctgataaattgctggaggctcaggctcacatctggaatcatacatttaactttataaattctatgtgccttaaatgtgcgattcaattagggataccggacattatcaagaagcatggccgtccgatgactcttgaggagctaaccgatgcactaccaatcaacaatgcaaagagcccgttcatctatcgtttgatgaggatcttgatacattctgggttctttattaaagcgaaagtaacagacaccgacgtggaaaagggttatatgctaacgagcgcctctgagctacttctggcaaatgaccctttctccatgaagactttcctactggcaatgttagatccaattctaacggctccctggcagcatttctctcaatggttccaatccaacgatgagaaccctttctatacctgtcacggaagagccttctgggagctagctgggcaggaaccgagacttaaccaattctttaatgaagcaatggctaacgatgcacgtcttgtgagcagcatcgtaattaaacactgcaaacacgtcttcagcggtctgaacagtttgcttgacgttggaggtggtactggaacgttcggcaaggcaatcgcagctgctttcccagatctgaaatgcacagtactagatcttccccacgttgttgacggattggacgtgggtggcaagaaccttgcctatattggcggtgatatgttcgaggacatacctccagcagatgccgtacttttgaaatggattttacatgattggacggatgaagaatgtgtcaggattctggaaaaatgcaaacaggctatcccgtcaaaagaaaatggaggcaaagttatcatcatagacatggttcgtaacgaccagcaaaaagaagcggatgatgacgcacgtaagagtatagaaactcagttattctttgatattttaatgatggtcgataccaaaggtagggaaaggaatgaaaaagagtgggccaaactgttctatgaagccggctttaaagattataagattacgcaggtagtaggactgagatctctaatagaggtatat

SEQ ID NO: 35 - DNA, 1074 bp, synthetic construct
  source 1..1074: mol_type = other DNA
atgtcatccgacgtacaggctggcgaactactacaggcgcaggctcagatatggaaccacatatttaacttcataaatagtatgtccttgaaatgtgctgtacaattagatattcccgacataattcacaagcatggtcagcccatgacactagcccaattaatacacgcccttccaatatctagcgctaaagctccattcgtctataggttaatgaggattttgattcatagtgggttcttcttgaaagctaagatcccaggcaatgagctagaggaaggttacgagttgacgagcgcgtctaagctactattgaaaagcaatccattttcagtgacgcctttcttgctgtctatgttagacccaattctaacggacccatggcaccagttttcccagtggtttcagtgcgatgacgaaacgccattttatacatgccaccagaaggcattctggaaactagctggtgaacaccccagggtgaatcaattcttcaacgaaggcatggcatccgacgcaagactaatcagtagcttggtcatgaagcactgtaagcatgttttcgaaggcttaaacagtctagtcgatgtaggtggaggcacaggaaccctagcgatgaccattgctgatgcttttccggatctgaaatgcacagtattagaccttcctcatgtggtggctgggcttgacggtagcagtaagaacttagcttatgttggtggagacatgtttgaggcgatcccacctactgacgctgttttgttgaagtggattcttcatgattggtccgatgaggaatgcgtccaaatattaaagaaatgcaaagaggccattccgagcaaaggtaaaggcggaaaggtgataataattgatatgttagtgaaggatcagcaaaggggagatgataaagacgaggcgatcgaaacccagttgttctttgacatgttgatgatggtattggtcaaagggcgtgaaaggaatcaaaaggattgggcaaagctattcttcgaggccgggttcaatgattacaagattacccctgtattgggactgcgttccttaatcgaggtatat

SEQ ID NO: 36 - DNA, 1080 bp, synthetic construct
  source 1..1080: mol_type = other DNA
atgtcatccgatgtgcaagccgacaaacttcttgaagcccaggctcatatttggaaccacacattcaactttataaactctatgtgcctaaaatgcgccattcagttgggcataccagacattattaagaaacatgggagacctatgacattggtcgaacttacggatgcactaccaatcaattccgcgaaatcaccattcatatataggttaatgagaatactgattcactcaggattcttcatcaaagtcaaagttaccgataccgatgtggagaaaggatacatgttaacgtctgcgtcagagctactactggctaatgaccccttcagtatgaaaaccttcttactagcaatgttagaccccatcctaactgatccttggcagcatttcagccaatggtttcaatcaaacgatgaaaacccgttttacacgtgccatgggagggctatttgggaacttgcaggccaggagccacgtctgaaccagttcttcaacgaggcgatggccaatgacgctaggcttgtgtctagcatcgttattaaacattgcaaacatgtgttcagcggcttaaatagcctattagacgttggtggaggtacgggtaccttcgctgaagcgatagctgccgcatttccagaccttaagtgtactgtgctagacttaccgcacgtcgtagacggattagacgtaggttccaagaatctggcctacataggaggtgatatgtttgaagatatacctccggcagacgcggtccttttgaagtggattttacatgattggtctgatgaggaatgcgttagaatcctggagaaatgtaaacaggcgataccctcaaaagagaacggaggtaaggtaatcatcatagatatggtcagaaatgaccaacagaaggaggccgatgacgacgcgcacaaaagcattgaaacgcagctattctttgacatcctaatgatggtggacacaaaaggcagggaaagaaatgagaaggagtgggcgaagttgttctacgaggcaggattcaaagactacaagatcacccaagtagtgggccttaggagccttatcgaggtctat

SEQ ID NO: 37 - DNA, 1077 bp, synthetic construct
  source 1..1077: mol_type = other DNA
atgtcatccgatgtacaggcggataagttattagaggcacaggcacatatatggaatcacatattcaacttcataaattcaatgtccttgaagtgtgccatccaaatgggtattcccgatataataaagaatcatggaaggccaatgacattgaaggagttaacggatgccttacctataaacaacgcgaagagtccgttcgtctataggttgatgagaatcttaattcactcaggattctttgctaaagctaaggttaccgataacgatgttgaggaaggatacgtactaacctccgcatccgaacttttattggcggatgacccattctcattaacgcctctgctactagcgatgctagatcccataatgacagatccctggcaacacttctctcagtggttccaaaataacgatgaaactccgttttatacttgccatggcaggactttctgggagttggctagtcaggagccgagattaaaccaattcttcaatgaggcgatggccagagacgcgtggctggtaggctctgtggtaattaaccattgtaaacatgttttcagcgggttaaattctctggtggatgtcggtggagggactggtacgcttgcgaaggcgatagctgacgctttcccagatttgaaatgtacagctcttgatctgccatacgtcgtggacgggttagacggttctagcaagaatctggtttacattggaggtgacatgtttgaggccattcctccggctgacgctgtcctacttaaatggatactacatgactggactgatgaagaatgcgttcgtattttagtaaaatgtaagcaggcaatcccgagtaaggagaacggtgggaaagtaatcattattgacatggttattatcattgatatggtgaagggtgcgaacgaccatagaagcattgagactcagttattcttcgacttgttgatgctagttgacgtcaagggtaaagagaggaatgaaaaggaaagagcgaaacttttctatgaagctggctttaatgattataaaataactcaagtcggagggcttaggaccttgatagaagtttac

SEQ ID NO: 38 - DNA, 801 bp, synthetic construct
  source 1..801: mol_type = other DNA
atgtcatccgccgagctatataatcgtcaagccaagcagtatagtgatagtagaccgacgtacccagaagagttatttcaattcatagcctccaagactccttgtcatgacctagcgtgggatgtcggaactggaagcggccaggctgcgaggtccttagcaggcatctataagaatgtggtcgcaaccgatataagccctaagcagctagaatttgcagtgaagttaccgaatataaggtatcattgtaccccaccaaaaatgccgatagctgaggttgaacaaaacatcgctgccgagtcctctgtggatctagtcacaatagccactgcgatccattggtttgacttaccaactttttatcagcaggtcaaatgggttttgaagaaacctcacggggttatcgcagcctggagctacatcaatgttgagatcaatacatgtagcgtcaacaccttgttgcaaaaactatatctggattgtgctccgtattgcgaaccacaagctaagtcagtcgctgatgacaaatatagaaccattgatttcccatttcagccggtagacgggcttgacaatacgggtccgttcgagttcaagatggaaaaattgatggatttggatgggttcttcacctttatcaagagctggagcctataccagaccgccaaagataaaggtgtagaattattgaatgacgatgtggttcaggactttactagggcatggaatgaagatggcaatcctcagaaaatagcgacgtttccaatataccttcgtattggcaaagtaggcaactggtat

SEQ ID NO: 39 - DNA, 1065 bp, synthetic construct
  source 1..1065: mol_type = other DNA
atgtcatccgattttgctagaaatggtgaccatactggtgaattgtttcaagctcaagcacatatttggaatcatttgtttaattttattaattctatgtcattgaaatgtgctattcaattgggtattccagatattattcataaacatggtcaaccaatggcattggctcaattaattgatgctttgccaattaataatgctaaagcacattttgtttacagattgatgagaattttgattcattcaggtttctttattaaagctaaaatgccagataatgaaggtcatgaaggttacgctttgacttcagcttctaaattgttgttagcaaatgatccattttctgttacaccatttttattggctatgttagatccaattttgactgatccgtggcatcatttttctcaatggtttcaagataatgaagaaacaccatttcattcatgtcatggtacttctatgtgggaattggctggtagacaaccaagattgaatcaatttttcaatgaaggtatggcatcagatgctagattggtttgtacaatggttattaaaaattgtaaagatgtttttatgggtttaaattctttgattgatgttggtggtggtacaggtactgttgctaaagcaattgctgatgcatttccacatttgaaatgttctgttttagatttgccacatgttgttgatggttctgaatcatctaaaaatttggcttacgttggtggtgacatgtttgaagctattccaccatctgatgcagctatgaatcaatggattttgcatgattggtcagatgaaaaatgtgttcaagttttgaaaaagtgtaaagaagcaattccatctaaagaaaaaggtggtaaagttattattattgatatgttggttaaaaatcaacaaaaaggtgacgataatgatgaaccaattgaaacacaattgtttttcgatatgttgatgatgattttgtacaaaggtagagaaagaaatcaaaaagattgggaaaaattatttttgcaagctggttttaattactataaaattattccagttttaggttaa

SEQ ID NO: 40 - DNA, 840 bp, synthetic construct
  source 1..840: mol_type = other DNA
atgtcatccagatttttggttaatggtggtttctttattcaacaagatcaatgttacgctttgacaactgcaggtagattgttgttaaaagatgaacatattaatgttactggtttggttgctttatctactgatccaattatgatgatgccgtggattttcttgactgaatggtttaaaaatgatgatccatctccatttgatactacatacggtaaatctttttgggattacactgctcaagaatctagatttggtaaagtttttgatgaagcaatggctaatgcatctaatttgccaattcaagttccaattactaaatgtagatttgtttttgaatcattgacttctttggctaatgttggtggtggtactggtaaagttgcaaaagctattgctaaaaattttccaaatttgaaatgtgttgtttgtgatttgccaagagttgttgctaatcaagaaggtactaaaaatttggattttgttgcaggtgacatgttggaaaaagttccaccagcaaatgctgcattgttaaaatgggttttgcatgattggtctgatgaacatggtttgaaaattttgaaaaattgtaaaatgtctattccaactagagaaaaaggtggtaaagttattattattgatattattattgaatctcataaagaagataatgaattggttgaaactcaaattggtatggatatgcaaatgttggttttatacgttgcaaaagaaagaactgaagaagaatgggctactttgtttttggatgctggtttttcttcatacaaagtttttccagttttgggtgtttcttgtttgattgaagtttatccataa

SEQ ID NO: 41 - DNA, 1098 bp, synthetic construct
  source 1..1098: mol_type = other DNA
atgtcatccgatttggctagaaatggtgaccaagctggtgaattattgcaagctcaagcacaaatttggaatcatatttttaattttattaattcaatgtcattgaaatgtgctgttcaattggatattccagatattattcataaacatggtcaaccaatgacattggcacaattaattcatgctttaccaatttcttcagcaaaagctccatttgtttacagattgatgagaattttgattcattctggtttctttttgaaagctaaaattccaggtaatgaattggaagaaggttacgaattgacatcagcttctaaattattgttgaaatctaatccattttctgttacaccatttttattgtctatgttagatccaattttgactgatccgtggcatcaattttcacaatggtttcaatgtgatgatgaaacaccattttatacatgtcatcaaaaagcattttggaaattagctggtgaacatccaagagttaatcaatttttcaatgaaggtatggcttcagatgcaagattgatttcatctttggttatgaaacattgtaaacatgtttttgaaggtttaaattctttagttgatgttggtggtggtacaggtactttggctatgacaattgcagatgcttttccagatttgaaatgtacagttttagatttgccacatgttgttgctggtttggatggttcatctaaaaatttggcttacgttggtggtgacatgtttgaagctattccaccaacagatgctgttttgttaaaatggattttgcatgattggtctgatgaagaatgtgttcaaattttgaaaaagtgtaaagaagcaattccatcaaaaggtaaaggtggtaaagttattattattgatatgttggttaaagatcaacaaagaggtgacgataaagatgaagctattgaaactcaattgtttttcgatatgttgatgatggttttggttaaaggtagagaaagaaatcaaaaagattgggctaaattgtttttcgaagctggttttaatgattataaaattactccagttttaggtttgagatctttgattgaagtttactattactaa

SEQ ID NO: 42 - DNA, 519 bp, synthetic construct
  source 1..519: mol_type = other DNA
atgtcatccgaaggtgctggtggtactttgcaaacatgttgttggtctccaggtccaagagctgtttctaaatgtaatggtcatgaatgttttccaagatggtttgcagctgttttggcaaaatacccagtttgggttatgaatgttgttccagctcattctgatccagatactttgtctattatttacgaaagaggttttattggtacttaccatgattggtgtgaagcattttctacttacccaagaacttacgatttgattcatgctgatggtgttttctctatttaccaagatagatgtgatattacttacattttattggaaatggatagaattttgagaccagaaggtactgttatttttagagatgttgttgaagttttggttaaaattaaagctattactgatggtatgagatgggaatcaagaattttggatcatgaatctggtccattcaatccacaaaagattttggttgcttttaaatcttactggactgcaaaagctaatgattaa

SEQ ID NO: 43 - DNA, 1089 bp, synthetic construct
  source 1..1089: mol_type = other DNA
atgtcatccgatactaatgttgaaagatcaaaattgttacatggtcaagctgaaatttggaaacatacttttagatttgctgattctatggctttgaaatgtgctgttgaattgggtattgcagatattattcattctcatggtcatccattaactttggcagaaattgctactaatattacaaattcatcttctccacatattccatatttgtctagaattatgagatttttggttagaaataagatttttacttctacagatattccatctgatcatggtggtggttctccatcaactacattgtatggtttaacagcagcttcttggtggttattgaatgataatgatggtttgtctttagctccatttttgttaatggaaaatcatccgtggttggtttctccgtggcatcaattatcatcttgtgttagagaaggtggtattgcatttcaaaaagctcatggtaaagaaatttgggaattggcttctgaaaatccagaatttaatacaatttttaatgatggtatgggttgtactggtaaaattacaatgaaagcaattttgtctggttttaaagatgcttggaactctattgaatcattggttgatgttggtggtggtacaggtcaaactattgctgaaattgttaaagtttacccacatattagaggtattaattttgatttgccacatgttgttgctgcagcaccaaaatatgatggtgtttgtcatgttggtggtgacatgtttgattctattccaactgctcaagcagtttttatgaaatggattatgcatgattggggtgacgaagattgtgttaaaattttgaaaatttgtagaaaagctattccagaaaagacaggtaaaatttttattgttgatgttgttttaaaaccagatggtgacggtttgtttgattcaattggtatgatttttgatttgttaatgattgctcattcatctggtggtaaagaaagaactgaaccagaatggaaaaagttattggaaaaaggtggttttccaagatacaatattactgaaattccagcttgtttttctattattgaagcatacccagtttaa

SEQ ID NO: 44 - DNA, 840 bp, synthetic construct; identical to SEQ ID NO: 40
  source 1..840: mol_type = other DNA

SEQ ID NO: 45 - DNA, 1053 bp, synthetic construct
  source 1..1053: mol_type = other DNA
atgtcatccgctactttggaaaattctgctgaattgttaagagcacaagctcatgtttggtcacaaacttttaattttaaaaattcagcatctttgaaatgtgctgttcaattgggtattgctgatgttattcaaaaacatggtaaaccaattactttgtctaaattggcagctgcattaccaattaatccatctaaagctaattacatttatagattgatgagaattttagttaatgctggtttctttgctcaagaaaaagaaggttattcattgacatctgcaggtagattgttattgtctgatgaaccagttaatgcaagagcttttattttggttgttttagatccagatactattaaaccgtggaatttcttgtctgattggtttcaaaatgatgatccatctccatttgatactgctcatggtaaaaatttttgggattttactgctgaagaaccaaaattttctaaattgtttaatgaaggtatggtttcagattctcatttgattactatggttttggttacagaatgtaaatacgtttttgaaggtttgacttcattagttgatgttggtggtggtactggtacagttgctagatcaattgctgaagcatttccaaatttgaaatgtacagtttttgatttgccacatgttgttgctaatcaagaaggtactttaaatttggattttgttgctggtaatatgttggaaaaagttccaccagcaaatgctattttattgaaatggattttgcatgattggtctgatgaagcatgtgttaaaattttgaaaaattgtaaaaaggctattccaggtagagatattggtggtaaagttattattattgatatggttatggaatctcaattggaagatgatgaatctactgaagctcaaatttgtttggatatgcaaatgttggttttatatagagctaaagaaagatctgaaaaagaatgggctaaattgttttcagatgctggttttccatcttataaagtttttccagctttgggtactagagcaattattgaagtttacccataa

SEQ ID NO: 46 - DNA, 1101 bp, synthetic construct
  source 1..1101: mol_type = other DNA
atgtcatccgatttggctagaaatggtgacagagctggtgaattgttacaagctcaagcacatatttggaatcatatttttaattatattaattcaatgtctttgaaatgtgctgttcaattgggtattccagatattattgaaaaacatggtcaaccaatgactttggctcaattgtgtcatgctttgtctatttcaaatactaaagcatcatttttgtacagattaatgagagttttgattcattctggtttctttattaaagctaaaattccaggtaatgaattggaagaaggttatgaattgacttctgcatcaaaattgttattgaaatctaatccattttcagttacaccatttttgttagcaatgttggctccaatttcaacagatttgtggcatcatttttctaaatggtttcaatctgatgatgaaactccatttcatacatgtcatggtacttctggttgggaaatggcttctcaagaaccgtggattaatcaatcttttaatgaagcaatggcttcagatgctagattgttatcatctattttgattgaacattgtggtcatgttttctctggtttaaattctttggttgatgttggtggtggtacaggtactttggctaaagcaattgctgatgcatttccagatttgaaatgtactgttttggatttgccatatgttgttgatggtttggatggtggtagaaagaatttggcttttattggtggtgacatgtttgaagctgttccaccagctgatgcagttttgttaaaatggacattacatgattggtcagatgaagattgtgttagaattttggaaaaatgtaaacaagctattccatctaaagaaaatggtggtaaagttattattattgatatggttattaatgatcaacaaaaaggtgctgatgatggtgaacataaatctattgaaactcaattgtttttcgatatgttgatgatggttgatacaaaaggtagagaaagaaatgaaaaagaatgggctgaaattttctcagctgcaggttttaaagattacaaaattactcatgcattgggtttaagatctttgattgaaatttattattaa

SEQ ID NO: 47 - DNA, 1350 bp, synthetic construct
  source 1..1350: mol_type = other DNA
atgtcatccggaagcgcagaggaagattatccttcctgcaagaggcccaaccagcatacccagacaaagcacaagttcaagatccttctggtcgttatactaaccaacttattaaccatttatatattcgcaggtccctcactgcccataacaagaaatttaagcctaccgacatgggattacactacaaaactgttacacgaactgaacgtcacaaaaagagaattagcgaatagtagagcacagatttcagacttgcagcaacacttgaaggcgtcaaatatattgactaaatcactacttactcagcttactcgtttgaatggagccatagatcagcacagcactatcgaaagtagccttttcaactacgctgatttgcttaccgatttaagttctgaagcaaaactgggcattgggccacataaactgcctttaggatactcccctagatctggttccgaggaaatttacagtagtgtaggtggcggctgtctgaggtacaaagaggaattagcccaatacatgacttacaaaatcggtggagaatgtccggtcgatgatgtctttgtgcagcgtcttatgctgaagggctgcgagccgcttccacgtagaagatgtcatccgaaaagccctgcaggttacaaagaaccaaccccacttccgaaatctctttggaccactcctccggacacatctatcatctgggacccatatacgtgcaagaactactcctgtctagtagagcgtaagaaattgcccgggttctatgattgtaaggactgttttgacctacaaggaagggaaattagccgttggctgtttgacaatggaggcttagacttcggtattgatcaagtcctagggacgaaaccacacggcacaatcaggatcggattggacatcggaggagggacgggcactttcgctgcccgtatgaaagaaaggaatgttaccattgtgactacctccatgaacctggatggaccttttaactctttcatagcgtccagaggattgatctcaatgcacgtttcagtaagccagcgtctacctttctttgaaaacactcttgacattgttcacagtatgcacattcttagcaactggattccggacaccatgcttgagttcacactgtatgacatataccgtgttctaagacctggagggatcttctggttggatcatttcttctgcctaggctcacagcttaacgcaacatacgtccccatgttagaccgtgttggttttaagaaacttcgttggaatgctggaatgaagctagatagaggcattgataaaaacgagtggtattttagttctttactggagaagcctacgtcattc

SEQ ID NO: 48 - DNA, 1095 bp, synthetic construct
  source 1..1095: mol_type = other DNA
atgtcatccgggagtgcggcaccggaacaatcaaatagtatggctggtgatgaggaagcctgcctatttgctatgcaattggcgagtgcttccgtactgcctatggtattgaaaagtgctatcgagcttgatctactagaactgatcaagaaagcgggtcaagacgcttacgtttctcctagtgaattggcagcgcagttacctactgaaaacccggatgcacccgtcatgctggacagaatcttaagactactggccagttactcagttctaaactgtactttacgtaatctgcccgacggtggggtagaaaggttatacggcctggcccctgtttgcaagttcctaactaagaatgaagacggggtttctatggctgcgttgttgcttatgaaccaagacaaaattcttatggagtcctggtatcacctaaaggacgcagtcctagagggaggaatccctttcaataaggcctacggcatgaccgcctttgagtaccacggcaccgacccgaggttcaacaaagtgtttaaccaaggtatgtccaaccattctacgattattatgaagaaaattcttgaaatctacaatggcttcgaagggcttaagacggttgttgatgttggtggaggtactggagcgactttgagcatgatcgtttccaagtatccggcaatcaagggaatcaactttgacctgccacatgtgacgggcgatgcgcctacttaccctggcattgagcacgttggtggcgatatgttcttctcagtcccgaagggagatgctattttcatgaagtggatttgccatgattggtcagatgagcattgcctgaaatttttgaagaattgttaccaagcgctacccgagaatggtaaagtgatactagctgaatgtttgttgcccgaagctcctgatgcgaccctgtccacccaaaacacagttcatgtggacgtaatcatgcttgcccataaccctggtggaaaagaaagaacagagaaagaatttgaggctcttgcaaaggaagcaggttttaagggattccgtaaaatatgttgcgcttataattcttggataatggaactattgaagtga

SEQ ID NO: 49 - DNA, 1098 bp, synthetic construct
  source 1..1098: mol_type = other DNA
atgtcatccaactctccagtccaagaccagatctcaaatacacccagcgaagaagaagatttctttaaggtgatgcaattatctagcggcttaattcttcctatgatcttaaaagctgccattgatctcgaattgtttgagcttatagcaaaagctggtcctggagctaaactttctgcggtcgaaattgcctcacaactctcgaccaaaaatccaaatgcagctgtcacgcttgatcggatgatgaagtttcttgccagtttctcttttcttaattgcagtttggtcacagatgcagaaggacatcctaggagattatacagttttgcaccgacatgcaaaaactttcttccggatgaagacggagtttcttttgctcccgtgttgatgttgcctgttgacaaagtacacttcgagtcttggtatcacttaaaagatgcagttcttgagggaggtattccgttcatcaaggctaacggggacgatgcatttgcacatccagttaaagacaccagattctatgaggtcttcaacaaggcattgcacaatcacgcatgcatagtcacgaaaaggcttcttaaagtctacaggggattcgagggagtgaaacagctggtggatgttggtggcggacatggtggcacactaggctgcatcgtttccaagtatcctcatattcatgctatcaactttgatgttcctccggtaatcgaaggtgccccaccacgcccaggtgtggagttcgttggtggagacatgtttgaaagagttcccaaaggagaggttcttctcatgaagtgggtacttcacgattggagtgatgaacattgcctgaagatactgaagaactgttatgaggctttaccggaatccggcaaactaatagtgatcgaattggtgctgccggaaactcccaacagcgatccgttgtcgaagattgggtatcaatttgacatgaatatgatgagcgtaaaccttggaggaaaagagagaacagagaaagaatttgaagatcttgcaaaacaatctggattcgcttctattaaactcatctgtcgcgcaggttgtgattgggttattgagttctataaggattaa

SEQ ID NO: 50 - DNA, 1845 bp, synthetic construct
  source 1..1845: mol_type = other DNA
atgtcatccgcgactaagggaaatccgggtgataacagatccagatctagcatgcccatgttcattgccgctggcctttgttgcgtattttatttgcttggtgcttggcagcgttctggatctggtaagggagataacatagcctacgagataacaaagaaagcagaagactgttcaatactagccaatctagagtttgaaactcatcacggagatagggcagcgaacgatagtaagctggaagtaaaagagtacgagccttgtgacgacaggtacatagactacacaccctgtcatgatcaagcaagagccatgacattccctagggagaacatgaactatagagagaggcactgccctccggagcaagagaaattccactgcctaatacctgctcccaagggatacgtcacacctttcagatggccgaagagtagagactatgtaccattcgctaacgctccacacaagtcattaaccgtagagaaggcggtgcaaaactgggtgcaatatgaaggtaatgtattccgtttcccaggtggaggcactcaattcccgcacggagccgacgcttatattgagcagttggcttctgtgatccccattaatgacggcacggtgcgtacagctctggatacaggttgcggtgtggcatcttggggtgcttatttattcaaaaagaatgttataacgatgagctttgccccgagggacagccacgaggcgcaggtccagttcgccttggaacgtggagtaccagcggtaataggcgtgctaggtacgatcaagctgccttatccttctagggcgttcgatatggctcattgttcacgttgtttgatcccttggggtgctaatgacggtaagtatatgatggaagtagatagagttctgagacccggaggctactggattttaagcggacctcctattaactggagaaattcttatgaagcgtggcagagaccaaaagaggaattggaagaagagcagaggaagatcgaagaaattgctgaactgttatgctgggaaaagaaacatgagaaaggtgaaacggctatctggagaaaaaggatcaacaatgagaactgcaatgaccgtgactctagggtgacgatgtgtaagttggcggacgcggatgatgtatggtataagaagatggaggtttgtgtaaccccttaccccgaaaccaactctcaggaagaagtcgccggtggagaattgaaaccctttcccgagaggcttaacgcgatccctcctagggtggcaggagcgtctgtcttaggcgtcaccatagactcattccaggaagataacaggctgtggaagaagcacgtgagcgcgtaccagaggatcaacaagttgctagattccgggagataccgtaacgtgatggacatgaacgcaggtctaggtgggtttgccgctgcaatcgaaagctctaatttatgggtgatgaacgttattcccaccatcgcagagaaagacaccagtggaatcatctttgaacgtggactgatcggtatttaccatgattggtgtgaagccttcagcacttatccaagaacatacgacctgattcatgctgagggcgtattttcactgtacaaggacaaatgcaacgccgaggatatattattagaaatggacagaatccttcgtcctgagggtgccgttatattcagggatcatgcagacgtgttgtctgaagtaaagaggattgtggcaggtatgaagtggaatacgaagatgtacgaccacgaggatggtcccttagtcccggaaaaaatacttgctgccgtaaaacaatattgggtcgcaggagatacaaacagcacttttagc

SEQ ID NO: 51 - DNA, 1140 bp, synthetic construct
  source 1..1140: mol_type = other DNA
atgtcatccgcaaccagcgagaatactcagcttaagtccgtctctccttcaagtatggaaaatggaaaagcgggtgaggaacaggatcactttagtgacgtcatgcagttagtcacctctgcagccctgccaatggttctttacactgttgtgaagttgaacctatttgaaataattgctaaggcaggacctggcgctaagttatctccttctgagattgcaagcagactaccagcaacgaagaatcctgatgctgcctccatgcttgacaggatgttgaggctattgtcctcacactcattactaacctgcgaaatcggcgaggtggctggtggaggtgaaacagaagtgggttacgagcgtgtgtacggtctgtctcccaccggtgagtacttaataccagacgaagagggcaacagtgttgcccctctagttgaacttctgttggataaagttctaaccgacacctggtacgaactgggtaatgcggttttagaaggtggtatcccgttcaatagggttcacgggacccacgcgttcgactacccttccaggaaccctgagtacaacgaattgtttaacaaggggatggttggtccgacagccattacaatgaagaagctactgcagcaatacaagggctttgaacacttacagaccctagttgatgtgggaggaggcctaggaatcactctacacaaaattgttagcaaataccccaacataagtgggatcaacttcgacctacctcatgtaatagaagatgcaccatcataccctggtgtcgagcatataggcggtgatatgtttgaaagtgtcccaggtggtgatgccatcttcatgaaaatggttcttcatgactggagcgacgaccactgtttaaaattactaaagaattgttttaaagccttgccggatcatggaaaagttatcgtagcagacctaattctaccgatgaaacccaacacctccgcattcgtaaaaggcatcttccaggctgatgtaataatgatgactcaaaatcctggaggcaaggaaagatcagagacagacgtacgtgcacttgcaatcggtgctgggttcaaagtggtaaagttggagtgccaagctggcaaccttggaatcttagagctgtacaag

SEQ ID NO: 52 - DNA, 1140 bp, synthetic construct
  source 1..1140: mol_type = other DNA
atgtcatccgaaacgtccgaaaatgctcaactgaagtctgcctctccaagttcaattgagaatggtaaggagggagaaaagcaggaccactttagccatgtaatgcagctagtaacgagcgccagtttaccaatggtgttgtacaccgctgttaaactgaacctatttgagataatcgccaaagctggaccagaagctaagctatctccaagtgaaattgcatatagacttcttacgaccaaaaatccagacgctgcgtctatgttggatagaatgcttagattactgtcctcacacagcctgctaacctgtgacatcgtggaagtcgctggtggtgccgaaatagatataggatatgaaagagtctatggcttgagtcccgttggtgaatacctaatcccggacgaggagggtaacagcgttgctccattggtggaactattactggataaagttctgatagacacctggtatgaacttggaaacgcagttttagaaggtggcataccgtttaaccgtgtccatggaacacatgccttcgattatccaagtcgtaaccccagatataatgagcttttcaacaatggaatggttggtccgacggctatcacgatgaaggaactattacaacaatacaaaggctttgaacttcttcaaaccctagtggacgtaggaggtggactgggtatcacattacacaagatcattgcaaaatacccatcaattagaggcatcaactttgacctaccacacgtcatcgaagatgctcctccataccctggaatggaacatattggtggtgatatgtttgagtccgtgccaggtggagacgctatctttatgaagatgatcttgcatgattggtccgacgaccattgtctaaagctgctaaagaactgctaccgtgctttaccagatcatgggaaagtgattgtcgttgatttaatattgcctgttaagccagacacgaacattttcgtgaaaggtattttccaagcagatgtaattatgatgacccagaaccctggtggcaaggagagatctgaagcagacgtgagggctctggcgattggtgctgggtttaagggcatcaagttggagtgctgcgctgggaacgtgggtattctggagttatataag

SEQ ID NO: 53 - protein, 366 aa, synthetic construct
MAAGKSPEEEHPVKAYGWAVKDRTTGILSPFKFSRRATGDNDIRIKILYCGICHTDLTSVKNEYEFLSYPLVPGMEIVGIATEVGSKVTKIKVGEKVAVAAYLGTCGKCYNCVNDLENYCPEVIIGYGTPYHDGTINYGGLSNETVVNERFVLRFPEKLSPAGGAPLLSAGITAYSAMRNHGLDKPGIQLGVVGLGGLGHLAVKFAKAFGVRVTVISTTPSKKDEAINNLGADAFLFSRDDKQMRAAIGTFDAIIDTLAVVHPIAPLLDLLRSHGKLVLVGAPSKPLELPTIPLLSGGKSLIGSAAGNVKQTQEMLDFAAEHDITANIEVIPIDYINTAMERLDKGDIRFRFVVDIENTLTPPPEP

SEQ ID NO: 54 - protein, 354 aa, synthetic construct
MAAGKSQEDGQTVKALGWAAREVSGAISPFDFSRRAPGERDVQVKILYCGICSFDTEMINNKFGFTRYPFVLGHEIVGVVSEVGRKVQKFKIGDKVGVGTMIGSCRTCYSCTHNLENYCPKVTLTEATSGGCSNLVIADEDFVFHWPVNLPLDLGAPLLCAGITVYSPLKNFELDKPGLRIGVVGLGGIGHIAVKFAKAFGAKVTVISSSESKKVEAIEKYGADSFLVSSDPGQMLAAAGTLDGVIDTVPAPHSILPFLDLLLPRGKLIILGAPMEPFVLPIYPLLQGGRVVAGSATGGLKQIQEMLHFAAEHNIVADGEVIPIDDINTAIKRIEKGDVKYRFVVDIGNTLKSA

SEQ ID NO: 55 - protein, 354 aa, synthetic construct
MAAGKCAQEEHTVKAFGWAAREASGALSPYGFSRRATGERDVRVKILYCGICRTDAEMISDKFCFTKYPHVPGHEIVGVVSEVGNKVQKFKVGAKVGVTGIIGCCRTCYSCTNGLESYCPNVALTEAGEGGCSNFIVLDEDFVFRWPEKLPLDLGAPLLCAGAASYSPLKNFGLDKPGLHIGIAGLGGMGHVAVKFAKAFGAKVTVISTSDNKKEEAIKKYGADAFLNSSNPEQMRAAAGTLAAIVDTIPSPHSLVPLLDLLLPHGKVIVLGAPSEPFVLPVMPLLQGGRVVAGSSGASLKQIQEMLDFAAEHNIVADAEVIPIDYINTAIKRIEKGDIKYRFVVDIGNTLKSA

SEQ ID NO: 56 - protein, 489 aa, synthetic construct
MAYTPENSSYSSILNTYIQNSRFISPEAPKPKVILTPVHESQVQTAIYCSKRHGMQIRIRSGGHDFEGSSYMSNVPFLVFDMFNFRSISVDLRSKTAWVGAGATLGETYYNIYQKNSTLTYSAGYWPTVGIGGHVSGGGCGPLVREYGLAADNVLDARLIDVKGRILDRKSMGEDLFWAIRGGMGANFGVILAYKIKLVEVPEKVVAFKVRRTLVQNATKLVHKWQFVAPKLPVRLIISLQIASINSDQTGGRTIQASFASVFRGEVDELLQIMEEHFPELGLKKEDIKEMSWIQYFSFYGDEPIDDVAHFLTNRVSTTKPYFKAKSDFVQKPIPEAGIEKIWKKLFEKDPLAAQLQWTPFGGKMDQISDSETPFPHRAGNSFIMFEVIFWNGTDKVLAQKHINWIRELYDVIGQYVDNNPRGAYADYRDLDLGVNNIDETSAEQARVWGKSYFKDNFDRLVNVKTRVDPNNFFKNEQSIPPRLAHSSS

SEQ ID NO: 57 - protein, 537 aa, synthetic construct
MAAMLRTRMLQFAFVLLFSFCGVSSSSQPETPFLQCLFHHSDDSTIGYIIYTPENSSYSSILNSCIQNSRFIVPETPKPKVILTPVSESQVQAAIYCSKKHGMQIRIRSGGHDFEGSSYTSNVPFLVLDMFNFRSISVDLQSKTAWVGAGATLGETYYHIYQKNSTLTYSAGYWPTVGIGGHVSGGGCGPLVREYGLAADNVLDARLVDVKGRILDRKSMGDDLFWAIRGGMGASFGVILAYKIKLVEVPEKVVAFKVRRTLEQNATKLVHKWQFVAPKLPVRLIISLQIASINSDQTGGRTIQASFASVFRGGVDELFQIMEEHFPELGLKKEDVKEMSWIQYFSFYADEPIDDVAHFLTNRVSTTKPYFKAKSDFVQKPIPEAGIEKIWKKLFEKDPLAAQLQWTPFGGKMDQISESETPFPHRAGNSFIMFEVIFWNGTDKVLAQKHINWIRKLYGVIGQYVDNNPRGAYADYRDLDLGVNNIDETSVEQARVWGKSYFKNNFDRLVNVKTRVDPDNFFKNEQSIPSRLAHSSF

SEQ ID NO: 58 - protein, 537 aa, synthetic construct
MATMLRTRMLQFAFVLLISFCCVSSSSQPEIPFLQCLFHHSDDSTIGNVMYTPENSSYSSILNTYIQNSRFISPEAPKPKVILTPVRESQVQTAIYCSKKHGMQIRIRSGGHDFEGSSYMSNVPFLVLDMFNFRSISVDLRSKTAWVGAGATLGETYYNIYQKNSTLTYSAGYWPTVGIGGHVSGGGCGPLVREYGLAADNVLDARLIDVKGKILDRKSMGEDLFWAIRGGMGANFGVILAYKIKLVEVPEKVVAFKVRRTLVQNATKLVHKWQFVAPKLPVRLIISLQIASINSDQTGGRTIQASFASVFRGEVDELLQIMEEHFPELGLKKEDIKEMSWIQYFSFYGDEPIDDVAHFLTNRVSTTKPYFKAKSDFVQKPIPEAGIEKIWKKLFEKDPLAAQLQWTPFGGKMDQISDSETPFPHRAGNSFIMFEVIFWNGTDKVLAQKHINWIRELYDVIGQYVDNNPRGAYADYRDLDLGVNNIDETSAEQARVWGKSYFKNNFDRLVNVKTRVDPNNFFKNEQSIPPRLAHSSS

SEQ ID NO: 59 - protein, 353 aa, synthetic construct
MAAGKCPQEEQTVKAFGWAAREVSGALSPYGFSRRATGERDVKINVLYSGMCNFDTEMINNKFGFTKYPYVLGHETVGVVSEVGSKVRKFKVGDKVGVGGLVGSCRKCYSCTNDLENYCTKAELTEAGAGGCSNFVVTDEDFVFHWPEKLPLDLGAPLLCAGVTTFSPLKTHGLDKPGMHVGVAGLGGLGHVAVKFLKAFGAKVTVISTSDNKKREAIEKYGADAFLVSSNPEQMQTAAGTLTGIIDTIPSAHSLVPLLNLLMPQGKLIVLGVPTEPFVLPVVPMIQGGKVVAGSSFGGLKLIQEMLDFAAEHNIVADAEVIPVDYINTAIKRIETGDIKYRFVIDVGNTLKS

SEQ ID NO: 60 - protein, 360 aa, synthetic construct
MAAEKSPEEEHPVKAFGLAAKDSSGILSPFNFSRRATGDHDVQLRVLYCGLCYYDTKMIKNKRGVTRYPFVFGHEIVGEVTEIGREVQKFKVGDKVGVGCMVASCRSCESCANNCENYCPNVSVTDGAFFFKTGEVLYGGCSDIMVADENFVIRWPENFPLDAGAPLLCAGITTYSPLRNFGLDKPGIHVGIYGLGGLGHVAVQFAKAFGAKVTVISSSDRKRMEAIEKLGADSFLVNSNLEEMQAAMGTMHGIIDTVPANHSLVPLLDLLKPQGKLIVVGGPEKPFELPVFPLLQGGRLVAGSATGGIKQTQEMIDFAAEHNILPHVEVVSVDYVNTAIERTERGDVKYRFVIDIGNTL

SEQ ID NO: 61 - protein, 317 aa, synthetic construct
MAAEKSPEEEQTVKAIGWAVLEAAGVLSPFEFSRRATGERDVQVKVLYCGICNFDTEMINNKFGFTRYPFVLGCTNNLENYCPKAVLIEANNGGCSNLVVADEDFVFHWPEKLPLDLGAPLLCAGITTYSPLKNFGLDKPGLHVGVAGLGGLGHVAIKFAKAFGAKVTVISSSDKKKKEAIEKYGADEFLVSSNPEQMQATAGTLNGIIDTVPSPHSLVPLLNLLLPQGKLIVLGAPIEPFVLPIYPLLQGGRVVAGSATGGLKQIQEMLHFAAEHSIAADAEVIPIDYINTAVERIEKGDIKYRFVVDIGNTLKSA

SEQ ID NO: 62 - protein, 360 aa, synthetic construct
MAAEQSPQDQHQVKVLGWAARDSSGILSPFKFSRRATGEHDVQLKVIYCGICTYDVEMIKNKWGMTNYPVVFGHEAVGVVTEIGSKVQKFKVGDTVGVGTQVGSCGECDCCTNDLNQYCPEMIATDGFGSNDVSCGACCDIMVVDENFVIVWPQNLPMDAGAPLLCAGITTYSPLRNFGLDKPRIHVGIVGLGGLGHVAVRFAKAFGAKVTVISTSMNKKQEALHKYGADSFVVSSEQEQMQAAVSTMNGIIDTSPVPHALVPLLKLLKPQGKLILIGAPDKPYELPVFPLIEGGRIVAGSATGSFRQTQEMIDFAAKHNIVADVEVIPTNYLNTAVERIEKGDVKYRFVIDLGNGLKLA

SEQ ID NO: 63 - protein, 354 aa, synthetic construct
MAAGKCAQEEHTVKAFGWAAREASGALSPYGFSRRATGERDVRVKILYCGICRTDAEMINNKFGFTTYPFVLGHEIVGVVSEVGSKVKKFQFGDKVGVGTMIGACRTCYSCTNNLENYCPKAVLIEANNGGCSNLVVADEDFVFRWPEKLPLDLGAPLLCAGITTYSPLKNFGLDKPGLHVGVAGLGGLGHIAVKFLKAFGAKVTVISSSDKKKKEAIEKYSADEFLVSSNPEQLQAAAGTLNGIIDTVPSPHSLVPFLNLLLPQGKLIVLGAPIEPFVLPIFPLLQGGRVVAGSATGGLKQIQEMLLFAAEHNIAADAEVIPIDYINTAVERIEKGDIKYRFVVDIRNTLKSA

SEQ ID NO: 64 - protein, 362 aa, synthetic construct
MAAGKTPETVHPVKAFGWAARDTSGELSPFKFSRRATGEHDVQFKVLYCGICHSDLHMIKNEWGMTVYPIIPGHEIVGVVTEVGNKVEKVKVGDKVGVGCLVGSCGNCNMCSQDLENYCPKQILTYSAIDTDGSITYGGYSDVMVANEHFIIRWPENLPLDAGAPLLCAGITTYSPLRYFGLDKPGMHVGIVGLGGLGHVAVKFAKAFGSKVTVISTSENKKKEAIEKLGADAFLNSRDPQQMQAAVGTFDGILDTVSAVHPIVPLINLVKSQGKVIMVGAPEKPLELATFPLLVGRKTLSGSAIGGLKETQEMIDFAAKHNILADVEVIPMNYVNTAMERLLKADVKYRFVIDIGNSLKSA

SEQ ID NO: 65 - protein, 362 aa, synthetic construct
MAAGKCAQEEHPVKSFGWAARDTSGILSPFKFSRRATDEHDVRLKVLYCGICHSDLHTIKDEWGITEYPVVPGHEIAGVVTEVGSKVQKFKVGDKVGVGCFVGSCRNCNMCDKNLENYCPAQIFTYGSPYTDGTPTYGGYSDMMVVDEHFVIRWPENFPLDAGAPLLCAGITTYSPLRYFELDKPGIHIGIVGLGGLGHVAVKFAKALGAKVTVISTSASKRQEAFEKLGADSFLLSSDPEQIQAAVGTMDGIIDTVSAVHPIVPLIDLLAPHGKLIVVGSPAKPLELPIYNLSIGRKIVGASSVGGLKETQEMIDFAAKHNILPDVEIIPIDYVNTAMERLARGDVKYRFVIDVGNTLKST

SEQ ID NO: 66 - protein, 362 aa, synthetic construct
MSSAEKSPEEEHPVKAFGLAAKDSSGILSPFNFSRRATGDHDVQLRVLYCGLCYYDTKMIKNKRGVTRYPFVFGHEIVGEVTEIGREVQKFKVGDKVGVGCMVASCRSCESCANNCENYCPNVSVTDGAFFFKTGEVLYGGCSDIMVADENFVIRWPENFPLDAGAPLLCAGITTYSPLRNFGLDKPGIHVGIYGLGGLGHVAVQFAKAFGAKVTVISSSDRKRMEAIEKLGADSFLVNSNLEEMQAAMGTMHGIIDTVPANHSLVPLLDLLKPQGKLIVVGGPEKPFELPVFPLLQGGRLVAGSATGGIKQTQEMIDFAAEHNILPHVEVVSVDYVNTAIERTERGDVKYRFVIDIGNTLY

SEQ ID NO: 67 - protein, 362 aa, synthetic construct
MSSAKTPEMVHPVRAFGWAARDTSGELSPFKFSRRATGEHDVQFKVLYCGICHSDLHMIKNEWGMTVYPIVPGHEIVGVVTEVGSKVEKVKVGDKVGVGCLVGSCGNCDMCSQDLENYCPKQILTYSAIDEDGSITYGGYSDVMVANEHFIIRWPENLPLDAGAPLLCAGITTYSPLRYFGLDKPGMHVGIVGLGGLGHVAVKFAKAFGAKVTVISTSENKKKEAIEKLGADAFLNSRDPQQMQAAVGTFDGILDTVSAVHPIVPLINLVKSQGKVIMVGAPEKPLELATFPLLVGRKTLAGSAIGGLKETQEMIDFAAKHNILAEVEVIPMNYVNTAMERLLKADVKYRFVIDIGNSLKSA

SEQ ID NO: 68 - protein, 362 aa, synthetic construct
MSSAKTPGTEHSVKAFGWAARDTSGELSPFKFSRRETGKHDVQFKVLYCGICHSDLHMVKNEWDMTRYPIVPGHEIVGVVTEVGSKVEKVKVGDKVGVGCLVGSCRNCEMCSQDLENYCAGQILTYSAIYTDGTITYGGYSDLMVADEHFVIRWPENFPLDAGAPLLCAGITTYSPLRYFGLDKPGTHVGVVGLGGLGHVAVKFAKAFGAKVTVISTSENKKKEAIEKLGADAFLVSRDPEQMQAAIGTMDGIIDTVSAVHPVVPLINLVKSQGKIVMVGAPEKPLEFATFPLIVGRKTLSGSAIGGLKETQEMIDFAAKHNILADVEVIPIDYVNTAMDRLLKADVKYRFVIDIGNSLKSA

SEQ ID NO: 69 - protein, 363 aa, synthetic construct
MSSAKSFEEEHPVKAFGWAARDSSGILSPFKFSRRATGKKDIRFKVLYTGICHSDLHHLKNDWGSSKYPLVPGHEIAGMVTEVGSEVKSFKVGDKIGVGCLIGSCGTCEDCADGLENYCPKKILTYDGAKYYDGTMTFGGYSDHMIVDEHFAIHWPENMPLDGGAPLLCAGITTYSPMKYFGLDKPGLHIGVVGLGGLGHVAVKFAKAFGAKVTVISSTPGKKEEAIKNFKADSFLVSRDTAAMQAAAGTLHGILDTVSAVHPILPLLSLLKNHSKYIMLGAPEKPLELPVFPMLMGRKIVAGSNIGGVKETEEMLNFAAEHGITADVEVIPIDYVNTAVERLGKNDVRYRFVIDVGNTLKAT

SEQ ID NO: 70 - protein, 355 aa, synthetic construct
MSSAGKCPQEEQTVKAFGWAAREVSGALSPYGFSRRATGERDVKINVLYSGMCNFDTEMINNKFGFTKYPYVLGHETVGVVSEVGSKVRKFKVGDKVGVGGLVGSCRKCYSCTNDLENYCTKAELTEAGAGGCSNFVVTDEDFVFHWPEKLPLDLGAPLLCAGVTTFSPLKTHGLDKPGMHVGVAGLGGLGHVAVKFLKAFGAKVTVISTSDNKKREAIEKYGADAFLVSSNPEQMQTAAGTLTGIIDTIPSAHSLVPLLNLLMPQGKLIVLGVPTEPFVLPVVPMIQGGKVVAGSSFGGLKLIQEMLDFAAEHNIVADAEVIPVDYINTAIKRIETGDIKYRFVIDVGNTLKSP

SEQ ID NO: 71 - protein, 355 aa, synthetic construct
MSSAGKCAQEEHTVKAFGWAAREASGALSPYGFSRRATGERDVRVKILYCGICRTDAEMISDKFCFTKYPHVPGHEIVGVVSEVGNKVQKFKVGAKVGVTGMIGSCRTCYSCTNNLENYCQKAVLIEANNGGCSNLVVADEDFVFRWPEKLPLDLGAPLLCAGITTYSPLKNFGLDKPGLHVGVAGLGGLGHIAVKFLKAFGAKVTVISSSDKKRKEAIEKYGADEFLVSSNPEQMQAAAGTLNGIIDTVPSPHSLVPFLNLLLPQGKLIVLGAPIEPFVLPIYPLLQGGKVVAGSATGGLKQIQEMLHFAAEHNIAADAEVIPIDYINTAVERIEKGDIKYRFVVDIGNTLKSA

SEQ ID NO: 72 - protein, 352 aa, synthetic construct
MSSAGKSPEEEQTVKAIGWAASEAAEVLSPCEFSRRTTGEHDVQVKVLYCGICNFDTEMINNKFGFTRYPFVLGHEIVGVVSEVGSKVKKFQVGDKAGVGTMIGTCRTCYSCTNNLENYCPKAVLIEANNGGCSNLVVADEDFVFRWPEKLPLDLGAPLLCAGITTYSPLKNFGLDKPGLHVGVAGLGGLGHVAVKFAKAFGAKVTVISSSDNKKKEAIEKYGADEFLVSSNIEQMQAAAGTLNGIIDTVPSPHSLVPLLNLLLPQGKLIVLGAPTEPFVLPIYPLLQGKSIMPSFCFAMICIGQKVINNVHYVLIVVYYRRKNCRWKCHWRFEANSRNAPFCSRTQHSSRC

SEQ ID NO: 73 - protein, 362 aa, synthetic construct
MSSAKSLEEQHPIKAFGWAARDVSGVLSPFSFSRRTTGEKDIQIKILYCGICHSDLHCVKNEWGFSNYPIVPGHEIVGVVTEVGSNITKFKVGDKVGVGCMVGSCLSCENCSNNLENYCKKVIPTYNSTYYDKTHTYGGYSDILVVNEHFVVRIPENMPLDAGAPLLCAGITTYSPLRYFGLDKPGMHIGVVGLGGLGHVAVKFAKAFGVKVTVISTSLHKKKKALEHLGADSFLVSTDKDQMQAAMESMDGILDTVSAVHPLLPLINLLKSHGKFVMVGAPNRPLELPVFPMLAGRKILAGSGIGGMKETQEMLDFAAEHNIVADVEVISMDYVNTAMERLARADVKHRFVIDIGNTLKTA

SEQ ID NO: 74 - protein, 363 aa, synthetic construct
MSSAKSFEEEHPVTAFGWAARDTSGFLSPFKFSRRVTGETDVRFKVLFTGICHSDLHHLKNDWGASKYPLVPGHEIVGVVTEVGSKVQKFKAGDKVGVGCLIGSCRTCEDCAEDLESYCPKKITTYDGPRYYDGSLTFGGYSDHMVVDEHFVIHWPEKLPLDGGAPLLCAGITTYSPMKYFGLDKPGLHIGVVGLGGLGHVAVKFAKAFGAKVTVISSSPSKKEEAIKNFGADSFLVSRDTEAMQAAAGTLHGILDTVSAVHPLLPLLSLLKNHSKYIMLGAPEKPLELPVFPMLMGRKIVAGSNIGGIKETEEMLNFAAEHGITADVEVIPMEYVNTAIERLGKNDVRYRFVIDVANTLKAA

SEQ ID NO: 75 - protein, 355 aa, synthetic construct
MSSAGKSPEKEQTVKATGWAASEVSGILSPIEFSRRATGDSDVQIKVLYCGICSFDTEMINNKFGFTRYPYVLGHETVGVVSEVGSKVRKFKVGDKVGVGGLVGSCGTCYGCTNGLENYCTKEQITEAGAGGCSNFMVMGEDFVFHWPEKLPLDLGAPLLCAGVTTYSPLKTHGLDKPGLHVGIAGLGGMGHVAVKFAKAFGAKVTVISTSDNKKEEAIKKYGADAFLNSSNPEQMRAAAGTLAAIVDTIPSPHSLVPLLDLLLPHGKVIVLGAPSEPFVLPVMPLLQGGRVVAGSSGASLKQIQEMLDFAAEHNIVADAEVIPIDYINTAIKRIEKGDIKYRFVVDIGNTLKSA

SEQ ID NO: 76 - protein, 355 aa, synthetic construct
MSSAGKSPEKEQTVKATGWAASEVSGILSPIEFSRRATGDSDVQIKVLYCGICSFDTEMINNKFGFTRYPYVLGHETVGVVSEVGSKVRKFKVGDKVGVGGLVGSCGTCYGCTNGLENYCTKEQITEAGAGGCSNFMVMGEDFVFHWPEKLPLDLGAPLLCAGVTTYSPLKTHGLDKPGLHVGVAGLGGLGHVAVKFLKAFGAKVTVISSSDNKKKEAIEKYGANAFLVSSNPEQMQAAAGTLSGIIDTIPSPHSLLPLLDLLLPQGKLIVLGAPIEPFVLPIYPLLQGGRVVAGSATGGLKQIQEMLDFAAEHNVAADGEVIPIDHVNTAVKRIETGNIRHRFVIDIGNTLKSA

SEQ ID NO: 77 - protein, 355 aa, synthetic construct
MSSAGKSPEEEQTVKAIGWAASEVSGVLSPFEVSRRATGERDVQVKVLYCGICSFDTEMINNKFGFTKYPFVLGHEIVGVASEVGSKVQKFQVGDKVGVGALIGSCRTCYSCTNDLENYCPKAVLTEANSGGCSNFVVADEDFVFRWPEKLPLDLGAPLLCAGITTYSPLKNFGLDKPGLHVGVAGLGGLGHVAVKFLKAFGAKVTVISSSDNKKKEAIEKYGANAFLVSSNPEQMQAAAGTLSGIIDTIPSPHSLLPLLDLLLPQGKLIVLGAPIEPFVLPIYPLLQGGRVVAGSATGGLKQIQEMLDFAAEHNVAADGEVIPIDHVNTAVKRIETGNIRHRFVIDIGNTLKSA

SEQ ID NO: 78 - protein, 361 aa, synthetic construct
MSSAEQSPEEQHPVKVLGWAARDSSGILSPFKFSRRATGEHDVQIKVIYCGICTYDVEMIKNKWGMTNYPVVFGHEAVGMVTEIGSKVQKFKVGDTVGVGTQVGSCGKCECCTNDLDQYCPEMIATDGFGSNDVSYGACCDIMVVDENFVIVWPQNLPMDAGAPLLCAGITTYSPLRNFGLDRPGIHVGVVGLGGLGHVAVRFAKAFGAKVTVISTSMNKKQEAVHKYGADSFLVSTDQEQMQVNTAWVAAIVDTIPSPHSLVPLLDLLLPHGKVIVLGAPSEPFVLPVMPLLQGGRVVAGSSGASLKQIQEMLDFAAEHNIVADAEVIPIDYINTAIKRIEKGDIKYRFVVDIGNTLKSA

SEQ ID NO: 79 - protein, 344 aa, synthetic construct
MSSERVEDLTELLAAQHHVGNQMLNFRKSASLKCAIELGIPDAINQHGKPITLCELVSALPINPSKANHIYRLMRFLSNAGFFILQDQGYALTAAGRLLLKEEPFNLRAFIFYMSDPAVVKPWNSLTEWFSNDDPFPFDTAHGKNFWAYAAEEPNFANLFNEAMANDSTLIVQVMMTQCKFVFDGLTSLADVGGGTGAVARAIAQNFPNLKCVVCDLPHVIADQEGTENLDFVAGDMLEKVPAADAILLKWILHDWSDEDCVKILKNCKEAIPGRDKGGKVIIIDMIMESQMKDDESVETQVGVNMQMLMGYGAKERSEKEWAKLFQDAGFSDYKVLPLLCVSH

SEQ ID NO: 80 - protein, 359 aa, synthetic construct
MSSDAMKSAELFKAQAHIFKQVFCFTNGASLKCAVQLGIPDAIDNHGKAMTLSELTDALPINPSKAPHIHRLMRILVTAGFFVEERLGNGKEEKANGYALTPSSRLLLKNKPLSLRASALTMLDPVTVKTWNALSEWFQNEDQTAFETAHGKNMWDFFAEDPGLSKKFNESMASDSQLVTEVLVTKCKFVFEGLTSMVDVGGGTGTVAGAIAKTFPSLRCTVFDLPHVVANLEPTENLDFVAGDMFGKIPPANAIFLKWVLHDWNDEDCVKILKNCKRAIPGKEKGGKVIIVDIIMETEKHDIDEFDYAKMCMDMEMLVLCNSKERTEKELAMLVSEAGFSGYKIFPVLGIRSLIEVYP

SEQ ID NO: 81 - protein, 360 aa, synthetic construct
MSSGSAEEEASLFAMQLVTSASLPMVLYTAVKLNLFEIIAKAGPGAKLSPSEIACRLPTTKNPDAASMLDRMLRLLSSHSLLTYDVVEVAGGAETDIGYERVYGLSPVGEYLIPDEEGNSVAPLVELLLDKVLINTWYELGNAVLEGGIPFNRVHGTHAFDYPSRNPRYNELFNNGMVGPTAITMKELLQQYKGFELLQTLVDVGGGLGITLHKIIAKYPSIRGINFDLPHVIEDAPPYPGMEHIGGDMFESVPGGDAIFMKMILHDWSDDHCLKLLKNCYRALPDHGKVIVVDLILPVKPDTNIFVKGIFQADVIMMTQNPGGKERSEADVRALAIGAGFKGIKLECCAGNVGILELYK

SEQ ID NO: 82 - protein, 348 aa, synthetic construct
MSSDVQSAELLRAQAHVWSQTFNFKNSASLKCAVQLGIADAIQKHGKPITLSELAAALPINPSKANYIYRLMRILVNAGFFAQEKEGYSLTAAGRLLLSDEPVNARAFILVVLDPDTMKPWNFLSDWFQNDDPSPFDTAHGKNFWDFTAEEPKFSKLFNEGMVSDSHLITMVLVTECKYVFEGLTSLVDVGGGTGTVARSIAEAFPNLKCTVFDLPHVVANQEGTLNLDFVAGNMLEKVPPANAILLKWILHDWSDEDCVKILKNCKKAIPGRDIGGKVIIIDMVMESQLEGDESTEAQICLDMQMLVLYRAKERSEKEWAKLFSDAGFPSYKVFPALGTRAIIEVYP

SEQ ID NO: 83 - protein, 348 aa, synthetic construct
MSSDVQSAELLRAQAHVWSQTFNFKNSASLKCAVQLGIADVIQKHGKPITLSKLAAALPINPSKANYIYRLMRILVNAGFFAQEKEGYSLTSAGRLLLSDEPVNARAFILVVLDPDTIKPWNFLSDWFQNDDPSPFDTAHGKNFWDFTAEEPKFSKLFNEGMVSDSHLITMVLVTECKYVFEGLTSLVDVGGGTGTVARSIAEAFPNLKCTVFDLPHVVANQEGTLNLDFVAGNMLEKVPPANAILLKWILHDWSDEACVKILKNCKKAIPGRDIGGKVIIIDMVMESQLEDDESTEAQICLDMQMLVLYRAKERSEKEWAKLFSDAGFPSYKVFPALGTRAIIEVYP

SEQ ID NO: 84 - protein, 358 aa, synthetic construct
MSSDVQAGELLQAQAHIWNHIFNFINSMSLKCAVQLDIPDIIHKHGQPMTLAQLIHALPISSAKAPFVHRLMRILIHSGFFVKAKIPGNELEEGYELTSASKLLLKSNPLSVTPFLLAMLDPILTDPWHHFSQWFQCDDETPFDTCHGKAFWKLAGEQPRLNQFFNEGMASDARLISSLVIKHCKHVFEGLNSLVDVGGGTGTLAMAIADAFPDLKCTVLDLPHVVAGLDGSSKNLAYVGGDMFEAIPPTDAVLLKWILHDWSDEECVQILKRCKEAIPSKGKGGKVIIIDILVKDQHRGDDNDEAIETQLFFDMLMMVLVKGRERNQKDWAKLFFEAGFNDYKITPVLGLRSLIEVY

SEQ ID NO: 85 - protein, 357 aa, synthetic construct
MSSDVHAGELLQAQAHIWNHIFNFINSMSLKCAIQLGIPDIIQKHGQPMTLAQLIHALPISNAKAPFVYRLMRILIHSGFFIKAKIPGNELEEGYELTPASKLLLKSNPYSVTPFLLAMLDPILTDPWHNFSQWFQSNDETPFYTCHGRELWELAGEKPRLNQFFNEGMASDARLISSLVIKHCKHVFEGLNSLVDIGGGTGTLANAIADAFPDLKCTVLDLPHVVAGLDGSSKNLAYVGGDMFEAIPPTDAVLLKWILHDWSDEECVQILKKCKEAIPSKEKGGKVIIIDMLMKNQQKGDDEDKAIETQLFFDMLMMVLVKGKERNQKDWVKLFFEAGFNDYKITPVLGLRSLIEI

SEQ ID NO: 86 - protein, 360 aa, synthetic construct
MSSDVQADKLLEAQAHIWNHTFNFINSMCLKCAIQLGIPDIIKKHGRPMTLEELTDALPINNAKSPFIYRLMRILIHSGFFIKAKVTDTDVEKGYMLTSASELLLANDPFSMKTFLLAMLDPILTAPWQHFSQWFQSNDENPFYTCHGRAFWELAGQEPRLNQFFNEAMANDARLVSSIVIKHCKHVFSGLNSLLDVGGGTGTFGKAIAAAFPDLKCTVLDLPHVVDGLDVGGKNLAYIGGDMFEDIPPADAVLLKWILHDWTDEECVRILEKCKQAIPSKENGGKVIIIDMVRNDQQKEADDDARKSIETQLFFDILMMVDTKGRERNEKEWAKLFYEAGFKDYKITQVVGLRSLIEVY

SEQ ID NO: 87 - protein, 358 aa, synthetic construct
MSSDVQAGELLQAQAQIWNHIFNFINSMSLKCAVQLDIPDIIHKHGQPMTLAQLIHALPISSAKAPFVYRLMRILIHSGFFLKAKIPGNELEEGYELTSASKLLLKSNPFSVTPFLLSMLDPILTDPWHQFSQWFQCDDETPFYTCHQKAFWKLAGEHPRVNQFFNEGMASDARLISSLVMKHCKHVFEGLNSLVDVGGGTGTLAMTIADAFPDLKCTVLDLPHVVAGLDGSSKNLAYVGGDMFEAIPPTDAVLLKWILHDWSDEECVQILKKCKEAIPSKGKGGKVIIIDMLVKDQQRGDDKDEAIETQLFFDMLMMVLVKGRERNQKDWAKLFFEAGFNDYKITPVLGLRSLIEVY

SEQ ID NO: 88 - protein, 360 aa, synthetic construct
MSSDVQADKLLEAQAHIWNHTFNFINSMCLKCAIQLGIPDIIKKHGRPMTLVELTDALPINSAKSPFIYRLMRILIHSGFFIKVKVTDTDVEKGYMLTSASELLLANDPFSMKTFLLAMLDPILTDPWQHFSQWFQSNDENPFYTCHGRAIWELAGQEPRLNQFFNEAMANDARLVSSIVIKHCKHVFSGLNSLLDVGGGTGTFAEAIAAAFPDLKCTVLDLPHVVDGLDVGSKNLAYIGGDMFEDIPPADAVLLKWILHDWSDEECVRILEKCKQAIPSKENGGKVIIIDMVRNDQQKEADDDAHKSIETQLFFDILMMVDTKGRERNEKEWAKLFYEAGFKDYKITQVVGLRSLIEVY

SEQ ID NO: 89 - protein, 359 aa, synthetic construct
MSSDVQADKLLEAQAHIWNHIFNFINSMSLKCAIQMGIPDIIKNHGRPMTLKELTDALPINNAKSPFVYRLMRILIHSGFFAKAKVTDNDVEEGYVLTSASELLLADDPFSLTPLLLAMLDPIMTDPWQHFSQWFQNNDETPFYTCHGRTFWELASQEPRLNQFFNEAMARDAWLVGSVVINHCKHVFSGLNSLVDVGGGTGTLAKAIADAFPDLKCTALDLPYVVDGLDGSSKNLVYIGGDMFEAIPPADAVLLKWILHDWTDEECVRILVKCKQAIPSKENGGKVIIIDMVIIIDMVKGANDHRSIETQLFFDLLMLVDVKGKERNEKERAKLFYEAGFNDYKITQVGGLRTLIEVY

SEQ ID NO: 90 - protein, 267 aa, synthetic construct
MSSAELYNRQAKQYSDSRPTYPEELFQFIASKTPCHDLAWDVGTGSGQAARSLAGIYKNVVATDISPKQLEFAVKLPNIRYHCTPPKMPIAEVEQNIAAESSVDLVTIATAIHWFDLPTFYQQVKWVLKKPHGVIAAWSYINVEINTCSVNTLLQKLYLDCAPYCEPQAKSVADDKYRTIDFPFQPVDGLDNTGPFEFKMEKLMDLDGFFTFIKSWSLYQTAKDKGVELLNDDVVQDFTRAWNEDGNPQKIATFPIYLRIGKVGNWY

SEQ ID NO: 91 - protein, 101 aa, synthetic construct
MSSAMNQWILHDWSDEKCVQVLKKCKEAIPSKEKGGKVIIIDMLVKNQQKGDDNDEPIETQLFFDMLMMILYKGRERNQKDWEKLFLQAGFNYYKIIPVLG

SEQ ID NO: 92 - protein, 354 aa, synthetic construct
MSSDFARNGDHTGELFQAQAHIWNHLFNFINSMSLKCAIQLGIPDIIHKHGQPMALAQLIDALPINNAKAHFVYRLMRILIHSGFFIKAKMPDNEGHEGYALTSASKLLLANDPFSVTPFLLAMLDPILTDPWHHFSQWFQDNEETPFHSCHGTSMWELAGRQPRLNQFFNEGMASDARLVCTMVIKNCKDVFMGLNSLIDVGGGTGTVAKAIADAFPHLKCSVLDLPHVVDGSESSKNLAYVGGDMFEAIPPSDAAMNQWILHDWSDEKCVQVLKKCKEAIPSKEKGGKVIIIDMLVKNQQKGDDNDEPIETQLFFDMLMMILYKGRERNQKDWEKLFLQAGFNYYKIIPVLG

SEQ ID NO: 93 - protein, 365 aa, synthetic construct
MSSDLARNGDQAGELLQAQAQIWNHIFNFINSMSLKCAVQLDIPDIIHKHGQPMTLAQLIHALPISSAKAPFVYRLMRILIHSGFFLKAKIPGNELEEGYELTSASKLLLKSNPFSVTPFLLSMLDPILTDPWHQFSQWFQCDDETPFYTCHQKAFWKLAGEHPRVNQFFNEGMASDARLISSLVMKHCKHVFEGLNSLVDVGGGTGTLAMTIADAFPDLKCTVLDLPHVVAGLDGSSKNLAYVGGDMFEAIPPTDAVLLKWILHDWSDEECVQILKKCKEAIPSKGKGGKVIIIDMLVKDQQRGDDKDEAIETQLFFDMLMMVLVKGRERNQKDWAKLFFEAGFNDYKITPVLGLRSLIEVYYY

SEQ ID NO: 94 - protein, 172 aa, synthetic construct
MSSEGAGGTLQTCCWSPGPRAVSKCNGHECFPRWFAAVLAKYPVWVMNVVPAHSDPDTLSIIYERGFIGTYHDWCEAFSTYPRTYDLIHADGVFSIYQDRCDITYILLEMDRILRPEGTVIFRDVVEVLVKIKAITDGMRWESRILDHESGPFNPQKILVAFKSYWTAKAND

SEQ ID NO: 95 - protein, 362 aa, synthetic construct
MSSDTNVERSKLLHGQAEIWKHTFRFADSMALKCAVELGIADIIHSHGHPLTLAEIATNITNSSSPHIPYLSRIMRFLVRNKIFTSTDIPSDHGGGSPSTTLYGLTAASWWLLNDNDGLSLAPFLLMENHPWLVSPWHQLSSCVREGGIAFQKAHGKEIWELASENPEFNTIFNDGMGCTGKITMKAILSGFKDAWNSIESLVDVGGGTGQTIAEIVKVYPHIRGINFDLPHVVAAAPKYDGVCHVGGDMFDSIPTAQAVFMKWIMHDWGDEDCVKILKICRKAIPEKTGKIFIVDVVLKPDGDGLFDSIGMIFDLLMIAHSSGGKERTEPEWKKLLEKGGFPRYNITEIPACFSIIEAYPV

SEQ ID NO: 96 - protein, 279 aa, synthetic construct
MSSRFLVNGGFFIQQDQCYALTTAGRLLLKDEHINVTGLVALSTDPIMMMPWIFLTEWFKNDDPSPFDTTYGKSFWDYTAQESRFGKVFDEAMANASNLPIQVPITKCRFVFESLTSLANVGGGTGKVAKAIAKNFPNLKCVVCDLPRVVANQEGTKNLDFVAGDMLEKVPPANAALLKWVLHDWSDEHGLKILKNCKMSIPTREKGGKVIIIDIIIESHKEDNELVETQIGMDMQMLVLYVAKERTEEEWATLFLDAGFSSYKVFPVLGVSCLIEVYP

SEQ ID NO: 97 - protein, 350 aa, synthetic construct
MSSATLENSAELLRAQAHVWSQTFNFKNSASLKCAVQLGIADVIQKHGKPITLSKLAAALPINPSKANYIYRLMRILVNAGFFAQEKEGYSLTSAGRLLLSDEPVNARAFILVVLDPDTIKPWNFLSDWFQNDDPSPFDTAHGKNFWDFTAEEPKFSKLFNEGMVSDSHLITMVLVTECKYVFEGLTSLVDVGGGTGTVARSIAEAFPNLKCTVFDLPHVVANQEGTLNLDFVAGNMLEKVPPANAILLKWILHDWSDEACVKILKNCKKAIPGRDIGGKVIIIDMVMESQLEDDESTEAQICLDMQMLVLYRAKERSEKEWAKLFSDAGFPSYKVFPALGTRAIIEVYP

SEQ ID NO: 98 - protein, 366 aa, synthetic construct
MSSDLARNGDRAGELLQAQAHIWNHIFNYINSMSLKCAVQLGIPDIIEKHGQPMTLAQLCHALSISNTKASFLYRLMRVLIHSGFFIKAKIPGNELEEGYELTSASKLLLKSNPFSVTPFLLAMLAPISTDLWHHFSKWFQSDDETPFHTCHGTSGWEMASQEPWINQSFNEAMASDARLLSSILIEHCGHVFSGLNSLVDVGGGTGTLAKAIADAFPDLKCTVLDLPYVVDGLDGGRKNLAFIGGDMFEAVPPADAVLLKWTLHDWSDEDCVRILEKCKQAIPSKENGGKVIIIDMVINDQQKGADDGEHKSIETQLFFDMLMMVDTKGRERNEKEWAEIFSAAGFKDYKITHALGLRSLIEIYY

SEQ ID NO: 99 - protein, 450 aa, synthetic construct
MSSGSAEEDYPSCKRPNQHTQTKHKFKILLVVILTNLLTIYIFAGPSLPITRNLSLPTWDYTTKLLHELNVTKRELANSRAQISDLQQHLKASNILTKSLLTQLTRLNGAIDQHSTIESSLFNYADLLTDLSSEAKLGIGPHKLPLGYSPRSGSEEIYSSVGGGCLRYKEELAQYMTYKIGGECPVDDVFVQRLMLKGCEPLPRRRCHPKSPAGYKEPTPLPKSLWTTPPDTSIIWDPYTCKNYSCLVERKKLPGFYDCKDCFDLQGREISRWLFDNGGLDFGIDQVLGTKPHGTIRIGLDIGGGTGTFAARMKERNVTIVTTSMNLDGPFNSFIASRGLISMHVSVSQRLPFFENTLDIVHSMHILSNWIPDTMLEFTLYDIYRVLRPGGIFWLDHFFCLGSQLNATYVPMLDRVGFKKLRWNAGMKLDRGIDKNEWYFSSLLEKPTSF

SEQ ID NO: 100 - protein, 364 aa, synthetic construct
MSSGSAAPEQSNSMAGDEEACLFAMQLASASVLPMVLKSAIELDLLELIKKAGQDAYVSPSELAAQLPTENPDAPVMLDRILRLLASYSVLNCTLRNLPDGGVERLYGLAPVCKFLTKNEDGVSMAALLLMNQDKILMESWYHLKDAVLEGGIPFNKAYGMTAFEYHGTDPRFNKVFNQGMSNHSTIIMKKILEIYNGFEGLKTVVDVGGGTGATLSMIVSKYPAIKGINFDLPHVTGDAPTYPGIEHVGGDMFFSVPKGDAIFMKWICHDWSDEHCLKFLKNCYQALPENGKVILAECLLPEAPDATLSTQNTVHVDVIMLAHNPGGKERTEKEFEALAKEAGFKGFRKICCAYNSWIMELLK

SEQ ID NO: 101 - protein, 365 aa, synthetic construct
MSSNSPVQDQISNTPSEEEDFFKVMQLSSGLILPMILKAAIDLELFELIAKAGPGAKLSAVEIASQLSTKNPNAAVTLDRMMKFLASFSFLNCSLVTDAEGHPRRLYSFAPTCKNFLPDEDGVSFAPVLMLPVDKVHFESWYHLKDAVLEGGIPFIKANGDDAFAHPVKDTRFYEVFNKALHNHACIVTKRLLKVYRGFEGVKQLVDVGGGHGGTLGCIVSKYPHIHAINFDVPPVIEGAPPRPGVEFVGGDMFERVPKGEVLLMKWVLHDWSDEHCLKILKNCYEALPESGKLIVIELVLPETPNSDPLSKIGYQFDMNMMSVNLGGKERTEKEFEDLAKQSGFASIKLICRAGCDWVIEFYKD

SEQ ID NO: 102 - protein, 615 aa, synthetic construct
MSSATKGNPGDNRSRSSMPMFIAAGLCCVFYLLGAWQRSGSGKGDNIAYEITKKAEDCSILANLEFETHHGDRAANDSKLEVKEYEPCDDRYIDYTPCHDQARAMTFPRENMNYRERHCPPEQEKFHCLIPAPKGYVTPFRWPKSRDYVPFANAPHKSLTVEKAVQNWVQYEGNVFRFPGGGTQFPHGADAYIEQLASVIPINDGTVRTALDTGCGVASWGAYLFKKNVITMSFAPRDSHEAQVQFALERGVPAVIGVLGTIKLPYPSRAFDMAHCSRCLIPWGANDGKYMMEVDRVLRPGGYWILSGPPINWRNSYEAWQRPKEELEEEQRKIEEIAELLCWEKKHEKGETAIWRKRINNENCNDRDSRVTMCKLADADDVWYKKMEVCVTPYPETNSQEEVAGGELKPFPERLNAIPPRVAGASVLGVTIDSFQEDNRLWKKHVSAYQRINKLLDSGRYRNVMDMNAGLGGFAAAIESSNLWVMNVIPTIAEKDTSGIIFERGLIGIYHDWCEAFSTYPRTYDLIHAEGVFSLYKDKCNAEDILLEMDRILRPEGAVIFRDHADVLSEVKRIVAGMKWNTKMYDHEDGPLVPEKILAAVKQYWVAGDTNSTFS

SEQ ID NO: 103 - protein, 380 aa, synthetic construct
MSSATSENTQLKSVSPSSMENGKAGEEQDHFSDVMQLVTSAALPMVLYTVVKLNLFEIIAKAGPGAKLSPSEIASRLPATKNPDAASMLDRMLRLLSSHSLLTCEIGEVAGGGETEVGYERVYGLSPTGEYLIPDEEGNSVAPLVELLLDKVLTDTWYELGNAVLEGGIPFNRVHGTHAFDYPSRNPEYNELFNKGMVGPTAITMKKLLQQYKGFEHLQTLVDVGGGLGITLHKIVSKYPNISGINFDLPHVIEDAPSYPGVEHIGGDMFESVPGGDAIFMKMVLHDWSDDHCLKLLKNCFKALPDHGKVIVADLILPMKPNTSAFVKGIFQADVIMMTQNPGGKERSETDVRALAIGAGFKVVKLECQAGNLGILELYK

SEQ ID NO: 104 - protein, 380 aa, synthetic construct
MSSETSENAQLKSASPSSIENGKEGEKQDHFSHVMQLVTSASLPMVLYTAVKLNLFEIIAKAGPEAKLSPSEIAYRLLTTKNPDAASMLDRMLRLLSSHSLLTCDIVEVAGGAEIDIGYERVYGLSPVGEYLIPDEEGNSVAPLVELLLDKVLIDTWYELGNAVLEGGIPFNRVHGTHAFDYPSRNPRYNELFNNGMVGPTAITMKELLQQYKGFELLQTLVDVGGGLGITLHKIIAKYPSIRGINFDLPHVIEDAPPYPGMEHIGGDMFESVPGGDAIFMKMILHDWSDDHCLKLLKNCYRALPDHGKVIVVDLILPVKPDTNIFVKGIFQADVIMMTQNPGGKERSEADVRALAIGAGFKGIKLECCAGNVGILELYK

SEQ ID NO: 105 - protein, 380 aa, synthetic construct
MSSEISENAQLKSASPSSIENGQEGEKQDHFSHVMQLVTSASLPMVLYTAVKLNLFEIIAKAGPGAKLSPSEIACRLPTTKNPDAASMLDRMLRLLSSHSLLTYDVVEVAGGAETDIGYERVYGLSPVGEYLIPDEEGNSVAPLVELLLDKVLINTWYELGNAVLEGGIPFNRVHGTHAFDYPSRNPRYNELFNNGMVGPTAITMKELLQQYKGFELLQTLVDVGGGLGITLHKIIAKYPSIRGINFDLPHVIEDAPPYPGMEHIGGDMFESVPGGDAIFMKMILHDWSDDHCLKLLKNCYRALPDHGKVIVVDLILPVKPDTNIFVKGIFQADVIMMTQNPGGKERSEADVRALAIGAGFKGIKLECCAGNVGILELYK

SEQ ID NO: 106 - protein, 371 aa, synthetic construct
MSSATSENTQLKSVSPSSMENGKVEEEEQLFSYAMQLVTSVSLPRVLLAAFRLRVFEIIEEAGPGAQLSASEIAAQVSGKNKDAAAMLDRMLRLLASHSVLTCGARSQGRVYGLAPVAKYFVQSKADGGGTLGSLLALIQDKVFIDSWFQLEDAVREGGVPFDRVHGTHAFEYPGRDPRFNEVFNKAMLDHTTIAINRILERYKGFENLKTLVDVGGGLGVTLNVITTKYPNLKGINFDLPHVIQRAPGYPGVEHIGGDMFESVPEGEAIFMKWILHDWDDGHCLKLLKNCYKALSDNGKVIVVDALVPVIPDTSPCVKTTCQSDLIMMTQNPGGKERSEPEFLALATAAGFKGIRLECYVCNLWVMEFYK

SEQ ID NO: 107 - protein, 371 aa, synthetic construct
MSSATSENTQLKSVSPSSMENGKVEEEEQLFSYAMQLVTSVSLPRVLLAAFRLRVFEIIAEAGPGAQLSASEIAAQVSGKNKDAAAMLDRMLRLLASHSVLSCGARSQGRVYGLAPVAKYFVQSKADGGGTLGSLLALIQDKVFIDSWYQLEDAVREGGVPFDRVYGTHAFEYPGRDPRFNEVFNKAMLDHTTIAINRILERYKGFENLKTLVDVGGGLGVTLNVITTKYPNLKGINFDLPHVIQRAPGYPGVEHIGGDMFESVPEGEAIFMKWILHDWDDGHCLKLLKNCYKALSDNGKVIVVDALVPVIPDTSPCVKTTCQSDLIMMTQNPGGKERSEPEFLALATAAGFKGIRLECYVCNLWVMEFYK

SEQ ID NO: 108 - protein, 377 aa, synthetic construct
MSSATSENTQLKSVSPSSMENGKAGEEEEHCSYALQLATSAALPVVLNAIIKLNVLEIIAKAGPGAQLSPSEIAAHLPTENSGAAVMLDRMLRLLASYSVLTCSVVGGGDKDNQHQRAYGLAPVAKYFLRDEDGGSLGSLLDLLQDKVLVDIWYELEPAVLKGGTAFNRTHGTHIFQYTGSDQKFNETFNKAMIDHTFIVGRKVLQFYKGFEHLKTLVDVGGGLGVNLSMITSKYPSIKGINFDLPHVVQNAPAYPGVEHVGGDMFESIPKGDAIFMKWILHDWDDDHCLTLLKNCYKALPDNGKVIAVDAILPMVPDNSASCKSNCQVDLFTLALYFPGGKERTEQEFLALAKEAGFKGIRLECFACNMWVIEFYK

SEQ ID NO: 109 - protein, 374 aa, synthetic construct
MSSATSENTQLKSVSPSSMENGKGSAQEEASLFAMQLASASVLPMVLKSAIELDLLELIAKAGPGAYVSPSKLAAQLPTHNPQAPVMLDRILRLLATYSVLNCKLRDLPDGGVERLYGLAPVCKFLTKNADGVSMAPLLLMNQDKVLMESWYHLKDAVIDGGIPFNKAYGMTAFEYHGTDPRFNKVFNQGMSNHSTITMKKILEVYKGFEGVKTVVDVGGGTGATLNMIIDKYPTIKGINFELPHVVEDAPSYPGVEHVGGDMFVSVPEGDAIFMKWICHDWSDDHCRKLLKNCYHALPEGGKVILAECILPDAPDTSLATQNVVHIDVIMLAHNPGGKERTEKEFEALAKGAGFKEFRKICSAVNTWILELCK

SEQ ID NO: 110 - protein, 377 aa, synthetic construct
MSSATSENTQLKSVSPSSMENGKAGEEEEHCSYALQLATSAALPVVLNSIIKLNVLDIIAKAGPGAQLSPSEIAAHLSPENSGAAVMLDRMLRLLASYSVLTCSVVGGGDKDNQHQRAYGLAPVAKYFLRDEDGGSLGSLLDLLQDKVLVDIWYELEPAVLKGGTAFNRTHGTHIFQYTGSDQKFNETFNKAMIDHTFIVVRKVLQFYKGFGHLKTLVDVGGGLGVNLGLITSKYPSIKGINFDLPHVVQNAPAYPGVEHVGGDMFESIPKGDAIFMKWILHDWDDEHCLKLLKNCYKALPDNGKVIAVDAILPMVPDNSASCKSNCQVDLFTLALYFPGGKERTEQEFLALAKEAGFKGIRLECFACNMWVIEFYK

SEQ ID NO: 111 - protein, 374 aa, synthetic construct
MSSATSENTQLKSVSPSSMENGKGSAEEEASLFAMQLASASVLPMVLKSAIELDLLELIAKAGPGAYVSPSKLAAQLPTHNPQAPVMLDRILRLLATYSVLNCKLKNLPDGGVERLYGLAPVCKFLTKNADGVSMAPLLLMNQDKVLMESWYHLKDAVIDGGIPFNKAYGMTAFEYHGTDPRFNKVFNQGMSNHSTITMKKILEVYKGFEGVKTVVDVGGGTGATLNMIIDKYPTIKGINFELPHVVEDAPFYPGVEHVGGDMFVSVPEGDAIFMKWICHDWSDDHCRKLLKNCYHALPEGGKVILAECILPDAPDTSLATQNVVHIDVIMLAHNPGGKERTEKEFEALAKGAGFKEFRKICSAVNTWIMELCK

SEQ ID NO: 112 - protein, 375 aa, synthetic construct
MSSATSENTQLKSVSPSSMENGKAGERQEELQKLLQLTGTLTVHVALTAAIELNLFEIMAKSGPNAQLSSAQIVSLLRTKNPDAPAILDRILQLLGSYSILKCTIVSNDQNGHTTKLFGLTPMAEYLVPDSNGISFAPAILMAREKAFAKCGYFLKDAILEGGTPFHKVNGMHLFEYIGSSSRLNEVFNRIMACHTSVFMGAVLETYKGFEDLKELVDVGGGHGTTLKMILSKYPRIRGINFDLPFVIKNAPACPGVEHVEGDMFQNVPSVEAILLKNVLHDWNDDRCLKLLKNCWDALPESGKVIAIDAIIPELPSTDSASQFAYISDVEILIAAPGGRERTFKEFNALATAAGFTACKLVARTSQYGVLEFYK

SEQ ID NO: 113 - protein, 337 aa, synthetic construct
MSSATSENTQLKSVSPSSMENGKAGEEGEQCSYAMQLATSAALPNVLNAIIRLNVLEIIARAGPGAQLSTSEIAAHLHTQNSNAAVMLDRMLRLLASYSVLTCSVVGGGGGGGDDKDDQHQRVYGLAPVAKYFLQNEDGGSLRPLLDLFQGKVLTDIWCELEPAVLEGGTAFNRKHGIHIFEYTGRDQKFNESFNKAMIDHTFIVVKNVLQFYRGFEHLKTLVDIGGGLGVTLGMITSKYPNIKGINFDLPHVVQNAPTYPDNGKVIAVDAILPTIPDNSASSKSNIQMDLFTLASYFPGGKERTEQEFLALANEAGFKGIRLECLVCNMWVMEFYK

SEQ ID NO: 114 - protein, 615 aa, synthetic construct
MSSVTKGNPGDSRNKSSVSMFIVAGLCCVFFLLGAWQRSGSGKGDNIAYEITKKAKACSILGNLEFETHHGNKAVNDSMLEVKEYKPCDDQYIDYTPCQDQARAMTFPREDMNYRERHCPSEQEKLHCLIPAPKGYVTPFRWPKSRDYVPYANAPHKSLTVEKAVQNWVQYEGNVFKFPGGGTQFPHGADAYIEQLASVIPINDGTVRTALDTGCGVASWGAYLFKKNVITMSFAPRDSHEAQVQFALERGVPAVIGVLGTIKLPYPSRAFDMAHCSRCLIPWGANDGKYMMEVDRVLRPGGYWILSGPPINWRNSYQAWQRPKEELQEEQRKIEEIAELLCWEKKHEKGETAIWRKRINEDYCSHGDSRVTKCRSVDADDIWYKKMENCVTPYPETRSQEEVAGGELKPFPERLHAIPPRVDSGSIPGVSIDSFQEDNRLWKKHMSAYRRINKLLDSGRYRNVMDMNAGLGSFAAAFESPKLWVMNVMPTIADRDTLGVIFERGLIGIYHDWCEAFSTYPRTYDLIHAEGVFSLYKDKCNMEDILLEMDRILRPEGAVILRDHADVLTEVKRIIAGMRWNMKMYDHEDGPLVPEKILVAVKKYWVAGDKNSTFS

SEQ ID NO: 115 - protein, 451 aa, synthetic construct
MSSGSGDDDHPSSKRPIQHTQTKYKLKILLLVILTNLLTIYVFTGPSLPIAQNLSLPTWDHTKLLNELNITKRELANSRAQNSDLQQHLKASNILTKSLLTELSRLNAAIDQHSSSKEGSSIFSFDDLLSDLSTEAKIGMGPQKLPLGYSPRSGTDELYPSVGGGCLRYKEELAQYMTYKIGGECPVDDVFAQRLMLKGCEPLPRRRCHPKSPAGYKEPNPLPKSLWTMPPDTSIVWDPYTCKSYGCLVERKKLPVFYDCKDCFDLEGREKSRWLFDNGGLDFGINEVLGTKPHGTIRIGLDIGGGTGTFAARMKENNVTIVTTSMNLDGPFNSFIASRGLISMHMSVSQRLSFFENTLDIVHSMHILSNWIPDTMLEFTLYDIYRVLRPGGIFWLDHFFCLGSQLNSTYVPMLDRVGFKKLRWNAGMKLDRGIDKNEWYFSALLEKPVKS

SEQ ID NO: 116 - protein, 1411 aa, synthetic construct
MSSESPGQWLEKALLDLCKKIETGLDLDGDIISGLVSYCELASPLDAKEYLDNIIGQEAGKNVTEEYLKRRGHSDNYNSTSDAPTSKLHAYVKPPSNEVLVSGTRKPSRAPKELAASSNQEKRAPTETVELRNMNKGNQGSSRKKKAGKIVSLAEAAKGSIVFQQGKPCSCQARRHRLVSNCLSCGKIVCEQEGEGPCTFCGALVLKEGSSYAGLEEGAVPLSDAEATAEAYTKRLVEYDRNSAARTTVIDDQSDYYEIEGNSWLSMEEKELLRKKKEEIEEAERTKRSKLIMTFDLVGRKVLLNEDEVSELEMENRILRPPDEREANRIKPNPTLKVQPIFVDPGTSRKQPVKGNHLNKGLSNGLCLEISGRVQHGTNELKHFMMDDKLETASNGGLWLGPSVNDSLQIVISLLRSARRIMATKGNSGDNKTRSSVSIFIVAGLCCFFYILGAWQRSGFGKGDSIAQEITKKADCSILSNLNFETHHGDEAGNDDSKSEVEIFKPCNDRYVDYTPCQDQMRAMTFPRENMNYRERHCPPEEEKLHCLIPAPKGYVTPFPWPKSRDYVPYANAPYKSLTVEKAVQNWIQYEGNVFRFPGGGTQFPHGADAYIDQLASVIPIDNGMVRTALDTGCGVASWGAYLFKRNVIAMSFAPRDSHEAQVQFALERGVPAVIGVLGTIKLPYPSRAFDMAHCSRCLIPWGANDGIYMTEVDRVLRPGGYWVLSGPPINWRTHYEAWERPKEELEEEQRKIEEIAKLLCWEKKHEKGDIAIWRKRINKQYCNERDSHVTMSESSNANDVWYKKMEACVTPYPESTSSEEVAGGELQPFPERLNAVPPRISRGSIPGISVESFQEDNNLWKKHLKAYKSINKIIDTGRYRNVMDMNAGLGSFAAAFESPKLWVMNVMPTIAERDTLGVIYERGLIGIYHDWCEAFSTYPRTYDLIHANGVFSLYKDKCNAEDILLEMDRILRPEGAVIFRDQVNVLIKVKRIVAGMRWNAKMEDHEDGPLVPEKILVAVKQYWVVAEETPCPITIGRIKTKESEISVFGADKYFNMKINCENPGLADNAGRKHGHQKEDCRADLQGLKPNFRSGTPSTISESSWNSQTALFPSLLRKSSQNKQKRAIGKRFFLGFGCSVPCLDKKSVYIDGSVSHGVVKGTKQSEPGLKSGEHVDFPILNFGMENLAVKKQLEVEKLEEEQRKSLEVFGSHMMRKGDEIAKNLERKLSMLTWDAIPAAQSLPRTLASSRLCDDIESDASSDLFELENISSTGQYSLLTRQASDSMSGCMTPTTQYAPSEASIEWSVVTASAADFSVASDYDAEHMISSNTSKMAKTKNINIGGKAPKGLPSGLLSCKSQKAVNVAETAAYRANEKTKSMSLGKPQADQTRVKDFEFSRVQHPFAARVIAP

SEQ ID NO: 117 - protein, 449 aa, synthetic construct
MSSGSPDDDNLSRKQTHKSINDQTKYKLKSLVLIILTNLFTIYIFTRPSLNLHPISKYDSFSDSSFLLHELNATMKQLETSRSQIIVLQQQLKSTNQLVNELLAQLNILNQSINRTPDSTLLNFNDLLDDLSNEAKLALGPHKLPLGYSPRSGTDEVYASVGGGCLMYKEDLAQYMTYRVGKECPVDDVFAQRLMLKGCEPLPRRRCHPKAPVGYVEPTPLPKSLWSTPPDTSIIWDPYTCKNYTCLIERRKFPGFYDCKDCFDLQFREKTRWQFDNGGLDFGMDQVMATRRQGTIRIGLDIGGGTGTFAARMKERNVTIVTTSMNLDGPFNSFIASRGLISMHVGVSQRLPFFENTLDIVHSMHILSNWIPDAMLEFTLYDIYRVLRPGGLFWLDHFFCLGSQLNVTYVPMLERVGFKKLRWNAGMKLDRGIDKNEWYFSALLEKPIS

SEQ ID NO: 118 - protein, 493 aa, synthetic construct
MSSAAAKGQEREMQKSYWIEHTTDLSVEAMMLDSKAADLDKEERPEVLSLLPPYEGKSILELGAGIGRFTGELAKKAGQIIALDFIEGAIKKNERINGHCKSAKFMCADVTSPELDFSAESMDLVFSNWLLMYLSDEEVEALAKRMVKWLKVGGHIFFRESCFHQSGDHKRKNNPTHYREPRFYTKVFKECHFRDESGNSFELTLVGCKCIGAYVKNKKNQNQICWTWKKVRSEDDRAFQRFLDNVQYKCTGILRYERVFGQGYVSTGGIDTTREFVAKLDLQPGQKILDVGCGIGGGDLYMAEKFDVHVVGIDLSINMVSFALERAIGLQCSVEFEVADCTKKTYPDDTFDVIYSRDTILHIQDKPALFKFFYKWLKPGGRVLISDYCKRAGSPSEDFARYIKQRGYDLHDVEAYGQMLKDAGFDEVIAEDRTSQFTNVLQTELDLVEKDREAFIRDFSEEDYDDIVGGWKAKLIRSSSGEQRWGLFIAKKN

SEQ ID NO: 119 - protein, 301 aa, synthetic construct
MSSGKSGRDWTQIYAIYGMDDWQTPFFLLIHAVFFSALSILFLLYFEPICFFTQHFFPFIGAGLARFVAGFTGSVTALSAVCLFFAAANFFYSSVSLHWDMAQRIVNAVPDWSSVKQALDLGCGRGILLNAVAMQLKKSGSSGRVVGLDPTRAHTNGPTTLSTLRTAGLEGVQEYVTCRAGDPRKLPFSDNYFDVVVSSVFVHTVGKEFGQKSAAAGAERMRVLGEVVRVLKSGGVGVVWDLVHVPEYVQRLQELKMEDIRVSERVTAFMLSSHIVSFKKPNQHFVGSNEVRVDWRFNNLC

SEQ ID NO: 120 - protein, 921 aa, synthetic construct
MSSAIGKARSNKRSSSSSRSSTLTTTILVALCIFGLWLLAPNTLVYPRTTTRTSATTSSQSSSMPDSHEIHPTEPIPKKSQPVFQDSQGDLPEDAIQTDHGATHNDHTDESPELPNQSSTGSKAQAEADNVSEQDSGPENNGDSDSSSDNNNEAEGDGGGGDGGNEEVDQKDSDVEGNPQELHGMESVEELEKQKKLETQNSEESSITQNQEAEKTREDQTKASANKDFEGKENQHTADDKKIENHDSSENKEHEISDADQQKRLEQHQQQEDDQIQHGKSQRTEGTTSISNQDVQSTVGGSTDPENHESQHNILHNKNATESISTEEADKKEKPSKWDTSDSFPSGDSSEIPKESRESKKSWSTQADESENQKERRKVGTDDQDGSSINGYKWQLCNVTAGPDYIPCLDNEEAIAKLQSRRHYEHRERHCPKEAPTCLVPLPKGYKRPIEWPQSRDKIWYYNVPHTKLADVKGHQNWVKVTGEFLTFPGGGTQFIHGAMHYIDFLEEAVPDIAWGKHSRVLLDVGCGVASFGGYLFEKDVLAMSFAPKDEHEAQVQFALERGIPAISAVMGTQRLPFPSRVFDVVHCARCRVPWHADGGALLLELNRVLRPGGYFVWSATPVYQTLEEDVKIWKEMSNLTVSMCWELVTIKKDKINSIGAAVYHKPDSNECYHKRKHNNPPMCKSDDDPDAAWYIPLQACMHKVPAEGNERGSDWPEEWPRRLQTPPYWLNRSQMGIYGKPAPEDFTADHEHWKQVVSKSYLSGLGISWSDVRNIMDMRAVYGGFAAALKDLKVWVLNVVNIDSPDTLPIIYERGLFGIYHDWCESFSTYPRTYDLLHADHLFSRLKKRCKLIPVIAEVDRIVRPGGKLIMRDDSSTIGEVENLLKSLHWEVHLTFSKNQEGILSAQKSEWRPDSYVASS

SEQ ID NO: 121 - protein, 343 aa, synthetic construct
MSSASSLVSGAEKFQLIGIAPAGLGFIGSDLHGKHFPKLSLVSCSGISGDRSKILVPRCSLSESRPTSQPRFIQHKKEAFWFYRFLSIIYDHVINPGHWTEDMRDEALEPADLSSRDMIVVDVGGGTGFTTLGIVKHVDAKNVTILDQSPHQLAKAKQKEPLKECKIIEGDAEDLPFRTDYADRYVSAGSIEYWPDPQRGIKEAYRVLKLGGKACLIGPVHPTFWLSRFFADMWMLFPKEEEYIEWFQKAGFKDVQLKRIGPKWYRGVRRHGLIMGCSVTGVKPASGDSPLQLGPKAEDVSKPVNPITFLLRFVLGAMAATYFVLVPIYMWLKDQIVPKGQPI

SEQ ID NO: 122 - protein, 630 aa, synthetic construct
MSSATLQPPVSQLPQKSPAPKLLNKPLFNILLLTIFCSLSYFIGSYTNSIYSISPSPSLIRRDTNCGFEVKNNLNTTVENVQEPLNFEPLHTLSLPMEDTPESHQPFPYCPKNYTNYCPCQDPNKERLFTIDRRFHRERHCPEKGEALRCLVQKPAGYRRPFPWPKSRDYAWFKNVPFKRLTEYKKSQNWVRQEGDKLFFPGGGTSFPKGVKGYIDLLKRVVPLNSGAIRTVLDVGCGVASFGASLMDYNIVTMSIAPRDIHEAQVQFALERGVPALLGVLSTYRLPFPSRCFDMIHCSRCLVQWTDYGGLYLMEIDRLLRPGGYWVLSGPPISWRISYKGWERNASDLEIEQNNLEDLARKLCWKKVRESDPIAVWQKPTNHIHCAQKLKTWKAPKFCSHDFPDHGWYKKMEPCITPLPKVEHVRSTSGGYLEKWPKRLKTAPPRIRSATVEGVTMRTFDEDNKIWKRRVLYYGSVLKFLFKGKYRNIMDMNSGLGGFAAVLSEYPAWVMNVVPYDAKNNTLGIVYERGLIGTYMDWCEPFSTYPRTYDLIHSDGLFSLYMEKCDILDILFEMYRILRPEGAIIIRDHVDVIVKIKEITDRMRWNSKISHNEISPFHSEKILFIDNSPE

SEQ ID NO: 123 - protein, 386 aa, synthetic construct
MSSDYNIVTMSIAPRDIHEAQVQFALERGVPALLGVLSTYRLPFPSRCFDMIHCSRCLVQWTDYGGLYLMEIDRLLRPGGYWVLSGPPISWRISYKGWERNASDLEIEQNNLEDLARKLCWKKVRESDPIAVWQKPTNHIHCAQKLKTWKAPKFCSHDFPDHGWYKKMEPCITPLPKVEHVRSTSGGYLEKWPKRLKTAPPRIRSATVEGVTMRTFDEDNKIWKRRVLYYGSVLKFLFKGKYRNIMDMNSGLGGFAAVLSEYPAWVMNVVPYDAKNNTLGIVYERGLIGTYMDWCEPFSTYPRTYDLIHSDGLFSLYMEKCDILDILFEMYRILRPEGAIIIRDHVDVIVKIKEITDRMRWNSKISHNEISPFHSEKILFIDNSPE

SEQ ID NO: 124 - protein, 348 aa, synthetic construct
MSSSKPSALDLATGLGGKIAKDDVISAVDKYEKYHGYYGGEEEERKANYTDMVNKYYDLVTSFYEYGWGESFHFAARMKGESLKESIKRHEHFLALQLGLKPGQKVLDVGCGVGGPLREIAQFSLTSVTGLNNNEYQISRGKALNRVAGVDRTCDFVKADFMKMPFPDNQFDAVYAVEATCHAPDAVGCYKEVYRVLKPGQCFAAYEWCMTDSFDPNNKEHQKIKAEIELGNGLPDIRLTGQCLEAVKKAGFEVIWAKDLAADSPVPWYLPLDTSHFSFSSFRLTAIGRLITKNLVKGLEYIGLAPSGSQRVQDFLEKAAEGLVAGAKKDIFTPMYFFLARKPLSGSE

SEQ ID NO: 125 - protein, 337 aa, synthetic construct
MSSASSLLSGAENLKHITGSHFPGSEHQLKYFPKKGPVSSAKSSKFSTLAPRCSLSSSRPVSQPRFIQHKQEAFWFYRFLSIVYDHIINPGHWTEDMRDEALEPADLYSRHLTVVDVGGGTGFTTLGIVQHVDAKNVTILDQSPHQLAKAKQKEPLKECKIIEGDAEDLPFRTDYADRYISAGSIEYWPDPQRGIKEAYRVLKIGGKACVIGPVYPTFWLSRFFADVWMLFPKEEEYIEWFQKAGFKDVKLKRIGPKWYRGVRRHGLIMGCSVTGVKPMSGDSPLQLGPKQEDVKKPVNRFVFVLRFILGAMAAAYFVLVPIYMWLKDQIIPKGMPI

SEQ ID NO: 126 - protein, 385 aa, synthetic construct
MSSAFCSQSKLNNLNFWIKTSIGSSTNNVGHDSMRKRTSRNHANLVVVKMAASTSGSGGQGTVAFEEGQLERPQWTGETLLSRLVRALISFKPLFSIMKFGARQVLISTAEKTNIPWREMTRQILESDVYKEMESIEDTSLVYPDYYLNPFHAYDEGNLSWLAAAEAEAATMSMVRRAIPDASSLDEANQIVRGNWLKAIEEHHNKYSQNVIIRDILDIGCSVGVSTRYLADKFPAAKLTGLDLSPYFLAVAQFKEKKSNPRKNTICWIHANGESTGLPSKSYDIVSIAYVFHECPAGAIKNIVKEAFRLLRPGGTFALTDNSPKSKILQELSPVLFTLMKSTEPFLDEYYLTDIEKVMTEAGFINVRTVLTDPRHRTVTATVPY

SEQ ID NO: 127 - protein, 824 aa, synthetic construct
MSSALGKYSRVDGRKPSTNYCSTVTIVVFVALCLVGLWMMTSSSVVPVQNSDVASQQESKNDVNENTRVSKSNANENDSDNSSTVNKNDNTSESSMNDNEGKSKQFEDNPGDLPEDATKGDNTVSSDQEEKNPNPQEKPTEENQEEKSGEENREEGGSKSEDGSKIEKENEENGELKSDEKVSEAGEANRSNASGGESRDGNKPDENFDETKDANKEDGQKVKNKMDTQSDQDSGVKKESDQTTEVFPSGAQSELLNETTTQNGAFSTQATESKNEKEAQQSSELENQSGTSWKLCNVTAKSDYIPCLDNLEAIRSLRSTKHYEHRERHCPDNPPTCLVAFPEGYQRSIEWPTSREKIWYHNVPHTKLAEVKGHQNWVKVTGEYLTFPGGGTQFKHGALHYIDFIQQSVPDIAWGKRSRVVLDVGCGVASFGGFLFDRDVLTMSLAPKDEHEAQVQFALERGIPAISAVMGTKRLPYPAGAFDIVHCARCRVPWHIEGGKLLLELNRLLRPGGYFVWSATPIYQKIPEDVEIWEAMKKLTKSMCWEVVSITKDRVNGVGIAVYRKPITNECYEQRSQNEPPLCQESDDPNAAWYVPLQVCMHKVPIAESERGSQWPELWPARLDKPPYWLSSSQVGVYGKPAPEDFATDFQHWRRVVTKSYVNGLGINWSAVRNVMDMRAVYGGFAAALKDMNVWVMNIVSIDAPDTLPIIYERGLFGIYHDWCESFSSYPRSYDLLHADHLFSKIKKKCNFNAFVAEVDRLLRPEGKIIVRDNVETINELESIFKSLHWDIRMTYSKNKEGLLCAQKTMWRPKEFETVTYAIA

SEQ ID NO: 128 - protein, 237 aa, synthetic construct
MSSEACLTPLPEVSDINEIAGGPLAKWPERLTAVPPRIVSGSLDGITAATFEEDTNLWKKRVAHYKVLDSQLAEPGRYRNLLDMNAYLGGFAAALTNDPVWVMNVVPVEAKLNTLGAIYERGLIGTYQTWCEAMSTYPRTYDFIHADAVFSLYQDRCDMEDILLEMDRILRPQGSVIIRDDVDILVNVKSVVDRLQWDSRMVDHEGGPHVREKLLIAVKQYWTAPAPSEDQEQSRTA

SEQ ID NO: 129 - protein, 603 aa, synthetic construct
MSSKHKDGKPSTQTGKSSRTVPMAIMFVVLCGLSFYLGGIFCSEKDKYASAKVEDTVESPKSTASGSLQVKDVSFPECGLEYQDYTPCTDPRRWKKYGLHRLTFLERHCPPAFERKECLVPLPDGYKIPIRWPKSRDQCWYRNVPYDWINKQKSNQHWLKKEGDKFLFPGGGTMFPNGVGRYVDLMQDLIPGMKDGTIRTAIDTGCGVASWGGDLLDRNILTVSLAPRDNHEAQVQFALERGIPAILGVISTQRLPFPSNSFDMAHCSRCLIPWTEFGGIYLLEIHRILRPGGFWVLSGPPVNYENRWRGWNTTIEEQKSDYDKLQDLLTSLCFKFYKKKDDISVWQKSLDNNCYKKFDSSDNYPTKCDDGTEPDSAWYTPLRPCVVVPNHKYKNLGLKSLPKWPQRLHVAPERVADVRGGSEGAFNHDDSKWKTRVKHYKKLLPAVGTDKIRNVMDMNTLYGGFAAALIDYPLWVMNVVSSYTSNTLAVVFDRGLIGTYHDWCEAFSTYPRTYDLLHLDGLFTAESHRCEIKYVLLEMDRILRPNGYAIIRESSYYLDAVATVAKRMRWGCRKVETEDKLDKEKILICQKKLWYSNTKKSSR

SEQ ID NO: 130 - protein, 441 aa, synthetic construct
MSSGRKYKEYITISSSLLHSFPSFSFLSFPFLGLVPMATWANLPYYLTLKIKKTNLYYMAMITVLCSMSYLIGIWQHGGLKLAANTATSTLPQLPCNPLKKKFTTIGFPLDFTAHHNADDLVPLSTSARVQQFPPCKPELSEYTPCEDAKRSLKFDRDKNIYRERHCPEKEELLKCRIPAPYGYKTPFRWPASRDMVWYANVPHKHLTVEKARQNWVQFKGDRFKFPGGGTMFPRGADAYIDDIGKLINLRDGSIRTAIDTGCGVASWGAYLLSRNILSVSFAPRDTHVAQVQFALERGVPALIGVMASIRLPYPSRAFDIAHCSRCLIPWGKFDGLFLIEVDRILRPGGYWILSGPPINWENHWKGWDRKPEDLKAEQDGIENVAKMLCWKKLIQKNDIAIWQKPTNHKHCKINRKVFKNPPFVRHKIQTKHGTPEWKPA

SEQ ID NO: 131 - protein, 493 aa, synthetic construct
MSSDLNKEQEREMQKSYWVEHTADLTVEAMMLDSKAADLDKEERPEVLSLLPPYDGKSILELGAGIGRFTGELAKKAGHLVALDFIESAIEKNESSNGHYKNAKFICADVTSPELSFPVESMDLIFSNWLLMYLSDEEVEALAERMVKWLKVGGHIFFRESCFHQSGDHKRKNNPTHYRVPRFYTKVFKECHLRDGSGNSFELTLVGCKCIGAYVRNKKNQNQICWVWQKVRSQDDRGFQRFLDNVQYKCNGILRYERVFGHGFVSTGGIETTREFVSKLDLQPGQKVLDVGCGIGGGDFYMADEYDVHVVGIDLSINMISFALERAIGLKCSVEFEVADCTKKTYPDGSFDVIYSRDTILHIQDKPALFKSFYKWLKPGGRVLISDYCKRSVSPSEDFAKYIKQRGYDLHDVEAYRQMLRDAGFDEVIAEDRTDQFIKVLQRELDSVEKDREAFIKDFSEEDYNDIVGGWKAKLIRSSSGEQRWGLFIAKKN

SEQ ID NO: 132 - protein, 354 aa, synthetic construct
MSSGSAEEEASLFAMQLASASVLPMVLKSAIELDLLELIAKAGPGAYVSPSKLAAQLPTHNPQAPVMLDRILRLLATYSVLNCKLKNLPDGGVERLYGLAPVCKFLTKNADGVSMAPLLLMNQDKVLMESWYHLKDAVIDGGIPFNKAYGMTAFEYHGTDPRFNKVFNQGMSNHSTITMKKILEVYKGFEGVKTVVDVGGGTGATLNMIIDKYPTIKGINFELPHVVEDAPFYPGVEHVGGDMFVSVPEGDAIFMKWICHDWSDDHCRKLLKNCYHALPEGGKVILAECILPDAPDTSLATQNVVHIDVIMLAHNPGGKERTEKEFEALAKGAGFKEFRKICSAVNTWIMELCK

SEQ ID NO: 133 - protein, 545 aa, synthetic construct
MSSIDDSESNVKEIKPCHPRYTDYTPCQDQNRAMKFPRENMNYRERHCPPQEEKLHCLIPAPKGYVTPFRWPKSRDYVPYANAPYKSLTVEKAIQNWIQYEGNVFRFPGGGTQFPQGADKYIDQLASVIPIDNGIVRTALDTGCGVASWGAYLWKRKVIAMSFAPRDSHEAQVQFALERGVPAVIGVLGTIKMPYPSRAFDMAHCSRCLIPWGANDGMYMKEVDRVLRPGGYWVLSGPPINWKTNYKAWQRSKEDLEEEQRKIEDIAKQLCWEKKSENAEIAIWQKRIDTDACRAEQENSGAAFCQAADPDDVWYKKMEVCITPSDRKGDEAYGERIKPFPERLYAIPPRIASGSVPGFSAESYEEDNKKWKKHVSAYKRINKILDSGRYRNIMDMNAGLGGFAAALESPKLWVMNVVPTIAEKNTLGVIYERGLIGIYHDWCEGFSTYPRTYDLIHANGLFSLYKDKCDFEDILLEMDRILRPEGAVIIRDEVDVLNRVQKIVRGIRWDFKLTDHEDGPLVPEKILIAVKKYWVAGENNSTSTQ

SEQ ID NO: 134 - protein, 263 aa, synthetic construct
MSSSGYQAKEYSTGRPTYPEEIFQFVASKTPCHDLAWDVGTGSGQAARSLARIYKNVVATDVSPEQLECAVKLPNIKYYCTPPKMSMTELEQNVATESSVDLVSIATAMHWFDLPTFYQQVKWVLKKPDGVIAAWSYTLPQINKNVDTLLRKLFFADAVQFFGPQAKLIIEENYRTIDFPFEPVDGLEHTGPFESKMEKLINLDGYFTLIKSWSAYKTAKEKGVELLSEDLVKEFTLAWNEDGKQEKIATWPLYMRIGKVGKQ

SEQ ID NO: 135 - protein, 238 aa, synthetic construct
MSSDVDVEAKMDAEKLKRGIAEFYDESSGIWEDIWGDHMHHGFYDPSSTVSLLDHRSAQIRMIEEALQFASVSDALNQPFPDGKFDLVWSLESGEHMPNKAKFVNELVRVAAPGATIIIVTWCHRNLSSSEQSLHPDEKKLLDKLCDAYYLPPWCSTDDYVKLLESFSLQDIKAADWTEYVAPFWPAVVSSALSWKGITSLILSGWKTIKGALAMLLMIKGYKKGLIKFAVITCRKPE

SEQ ID NO: 136 - protein, 512 aa, synthetic construct
MSSAKDYNGSPKPHHLETQRKRLTWILAVSGLCVLFYVLGAWQSTNPAPSSQSDVWERVGCQDGSPDQKVNNPDLSTSTLSLSSSWSSTPLDFESHHQLEVNSSEEFRKFPPCDTSYTEYTPCQDPPRGRKFDRNMLKYRERHCPEKEELLHCLIPAPPNYKTPFKWPQRRDYAWYANIPHKHLSVEKANQNWIQFEGDRFKFPGGGTMFPRGADAYIDDINELIPLSNGTIRTAVDTGCGVASWGAYLFKRNIIAMSFAPRDTHEAQVWFALERGVPAMIGVMGSQRLPYPARAFDMAHCSRCLIPWHKYGGLHLIEVDRILRPGGYWILSGPPIRWKNYWRGWERTQEDLKQEQDAIEDVAKNICWKKVIEKDDLAVWQKPINHIECIKSRAIHHATPMCKSDNADVAWYKDMETCISPLPEVSNKDEVAGGSLEKWPERVFAVPPRISSGSVAGITMENFRDDNDVWKERVAHYKRVVGPLDQGQYRNVMDMNAFLGGLQQFWQNIQCG

SEQ ID NO: 137 - protein, 625 aa, synthetic construct
MSSTRGRSDGAQKKRLLTSVGIIAIFLLFLYVYFGSKSNGESALEYGSRSLRKLGSSYLGGDEDGDLGSKQDDASSKFALDDGEDGLVPKSFLVCDDRHSELIPCLDRHLIYQMRLKLDLSLMEHYERHCPLPERRFNCLIPPPSGYKVPIKWPKSRDEVWKANIPHTHLASEKSDQNWMVVKGEKIIFPGGGTHFHYGADKYIASIANMLNFSDNNLNNEGRLRTVLDVGCGVASFGAYLLSSDIIAMSLAPNDVHQNQIQFALERGIPAYLGVLGTMRLPYPSRSFELAHCSRCRIDWLQRDGILLLELDRLLRPGGYFAYSSPEAYAQDEEDLRIWREMSALVERMCWKIAAKRNQTVIWVKPLTNDCYMEREPGTQPPLCQSDNDPDAVWGVKMEGCITRYSDHDHRTRGSGLAPWPARLTSPPPRLADFGYSSEMWEKDMQVWRRRVENYWNLLSPKISSDSLRNVMDMKANLGSFGAALKEKDVWVMNVVPEDGPNTLKLVYDRGLIGTIHNWCEAFSTYPRTYDLLHAWSVLSDIEKKGCSGEDLLLEMDRILRPTGFVIIRDKQPVIDFVKKFLGALHWEAVGTADSSSDPDQEGDELVFVIQKKLWLTSGSFRDTE

SEQ ID NO: 138 - protein, 254 aa, synthetic construct
MSSATLQPPVSQLPQKSPAPKLLNKPLFNILLLTIFCSLSYFIGSYTNSIYSISPSPSLIRRDTNCGFEVKNNLNTTVENVQEPLNFEPLHTLSLPMEDTPESHQPFPYCPKNYTNYCPCQDPNKERLFTIDRRFHRERHCPEKGEALRCLVQKPAGYRRPFPWPKSRDYAWFKNVPFKRLTEYKKSQNWVRQEGDKLFFPGGGTSFPKGVKGYIDLLKRVVPLNSGAIRTVLDVGCGVSINGCSKIFIVFVRF

SEQ ID NO: 139 - protein, 523 aa, synthetic construct
MSSEPSTGKPSFFRNILVRLFLFCVFILGVRFAYVVTIRGESCDLGDFCFFSLPDNLNTIVSGVGQLGTKSFSSSAIIAVGKSAPAKPHKLPDLWATQGFQKAAQFYSSVFQDLISEGFLTPNSKALCVDTPTGADVFALKEIGVSDSIGIFKKSSKPLVISGQAIKQPFGDDKFDFVFSGAGMIEKSVKAADFAAEVCRTLKPEGFFVVHTGSKDTYSYNSFLDLFNCCKLVKFRDIDGFDSKLPFIREIVMQKKFNENVDDEIQMPGGNSEDKCFVPGYKQELIRNAEPLIVEEPKKPWITLKKNIQNIKYLPSMADISFKQRYVYVDVGARSYGSSIVSWFKKQYPKQNRTFEIYAIEADKHFHEEYKYKKGVKLLPYAAWVRNETLFFEINEDPIDKDVVKGRGMGRIQPVQSSSGVSDGNVDEIQGFDFAEWLKSTVSEKDFVVMKMDVEGTEFDLIPRLIETGTICLIDEVFLECHYNRWQKCCPGERTSKYQKTYDQCLELFTSLRESGVLVHQWW

SEQ ID NO: 140 - protein, 704 aa, synthetic construct
MSSSRPLHRGVSGGGRFSPNSHDLWDDDDSQMKDKAEKEDGDRKSTNADHTYLSIRQPFRFLLADNSSSKHGVTKDGFVSDPFSPSPGVSRNRHKFILLLLRFGLVVIVMLALAGSFWWTISITTSSRGQIYRGYRRLQQKLVSDLVDVGKLSLGSTRLKDLEYCSPESEHYVPCFNVSENLALGISGGNEYDRHCGPGSRQSCFVLPPVNYKTPLRWPWGRDLIWVANINITAQEVRSSGSLTKRMMMYDEDQISFRSASPGYDDMEDYAHQIAEMIGLRNESNFVQSGVRTILDIGCGYGNLGAHLFSNELLTICIANYEASGSQVQLTLERGLPAMISSFTSKQLPYPSLSFDMIHCAGCGIDWDQKDGILLVEVDRVLRPGGYFVWTSQLTSSQQFLRKNENQKEWNFVHGFAKSLCWEMLSQQVDTVVWKKTSKKNCYSARKSGSGPSLCSRGHDVESPHYRPLQGCIGGTQSQRWIPIEERTKWPARARLNAKELAIHGLNSDDFAEDTLNWNSAVRNYWSLLSPVIFSDHPKRPGDEDPSPPFNMVRNVLDMNAHFGGFNAALLEAKKSVWVMNVIPTTGNNHLPLILDRGFVGVLHDWCEAFPTYPRTYDLVHGEGFLSLEFGEEQRCEMLDVFTEIDRILRPEGWFILRDTTPLIESARTLTARLKWDARMIEIENNSDERLLICQKPFAKRQAM

SEQ ID NO: 141 - protein, 102 aa, synthetic construct
MSSKWILHDWDDGHCLKLLKNCYKALSDNGKVIVVDALVPVIPDTSPCVKTTCQSDLIMMTQNPGGKERSEPEFLALATAAGFKGIRLECYVCNLWVMEFYK

SEQ ID NO: 142 - protein, 140 aa, synthetic construct
MSSKIHSRSLCPSRSYGGQEVEVFQIARTMMQSIILYSIICSLHCFHAFPSSNSSTPAYNALWNKSLTLPNVTNNRYFLFIFTPFSKHTKVKHATTTIVKLNNYISIVNDVRNLSVIDFIFVIYIHSSRCHYTYRISSNK

SEQ ID NO: 143 - protein, 385 aa, synthetic construct
MSSQPQRGRKRERERDREEMESVQSNSSSSDQFAMKGGDDDFSYTKNSTHQRDAIQATKFFIQESIAEKLDVNKFCGKAFCVADLGCSVGPNTLIAMQNIVEAVELKFKNRKGFHSPTIPEFQVFFNDHTVNDFNTLFRSLPTGHDKRYYGVGVPGSFYGRLFPCDSIHIMHTSFSTPFLSQVPKEVMDKNSAAWNKGRIHHNYAKADVLKAYEAQHAEDIDCFLTARAKELVHGGLLMDVTSFRPDGVPHTHVLTNIGMEVLGYCLMDLAKKGLIDEENVDSYNVPVYLQSPEELKQAVQRNKYFSIEKMESVPMMIDSDVSAKAQQYSLGMRAVMGDVIREQFGAEIVDKLFDLFKKKLEEHPNFAKGVVLDMFVLLKRNAED

SEQ ID NO: 144 - protein, 366 aa, synthetic construct
MSSESSQSNNRFCDQFATKGGDDDFSYTKNSIHQRDAIEATKLLIQEAIAEKLDINKFCGKTFCIAHLGCSTGPTTLTAMQNIIEAVELKFQNQKGFNFPNFPEFQVFFNDHTVNDFNALFRSLPTGHDKRFYGVGVPGSLYGRLFPCNSIHITHTSFSTPFLSLVPKEVMDKNSAAWNEGKIHYNHAKEDVLKAYEGKHAEDMDCFFMARAKELVHGGLMLIVTSLRPDGIPHVLTNIGMEVLGSCLMDLAKKGLIDEENVDSFNVPVYLESPEELKQAIQRNKYFSMEKVESVPMIMDSDISCRAQQYSLAIRAVMEDVFREQFEAEILDQLFDLFKKKLEGHPNFASAVVLDLFVLLKRNAED

SEQ ID NO: 145 - protein, 368 aa, synthetic construct
MSSESLPGNNRSYDQFAMKGGDDDFSYTKNSIHQRDAIEATKLLIEEAVAEKLDISNFCGKAFCIADLGCSVGPNTLIAMQNIVEEVKLKFRNRKGLNFPNIPEFQVFFNDHTVNDFNALFRSLPTGHDKSYYGVGVPGSFYGRLFPCDTIHIMHTSFSTPFLSQVPKEVMEKNSAAWNKGRIHYNHAKEDVLKAYEGKHAEDMDCFFMARAKELVHGGLMLIVTSLRPDGVPHTRVLTNIGMEVLGSCLMELAKKGLIDEEKVDSFNAPVYLESPEELKQAIQKNKYFSIEKMETVPMIMDSDISCRAQQYSLAIRAVMEDVFREQFEAEILDQLFDLFKKKLEGHPNFASAVVLDLFVLLKRNAED

SEQ ID NO: 146 - protein, 383 aa, synthetic construct
MSSGAPKKKRKVGSSGGMESVQSNSSSSDQFAMKGGDDDFSYTKNSTHQRDAIQATKFFIQESIAEKLDVNKFCGKAFCVADLGCSVGPNTLIAMQNIVEAVELKFKNRKGFHSPTIPEFQVFFNDHTVNDFNTLFRSLPTGHDKRYYGVGVPGSFYGRLFPCDSIHIMHTSFSTPFLSQVPKEVMDKNSAAWNKGRIHHNYAKADVLKAYEAQHAEDIDCFLTARAKELVHGGLLMDVTSFRPDGVPHTHVLTNIGMEVLGYCLMDLAKKGLIDEENVDSYNVPVYLQSPEELKQAVQRNKYFSIEKMESVPMMIDSDVSAKAQQYSLGMRAVMGDVIREQFGAEIVDKLFDLFKKKLEEHPNFAKGVVLDMFVLLKRNAED

SEQ ID NO: 147 - protein, 385 aa, synthetic construct
MSSQPQRGRKRERERDREEMESLQNNNRYCDQFAMIGGDDDFSYTKNSIHQRDAIEATKFLIQGSIAEKLDINNFCGKAFCVADLGCSVGPNTLIAMQNIVDAVELKFKNRKGFHSPTIPEFQVFFNDHTVNDFNTLFRSLPTGHDKRYYGVGVPGFFLRSIISLDSIHIMHTSFSTPFLSQVPKEVMDKNSAAWNKGRIHHNYAKADVLKAYEAQHAEDIDCFLTARAKELVHGGLLMDVTSFRPDGVPHTHVLTNIGMEVLGYCLMDLAKKGLIDEENVDSYNVPVYLQSPEELKQAVQRNKYFSIEKMESVPMMIDSDVSAKAQQYSLGMRAVMGDVIREQFGAEIVDKLFDLFKKKLEEHPNFAKGVVLDMFVLLKRNAED

SEQ ID NO: 148 - protein, 382 aa, synthetic construct
MSSQPQRGRKRERERDREEMYKAPFVLCAAVAVITDHMNLSGAGNYGRDAIEATKFLIQESIAEKLDINKFCGKAFCVADLGCSAGPNTLIAMQNIVEAVELKFKNRKGFHSPNIPEFQVFFNDHTVNDFNTLFRSLPTGHDKRYYGVGVPVLFTVDYFLVTLFTHIMHTSFSTPFLSQVPKEVMDKNSAAWNKGRIHHNYAKADVLKAYEAQHAEDIDCFLTARAKELVHGGLLMDVTSFRPDGVPHTHVLTNIGMEVLGYCLMDLAKKGLIDEENVDSYNVPVYLQSPEELKQAVQRNKYFSIEKMESVPMMIDSDVSAKAQQYSLGMRAVMGDVIREQFGAEIVDKLFDLFKKKLEEHPNFAKGVVLDMFVLLKRNAED

SEQ ID NO: 149 - protein, 291 aa, synthetic construct
MSSQNIVEAVELKFKNRKGFHSPTIPEFQVFFNDHTVNDFNTLFRSLPTGHDKRYYGVGVPGSFYGRLFPCDSIHIMHTSFSTPFLSQVPKEVMDKNSAAWNKGRIHHNYAKADVLKAYEAQHAEDIDCFLTARAKELVHGGLLMDVTSFRPDGVPHTHVLTNIGMEVLGYCLMDLAKKGLIDEENVDSYNVPVYLQSPEELKQAVQRNKYFSIEKMESVPMMIDSDVSAKAQQYSLGMRAVMGDVIREQFGAEIVDKLFDLFKKKLEEHPNFAKGVVLDMFVLLKRNAED

SEQ ID NO: 150 - protein, 501 aa, synthetic construct
MAALPLLIFSSLFLALLLKLLLKHKNNRNSKLPPCPPKLPIIGNLHLLGNMPQESLHQLAKKYGPVIFLQLGEVPTIVISSARLAKEAMKTFDLALSSRPQIFSAKHLFYNCTDVVFSPYGAYWRNIRKICILELLSAKRVQSFSYVREEEVAHLVQRVAEAYPGTTNLTKMLGLYANNLVCRVAFGKDFSQGGDNHRHNFQKLLEEYQVLLGGFSIGDFFPSMEFVHKFTGMESRLKDTFRRFDQFFDEILQEHRNPESRKEHKDLVDVLLEIQKNGDPETPLTTDNIKSIILDMFAAGTDTNFITLDWAMTELMTNPGVLKRAQAEVRNIVGERRSVSENDLPQLPYVKAVVKETFRLHPPAPVLVPRESMEDVTIDGYDIPAKTRFFINAWAIGRDPKSWENPDVFEPERFMNSHIDFKGQDYELIPFGAGRRSCPAITFSAVTVELALAQLLHSFDWELPPGVEAKDLDMTEVFGITMHKIYPLMVVAKPYFSQGLN

SEQ ID NO: 151 - protein, 501 aa, synthetic construct
MAALPLLIFSSLFLALLLKLLLKHTKLVKLPPGPRPLPIIGNLHQLNGSLPHRILTDLAKKYGPLMHLQLGEVSTVIVSSPAVAKEVMKDHDIIFCYRPLLLSPAILNYGGTDIAFAPYGEYWRQLRKICTIELLSSKRVQTFRSIREQEVSNLFRSIKSEEGSVVNLSKMIYSLTYSITAQAAFGKESKYQDKFISVMQDAIKLMGGFNIADVYPSIKILEKITGIRHKLEETHNQIDEIVQNILIEHKAKRAESKLGNRGAKEDLVDVLLNIQSSGEFEPPLTDDNLKAVIYDIFSAGGETSSTTVIWAMSEMIRNPKVMKKAQDEVREVFAGNGNVDESRLHELKYLQAVIKETMRLNPAVPLLLPRECSEHCQINGFDVPAKTRVIVNAWAIGRDPKQWTEPEKFKPDRFLDSEIDFRGKTFSFIPFGAGRRICPGISFALPLVELPLAQLLYHFDWKLPGQSKPDELDMTEIFGLTVGRKHDLNLVPIPYRPCSVL

SEQ ID NO: 152 - protein, 501 aa, synthetic construct
MAELLFTLCALLVFFSFAVKIVRKTKPVKLPPGPRPLPIIGNLHQLNGSLPHRILRNLAKKYGPLMHLQLGEVSTVIISSPALAKEIMNEHDIVFCYRPFLLSPSILNYGCADIAFAPYGEYWRQLRKICTMELLTSRRVQTFRSIREVEVSNLIRSISSEEGSVVNLSKKIYSLTYSITAQAAFGKRSKYHDKFISIMQDVVKLMGGFKIADMYPSLKILEKLTGIRHKLEMVHNQVDEVIENILSEHKVKRADSNLGNREAKEDLVDVLLNIQSSGEFGVPLTDHNIKAVIYDVFSAGGETSSTTVIWTMSEMIRNPKVMKKAQDEVREVFAGNGNVDESRLHELKYLQAVIKESMRLHPAAPLLLPRECSEHCQINGYDIPAKTRVIVNAWAIGRDSKAWTEPEKFNPNRFLDSEIDFRGKTFSFIPFGAGRRICPGISFALPLVELPLAQLLYHFDWKLPGQSKPEELDMTEIFGLAVGRKHDLKLIPIPYRPCSRE

SEQ ID NO: 153 - protein, 223 aa, synthetic construct
MAIAKDVLVTHHPSFADRPNNLATEIIRYDQEDMGFSPYGDYWKQMRKICMMQLLSMTNVASFSSIRQDEISKLVEAIRSTPGVPVNLTEKIFACTSSIICRAAFGQVCQDKENMIKNLKIAFVLLAGFNLADVFPSLKFIPIITGFKQKLIKVHHKMDEILGDVIKEHILKHENGKKCNAESGDEDLIDVLLQQPKNGNLHIPITARNIKALILVSTISLCF

SEQ ID NO: 154 - protein, 446 aa, synthetic construct
MALQLGSVKTLVVSSAEAAHEIMKTHDLNFLNRPESKVNRKLLYDFKDLSVAPYGEYWRQMKSICVLHLLSNKRVQSIRYIREEETSFLITKIREGSLASPSSGAVNLSEMFVTLTNDVVSRAAFGRKYSEGESGRKFRKLMSEFVSVLGGFDFGTVLPCLGWIDRLNGLTARVERIAKEMDKFLEGVLEEHLDSDRKNNESRVVMTRKIHGENGEDFADVLLGIYKNKAAGISIDRDSIKGLLLDIFAGGTDTTYTVLEWAMTELLRHPKALKELQDEVRGLVGGGREITEQDLEKMKYLKAVIKETLRLHPPIPLLVPRKAINDAKIMDYDISAGTMVITNAFAIGRDPSLWEEPDEFRPERFLNSCIDFRGHDFQLIPFGSGRRGCPGISFAMATNEHVLANLVLKFDWDLPDGAKGTDLDMTECTGLTIHRKFPIFAVATPV

SEQ ID NO: 155 - protein, 340 aa, synthetic construct
MAFMTLTNDVMSRAAFGWICSEGESGRKFRKLMSESARVFGVFDFGTYLPCLGQIDSVNGLMVRVERIAKEMDEFLERVLEEHLDSDRKNNESRVVMTSKIHGENGEDFADVLFGIYKNKAAGSSIDRDSIKALLLDMFAGGTDTNYTFLEWAITELLRHPKALKELQDEVRGLVDGGREITEQDSEKMKYLKAVIKETLRLHPPLPLSVRKAINDVKIMDYDISAGTTVITNAFAIGRDPSLWEEPDEFRPERFLNCCVDFRGHDFQLIPFGSGRRGCTGISFAMATNEHVLANLVLKFDWDLPDGAKGTDLNMTKSTGLTIHRKNSSSCCCNSSLLTQ

SEQ ID NO: 156 - protein, 374 aa, synthetic construct
MAELLSPKRVQTFRSIREGEVLNMIKSIALHMGSSVDLSRKIFSLPYGITAQAAFGKKGKYQEQYIMLIDEMVKLSGGFSLVDVYPSIKLLERISGTRTKLEKLHKQIDGILQIIVNEHKKKLEKEELGSGEKKVDLVDVLLNIGKRGEFGSHLADNNIKAIIYDIFSAGSETSSTTVEWAISEMIKSRTIMKRAQDEVRNIFKERGNVDESNLHELKYLQAVIKETMRLHPGAPLLLPKESIEKCEVKGYEIPAKTRVIINAWALGRDPMYWSEAEKFNPERFLDSELDFRGNHFEYIPFGAGRRICPGISFALPNTELPLAQLLFHFDWKLPGDLKQEQLDMTEVFGVTVRRKNDLRLIPVPYSHSSLNKDL

SEQ ID NO: 157 - protein, 257 aa, synthetic construct
MALAKIVMKSKNKNSGKKLPPGPRQVPIIGNMHQLYGPLPHHKLRDLAKKYGPLMHLQLGEISTIVVSSPEFAKEVYRTHDIIFAERAELLSISIVSYNFLGIVFAPYADYWRQLRKICTMELLSPKRVQTFRSIWEGEVLNMIKSIALQKGSSVNLSRKIFSLTYGITAQAAFGKKGKYQEQYVMLVDEMVKLSGGFSLVDLYPSIKLLERISGTRTKLEILHKQIDGILQTIVNEHKKKLEKEELGSGEKKSLSG

SEQ ID NO: 158 - protein, 521 aa, synthetic construct
MAAIFSKIGIVGAGISGIAAAKQLRHYKPVVFEATDSLGGVWRHCSYRSTKLQTPRCDYEFSDFPWTERDNSSFPSYMEILDYLNSYASNFGVLELIKFNSKVVEIKFTGNRDQSNYSDSGSNSKEYGNLLGGQPVWEVAVQTSDSDTIEWYAFEFLVICTGKYGDIPIIPKFPNNKGPEVFRGQVMHTLDYSKLDEEASVNLLKDKKVVVIGYKKSAIDLAVECAKANEGPEGKPCTVVVRTLHWTVPHYSIWGLPFYLFYSTRASQFLHERPNQGILRNILCKLLSPVRKAMSKIIESYLVWKLPLKKYGLKPDHPFEEDYGSCQMAILPEEFFAEADKGMIEFKRASQWWFCEGGVQFDDNSKLEADVVLLATGFDGKRKLRSILPEPFRSLIETDGVMPLYRGTIHPLIPNMAFVGYIESVSNLHTAEIRCIWLSRLVENYFKLPSLENMLEQTQKEMEIMRRTTRFFKRSCISTFSINHSDEICEEMGWKSWRKNSWLAEAFSPYNSQDYQEKKVN

SEQ ID NO: 159 - protein, 329 aa, synthetic construct
MAAMSKVLSLFFLQFLCVLCISDLSNGQGLDVNFYKKTCPSLEDIVKDTTARFISRAPSLAAPLLRMHFHDCFVRGCDGSVLLNSTSSNQSEKAAIPNQSLRGFQVIDAVKSELEKKCPGVVSCADIVALVARDAITLIKGPSWQVELGRRDGRVSIASQVLNKLPSPFMNIAQLKASFQSLGLSVKDLAVLSGGHTIGISHCASVNNRLYNFTGKGDTDPTLDPKYVATLKRKCQPGDVTTILEMDPGSAKTFDTDYFTTVSKRRGLFQSDAALLDDTETNTYVQQHLSSSGSGSFFDDFGVPMVKMGRIGVLTGNQGEIRKQCALVN

SEQ ID NO: 160 - protein, 329 aa, synthetic construct
MAAFPRHLAVFMLLQIILFPTAFQHCNAQGLKLGFYKKTCPGAEATVKQIMTGVISRAPTLAAPLLRMHFHDCFVRGCDGSVLLNSTKNNKSEKEAIPNQSLRGFQIIDNIKSALEKKCPGVVSCADILALVARDAVSLLNGPFWIVPLGRRDGKVSIMNEALTNLPPPFANITQLKAFFSAKGLNTKDLAVLSGGHTIGTAHCLGFTNRLYNFTGKGDTDPSLDPNYIPLLKRKCKPGDVTTLVEMTPGTFKTFDEDYYTLITKRRGLFQSDAALLDDSQTRAYVKLQAATHGSTFFKDFAVSMVKMGKVGVLTGKAGEIRRHCALVN

SEQ ID NO: 161 - protein, 316 aa, synthetic construct
MAASSIVPTIDLDPFLREGDEEAKKNAIEEICQACSEYGFFQVVNHGVPLDLMTRATDMFTEFFNYPIEEKFKSISKPGVSPPPGYFNSPDHSKEGNEHFLILTPSLGLNVYPENPPEFRKVLEEIFPNLAKLALLIERIVSVSLGLAPNFLKEYNNDRSCDFLMGLHYFPAEETENNGKSAHEDGNCITFVFQDEVGGLEVLKNGQWIPITPAQGTLVVNIGDVVQVLSNNKFKSATHRVVRPKGKSRYSYVFFHNLHGDKWVEPLPQFTKDIGESAKYRGFVYKEYMQHRIRNRTHPPARPEDLIHITHYSIST

SEQ ID NO: 162 - protein, 317 aa, synthetic construct
MAPLLQMPTVDLSSFFTGDDVGQKNVLSKISQACSDYGFFQIVNHGLCLDLMNRALEISRVFFESPTEEKLKCSPRPGASPPAGYNRKPNPEYEFSEYFIMFPPGSDFNRFPPNLPQLREVMEELFSQFLKTASVISSILIESLGLPQSIDEGFNFADRSSDLLIALFYLSAKEKENMGVNPHKDVSCFTMVLQDEVGGLGVEKDGEWLPLGPTEGALIVNIADVLQVLTNDKFKSPNHRVLRPKARSRSSFAFFHDLPGKTWLEPLPEFTREIGELPKYRKFLYKDYKQMRVRNKSNPHSRLEDEITISHYAIASK

SEQ ID NO: 163 - protein, 501 aa, synthetic construct
MAALPLLIFASVSLIVVLKFVSKPKNNRNSKLPPCPPKLPIIGNLHLLGNMPQESLHQLAKKYGPVIFLQLGEVPTIVISSARLAKEAMKTFDLALSSRPQIFSAKHLFYNCTDVVFSPYGAYWRNIRKICILELLSAKRVQSFSYVREEEVAHLVQRVAEAYPGTTNLTKMLGLYANNLVCRVAFGKDFSQGGDNHRHNFQKLLEEYQVLLGGFSIGDFFPSMEFVHKFTGMESRLKDTFRRFDQFFDEILQEHRNPESRKEHKDLVDVLLEIQKNGDPETPLTTDNIKSIILDMFAAGTDTNFITLDWAMTELMTNPGVLKRAQAEVRNIVGERRSVSENDLPQLPYVKAVVKETFRLHPPAPVLVPRESMEDVTIDGYDIPAKTRFFINAWAIGRDPKSWENPDVFEPERFMNSHIDFKGQDYELIPFGAGRRSCPAITFSAVTVELALAQLLHSFDWELPPGVEAKDLDMTEVFGITMHKIYPLMVVAKPYFSQGLN

SEQ ID NO: 164 - protein, 501 aa, synthetic construct
MAALPLFIFASISLVPVLVILLMKKDKRSVKLPPSPPKLPIIGNLHQIGNMPHESFHNLAKKFGPIMFLWLGQVPTVVISSARLAKEALKTSDLALSSRPELFSAKHLHYNCTDLAFSPYGTYWRNIRKITTLQLLSAKRVQSFSFVRKDEVAHLVKRITESYPSTINLSHMLEVYANNNICRVAFGKDFSQGGDNVRHNFQKVLEEYQMLLGGFSIGDYFPSMEFLHKFTGMESRLKDTFRRFDHICDKILEEHRKTENRKDQKDLVDVLLEIQHNGDLETPLTTDNIKAIIIDMFVGGTDTNFITLDWTMTELIINPKVMQRAQAEVRSIVGERRFVSENDLSHLGYMKAVVKETFRLHPPAPLLAPRESIQDVTIDGYNIPAKTRFFINAWAIGRDSELWENPEMFEPERFMNSHIDFKGQDFELIPFGAGRRICPAISFTVATVELALAQLLHGFDWELPPGVEAKDLDMTEAFGITMHRTSPLMVVAKPYFSDHLN

SEQ ID NO: 165 - protein, 501 aa, synthetic construct
MAALPLFIFASISLVLVLVILSMKKDKRSVKLPPSPPKLPIIGNLHQIGNMPHESFHNLAKKFGPIMFLWLGQVPTVVISSARLAKEALKTFDLALSNRPELFSAKHLHYNCTDLAFSPYGTYWRNIRKITTLQLLSAKRVQSFSFVRKDEVVHLVKRITESYPSTINLSHVLEVYANNNICPVAFGKDFSQGGDNVRHNFQKVLEEYQMLLGGFSIGDYFPSMEFLHKFTGMESRLKDTLRRFDHICDKILEEHRKTENRKDQKDLVDVLLEIQHNGDLETPLTTDNIKAIIIDMFVGGTDTNSITLDWTMTELIMNPKVMQRAQAEVRSIVGERRFVSENDLSHLGYMKAVVKETFRLHPPAPLLAPRESMQDVTIDGYNIPAKTRFFINAWAIGRDSELWENPEMFEPERFMNSHIDFKGQDFELIPFGAGRRICPAISFTVATVELALAQLLHSFDWELPPGVEAKDLDMTEAFGITMHRTSPLMVVAKPYFSDHMN

SEQ ID NO: 166 - protein, 497 aa, synthetic construct
MAALPLFIFASISLVPVLVILLMKKDKRSVKLPPSPPKLPIIGNLHQIGNMPHESFHNLAKKFGPIMFLWLGQVPTVVISSARLAKEALKTSDLALSSRPELFSAKHLHYNCTDLAFSPYGTYWRNIRKITTLQLLSAKRVQSFSFVRKDEVAHLVKRITESYPSTINLSHMLEVYANNNICRVAFGKDFSQGGDNVRHNFQKVLEEYQMLLGGFSIGDYFPSMEFLHKFTGMESRLKDTFRRFDHICDKILEEHRKTENRKDQKDLVDVLLEIQHNGDLETPLTTDNIKDMFVGGTDTNFITLDWTMTELIINPKVMQRAQAEVRSIVGERRFVSENDLSHLGYMKAVVKETFRLHPPAPLLAPRESIQDVTIDGYNIPAKTRFFINAWAIGRDSELWENPEMFEPERFMNSHIDFKGQDFELIPFGAGRRICPAISFTVATVELALAQLLHGFDWELPPGVEAKDLDMTEAFGITMHRTSPLMVVAKPYFSDHLN

SEQ ID NO: 167 - protein, 501 aa, synthetic construct
MAALPLLIFSSLFLALLLKLLLKHKNNRNSKLPPCPPKLPIIGNLHQIGNMPHESFNNLAKKFGPIMFLWLGQVPTVVISSARLAKEALKTFDLALSSRPELFSAKHLHYNCTDLAFSPYGTYWRNIRKITTLQLLSAKRVQSFSFVRKDEVAHLVKRITESYPSTINLSHMLEVYANNNICRVAFGKDFSQGGDNVRHNFQKVLEEYQMLLGGFSIGDYFPSMEFLHKFTGMESRLKDTFRSFDHICDKILEEHRKTENRKDQKDLVDVLLEIQHNGDLETPLTTDNIKAIIIDMFVGGTDTNLITLDWTMTELIINPKVMQRAQAEVRSIVGERRFVSENDLSHLGYMKAVVKETFRLHPPAPLLVPRESIQDVTIDGYYIPAKTRFFINAWAIGRDPELWENPEMFDPERFMNSHIDFKGQDFELIPFGAGRRICPAITFSIANVELALAQLLHSFDWELPPGVEAKDLDMTEAFGITMHRISPLMVVAKPYLSDYLN

SEQ ID NO: 168 - protein, 497 aa, synthetic construct
MAALPLFIFASISLVLVLVILSMKKDKRSVKLPPSPPKLPIIGNLHQIGNMPHESFHNLAKKFGPIMFLWLGQVPTVVISSARLAKEALKTFDLALSNRPELFSAKHLHYNCTDLAFSPYGTYWRNIRKITTLQLLSAKRVQSFSFVRKDEVVHLVKRITESYPSTINLSHVLEVYANNNICPVAFGKDFSQGGDNVRHNFQKVLEEYQMLLGGFSIGDYFPSMEFLHKFTGMESRLKDTLRRFDHICDKILEEHRKTENRKDQKDLVDVLLEIQHNGDLETPLTTDNIKDMFVGGTDTNSITLDWTMTELIMNPKVMQRAQAEVRSIVGERRFVSENDLSHLGYMKAVVKETFRLHPPAPLLAPRESMQDVTIDGYNIPAKTRFFINAWAIGRDSELWENPEMFEPERFMNSHIDFKGQDFELIPFGAGRRICPAISFTVATVELALAQLLHSFDWELPPGVEAKDLDMTEAFGITMHRTSPLMVVAKPYFSDHMN

SEQ ID NO: 169 - protein, 501 aa, synthetic construct
MAALPLLIFASLSLIVVLKFLLKPKNNRNSKLPPCPPKLPIIGNLHLLGNMPQESLHQLAKKYGPIIFLQLGEVPTVVISSARLAKEAMKTFDLALSSRPQIFSAKHLFYNCTDVVFSPYGAYWRNIRKICILELLSAKRVQSFSYVREEEVAHLVQRVAEAYPGTTNLTKMLGLYANNLVCRVAFGKDFSQGGDNHRHNFQKLLEEYQVLLGGFSIGDFFPSMEFVHKFTGMESRLKDTFRRFDQFFDEILQEHRNPESRKEHKDLVDVLLEIQKNGDPETPLTTDNIKAIILDMFAAGTDTNFITLDWAMTELMTNPGVLKRAQAEVRNIVGERRSVSENDLPQLPYVKAVVKETFRLHPPAPVLVPRESMEDVTIDGYDIPAKTRFFINAWAIGRDPKSWENPDVFEPERFMNSHIDFKGQDYELIPFGAGRRSCPAITFSAVTVELALAQLLHSFDWELPPGVEAKDLDMTEVFGITMHKIYPLMVVAKPYFSQGLN

SEQ ID NO: 170 - protein, 517 aa, synthetic construct
MAALIEDHSTQASVFILMELFLTLCALLAFFSIALKIVRKTKLVKLPPGPRPLPIIGNLHQLNGSLPHRILTDLAKKYGPLMHLQLGEVSTVIVSSPAVAKEVMKDHDIIFCYRPLLLSPAILNYGGTDIAFAPYGEYWRQLRKICTIELLSSKRVQTFRSIREQEVSNLFRSIKSAEGSVVNLSKMIYSLTYSITAQAAFGKESKYQDKFISVMQDAIKLMGGFNIADVYPSIKILEKITGIRHKLEETHNQIDEIVQNILIEHKAKRAESKLGNRGAKEDLVDVLLNIQSSGEFEPPLTDDNLKAVIYDIFSAGGETSSTTVIWAMSEMIRNPKVMKKAQDEVREVFAGNGNVDESRLHELKYLQAVIKETMRLNPAVPLLLPRECSEHCQINGYDVPAKTRVIVNAWAIGRDPKQWTEPEKFKPDRFLDSEIDFRGKTFSFIPFGAGRRICPGISFALPLVELPLAQLLYHFDWKLPGQSKPDELDMTEIFGLTVGRKHDLNLVPIPYRPCSVL

SEQ ID NO: 171 - protein, 508 aa, synthetic construct
MAALFEWEKEGFPYPYLLIFASLSLIVVLKFLLKPKNNRNSKLPPCPPKLPIIGNLHLLGNMPQESLHQLAKKYGPIIFLQLGEVPTVVISSARLAKEAMKTFDLALSSRPQIFSAKHLFYNCTDVVFSPYGAYWRNIRKICILELLSAKRVQSFSYVREEEVAHLVQRVAEAYPGTTNLTKMLGLYANNLVCRVAFGKDFSQGGDNHRHNFQKLLEEYQVLLGGFSIGDFFPSMEFVHKFTGMESRLKDTFRRFDQFFDEILQEHRNPESRKEHKDLVDVLLEIQKNGDPETPLTTDNIKAIILDMFAAGTDTNFITLDWAMTELMTNPGVLKRAQAEVRNIVGERRSVSENDLPQLPYVKAVVKETFRLHPPAPVLVPRESMEDVTIDGYDIPAKTRFFINAWAIGRDPKSWENPDVFEPERFMNSHIDFKGQDYELIPFGAGRRSCPAITFSAVTVELALAQLLHSFDWELPPGVEAKDLDMTEVFGITMHKIYPLMVVAKPYFS

SEQ ID NO: 172 - protein, 321 aa, synthetic construct
MAALIEDHSTQASVFILMELFLTLCALLAFFSIALKIVRKTKLVKLPPGPRPLPIIGNLHQLNGSLPHRILTDLAKKYGPLMHLQLGEVSTVIVSSPAVAKEVMKDHDIIFCYRPLLLSPAILNYGGTDIAFAPYGEYWRQLRKICTIELLSSKRVQTFRSIREQEVSNLFRSIKSAEGSVVNLSKMIYSLTYSITAQAAFGKESKYQDKFISVMQDAIKLMGGFNIADVYPSIKILEKITGIRHKLEETHNQIDEIVQNILIEHKAKRAESKLGNRGAKEDLVDVLLNIQSSGEFEPPLTDDNLKAVIYVSLFYSPSHIK

SEQ ID NO: 173 - protein, 465 aa, synthetic construct
MAALLDPWQKESFLHPFIFASISLVPVLVILLMKKDKRSVKLPPSPPKLPIIGNLHQIGNMPHESFHNLAKKFGPIMFLWLGQVPTVVISSARLAKEALKTSDLALSSRPELFSAKHLHYNCTDLAFSPYGTYWRNIRKITTLQLLSAKRVQSFSFVRKDEVAHLVKRITESYPSTINLSHMLEVYANNNICRVAFGKDFSQGGDNVRHNFQKVLEEYQMLLGGFSIGDYFPSMEFLHKFTGMESRLKDTFRRFDHICDKILEEHRKTENRKDQKDLVDVLLEIQHNGDLETPLTTDNIKAIIIDMFVGGTDTNFITLDWTMTELIINPKVMQRAQAEVRSIVGERRFVSENDLSHLGYMKAVVKETFRLHPPAPLLAPRESIQDVTIDGYNIPFGAGRRICPAISFTVATVELALAQLLHSFDWELPPGVEAKDLDMTEAFGITMHRTSPLMVVAKPYFSDHLN

SEQ ID NO: 174 - protein, 536 aa, synthetic construct
MAANVKANKHNIVCILCFLFLTMAKQIFVLDLPFSSLFPLALLLLTLVIWFYASSRPQKRLPPSPPKLPIVGHLFRLGIYPHRTLQRLSKKYGPLMMIHLGSKPILVASSADVACEIMKNHDLVFANRPKRSINSKLLYDCKDIATAQYGEYWRQIRSICVLQLLSNKRVQSFRHVREEETSMMIQKIRERCSSSSSSLINLSDALITLTNDVICRVALGRKYSNDENGRKSMKIMKEFGELLGTFSVGDYLPWLAWVNRFNGLDAKLEKVAKHIDQFLEGVIEEHKFRKKGEDYVDSTEKKALDLVDILLDIQKDNSVGLALELDSVKAIILDMFAGGTDTSYTAMEWAMSELLRDPKTLEKLQVEVRKVAQGKSEITEDDLDKMHYLKAVIKETLRLHTPIPLLVPRESTQHVKILGYDIPAGTEVMVNAWAIARDPVQWKKPEEFLPERFLNSTIDFRGFNFEFIPFGAGRRGCPGATFAIAVNELALAKLLHEFDFALPDGAKPEDLDMSDATGFTVHRKFPLLAVAIPHSC

SEQ ID NO: 175 - protein, 539 aa, synthetic construct
MAAVRILKKKKFQFTILNTKKEMSIFQFEEITAGLSKHFPSLLLLLLISLLLLRWKNSTSDTTQNLPPSPPQLPIIGNLHQLGSIPQRSLKSLAQKYGSLMLLRLGSKPVLIISSADAAEEVLKTHDLIFANRPKPDFVGRLLYNFKDIAFSPYGGYWRHIRSICVLQLLTNKRVQSFRNIREEEVELMLEKIRESCASSSIILIGEILATLTNNIVSRVATGKRYSEEESGSKFKDLFEEFSMLLGVFNVGDYIPWLAWINHINGLEAKVKRVGKDFDEYMDKIVQEGIIRQEKRGSNGGDHEKEQQNLVDVLLELQRTNSAGFAIGRDSIKAIITNMFVGGTDTTFSLLEWAISELLRNTNAMHKLQKEVRQFLGRKSRITEDDLENLHYLKAVIKEALRLHPPAPLLIPRESSKAVKLMGYNIAAGTQVIINAWAIGRDPKLWDDAGEFRPERFLNSSVDINGQHFELIPFGSGRRSCPGTAFAMITAELALANLICNFDFELAGGARPEDLDMTEAPGIITPRKVPLRLVASLPN

SEQ ID NO: 176 - protein, 515 aa, synthetic construct
MAVSFPAFQDEAKRWLSNHSVAVSLLLPSFMIFILFLSKWWFLTTSKKKNLPPSPPRLPIIGNLHQIGSVAHRSFKSLAEKHGPLMLLHVGRQKLLVVSSAETAREVLKTHDVAFCGRPDSEVTRRIFYNLKNISLSPYGDYWKIVRSIAVNQILNQKRVQSFRSVREEEISLLVERIKESCASSSVICMNTLLTTLVNDIVSRITIGKRSSGKGGSRFAELLFASTELFGSFLAGDFFPGLGWLGYITGFEAKTKKIAKDLDQCLENLIEEQINRNKRGDNQGKDNQNFLQVLLEIQRTDSSGHALDRESIKAIIMDMVAGGFDTYSLLEWATSLLVKHPDVMKKLQNEVKEVAGSKSFISEDDLSKLQYLKAVIKETFRLCPGAFIVRVSTKDVKIMGYDIAAGTQVLVNALAIGRDPTLWEKPEEFRPERFLNSSIDLKGHHFELLPFGSGRRSCPGSTFALVIDELVLANLVCNFNYALPGGAKAEDLDMTEAPGAMPRRRTPLLLVPSLC

SEQ ID NO: 177 - protein, 501 aa, synthetic construct
MAELFLTLCALLAFFSIALKIVRKTKLVKLPPGPRPLPIIGNLHQLNGSLPHRILTDLAKKYGPLMHLQLGEVSTVIVSSPAVAKEVMKDHDIIFCYRPLLLSPAILNYGGTDIAFAPYGEYWRQLRKICTIELLSSKRVQTFRSIREQEVSNLFRSIKSAEGSVVNLSKMIYSLTYSITAQAAFGKESKYQDKFISVMQDAIKLMGGFNIADVYPSIKILEKITGIRHKLEETHNQIDEIVQNILIEHKAKRAESKLGNRGAKEDLVDVLLNIQSSGEFEPPLTDDNLKAVIYDIFSAGGETSSTTVIWAMSEMIRNPKVMKKAQDEVREVFAGNGNVDESRLHELKYLQAVIKETMRLNPAVPLLLPRECSEHCQINGYDVPAKTRVIVNAWAIGRDPKQWTEPEKFKPDRFLDSEIDFRGKTFSFIPFGAGRRICPGISFALPLVELPLAQLLYHFDWKLPGQSKPDELDMTEIFGLTVGRKHDLNLVPIPYRPCSVL

SEQ ID NO: 178 - protein, 501 aa, synthetic construct
MAELFLTLCALLAFFSIAIKIVRKTKPVKLPPGPRPLPIIGNLHQLNGSLPHRILTDLAKKYGPLMHLQLGEVSTVIVSSPAVAKEVMKDHDIIFCYRPLLLSPAILNYGGTDIAFAPYGEYWRQLRKICTIELLSSKRVQTFRSIREREVSNLFRSIKSEEGSVVNLSKRIYSLTYSITAQAAFGKESKYQDKFISVMQDAIMLMGGFNIADVYPSIKILEKITGIRHKLEEVHNQVDEIVQNILIEHKAKRAESKLGNRGAKEDLVDVLLNIQSSGEFEAPLTDDNLKAVIYDIFSAGGETSSTTVIWAMSEMIRNPKVMKKAQDEVREVFAGNGNVDESRLHELKYLQAVIKETMRLNPAVPLLLPRECSEHCQINGFDVPAKTRVIVNAWAIGRDPKQWTEPEKFKPDRFLDSEIDFRGKTFSFIPFGAGRRICPGISFALPLVELPLAQLLYHFDWKLPGQLKPDELDMTEIFGLTVGRKHDLNLVPIPYRPCSVL

SEQ ID NO: 179 - protein, 535 aa, synthetic construct
MAISSSIKSILLKLTIAQLALFFLQSSMDFIIFFGALLLFYLMVAKIHRKSTGENSKLNLPPGPKPLPIIGNMHQLHGSPIHHLLRDLAKKYGPLMHLKLGEISAIIVTSPEMAKEIYKTNDLLFASRPSTHLAFKIISYNFTGIIYSAYGNHWRQLRKICTAELLSPKRVQTFKSIREDEVFNLIQLIYSQKGSIINLSRCIFLLSYSITSRIAFGKRNKYTERFIQLVEEINKLVSGFSLADMYPSVKLLQVLSIMRYKLEIAHRQVDQILENIINDHKQKITEAKQEGGERKEDFVDVLLIIQEGGDSEPQLTDAAIKAVIFDIFSAGSETSSTAIEWALSELIRNPEVIKRAQDEVRNVYDERGNVDESSLHELKYLHAIFKETLRLHPSAPLLLPRECGEQCEINGYVIPAKARILVNAWAIGRDPMYWTEAEKFIPERFLDSEIDFKGNNFEYIPFGAGRRICPGISFSQPHVELVLAQLLFHFDWKLPGELKQEELDMTEDFGVTTKRSNDLHLIPIPYNRSCLRMDA

SEQ ID NO: 180 - protein, 503 aa, synthetic construct
MAEFIIFLGALLLFYFVISLGKSSKLKLPPGPKQLPIIGNMHQLHGSQLHHLLRDLAKIYGPLMHLKLGEISTIIVTSPEMAREIYKTNDLLFASRPSHYLAFKIFSYNFADIAFSPYGNYWRQMRKICTLELLSTKRVQTFQSIREDEVFNLMKSISTQKGSIINLSRSIFTVIYTITSRAAFGKRNKDTERYIRLVEEVTKLASWFSLADVYPSVKLFQLMSTVRHKAAIVHKQVDEIIENILNDHKEKIEEAKQASGEAKEDLVDVLLSIQNSGDFEPQLTDTNIKAIIFDMFTAGGETSSTVVEWAISEMIKNPETIKRAQNEVRNAFDEKGNVDESKLHELKYLNAIIKETLRLHPSAPLLVPRECTEQCEINGYVIPAKARLFVNAWAIGRDPKYWIDAENFNPERFLDSKVDYRGNDFEYIPFGAGRRICPGITFSRPGIELALAQLLFHFDWKVPGELKQEELDMTENFGMTVRRKNDLNLIPVLYNRSCLSKDN

SEQ ID NO: 181 - protein, 512 aa, synthetic construct
MAALFEWEKEGFPSPYLLIFASVSLIVVLKFVSKPKNNRNSKLPPCPPKLPIIGNLHLLGNMPQESLHQLAKKYGPVIFLQLGEVPTIVISSARLAKEAMKTFDLALSSRPQIFSAKHLFYNCTDVVFSPYGAYWRNIRKICILELLSAKRVQSFSYVREEEVAHLVQRVAEAYPGTTNLTKMLGLYANNLVCRVAFGKDFSQGGDNHRHNFQKLLEEYQVLLGGFSIGDFFPSMEFVHKFTGMESRLKDTFRRFDQFFDEILQEHRNPESRKEHKDLVDVLLEIQKNGDPETPLTTDNIKSIILDMFAAGTDTNFITLDWAMTELMTNPGVLKRAQAEVRNIVGERRSVSENDLPQLPYVKAVVKETFRLHPPAPVLVPRESMEDVTIDGYDIPAKTRFFINAWAIGRDPKSWENPDVFEPERFMNSHIDFKGQDYELIPFGAGRRSCPAITFSAVTVELALAQLLHSFDWELPPGVEAKDLDMTEVFGITMHKIYPLMVVAKPYFSQGLN

SEQ ID NO: 182 - protein, 501 aa, synthetic construct; identical to SEQ ID NO: 177

SEQ ID NO: 183 - protein, 501 aa, synthetic construct
MAELFLTLCALLAFFSIAIKMVKNSKPDNLPPGPMQLPIIGNIHQLFGSLAYRILTGLAKKHGPLMHLQLGEISTIVVSLAEVAKEVMNEHDIIFASRPNLLGTRIHSYNATKIAFSPYGDYWRQLCKICTMELLSPKRVETFRSIREEAVLDMIKSIALQRGSDVNLSKEIFSLTYRITAQAAFGKRNKHQEEFISVMVDIVELVAGFNIADMYPSVKILERISGMRQKLERTHKQVDEILENILTEHKGKRDGLQPGKGDSKEDLVDVLLKVQKSGEFGAPLSNNNLKAVILDIFSAGDESSSTTAQWTMLEMMKNPGTIKRSQEEVREVFGDKGNVDESRLHELKYLQAVIKDALRLRPPVPLLLPRECAEPCEIHGYEIPAKARMIVNAWAIGRDSRRWTELEKFISERFLDSDYDFRGQNFCYIPFGARRRICPGIAFALPNVELLLAQLLYHFDWKLPGEIKEQQLDMTETFGLAVRPKHDVNLVPISFSPSDEN

SEQ ID NO: 184 - protein, 509 aa, synthetic construct
MADLIIFLSAFLLFYLVVVKLLRKPTGKNSKLNIPPGPKPLPIIGNMHQLHGSPIHHLMRDLAKKYGPLMHLKLGEISAIIVTSPEMAKEIYKTNDLLFASRPSSHLAFKIISYNFADIIFSAYGNHWRQLRKICTVELLSPKRVQTFKSVREYEVFKLIQSISSKRGSIINLSRSIFPLSYSITSRIAFGKRNKYTERFIQIVEEINKLVSGFSLADMYPSVKLLRVMSIMRYKLVIAHKQVDQILENILNDHKQNVTEAKQEGGERKEDLVDVLLNIQEGGDFEPELTGTAIKAVLFDIFSAGSETSSTAIEWALSEMIRNPEVIRRAQDEVRNVYDERGNVDESSLHELKYLRAIIKETLRLHPSAPLLLPRECSEQCEINGYVIPAKARIIVNAWAIGRDPMYWAEAEKFNPERFLDSEIDFKGNNLEYIPFGAGRRICPGISFSQPHMELVLAQLLFHFDWKLPGELKQEELDMTEDFGVTIRRTNDLHLIPIPYNRSCLRMDG

SEQ ID NO: 185 - protein, 501 aa, synthetic construct
MAELFLTLCALLAFFSIAIKMVAITLKNSKPRDLPPGPMQLPIIGNIHQLFGSLPHRILTDQAKKYGPLMHLQLGEISTFVVSSAEVAKEVMNEHDITFASRPNLLGTRIISYNATDIAFSTYGDYWRQLRKICTMELLSPKRVQTFRSIREEAVLDMVKSLALQRGSIVNLSKEIFSLTYRITAQAAFGKQNKHQEEFISVMVDIVELVAGFNIADMYPSVKILERISGMSQKLERTHKQVDEILENILAEHKGKRDGLHPVKGEVKEDLVDVLLKVQNSGDFGAPLSDSNPKRSSYAGGETSSTTAQWSMLEMMKNPGIIKRAQEEVREVFGDKGNVDESRLHELKYLPAVIKETLRLHPPVPLLLPRECGEPCEIHGYEIPAKARVLVNAWAIGRDPRHWTEPDKFIPERFLDSDYDFRGQNFCYIPFGAGRRICPGIAFGLPNVELPLAQLLYHFDWNLPGEIGEQQVDMTETFGLAVRPKHGVHLVPFSFSNSSEN

SEQ ID NO: 186 - protein, 688 aa, synthetic construct
MTTLLNPYFGEFGGMYVPQILMPALRQLEEAFVSAQKDPEFQAQFNDLLKNYAGRPTALTKCQNITAGTNTTLYLKREDLLHGGAHKTNQVLGQALLAKRMGKTEIIAETGAGQHGVASALASALLGLKCRIYMGAKDVERQSPNVFRMRLMGAEVIPVHSGSATLKDACNEALRDWSGSYETAHYMLGTAAGPHPYPTIVREFQRMIGEETKAQILEREGRLPDAVIACVGGGSNAIGMFADFINETNVGLIGVEPGGHGIETGEHGAPLKHGRVGIYFGMKAPMMQTEDGQIEESYSISAGLDFPSVGPQHAYLNSTGRADYVSITDDEALEAFKTLCLHEGIIPALESSHALAHALKMMRENPDKEQLLVVNLSGRGDKDIFTVHDILKARGEIGGGSGEGRGSLLTCGDVEENPGPMERYESLFAQLKERKEGAFVPFVTLGDPGIEQSLKIIDTLIEAGADALELGIPFSDPLADGPTIQNATLRAFAAGVTPAQCFEMLALIRQKHPTIPIGLLMYANLVFNKGIDEFYAQCEKVGVDSVLVADVPVEESAPFRQAALRHNVAPIFICPPNADDDLLRQIASYGRGYTYLLSRAGVTGAENRAALPLNHLVAKLKEYNAAPPLQGFGISAPDQVKAAIDAGAAGAISGSAIVKIIEQHINEPEKMLAALKVFVQPMKAATRS

SEQ ID NO: 187 - protein, 131 aa, synthetic construct
MAAEQTTFTRSEIEKNNDKDKVLIILHDKVFNVHRFLNEHPGGEEVLLDHKGKDGSEDFDDVGHSKDAMELMKTYQVGVLVESERSNKPPKKGWVAGYNTKQSEKYVQPGVPFYLLIGGIALVVALFFYAY

SEQ ID NO: 188 - protein, 135 aa, synthetic construct
MAASDQKLHKFDEVSKHNKTKDCWLIINGKVYDVTPFMDDHPGGDEVLLSATGKDATNDFEDVGHSDSAREMMDKYYIGEMDMATVPLKRTYIPPQQAQYNPDKTPEFVIKILQFLVPLLILGLAFAVRHYTKEK

SEQ ID NO: 189 - protein, 679 aa, synthetic construct
MAEKTEAILETEHISEASDSLFSSLDIFLVALIVGGISWYFINKYKKTTTVTSERSYSISPSPITLENPSDSSFIKKLQSSGRSLVVFYGSQTGTAEEFAGRLAKEGARYRLKGMVADLEDYEMEELVNLKNIPNSLAVFCLASYGEGEPTDNAREFCDWLQNSEPDLKGLTYTVFGLGNKSYEYYNRVAINVDKRLKELGATRIYDLGLGDDDANIEDDFITWKDQFWPTVCEHFGIEPTEEEINMHQYRLEEFEEAPDKVFVGEMAKLDSLKNQKPPHDAKNPFLAKVKVNRDLNNSDRSCLHIEFDIKGSKMRYDSGDHIAVYPTNDSELVEKIGKLCAKNLDTIFTLINNDEESNKKHPFPCPCSYRTALTHYLDITQNPRTHVLKELAEYCTDEANKEKLRLMSSTTPEGKALYQSWIIEDNRSVVHVIEDMPCCRPSLDYLCELLPRLQPRFYSISSSAKLYPDTVHISAVVVEYDTPTGRHNKGVATTWLKEKVPDPEKEAPVVPVFIRKSQFRLPTKPETPIIMIGPGTGLAPFRGFIQERNICKSEGKPVGETVLYFGCRKRSEDFIYGDELLEYEKNGLLTLHLAFSRDQPHKVYVTHLLQQHADEVWRVIGENNGHLYICGDAKSMAVDVRVILTKILQEKGQMTEQEAVAFLKKMETQRRYSADVWS

SEQ ID NO: 190 - protein, 682 aa, synthetic construct
MAENSEGVKIESTVVTEEPLISALDIALFVVIITVAYFWYFKRDKQSSSSEKKPYTIQPSSLSSIEQTSNSSFIKKLKSTGRSLVVFYGSQTGTAEEFAGRIAKEGARYKMKGMVADPEECDMADLVEMKEIQKSMAIFCVATYGEGDPTDNAMDFYEWLQNGDADLEGLNYAVFGLGNKTYEHYNEIAIYIDQRLEELGATRVHEIGLGDDDANIEDDFVSWKEKLWESVCSHYGIEETGEESNIRQYKLVDCSEILPERIFSGEISRLKSYENQRFPFDVKNPYLSKIAVNRELHKSGDRSCMHIEFDIDGSKMRYDTGDHVAVYPKNSSELVEKIGELLNADLDTVFSLLNTDEESSKKHPFPCPCTYRTALTYYLDITSNPRTHIMKELIEYASDPKDQEKLKLMASSTPEGKKEFHDWILRDNRNIVHILEDLPSVKPDLDHLCELLPRLQCRYYSISSSPKVYPKSIHITAVLVNYTTPTNRVNKGVATNLLAQLKPTNDELPQPTIPIYIRRSQFRLPPKSQTPIIMIGPGTGLAPFRGFIQERDYARKEGREIGEMVLYFGCRKKDEDFIYENELQEYVANGTLTKLHLAFSRDQPEKIYVTHLLEQNADELWNIIGEKNGHLYVCGDARSMAKDVHNIIVKVVMEKGQMTNSQALNYVKKMEQQKRYSADVWS

SEQ ID NO: 191 - protein, 715 aa, synthetic construct
MADSSSEKLSPFELMSAILKGAKLDGSNSSDSGVAVSPAVMAMLLENKELVMILTTSVAVLIGCVVVLIWRRSSGSGKKVVEPPKLIVPKSVVEPEEIDEGKKKFTIFFGTQTGTAEGFAKALAEEAKARYEKAVIKVIDIDDYAADDEEYEEKFRKETLAFFILATYGDGEPTDNAARFYKWFVEGNDRGDWLKNLQYGVFGLGNRQYEHFNKIAKVVDEKVAEQGGKRIVPLVLGDDDQCIEDDFAAWRENVWPELDNLLRDEDDTTVSTTYTAAIPEYRVVFPDKSDSLISEANGHANGYANGNTVYDAQHPCRSNVAVRKELHTPASDRSCTHLDFDIAGTGLSYGTGDHVGVYCDNLSETVEEAERLLNLPPETYFSLHADKEDGTPLAGSSLPPPFPPCTLRTALTRYADLLNTPKKSALLALAAYASDPNEADRLKYLASPAGKDEYAQSLVANQRSLLEVMAEFPSAKPPLGVFFAAIAPRLQPRFYSISSSPRMAPSRIHVTCALVYEKTPGGRIHKGVCSTWMKNAIPLEESRDCSWAPIFVRQSNFKLPADPKVPVIMIGPGTGLAPFRGFLQERLALKEEGAELGTAVFFFGCRNRKMDYIYEDELNHFLEIGALSELLVAFSREGPTKQYVQHKMAEKASDIWRMISDGAYVYVCGDAKGMARDVHRTLHTIAQEQGSMDSTQAEGFVKNLQMTGRYLRDVW

SEQ ID NO: 192 - protein, 697 aa, synthetic construct
MAHNMDLPTVDASQPKAIRMAGSPMLENESGPEDEQPFLSITDIMLILGILLAMMFVYVRSRMRREAAPAPKSYSIQPTSFAVAPSSENSFIKKLKTSGRSLVVFYGSQTGTAEEFAGRLAKEGIRYKMKGMVADPEECNMEELVHLKTIPNSLAVFCLATYGEGDPTDNAMEFIDWLKNGDADLTELNYAVFGLGNKTYEHYNEVGIYVDHRLEQLGATRVFELGLGDDDANIEDDFITWKDKFWPAVCEFFGIEGAGEDVSIRQYKLTEHVDMPAERIYTGEFARLHSFKNQRPPYDAKNPFLAPVIVNRELHGPTSERSCMHIEFYIEDSKIRYDAGDHLAVYPVNNAKLVNKIGEQCGANLDIVFTLTNTDEESTKKHPFPCPCTYRTALTYYLDITSNPRTHILKELAEYATDPADKEKLKLMASTTAEGKAAYQQWIIQENRNIVHILEDIPSLKPPLDHLCEILPRLQCRYYSISSSPKLHPTTIHITAVVVEYKTPTGRINRGVTTSWLKEKHPSDPPCLVPIFLRKSQFRLPTRPNIPIIMVGPGTGLAPFRGFIQERDFARKEGKEVGDTILYFGCRKSQEDFLYRDELEEYVKNGTLTLHTAFSREQSHKVYVTHLLEKNKEEIWHVIGEQNGHIYVCGDARNMARDVHNILLKVVMERGNMSELDAANYIKKMESQKRYSSDVWS

SEQ ID NO: 193 - protein, 588 aa, synthetic construct
MAEVEKTEKNIAEDDESKIIYRGWKVMPFIIGNETFEKLGIVGSSSNLVIYLTTVFNMKSITAAKVVNIYGGTSNFGTIVAAFLCDSYFGRYKTLSFAMIACFLGSVAMDLTAVIHPLHPAQCAKEIGSVCNGPSIGQIMFLAGAMVLLVIGAGGIRPCNLPFGADQFDPKTKEGKRGIESFFNWYFFTFTFAQMVSLTLIVYVQSNVSWSIGLAIPAILMLLGCIIFFAGSKLYVKVKASGSPIHSITRVIVVAIKKRRLKPVGPNELYNYIASDFKNSKLGHTEQFRFLDKSAIQTQDDKLNKDGSPVDAWKLCSMQQVEEVKCVIRVLPVWLSAALFYLAYIQQTTYTIFQSLQSDRRLGPGSFQIPAGSYTVFLMLGMTIFIPIYDRVLVPFLRKYTGRDGGITQLQRVGAGLFLCITSMMVSAIVEQYRRKVALTKPTLGLAPRKGAISSMSGMWLIPQLVLMGIADALAGVGQMEFYYKQFPENMRSFAGSLYYCGIGLASYLSTFLLSAVHDTTEGFSGGSWLPEDLNKGRLEYFYFLVAGMMTLNLAYFLLVSHWYRYKDVVAKDKDMDKTSAEFDKVSV

SEQ ID NO: 194 - protein, 596 aa, synthetic construct
MAAEKSSSIDATENGFVKIKEPNHRGVKAMPFIVGNETFEKLGTLGTSANLLIYLTTVFHMKSITATNLINIFNGTCNFGTLLGAFFSDTYFGRYKTLGFASVSSTLGMLILTLTAAISKLHPPSCEKNSKACSGPTSGQFAFLFTAFLFLIIGASGIRPCNLAFGADQFNPNTESGRKGTNSFFNWYYFTFTFAMMVSLTVIVYVQSSISWALGLAIPTFLMLLSCVFFFVGSRIYVKVLPEGSPLTSMMQVIVAAFRKRKLNLPDHQPWDSLFNYISPSSINSKLSYTDQFRWLNKAAIKTSKDHLYAEGSTANPWRLCSMQQVEEVKCVIRSIPIWTSGVLYYVVLNQMSTYLVFQALQSDRRIGKTNFKIPAASYTVFSMLSLSIWIPIYDRIIVPFLRKITKTEQGITILQKMGFGLVIAVITMVVSAIIEDKRRVIALTRPIVGIEERKGGISSLSGFWLVPQLALAGISEAFTVIAQVEFFYKQFPENMRSFGGSFLFCGFAISSYLSSFLISIVHKVTRNSSGGNWLDEDLNKARLDYFYYLVAGLEVLNFGYFLVCAHWYRYKETQSGNEIAMEQILDEPKKNLDVV

SEQ ID NO: 195 - protein, 589 aa, synthetic construct
MAEENQEHKAIKNHEPNYRGIKAMPFVIGNETFEKLGTIGTSTNLLIYLTSVFHMKTITATNLVNIFNGTCNFGTLVGAFLCDTYFGRYNTLGFASVSSFLGMLVLTLTAAIKNLHPEACGNEEGSKCISPSGGQMAFLLSGFAFLIVGASGIRPCNLAFGADQFNPNTDSGRRGINSFFNWYYFTYTFAMMVSLTIIVYVQSNISWSIGLAIPTFLMFLSCATFFVGTRIYVRVLPEGSPLISVARVIVSAFKKRKLELPDEPKLSLFNHSASSTSSINSKISYTDQFRFLNKAAIITSEDEFNSDGSVKNPWRICTIQQVEEVKCLLKLIPVWISCIIYYISVVQQQNYVVFQALQSDRRLFGTSKFKIPAASYIVIAMLALTIWIPIFDRIIVPCLQKITGKEGGITILQRMGIGMVLSVLTMITSALVESKRRKFALNRPTLGFEPRKGAISSMSGVWLIPQLALSGLSEAFTIIGENELFYKECPENMRSIAMALVFVGFAGSSYLSSLLSSIVQKTTGGNESWLAEDLNRGRLDYFYYLIGALETLNLIYFLVCSKWYKYKQVEKIPQVAMEKLNNVEEKSLV

SEQ ID NO: 196 - protein, 594 aa, synthetic construct
MAEKNEKLVPNGTKEDGKVAAKNEPNYRGIKAMPFVLGNETCEKLGTIGTSSNLSVYLSTVFHMNTMSANNVINIFNGTCYLGSLVGAFLCDTYFGRYKTLGLGSVASFLGMLMLTLTAAISTLHPTNCDNSGDCKGPSAGQMAFLVGGFVLMVIGASGIRPCNLAFGADQFNPNTESGRRGINSFFNWYYFTYTFAVMVSATIIVYVQSDISWSIGFAIPTSLMFLSCALFFAGTWLYVKVLPQGSPMTSLVQVIVVSVKKRRLKLPGEPSLLFNHIPSNAINSKLPRTNNFRFLDKAAILTPEDKVNPDGSAANKWKLCSIQQVEEVKCVLRVLPIWIAGVIFQISVTQQSNYVPFQAFQSDRHLIKGNKFQIPAGTYNVFSMLALTIWLPIYDRIIIPLLRRWTKKEDGITLLQRMGVGIVLSILSMLVSALVESRRRNLALTRPTLGIQEGQGSISSMSALWFVPQLALMGLAEGFTFIGENELFYKEFPENMRSIAASLVLCGVAVASYLTSFIAEIVHKTTRSGTRDWLAQDLNKARLDYFFYMVAVLEALNLVYFLVCAKWYRYKGTGDLGKLEVAMEKLETEKHLV

SEQ ID NO: 197 - protein, 683 aa, synthetic construct
MLNHPSQGSDDAQDEKQGDFPVIEEEKTQAVTLKDSYVSDDVANSTERYNLSPSPEDEDFEAPTEEEMQTLRHVGGKNEPNYRGIKAMPFVLGNETCEKLGTIGTSSNLSVYLSTVFHMNTMSANNVINIFNGTCYLGSLVGAFLCDTYFGRYKTLGLGSVASFLGMLMLTLTAAISTLHPTNCDNSGDCKGPSAGQMAFLVGGFVLMVIGASGIRPCNLAFGADQFNPNTESGRRGINSFFNWYYFTYTFAVMVSATIIVYVQSDISWSIGFAIPTSLMFLSCALFFAGTWLYVKVLPQGSPMTSLVQVIVVSVKKRRLKLPGEPSLLFNHIPSNAINSKLPRTNNFRFLDKAAILTPEDKVNPDGSAANKWKLCSIQQVEEVKCVLRVLPIWIAGVIFQISVTQQSNYVPFQAFQSDRHLIKGNKFQIPAGTYNVFSMLALTIWLPIYDRIIIPLLRRWTKKEDGITLLQRMGVGIVLSILSMLVSALVESRRRNLALTRPTLGIQEGQGSISSMSALWFVPQLALMGLAEGFTFIGENELFYKEFPENMRSIAASLVLCGVAVASYLTSFIAEIVHKTTRSGTRDWLAQDLNKARLDYFFYMVAVLEALNLVYFLVCAKWYRYKGTGDLGKLEVAMEKLETEKHLVEEEDEFDLNPISAPKANDIEILEPMDSLRSTAKY

SEQ ID NO: 198 - protein, 649 aa, synthetic construct
MLNHPSQGSDDAQDEKQGDFPVIEEEKTQAVTLKDSYVSDDVANSTERYNLSPSPEDEDFEAPTEEEMQTLRHVGGKNEPNYRGIKAMPFVLGNETCEKLGTIGTSSNLSVYLSTVFHMNTMSANNVINIFNGTCYLGSLVGAFLCDTYFGRYKTLGLGSVASFLGMLMLTLTAAISTLHPTNCDNSGDCKGPSAGQMAFLVGGFVLMVIGASGIRPCNLAFGADQFNPNTESGRRGINSFFNWYYFTYTFAVMVSATIIVYVQSDISWSIGFAIPTSLMFLSCALFFAGTWLYVKVLPQGSPMTSLVQVIVVSVKKRRLKLPGEPSLLFNHIPSNAINSKLPRTNNFRFLDKAAILTPEDKVNPDGSAANKWKLCSIQQVEEVKCVLRVLPIWIAGVIFQISVTQQSNYVPFQAFQSDRHLIKGNKFQIPAGTYNVFSMLALTIWLPIYDRIIIPLLRRWTKKEDGITLLQRMGVGIVLSILSMLVSALVESRRRNLALTRPTLGIQEGQGSISSMSALWFVPQLALMGLAEGFTFIGENELFYKEFPENMRSIAASLVLCGVAVASYLTSFIAEIVHKTTRSGTRDWLAQDLNKARLDYFFYMVAVLEALNLVYFLVCAKWYRYKGTGDLGKLEVAMEKLETEKHLV

SEQ ID NO: 199 - protein, 424 aa, synthetic construct
MGGASASGVDNKFNKERVIAIGEIMRLPNLNSLQVVAFINSLRDDPSQSANLLAEAKKLNDAQAPKGGSSASSAGGSSVDNKFNKEAQTAGVEIMELPNLNTRQLLAFIQSLRDDPSQSANLLAEAKKLNDAQAPKTSGGSSASSAGGSSVDNKFNKERVIAIGEIMRLPNLNSLQVVAFINSLRDDPSQSANLLAEAKKLNDAQAPKGGSSASSAGGSSVDNKFNKEAQTAGVEIMELPNLNTRQLLAFIQSLRDDPSQSANLLAEAKKLNDAQAPKTSGSGASGSGAGGSSVDNKFNKERVIAIGEIMRLPNLNSLQVVAFINSLRDDPSQSANLLAEAKKLNDAQAPKGSGASGSGAGGSSVDNKFNKEAQTAGVEIMELPNLNTRQLLAFIQSLRDDPSQSANLLAEAKKLNDAQAPKTS

SEQ ID NO: 200 - protein, 70 aa, synthetic construct
MVDNKFNKELGWATWEIFNLPNLNGVQVKAFIDSLRDDPSQSANLLAEAKKLNDAQAPKGSSSSGSSSSG

SEQ ID NO: 201 - protein, 70 aa, synthetic construct
MVDNKFNKETIQASQEIRLLPNLNGRQKLAFIHSLLDDPSQSANLLAEAKKLNDAQAPKGSSSSGSSSSG

SEQ ID NO: 202 - protein, 56 aa, synthetic construct
MQLLRCFSIFSVIASVLAQELTTICEQIPSPTLESTPYSLSTTTILANGKLDKRGS

SEQ ID NO: 203 - protein, 72 aa, synthetic construct
MKVLDLLTVLSASSLLSTFAAAESTATADSTTAASSTASCNPLKTTGCTPDTALATSFSEDFSSSSLDKRGS

SEQ ID NO: 204 - protein, 51 aa, synthetic construct
MLSLKTLLCTLLTVSSVLATPVPARDPSSIQFVHEENKKRYYDYDHGSLGE

SEQ ID NO: 205 - protein, 33 aa, synthetic construct
MKFAYSLLLPLAGVSASVINYKRDGDSLDKRGS

SEQ ID NO: 206 - protein, 66 aa, synthetic construct
MKLQSLLVSAAVLTSLTENVNAWSPNNSYVPANVTCDDDINLVREASGLSDNETEWLKKRDAYTKE

SEQ ID NO: 207 - protein, 20 aa, synthetic construct
MLLQAFLFLLAGFAAKISAS

SEQ ID NO: 208 - protein, 99 aa, synthetic construct
MKLSATTLTAASLIGYSTIVSALPYAADIDTGCTTTAHGSHQHKRAVAVTYVYETVTVDKNGQTVTPTSTEASSTVASTTTLISESSVTKSSSLDKRGS

SEQ ID NO: 209 - protein, 92 aa, synthetic construct
MRFPSIFTDVLFAASSALATPVNTTTEDETAQIPAEAVIGYSDLEGDFDVAVLPFSNSTNNGLLFINTTIASIAAKEEGVSLEKREATNFGS

SEQ ID NO: 210 - protein, 100 aa, synthetic construct
MEEQREILEQLKKTLQMLTVEPSKNNQIANEEKEKKENENSWCILEHNYEDIAQEFIDFIYKNPTTYHVVSFFAELLDKHNFKYLSEKSNWQDSIGEDGG

SEQ ID NO: 211 - protein, 75 aa, synthetic construct
MFRIQLRTMSSKTCKSDYPKEFVSFLNSSHSPYHTVHNIKKHLVSNGFKELSERDSWAGHVAQKGKYFVTRNGSS

SEQ ID NO: 212 - protein, 297 aa, synthetic construct
MSDSSPTINFINFNQTGTCISLGTSKGFKIFNCEPFGKFYSEDSGGYAIVEMLFSTSLLALVGIGDQPALSPRRLRIINTKKHSIICEVTFPTSILSVKMNKSRLVVLLQEQIYIYDINTMRLLHTIETNPNPRGLMAMSPSVANSYLVYPSPPKVINSEIKAHATTNNITLSVGGNTETSFKRDQQDAGHSDISDLDQYSSFTKRDDADPTSSNGGNSSIIKNGDVIVFNLETLQPTMVIEAHKGEIAAMAISFDGTLMATASDKGTIIRVFDIETGDKIYQFRRGTYATRIYSIS

SEQ ID NO: 213 - protein, 57 aa, synthetic construct
MIALPVEKAPRKSLWQRHRAFISGIVALIIIGTFFLTSGLHPAPPHEAKRPHHGKGP

SEQ ID NO: 214 - protein, 35 aa, synthetic construct
MFSLKALLPLALLLVSANQVAAKVHKAKIYKHELS

SEQ ID NO: 215 - protein, 99 aa, synthetic construct
MKLENTLFTLGALGSISAALVIPNLENAADHHELINKEDHHERPRKVEFTKDDDEEPSDSEDKEHGKFHKKGRKGQDKESPEFNGKRASGSHGSAHEGG

SEQ ID NO: 216 - protein, 164 aa, synthetic construct
MKAFTSLLCGLGLSTTLAKAISLQRPLGLDKDVLLQAAEKFGLDLDLDHLLKELDSNVLDAWAQIEHLYPNQVMSLETSTKPKFPEAIKTKKDWDFVVKNDAIENYQLRVNKIKDPKILGIDPNVTQYTGYLDVEDEDKHFFFWTFESRNDPAKDPVILWLNGG

SEQ ID NO: 217 - protein, 75 aa, synthetic construct
MTKNFIVTLKKNTPDVEAKKFLDSVHHAGGSIVHKFDIIKGYTIKVPDVLHLNKLKEKHNDVIENVEEDKEVHTN

SEQ ID NO: 218 - protein, 78 aa, synthetic construct
MKSFITRNKTAILATVAATGTAIGAYYYYNQLQQQQQRGKKNTINKDEKKDTKDSQKETEGAKKSTAPSNPPIYPVSS

SEQ ID NO: 219 - protein, 117 aa, synthetic construct
MAMFCTFFEKHHRKWDILLEKSTGVMEAMKVTSEEKEQLSTAIDRMNEGLDAFIQLYNESEIDEPLIQLDDDTAELMKQARDMYGQEKLNEKLNTIIKQILSISVSEEGEKEGSGSG

SEQ ID NO: 220 - protein, 171 aa, synthetic construct
MADRDRSGIYGGTYGQHQQGGGRPMGEQVKGMIQDKGPTASQALTVATLFPLGGLLLVLSGLALAASVVGLALATPVFLLFSPVLVPAALLIGTAVTGFLTSGALGLGGLSSLTCLANTARQAFQRTPDYVEEARRRMAEAAAHAGQKTAQAGHAIQSRAQEAASGGAGAG

SEQ ID NO: 221 - protein, 93 aa, synthetic construct
MSTLQRRRVNRADSGDTSSIHSSANNTKGDKIANIAVDGDDDNGTNKKIAYDPEESKLRDNINIPTLTLMEEVLLMGLRDREGYLSFWNDSIS

SEQ ID NO: 222 - protein, 88 aa, synthetic construct
MSLPLRTIVHLVKPFACTARFSARYPIHVIVVAVLLSAAAYLSVTQSYLNEWKLDSNQYSTYLNIKPDELFEKCTHYYRSGGNAAIRS

SEQ ID NO: 223 - protein, 30 aa, synthetic construct
MDSKGSSQKGSRLLLLLVVSNLLLCQGVVS

SEQ ID NO: 224 - protein, 23 aa, synthetic construct
MKAFTSLLCGLGLSTTLAKAISL

SEQ ID NO: 225 - protein, 73 aa, synthetic construct
MSATKSIVGEALEYVNIGLSHFLALPLAQRISLIIIIPFIYNIVWQLLYSLRKDRPPLVFYWIPWVGSAVVYG

SEQ ID NO: 226 - protein, 46 aa, synthetic construct
MPFGIDNTDFTVLAGLVLAVLLYVKRNSIKELLMSDDGDITAVSSG

SEQ ID NO: 227 - protein, 33 aa, synthetic construct
MELTMASTMSLALLVLSAAYVLVALRRSRSSSS

SEQ ID NO: 228 - protein, 70 aa, synthetic construct
MSSVAENIIQHATHNSTLHQLAKDQPSVGVTTAFSILDTLKSMSYLKIFATLICILLVWDQVAYQIKKGS

SEQ ID NO: 229 - protein, 25 aa, synthetic construct
MEELLKQALQQAQQLLQQAQELAKK

SEQ ID NO: 230 - protein, 251 aa, synthetic construct
MVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSALSKDPNEKRDHMVLLEFVTAAGITLGMDELYKGGSGSAGSGGSS

SEQ ID NO: 231 - protein, 387 aa, synthetic construct
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGR

SEQ ID NO: 232 - protein, 6 aa, synthetic construct
HHHHHH

SEQ ID NO: 233 - DNA, 1140 bp, synthetic construct
  source 1..1140: mol_type = other DNA
atgtcatccgaaatttcagagaatgctcagcttaaatcagcttcaccctccagcattgagaatggacaggagggtgagaagcaggatcactttagtcacgttatgcagttggttacatccgctagtcttccgatggtcctttatacggcagtcaagttgaatttgtttgagatcatagccaaagcaggtccaggtgctaagttaagtccgagtgaaatagcctgccgtctaccgacgacaaagaaccctgatgctgcctccatgctggatcgtatgctacgtttactatcctctcatagtcttctaacctatgatgtggttgaggtagcaggtggagcggaaacggacatcgggtatgaaagggtctatggcctgtcccctgtgggagaatatctaataccagacgaagagggaaattctgtcgctccacttgtcgaattgttactggacaaggtactaattaatacttggtatgaacttggtaatgctgtactggaaggagggatacctttcaatcgtgtccacggaacgcacgcattcgattaccctagtaggaacccaaggtacaatgaacttttcaataatggtatggttggtccaacggcaatcaccatgaaggagttacttcagcaatataagggatttgaactactacaaacattagtggacgttggtggtggtctaggtataacactgcacaaaatcatcgcgaaatatccgagcatcaggggaattaactttgacctgccgcacgtcatcgaagacgctcctccctatcctggaatggaacatataggtggcgatatgtttgaatcagtccctggaggcgatgctatttttatgaaaatgatacttcatgattggtccgatgatcattgccttaaactacttaagaattgctatagagctctacctgatcatggaaaggttatagtcgtagatctaattcttcccgtaaaacctgacaccaacatctttgtcaaaggtattttccaggccgatgtgatcatgatgactcaaaaccctggtggaaaggaacgttccgaagcagacgtgcgtgcgttggcaataggtgcaggatttaagggaataaaacttgagtgttgtgcagggaacgtaggtatactggagttgtataag

SEQ ID NO: 234 - DNA, 1113 bp, synthetic construct
  source 1..1113: mol_type = other DNA
atgtcatccgcaacaagcgagaatacgcagttaaagagtgtttccccatcaagcatggagaacggaaaagtagaggaggaagagcagcttttctcttacgcaatgcagctggtaacatcagtaagtctaccgagagttcttttagctgccttcaggctaagagtattcgaaataatcgaagaagctggaccaggtgctcagttatcagccagtgagatagccgcacaggtttccggaaagaataaagacgcagctgcaatgctagaccgtatgttacgtctgcttgcctcacatagcgttttaacatgcggagcgagaagccagggtcgtgtatacggtttggcacctgttgctaaatatttcgttcagtccaaggcagacggaggtggcacccttggctcattactggctttaatacaggataaggtatttattgattcatggttccaactagaagatgccgtaagagagggaggtgtaccttttgatagagttcacgggacgcacgcatttgagtacccaggacgtgaccctagattcaatgaggtgtttaataaggccatgcttgatcataccaccatcgcaattaacaggatcttagaaaggtataaaggtttcgaaaaccttaagaccctggtcgatgtaggtggaggcttgggagttactttgaacgttataacaactaagtacccaaatcttaagggaatcaacttcgatctaccacacgttattcagagagcacccggttacccaggtgtcgaacatattggtggagatatgtttgaatctgttcctgaaggagaggcgatcttcatgaaatggattttacacgattgggacgatgggcattgcttgaaactgttaaagaattgctataaagccttatctgacaatggcaaagttatcgtggttgatgcgctggtgccagtaataccagatacaagtccttgtgtcaaaacaacctgccagagtgatttaataatgatgacacagaaccctggtgggaaagaacgtagtgaacctgagtttctggccctagctactgctgcagggtttaaaggaatcagactggaatgctacgtatgtaacctttgggtgatggaattttataaa

SEQ ID NO: 235 - DNA, 1113 bp, synthetic construct
  source 1..1113: mol_type = other DNA
atgtcatccgccactagcgaaaacacacagcttaaaagtgtctctccatcttcaatggagaacgggaaggtcgaagaggaggagcagttattttcatacgcaatgcaattggtgacctcagtctccttgccgagggttttgcttgcagcgtttagactacgtgtatttgagataattgctgaagctggaccaggtgcacagttatcagcctctgaaatcgctgcacaagtgagtggtaagaacaaggatgctgctgcaatgcttgacagaatgcttcgtttattggccagtcatagtgttctaagttgtggtgcgagatcccagggaagagtttatggattagctcctgtcgccaagtacttcgttcagtcaaaggcagatggaggtgggacgctgggtagcctattagcacttattcaagacaaggttttcatcgattcttggtatcagctagaggatgcggtcagggaaggtggagtcccctttgatcgtgtctatggtacacacgcttttgagtatcccggtcgtgacccaagattcaatgaggttttcaataaggccatgctagatcatactacaatcgccatcaacaggattctggaacgttacaaaggattcgaaaaccttaaaacactggtggacgtcggaggcggtttaggagtaacgctgaacgtgatcactaccaaatatcctaatttgaaaggcataaattttgacctaccccacgtaattcagagggcacccggctacccgggagttgagcatattggaggtgatatgttcgagagtgtcccagaaggtgaagctatctttatgaagtggatacttcatgactgggatgacggacattgtctaaaattattaaagaattgctataaggcgcttagcgataatgggaaggtaatcgtagtagacgctcttgttccggtcatcccagatacgtctccttgcgtaaagactacgtgtcaaagtgatttaatcatgatgactcagaatccaggtgggaaggagaggagtgagccagaatttcttgcactagcgacggctgcaggtttcaaggggatcagactggagtgttatgtttgtaacctgtgggtaatggagttctacaag

SEQ ID NO: 236 - DNA, 1131 bp, synthetic construct
  source 1..1131: mol_type = other DNA
atgtcatccgcgacgagcgagaatacacagcttaaaagcgtatcacccagttcaatggaaaacggcaaggctggtgaagaggaagagcactgttcatatgccctgcaactggccactagcgctgcgcttcccgttgttctaaacgcgattattaagttaaatgtgcttgaaataatcgccaaagccggtccaggcgctcaattaagtccaagtgaaattgcggctcacctgccaacggagaactcaggtgctgccgtaatgcttgacagaatgttacgtcttctggccagctactcagtgcttacttgcagtgtagttggaggtggtgataaggataaccagcaccagagagcgtatgggttggctcccgtagccaaatatttcttgagggatgaggacggaggtagtttaggctccctactggacctacttcaggataaagtgttggttgatatttggtatgagttggagcctgcggtcttgaaaggtggcactgcgttcaaccgtacgcatgggacacacatatttcaatacacgggtagcgatcaaaagtttaacgaaacatttaataaagctatgattgatcacacgttcatagtgggaaggaaagttctacaattctataaagggttcgagcatctaaagacgcttgttgatgtcggaggtggtctaggcgtcaatttatcaatgattacttcaaaatatccttctattaaagggattaattttgatttacctcacgttgtgcagaatgcacccgcttatccaggcgtagagcatgtcggaggtgatatgttcgaatcaattccaaagggagacgccattttcatgaagtggattcttcacgattgggatgacgaccattgcctgaccctacttaagaactgctataaagcgttaccagataacgggaaggtaattgctgtggacgctattttacctatggtcccggataacagcgcatcttgtaaatccaattgccaagtggatttattcacgctggctttatatttccccggtggaaaggaaaggacggaacaagaattcttagctttggcaaaggaagcgggtttcaaaggcatcagattggagtgttttgcttgtaatatgtgggttatagagttctacaag

SEQ ID NO: 237 - DNA, 1122 bp, synthetic construct
  source 1..1122: mol_type = other DNA
atgtcatccgcaacctcagagaatacacagttaaagagcgtttctccatcaagtatggagaacggtaagggttcagcacaagaggaagcctccctgtttgcgatgcaacttgcgtccgcatctgtattgccgatggttctgaagagtgcgatagaactagacttgttagaacttattgctaaagcgggtccgggtgcatacgtttcaccgtccaaattagctgcccaattacccacccataaccctcaagcaccagttatgctagacagaatcctgcgtctattagctacctatagtgtgttaaactgtaagctacgtgacttacctgatggtggggtcgagaggctatacggccttgctccagtctgtaagttccttaccaagaacgcagatggtgtgagtatggctccattgctgttgatgaatcaagacaaggttcttatggagagctggtatcatcttaaggacgcagtgattgacggaggtataccgttcaataaggcctatgggatgaccgccttcgagtaccatggaactgacccacgtttcaacaaagtcttcaatcagggtatgtcaaatcatagcactataacaatgaagaaaattctggaagtctacaaagggttcgagggagttaagactgtggtagatgtaggtggaggaaccggtgcgacgttaaatatgatcatagataagtatcccaccatcaaggggatcaatttcgagttaccacatgtggtagaagatgctccatcttacccaggtgtagaacacgttggtggtgacatgtttgtctctgtgccagagggagacgctatctttatgaagtggatatgccatgactggtcagacgatcattgtcgtaagttgctaaagaactgctatcacgcactgcccgaaggtggcaaagtcattctggctgaatgtattttaccagatgctcctgacacctccctggctacgcaaaatgtcgtccacatagatgttatcatgttagcccataatccgggtgggaaagagagaacggagaaggagtttgaggccttagccaaaggtgcgggctttaaggagttcagaaaaatatgttcagcagtaaacacatggatcttagagctgtgtaaa

SEQ ID NO: 238 - DNA, 1131 bp, synthetic construct
  source 1..1131: mol_type = other DNA
atgtcatccgctacaagcgagaatacgcaattaaaatcagtgtctccctcatcaatggagaacggtaaagcaggtgaggaggaagagcactgttcctatgcattacaattagccacttctgctgcacttcctgtcgtgttgaatagcatcataaaacttaacgtattggacataattgcaaaagctggcccaggggctcagctgtccccgtctgagattgctgcccacctaagtccggagaactctggtgcggcagtgatgctagacagaatgttgaggcttctggcctcctactccgtattgacatgcagcgtggtgggtggtggagacaaggataatcaacaccaaagggcgtacggtttggctccagtcgccaaatacttcttgagagatgaggatggagggtcattaggtagcctacttgacttattgcaggataaggtacttgtagatatatggtatgagctggaaccagctgtgttgaaaggtggaacagccttcaatagaacccacggaacgcatattttccagtacaccggctccgatcagaaattcaacgagacgttcaacaaggcgatgattgatcatactttcatagtggttaggaaagttcttcagttctacaaaggcttcggacaccttaaaaccctagtcgatgtaggaggtggattaggcgttaatcttgggctgatcacttcaaagtatccgtctatcaagggaattaatttcgatttgccccacgtcgtgcagaacgctccagcgtatccgggtgtggagcacgtcggtggagatatgttcgaaagcattcctaagggggacgctatctttatgaaatggattttgcacgactgggatgatgaacactgcctaaagttattgaagaactgctataaggccttgcccgacaatggaaaagttatcgccgttgacgccatccttcctatggtcccagacaattcagcatcctgtaagagtaattgccaggttgacttgttcaccttggccttgtacttccccggtggcaaggaacgtacggaacaggagttcttagccctagccaaagaggctggcttcaagggtatacgtttagaatgcttcgcttgcaacatgtgggtaattgagttctacaaa

SEQ ID NO: 239 - DNA, 1122 bp, synthetic construct
  source 1..1122: mol_type = other DNA
atgtcatccgcgacaagtgagaatacgcaactgaaaagtgtgtctccatccagtatggaaaatggaaagggtagtgcggaagaggaagctagtttgttcgccatgcaattagccagtgcttccgtacttccgatggttcttaaatccgctatcgagctggatttactggaacttattgcaaaagctggtcccggagcgtatgtttccccgagcaagctggctgcgcagcttccgacccacaatccgcaagcacctgttatgcttgatagaatcctaagattgctggcaacttattccgtcctaaactgcaagttaaagaacctgcccgacggaggtgttgagaggctatacggattagcaccggtgtgcaaatttctaaccaagaatgcagacggcgtctcaatggcaccactgctgttaatgaatcaagacaaagtgttaatggagtcctggtaccacttgaaggacgcggtgatagacggaggtatcccatttaacaaggcgtatgggatgacggcatttgaataccacggaacagatccacgtttcaacaaggtgtttaatcagggaatgtctaatcactctacaattacgatgaagaagatattagaggtttacaaaggctttgagggagtaaagacagtggtcgatgttggaggtggcacaggtgcgacattaaacatgattatcgacaagtaccctactataaagggtataaacttcgaattacctcacgtggttgaagatgcccctttctatcctggggtggaacatgtagggggtgacatgtttgtctctgtgcccgaaggagacgctatatttatgaaatggatatgtcatgattggtccgatgatcactgtagaaagttattgaagaattgctaccacgctttgccagaaggtggcaaggtaatcttagcagagtgcatactgccggatgctcccgatacatctcttgcgacccagaacgtggtccacatagacgtaatcatgctggcacacaatcctggtgggaaagaaagaactgagaaagaatttgaggctttagcaaaaggtgcaggattcaaggagttcagaaagatatgttcagctgtcaatacatggattatggagctatgcaag

SEQ ID NO: 240 - DNA, 1125 bp, synthetic construct
  source 1..1125: mol_type = other DNA
atgtcatccgccacatcagaaaacacccagttaaaatctgtgagccctagttctatggagaacgggaaggctggcgagaggcaggaagaattacaaaagctcttacaattaactgggaccttgacggtccacgtagcgttaactgccgcgatagagttaaatctattcgagattatggccaagagtggaccaaacgcgcagttatccagtgcacagattgtcagtcttctaagaactaagaatcctgatgctccggctatcttagataggatattacaattacttggttcctatagcatattgaagtgcacgatagtgtccaacgatcagaatggccacacaactaagctcttcggattgacgcccatggctgaatatttagtgcccgattcaaatggtatatccttcgccccagctatcttaatggcgagggaaaaggcattcgctaaatgcggctatttccttaaagacgccatattagagggaggtacgccttttcataaagtaaacgggatgcacttattcgagtacatcgggtcaagctcccgtctaaacgaggtctttaacagaatcatggcctgccatacctctgtgttcatgggagcggttctagaaacttataagggtttcgaggaccttaaagaattggtggacgttggaggtggtcacggcacgacgctgaagatgattctgtctaagtacccacgtataagaggtatcaattttgatctgcccttcgtaattaagaatgcgcctgcctgtccgggagtcgagcacgtagaaggagacatgtttcaaaatgtaccgtccgtagaggcaatattacttaagaacgtgctgcatgactggaacgatgataggtgcttgaagctactgaagaactgttgggacgcactgccagagagcggaaaagtaattgcgattgacgctatcatcccagagttaccgtcaacggacagtgcgtcacagttcgcgtacattagtgatgtcgaaatacttatcgctgcaccaggtggaagagaaagaactttcaaggagtttaacgcactagccacggcagctggtttcactgcctgtaagttggtcgctagaacttcccaatacggagtgctggaattttacaaa

SEQ ID NO: 241 - DNA, 1011 bp, synthetic construct
  source 1..1011: mol_type = other DNA
atgtcatccgcaacgtctgagaatacacagttaaagagtgtttcaccaagttctatggagaacggtaaggcaggtgaggagggtgaacaatgttcatacgctatgcaattagccacctccgctgctcttcccaatgttttgaacgcaatcattaggctaaacgttttggaaataatagcgagggcaggtccaggcgctcagctatctactagcgagatagctgcgcacctgcatactcagaacagcaacgccgcagtaatgttagatcgtatgctgagacttctggcatcttacagcgttcttacctgctcagttgttggtggaggcggaggtggaggtgacgataaggacgatcagcaccaaagagtctatggactggctcccgttgcaaagtatttcttgcaaaatgaagacggcggtagcctaaggcccctgctagacctattccaaggaaaggtgctgacggatatttggtgcgaattagaaccagcagtgttggagggtggcacggcatttaacaggaaacacggcatccatatctttgaatacacgggtagagatcaaaaattcaatgagagttttaacaaggcaatgatcgaccacaccttcatcgttgttaagaatgtattgcagttttatcgtgggtttgaacatttaaagacccttgtggacatcggcggtggacttggcgtcactctgggcatgataacaagcaagtaccctaacataaaaggcataaatttcgaccttccacatgttgttcaaaacgctcccacttatcccgataacggtaaggtgatagcggtagatgccatactacctacaatcccggacaattcagcaagtagtaaatccaatattcagatggatcttttcaccctagcatcatatttccctggtggtaaggaaaggactgagcaagagttcttagcgttggctaatgaggctggcttcaagggaatccgtttagaatgtctggtatgtaatatgtgggtcatggagttttacaag

SEQ ID NO: 242 - DNA, 1848 bp, synthetic construct
  source 1..1848: mol_type = other DNA
atgtcatccgttacaaaaggtaatccaggtgactctagaaataagtcatctgtttcaatgtttattgttgctggtttgtgttgtgttttctttttattgggtgcttggcaaagatcaggttctggtaaaggtgacaatattgcttacgaaattactaaaaaggctaaagcatgttctattttgggtaatttggaatttgaaactcatcatggtaataaggctgttaatgattctatgttggaagttaaagaatataaaccatgtgatgatcaatacattgattacacaccatgtcaagatcaagctagagcaatgacttttccaagagaagatatgaattatagagaaagacattgtccatcagaacaagaaaaattgcattgtttaattccagcaccaaaaggttacgttactccattcagatggccaaaatcaagagattatgttccatacgcaaatgctccacataaatcattgactgttgaaaaagcagttcaaaattgggttcaatatgaaggtaatgtttttaaatttccaggtggtggtactcaatttccacatggtgcagatgcttacattgaacaattggcttcagttattccaattaatgatggtacagttagaacagcattggatactggttgtggtgttgcatcttggggtgcttatttgtttaaaaagaatgttattactatgtcttttgctccaagagattctcatgaagctcaagttcaatttgctttagaaagaggtgttccagctgttattggtgttttaggtactattaaattaccatatccatctagagcatttgatatggctcattgttctagatgtttgattccgtggggtgctaatgatggtaaatacatgatggaagttgatagagttttgagaccaggtggttactggattttatcaggtccaccaattaattggagaaattcttaccaagcatggcaaagaccaaaagaagaattgcaagaagaacaaagaaaaattgaagaaattgctgaattgttatgttgggaaaagaaacatgaaaaaggtgaaactgcaatttggagaaaaagaattaatgaagattattgttctcatggtgactcaagagttactaaatgtagatctgttgatgctgatgatatttggtataaaaagatggaaaattgtgttactccatatccagaaactagatctcaagaagaagttgctggtggtgaattgaaaccatttccagaaagattgcatgctattccaccaagagttgattcaggttctattccaggtgtttctattgattcatttcaagaagataatagattgtggaaaaagcatatgtctgcttacagaagaattaataagttgttagattcaggtagatacagaaatgttatggatatgaatgctggtttgggttcttttgctgctgcatttgaatctccaaaattgtgggttatgaatgttatgccaactattgctgatagagatactttgggtgttatttttgaaagaggtttgattggtatttatcatgattggtgtgaagcattttctacttatccaagaacatacgatttgattcatgctgaaggtgttttctctttgtacaaagataaatgtaatatggaagatattttattggaaatggatagaattttgagaccagaaggtgctgttattttgagagatcatgctgatgttttgacagaagttaaaagaattattgctggtatgagatggaatatgaaaatgtatgatcatgaagatggtccattagttccagaaaagattttagttgcagttaaaaagtactgggttgctggtgacaaaaattcaactttttcttaa

SEQ ID NO: 243 - DNA, 1356 bp, synthetic construct
  source 1..1356: mol_type = other DNA
atgtcatccggttctggtgacgatgatcatccatcttcaaaaagaccaattcaacatactcaaacaaaatacaaattgaaaattttgttattggttattttaactaatttgttaacaatttacgtttttacaggtccatctttgccaattgctcaaaatttgtctttaccaacatgggatcatactaaattattgaatgaattaaatattacaaaaagagaattggctaattctagagcacaaaattctgatttgcaacaacatttgaaagcatcaaatattttgactaaatctttattgactgaattatcaagattgaatgctgcaattgatcaacattcatcttctaaagaaggttcatctattttctcatttgatgatttgttatcagatttgtctacagaagctaaaattggtatgggtccacaaaaattaccattgggttactcaccaagatctggtactgatgaattgtatccatctgttggtggtggttgtttgagatacaaagaagaattagctcaatatatgacttacaaaattggtggtgaatgtccagttgatgatgtttttgcacaaagattaatgttgaaaggttgtgaaccattgccaagaagaagatgtcatccaaaatctccagcaggttataaagaaccaaatccattgccaaaatctttgtggactatgccaccagatacatctattgtttgggacccatatacttgtaaatcttacggttgtttggttgaaagaaagaaattgccagttttctatgattgtaaagattgttttgatttggaaggtagagaaaaatctagatggttgtttgataatggtggtttggattttggtattaatgaagttttgggtacaaaaccacatggtactattagaattggtttagatattggtggtggtacaggtacttttgctgcaagaatgaaagaaaataatgttactattgttactacatctatgaatttggatggtccattcaattcttttattgcttcaagaggtttgatttctatgcatatgtcagtttctcaaagattgtcatttttcgaaaatactttggatattgttcattcaatgcatattttgtcaaattggattccagatactatgttggaatttactttgtatgatatttacagagttttgagaccaggtggtattttctggttggatcatttcttttgtttgggttctcaattaaattctacttacgttccaatgttggatagagttggttttaaaaagttgagatggaatgctggtatgaaattggatagaggtattgataaaaatgaatggtacttttctgctttattggaaaaaccagttaaatcataa

SEQ ID NO: 244 - DNA, 4236 bp, synthetic construct
  source 1..4236: mol_type = other DNA
atgtcatccgaatctccaggtcaatggttggaaaaagcattattggatttgtgtaaaaagattgaaactggtttagatttggatggtgacattatttctggtttagtttcttattgtgaattagcatctccattagatgcaaaagaatacttagataatattattggtcaagaagcaggtaaaaatgttacagaagaatacttaaaaagaagaggtcattctgataattacaattctacttctgatgctccaacttcaaaattgcatgcatatgttaaaccaccatcaaatgaagttttagtttctggtactagaaaaccatcaagagcaccaaaagaattggctgcatcttcaaatcaagaaaagagagcaccaactgaaacagttgaattgagaaatatgaataagggtaatcaaggttcttcaagaaagaaaaaagctggtaaaattgtttctttggctgaagctgctaaaggttctattgtttttcaacaaggtaaaccatgttcatgtcaagcaagaaggcatagattggtttctaattgtttatcttgtggtaaaattgtttgtgaacaagaaggtgaaggtccatgtacattttgtggtgcattagttttgaaagaaggttcttcttatgcaggtttagaagaaggtgcagttccattgtctgatgctgaagcaactgcagaagcatatacaaaaagattggttgaatacgatagaaattctgcagctagaactacagttattgatgatcaatctgattactacgaaattgaaggtaattcttggttgtcaatggaagaaaaagaattattgagaaagaaaaaagaagaaattgaagaagcagaaagaactaaaagatcaaaattgattatgacttttgatttggttggtagaaaagttttgttaaatgaagatgaagtttcagaattagaaatggaaaatagaattttgagaccaccagatgaaagagaagctaatagaattaaaccaaatccaactttgaaagttcaaccaatttttgttgatccaggtacttcaagaaaacaaccagttaaaggtaatcatttgaataagggtttatcaaatggtttgtgtttggaaatttctggtagagttcaacatggtactaatgaattaaaacattttatgatggatgataaattggaaactgcttctaatggtggtttatggttgggtccatctgttaatgattctttgcaaattgttatttcattgttaagatctgcaagaagaattatggctactaaaggtaattctggtgacaataagactagatcatctgtttcaatttttattgttgcaggtttatgttgtttcttttatattttgggtgcatggcaaagatctggttttggtaaaggtgactctattgcacaagaaattactaaaaaggcagattgttcaattttatctaatttgaattttgaaactcatcatggtgacgaagctggtaatgatgattcaaaatctgaagttgaaatttttaaaccatgtaatgatagatacgttgattatacaccatgtcaagatcaaatgagagctatgacttttccaagagaaaatatgaattacagagaaagacattgtccaccagaagaagaaaaattgcattgtttaattccagctccaaaaggttacgttacaccatttccgtggccaaaatctagagattacgttccatacgctaatgcaccatacaaatcattgacagttgaaaaagctgttcaaaattggattcaatacgaaggtaatgtttttagatttccaggtggtggtactcaatttccacatggtgcagatgcttacattgatcaattagcatcagttattccaattgataatggtatggttagaacagctttggatacaggttgtggtgttgcatcttggggtgcttacttgtttaaaagaaatgttattgctatgtcttttgcaccaagagattcacatgaagctcaagttcaatttgcattggaaagaggtgttccagctgttattggtgttttgggtacaattaaattgccatatccatctagagcatttgatatggctcattgttcaagatgtttgattccgtggggtgcaaatgatggtatttatatgacagaagttgatagagttttgagaccaggtggttattgggttttgtctggtccaccaattaattggagaacacattatgaagcatgggaaagaccaaaagaagaattagaagaagaacaaagaaaaattgaagaaattgcaaaattattgtgttgggaaaagaaacatgaaaaaggtgacattgctatttggagaaaaagaattaataagcaatattgtaatgaaagagattctcatgttacaatgtcagaatcatctaatgctaatgatgtttggtataaaaagatggaagcatgtgttactccatatccagaatctacttcatctgaagaagttgctggtggtgaattgcaaccatttccagaaagattaaatgctgttccaccaagaatttctagaggttcaattccaggtatttctgttgaatcatttcaagaagataataatttgtggaaaaagcatttgaaagcatacaaatcaattaataagattattgatacaggtagatacagaaatgttatggatatgaatgcaggtttgggttcttttgctgcagcttttgaatctccaaaattatgggttatgaatgttatgccaactattgcagaaagagatactttgggtgttatttacgaaagaggtttgattggtatttaccatgattggtgtgaagcattttctacttatccaagaacatacgatttgattcatgctaatggtgttttctcattgtacaaagataaatgtaatgctgaagatattttgttagaaatggatagaattttaagaccagaaggtgctgttatttttagagatcaagttaatgttttgattaaagttaaaagaattgttgctggtatgagatggaatgctaaaatggaagatcatgaagatggtccattggttccagaaaagattttagttgcagttaaacaatattgggttgttgctgaagaaactccatgtccaattacaattggtagaattaaaacaaaagaatctgaaatttctgtttttggtgctgataaatactttaatatgaaaattaattgtgaaaatccaggtttagctgataatgcaggtagaaaacatggtcatcaaaaagaagattgtagagctgatttgcaaggtttgaaaccaaattttagatctggtactccatcaactatttctgaatcatcttggaactcacaaactgcattgtttccatcattattgagaaaatcatctcaaaataagcaaaagagagctattggtaaaagatttttcttgggttttggttgttctgttccatgtttggataaaaagtctgtttacattgatggttcagtttctcatggtgttgttaaaggtactaaacaatctgaaccaggtttgaaatcaggtgaacatgttgattttccaattttgaattttggtatggaaaatttggcagttaaaaagcaattggaagttgaaaaattggaagaagaacaaagaaaatctttagaagtttttggttctcatatgatgagaaaaggtgacgaaattgctaaaaatttggaaagaaaattatcaatgttgacttgggatgcaattccagcagctcaatctttgccaagaacattagcttcatctagattatgtgatgatattgaatcagatgcttcttcagatttgtttgaattggaaaatatttcatctactggtcaatattcattgttaacaagacaagcatctgattcaatgtcaggttgtatgactccaacaactcaatatgcaccatctgaagcatcaattgaatggtcagttgttactgcatctgcagctgatttttctgttgcttctgattacgatgctgaacatatgatttcatctaatacttctaaaatggctaaaactaaaaatattaatattggtggtaaagctccaaaaggtttgccatcaggtttattgtcatgtaaatctcaaaaagcagttaatgttgctgaaactgcagcttacagagctaatgaaaagactaaatcaatgtctttgggtaaaccacaagcagatcaaacaagagttaaagattttgaattttcaagagttcaacatccatttgctgctagagttattgctccataa

SEQ ID NO: 245 - DNA, 1350 bp, synthetic construct
  source 1..1350: mol_type = other DNA
atgtcatccggttctccagatgatgataatttgtcaagaaaacaaactcataaatcaattaatgatcaaactaaatataaattgaaatctttggttttaattattttgactaatttgtttacaatttatatttttactagaccatctttaaatttgcatccaatttcaaaatacgattcattttctgattcttcatttttattgcatgaattaaatgcaactatgaaacaattagaaacttctagatcacaaattattgttttgcaacaacaattgaaatctactaatcaattggttaatgaattgttagctcaattgaatattttgaatcaatctattaatagaactccagattctactttattgaattttaatgatttgttagatgatttgtctaatgaagcaaaattggctttaggtccacataaattaccattgggttactcaccaagatctggtacagatgaagtttacgcatcagttggtggtggttgtttgatgtacaaagaagatttggctcaatatatgacttacagagttggtaaagaatgtccagttgatgatgtttttgctcaaagattgatgttaaaaggttgtgaaccattgccaagaagaagatgtcatccaaaagctccagttggttacgttgaaccaactccattgccaaaatctttgtggtcaactccaccagatacttctattatttgggacccatacacatgtaaaaattacacttgtttgattgaaagaagaaaatttccaggtttttacgattgtaaagattgttttgatttgcaattcagagaaaagacaagatggcaatttgataatggtggtttggattttggtatggatcaagttatggctactagaaggcaaggtacaattagaattggtttggatattggtggtggtactggtacatttgctgcaagaatgaaagaaagaaatgttactattgttacaacatcaatgaatttggatggtccattcaattcttttattgcttctagaggtttgatttctatgcatgttggtgtttctcaaagattgccatttttcgaaaatactttggatattgttcattctatgcatattttgtctaattggattccagatgcaatgttagaatttactttgtatgatatttacagagttttgagaccaggtggtttattttggttggatcatttcttttgtttgggttcacaattgaatgttacttacgttccaatgttggaaagagttggttttaaaaagttgagatggaatgctggtatgaaattggatagaggtattgataaaaatgaatggtacttttctgctttattggaaaaaccaatttcataa

SEQ ID NO: 246 - DNA, 1482 bp, synthetic construct
  source 1..1482: mol_type = other DNA
atgtcatccgctgctgcaaaaggtcaagaaagagaaatgcaaaaatcatactggattgaacatacaactgatttgtcagttgaagctatgatgttggattctaaagcagctgatttggataaagaagaaagaccagaagttttgtctttattgccaccatacgaaggtaaatctattttggaattaggtgcaggtattggtagattcactggtgaattggctaaaaaggctggtcaaattattgctttggattttattgaaggtgctattaaaaagaatgaaagaattaatggtcattgtaaatcagctaaattcatgtgtgctgatgttacttctccagaattagatttttcagcagaatctatggatttggttttctcaaattggttattgatgtacttgtctgatgaagaagttgaagcattagctaaaagaatggttaaatggttgaaagttggtggtcatattttctttagagaatcatgttttcatcaatctggtgaccataaaagaaagaataatccaacacattacagagaaccaagattttacactaaagtttttaaagaatgtcattttagagatgaatctggtaattcttttgaattaacattggttggttgtaaatgtattggtgcatacgttaaaaataagaaaaatcaaaatcaaatttgttggacttggaaaaaggttagatctgaagatgatagagcatttcaaagatttttggataatgttcaatacaaatgtactggtattttgagatacgaaagagtttttggtcaaggttatgtttctactggtggtattgatactacaagagaatttgttgctaaattggatttgcaaccaggtcaaaagattttggatgttggttgtggtattggtggtggtgacttgtacatggctgaaaaatttgatgttcatgttgttggtattgatttgtctattaatatggtttcttttgctttggaaagagctattggtttacaatgttcagttgaatttgaagttgcagattgtactaaaaagacttacccagatgatacatttgatgttatttactctagagatactattttgcatattcaagataaaccagctttgtttaaatttttctataaatggttaaaaccaggtggtagagttttaatttctgattactgtaaaagagctggttcaccatctgaagattttgcaagatacattaaacaaagaggttacgatttgcatgatgttgaagcatacggtcaaatgttgaaagatgctggttttgatgaagttattgcagaagatagaacatctcaattcactaatgttttgcaaacagaattggatttggttgaaaaagatagagaagcattcattagagatttttcagaagaagattatgatgatattgttggtggttggaaagctaaattgattagatcatcttctggtgaacaaagatggggtttgtttattgctaaaaagaattaa

SEQ ID NO: 247 - DNA, 906 bp, synthetic construct
  source 1..906: mol_type = other DNA
atgtcatccggtaaatcaggtagagattggactcaaatttacgctatttacggtatggatgattggcaaactccatttttcttattgattcatgctgttttcttttcagctttgtctattttatttttgttatactttgaaccaatttgtttctttacacaacatttctttccattcattggtgcaggtttagcaagatttgttgctggttttactggttctgttactgctttgtctgctgtttgtttgtttttcgcagctgcaaatttcttttactcttcagtttctttacattgggatatggctcaaagaattgttaatgctgttccagattggtcatctgttaaacaagcattggatttgggttgtggtagaggtattttattgaatgctgttgcaatgcaattgaaaaagtcaggttcttcaggtagagttgttggtttagatccaactagagcacatactaatggtccaactacattatctactttgagaacagctggtttggaaggtgttcaagaatatgttacttgtagagctggtgacccaagaaaattgccattttcagataattactttgatgttgttgtttcatctgtttttgttcatactgttggtaaagaatttggtcaaaaatctgctgcagctggtgcagaaagaatgagagttttgggtgaagttgttagagttttgaaatcaggtggtgttggtgttgtttgggatttggttcatgttccagaatacgttcaaagattgcaagaattgaaaatggaagatattagagtttctgaaagagttacagcttttatgttatcttcacatattgtttcttttaaaaagccaaatcaacattttgttggttcaaatgaagttagagttgattggagattcaataatttgtgttaa

SEQ ID NO: 248 - DNA, 2766 bp, synthetic construct
  source 1..2766: mol_type = other DNA
atgtcatccgcaattggtaaagcaagatctaataagagatcttcatcttcttctagatcatctactttgacaactactattttggttgcattgtgtatttttggtttgtggttattggcaccaaatacattggtttacccaagaactacaacaagaacttctgcaactacatcatctcaatcatcttcaatgccagattcacatgaaattcatccaacagaaccaattccaaagaaatcacaaccagtttttcaagattctcaaggtgacttgccagaagatgcaattcaaacagatcatggtgctacacataatgatcatactgatgaatctccagaattaccaaatcaatcttcaactggttctaaagctcaagcagaagctgataatgtttctgaacaagattctggtccagaaaataatggtgactctgattcatcatctgataataataatgaagctgaaggtgacggtggtggtggtgacggtggtaatgaagaagttgatcaaaaagattctgatgttgaaggtaatccacaagaattgcatggtatggaatcagttgaagaattggaaaaacaaaagaaattagaaacacaaaattcagaagaatcttcaattactcaaaatcaagaagctgaaaagactagagaagatcaaacaaaagcatcagctaataaggattttgaaggtaaagaaaatcaacatactgctgatgataaaaagattgaaaatcatgattcttcagaaaataaggaacatgaaatttctgatgctgatcaacaaaagagattggaacaacatcaacaacaagaagatgatcaaattcaacatggtaaatctcaaagaacagaaggtacaacttcaatttctaatcaagatgttcaatcaactgttggtggttctactgatccagaaaatcatgaatcacaacataatattttacataataagaatgctacagaatcaatttctactgaagaagctgataaaaaggaaaaaccatctaaatgggatacatcagattcttttccatctggtgactcttctgaaattccaaaagaatctagagaatctaaaaagtcttggtcaactcaagcagatgaatctgaaaatcaaaaagaaagaagaaaagttggtactgatgatcaagatggttcttcaattaatggttacaaatggcaattatgtaatgttactgcaggtccagattacattccatgtttggataatgaagaagctattgctaaattgcaatctagaaggcattatgaacatagagaaagacattgtccaaaagaagctccaacttgtttggttccattgccaaaaggttataaaagaccaattgaatggccacaatctagagataaaatttggtactataatgttccacatacaaaattagctgatgttaaaggtcatcaaaattgggttaaagttactggtgaatttttgacttttccaggtggtggtactcaattcattcatggtgctatgcattacattgatttcttggaagaagctgttccagatattgcttggggtaaacattctagagttttattggatgttggttgtggtgttgcttcatttggtggttacttgtttgaaaaagatgttttagcaatgtcatttgcaccaaaagatgaacatgaagctcaagttcaatttgcattggaaagaggtattccagcaatttcagctgttatgggtacacaaagattgccatttccatcaagagtttttgatgttgttcattgtgcaagatgtagagttccgtggcatgctgatggtggtgcattattgttggaattaaatagagttttgagaccaggtggttattttgtttggtctgcaacaccagtttaccaaacattagaagaagatgttaaaatttggaaagaaatgtcaaatttgactgtttctatgtgttgggaattggttacaattaaaaaggataaaattaattcaattggtgctgcagtttaccataaaccagattcaaatgaatgttaccataaaagaaaacataataatccaccaatgtgtaaatcagatgatgatccagatgcagcttggtacattccattgcaagcatgtatgcataaagttccagctgaaggtaatgaaagaggttctgattggccagaagaatggccaagaagattgcaaactccaccatattggttgaatagatctcaaatgggtatttacggtaaaccagctccagaagattttacagctgatcatgaacattggaaacaagttgtttcaaaatcttatttgtcaggtttaggtatttcttggtctgatgttagaaatattatggatatgagagctgtttatggtggttttgcagctgcattgaaagatttgaaagtttgggttttgaatgttgttaatattgattcaccagatactttgccaattatttatgaaagaggtttgtttggtatttatcatgattggtgtgaatctttttctacatatccaagaacttacgatttgttacatgctgatcatttgttttctagattgaaaaagagatgtaaattgattccagttattgctgaagttgatagaattgttagaccaggtggtaaattgattatgagagatgattcttcaactattggtgaagttgaaaatttgttaaaatctttgcattgggaagttcatttgactttttctaaaaatcaagaaggtattttgtctgctcaaaaatctgaatggagaccagattcttacgttgcatcatcataa

SEQ ID NO: 249 - DNA, 1032 bp, synthetic construct
  source 1..1032: mol_type = other DNA
atgtcatccgcttcatctttggtttctggtgctgaaaaatttcaattgattggtattgctccagcaggtttaggttttattggttctgatttgcatggtaaacattttccaaaattgtctttagtttcatgttctggtatttctggtgacagatctaaaattttggttccaagatgttctttgtcagaatctagaccaacatctcaaccaagattcattcaacataaaaaggaagcattttggttttatagatttttgtctattatttacgatcatgttattaatccaggtcattggactgaagatatgagagatgaagcattggaaccagctgatttgtcatctagagatatgattgttgttgatgttggtggtggtacaggttttactacattgggtattgttaaacatgttgatgctaaaaatgttacaattttggatcaatctccacatcaattagctaaagcaaaacaaaaagaaccattgaaagaatgtaaaattattgaaggtgacgcagaagatttgccattcagaactgattacgctgatagatacgtttctgctggttctattgaatactggccagatccacaaagaggtattaaagaagcatacagagttttaaaattgggtggtaaagcatgtttgattggtccagttcatccaactttttggttgtctagatttttcgctgatatgtggatgttgtttccaaaagaagaagaatacattgaatggtttcaaaaagctggttttaaagatgttcaattgaaaagaattggtccaaaatggtacagaggtgttagaaggcatggtttgattatgggttgttctgttactggtgttaaaccagcttctggtgactctccattacaattgggtccaaaagctgaagatgtttcaaaaccagttaatccaattacatttttgttaagatttgttttgggtgcaatggctgcaacttactttgttttggttccaatttatatgtggttgaaagatcaaattgttccaaaaggtcaaccaatttaa

SEQ ID NO: 250 - DNA, 1893 bp, synthetic construct
  source 1..1893: mol_type = other DNA
atgtcatccgctactttgcaaccaccagtttcacaattgccacaaaaatctccagctccaaaattgttaaataagccattgtttaatattttgttgttaacaattttctgttctttgtcatattttattggttcttacactaattcaatttactcaatttctccatcaccatctttgattagaagagatacaaattgtggttttgaagttaaaaataatttgaatactacagttgaaaatgttcaagaaccattgaattttgaaccattgcatacattgtctttaccaatggaagatactccagaatctcatcaaccatttccatattgtccaaagaattatacaaattactgtccatgtcaagatccaaataaggaaagattgtttactattgatagaagatttcatagagaaagacattgtccagaaaaaggtgaagcattgagatgtttagttcaaaaaccagctggttatagaaggccatttccgtggccaaaatctagagattacgcatggtttaaaaatgttccattcaaaagattgactgaatataaaaagtctcaaaattgggttagacaagaaggtgacaaattgtttttcccaggtggtggtacatcttttccaaaaggtgttaaaggttatattgatttgttgaaaagagttgttccattaaattcaggtgctattagaactgttttggatgttggttgtggtgttgcttcttttggtgcttctttaatggattacaatattgttactatgtctattgctccaagagatattcatgaagctcaagttcaatttgctttggaaagaggtgttccagcattattgggtgttttatcaacttacagattaccatttccatctagatgttttgatatgattcattgttctagatgtttggttcaatggactgattacggtggtttatacttgatggaaattgatagattgttgagaccaggtggttattgggttttgtcaggtccaccaatttcttggagaatttcttacaaaggttgggaaagaaatgcttcagatttggaaattgaacaaaataatttggaagatttggctagaaaattgtgttggaaaaaggttagagaatctgatccaattgcagtttggcaaaaaccaacaaatcatattcattgtgctcaaaaattgaaaacttggaaagctccaaaattttgttctcatgattttccagatcatggttggtacaaaaagatggaaccatgtattacaccattaccaaaagttgaacatgttagatcaacatctggtggttacttagaaaaatggccaaaaagattaaaaactgctccaccaagaattagatcagctactgttgaaggtgttactatgagaacttttgatgaagataataagatttggaaaagaagagttttgtactatggttctgttttgaaatttttgtttaaaggtaaatacagaaatattatggatatgaactctggtttgggtggttttgctgcagttttgtctgaatatccagcttgggttatgaatgttgttccatacgatgctaaaaataatacattgggtattgtttacgaaagaggtttgattggtacttatatggattggtgtgaaccattttctacttacccaagaacttatgatttgattcattctgatggtttgttttctttgtacatggaaaaatgtgatattttagatattttgtttgaaatgtatagaattttgagaccagaaggtgctattattattagagatcatgttgatgttattgttaaaattaaagaaattacagatagaatgagatggaactcaaaaatttctcataatgaaatttcaccatttcattcagaaaagattttgtttattgataattctccagaataa

SEQ ID NO: 251 - DNA, 1161 bp, synthetic construct
  source 1..1161: mol_type = other DNA
atgtcatccgattacaatattgttactatgtctattgctccaagagatattcatgaagctcaagttcaatttgctttggaaagaggtgttccagctttgttaggtgttttgtctacttacagattgccatttccatctagatgttttgatatgattcattgttctagatgtttggttcaatggactgattacggtggtttatacttgatggaaattgatagattattgagaccaggtggttattgggttttgtctggtccaccaatttcttggagaatttcttacaaaggttgggaaagaaatgcttctgatttggaaattgaacaaaataatttggaagatttggctagaaaattatgttggaaaaaggttagagaatctgatccaattgctgtttggcaaaaaccaacaaatcatattcattgtgcacaaaaattgaaaacttggaaagctccaaaattttgttctcatgattttccagatcatggttggtataaaaagatggaaccatgtattactccattgccaaaagttgaacatgttagatctacatcaggtggttatttggaaaaatggccaaaaagattgaaaactgctccaccaagaattagatctgcaacagttgaaggtgttacaatgagaacttttgatgaagataataagatttggaaaagaagagttttgtattacggttctgttttgaaatttttgtttaaaggtaaatacagaaatattatggatatgaactctggtttgggtggttttgctgcagttttgtctgaatatccagcttgggttatgaatgttgttccatacgatgctaaaaataatactttgggtattgtttacgaaagaggtttgattggtacttatatggattggtgtgaaccattttctacttacccaagaacttacgatttgattcattctgatggtttgttttctttatatatggaaaaatgtgatattttggatattttgtttgaaatgtacagaattttgagaccagaaggtgctattattattagagatcatgttgatgttattgttaaaattaaagaaattactgatagaatgagatggaactcaaaaatttctcataatgaaatttctccatttcattctgaaaagattttgtttattgataattcaccagaataa

SEQ ID NO: 252 - DNA, 1047 bp, synthetic construct
  source 1..1047: mol_type = other DNA
atgtcatcctctaaaccatcagctttagatttggctacaggtttgggtggtaaaattgctaaagatgatgttatttctgcagttgataaatacgaaaaatatcatggttactatggtggtgaagaagaagaaagaaaagctaattatactgatatggttaataagtactatgatttggttacttctttttacgaatatggttggggtgaatcttttcattttgcagctagaatgaaaggtgaatctttgaaagaatctattaaaagacatgaacatttcttggctttgcaattaggtttgaaaccaggtcaaaaagttttggatgttggttgtggtgttggtggtccattaagagaaattgcacaattttcattgacttctgttacaggtttgaataataatgaatatcaaatttctagaggtaaagcattgaatagagttgctggtgttgatagaacttgtgattttgttaaagctgattttatgaaaatgccatttccagataatcaatttgatgctgtttatgcagttgaagctacttgtcatgctccagatgcagttggttgttacaaagaagtttacagagttttaaaaccaggtcaatgttttgctgcatacgaatggtgtatgacagattcttttgatccaaataataaggaacatcaaaagattaaagctgaaattgaattgggtaatggtttgccagatattagattaactggtcaatgtttggaagctgttaaaaaggctggttttgaagttatttgggctaaagatttggcagctgattctccagttccgtggtacttgccattggatacatctcatttttctttttcatcttttagattgactgctattggtagattgattactaaaaatttggttaaaggtttggaatatattggtttggcaccatctggttcacaaagagttcaagatttcttggaaaaagctgcagaaggtttggttgcaggtgctaaaaaggatatttttactccaatgtactttttcttggctagaaaaccattgtctggttcagaataa

SEQ ID NO: 253 - DNA, 1014 bp, synthetic construct
  source 1..1014: mol_type = other DNA
atgtcatccgcttcttcattgttgtctggtgcagaaaatttgaaacatattactggttctcattttccaggttctgaacatcaattgaaatactttccaaagaaaggtccagtttcttcagctaaatcttcaaaattttcaactttggctccaagatgttctttgtcatcttcaagaccagtttcacaaccaagattcattcaacataaacaagaagcattttggttttacagatttttgtctattgtttatgatcatattattaatccaggtcattggactgaagatatgagagatgaagcattagaaccagctgatttgtactctagacatttgactgttgttgatgttggtggtggtactggttttactacattgggtattgttcaacatgttgatgctaaaaatgttacaattttggatcaatctccacatcaattggctaaagcaaaacaaaaagaaccattgaaagaatgtaaaattattgaaggtgacgctgaagatttgccattcagaactgattacgctgatagatacatttctgctggttcaattgaatactggccagatccacaaagaggtattaaagaagcatacagagttttgaaaattggtggtaaagcatgtgttattggtccagtttatccaacattttggttgtctagatttttcgctgatgtttggatgttgtttccaaaagaagaagaatacattgaatggtttcaaaaagctggttttaaagatgttaaattgaaaagaattggtccaaaatggtatagaggtgttagaaggcatggtttgattatgggttgttcagttactggtgttaaaccaatgtctggtgactctccattacaattgggtccaaaacaagaagatgttaaaaagccagttaatagatttgtttttgttttgagattcattttgggtgcaatggctgctgcatattttgttttggttccaatttacatgtggttgaaagatcaaattattccaaaaggtatgccaatttaa

SEQ ID NO: 254 - DNA, 1158 bp, synthetic construct
  source 1..1158: mol_type = other DNA
atgtcatccgctttttgttcacaatctaaattgaataatttgaatttttggattaaaacatctattggttcatctacaaataatgttggtcatgattcaatgagaaaaagaacttctagaaatcatgcaaatttggttgttgttaaaatggcagcttcaacatctggttcaggtggtcaaggtactgttgcttttgaagaaggtcaattggaaagaccacaatggactggtgaaacattgttatctagattggttagagctttgatttcttttaaaccattgttttctattatgaaatttggtgctagacaagttttgatttctacagcagaaaagacaaatattccgtggagagaaatgacaagacaaattttggaatctgatgtttataaagaaatggaatctattgaagatacttcattggtttacccagattattacttgaatccatttcatgcttatgatgaaggtaatttgtcttggttggcagctgctgaagcagaagctgctactatgtctatggtgagaagagctattccagatgcttcttcattggatgaagctaatcaaattgttagaggtaattggttgaaagctattgaagaacatcataataagtactctcaaaatgttattattagagatattttggatattggttgttctgttggtgtttctactagatatttggctgataaatttccagcagctaaattgacaggtttagatttgtctccatactttttggcagttgctcaattcaaagaaaagaaatctaatccaagaaagaatactatttgttggattcatgctaatggtgaatctacaggtttgccatctaaatcatacgatattgtttcaattgcttacgtttttcatgaatgtccagctggtgcaattaaaaatattgttaaagaagcattcagattgttgagaccaggtggtacatttgcattgactgataattctccaaaatcaaaaattttacaagaattgtctccagttttgtttacattgatgaaatctacagaaccatttttggatgaatattacttgacagatattgaaaaagttatgactgaagctggttttattaatgttagaactgttttgacagatccaagacatagaacagttactgctacagttccatactaa

SEQ ID NO: 255 - DNA, 2475 bp, synthetic construct
  source 1..2475: mol_type = other DNA
atgtcatccgcattgggtaaatactcaagagttgatggtagaaaaccatctacaaattattgttcaactgttacaattgttgtttttgttgctttgtgtttagttggtttgtggatgatgacttcttcatctgttgttccagttcaaaattctgatgttgcttctcaacaagaatctaaaaatgatgttaatgaaaatactagagtttctaaatctaatgctaatgaaaatgattcagataattcttcaacagttaataagaatgataatacttctgaatcatctatgaatgataatgaaggtaaatctaaacaatttgaagataatccaggtgacttgccagaagatgctacaaaaggtgacaatactgtttcatctgatcaagaagaaaagaatccaaatccacaagaaaaaccaactgaagaaaatcaagaagaaaaatcaggtgaagaaaatagagaagaaggtggttcaaaatctgaagatggttctaaaattgaaaaagaaaatgaagaaaatggtgaattgaaatctgatgaaaaagtttcagaagctggtgaagctaatagatctaatgcttctggtggtgaatctagagatggtaataagccagatgaaaattttgatgaaacaaaagatgctaataaggaagatggtcaaaaagttaaaaataagatggatactcaatctgatcaagattcaggtgttaaaaaggaatcagatcaaactacagaagtttttccatctggtgcacaatcagaattgttaaatgaaacaactacacaaaatggtgcattttctacacaagctactgaatctaaaaatgaaaaagaagctcaacaatcatctgaattggaaaatcaatcaggtacttcttggaaattgtgtaatgttactgcaaaatctgattatattccatgtttagataatttggaagctattagatctttaagatcaactaaacattacgaacatagagaaagacattgtccagataatccaccaacttgtttggttgcttttccagaaggttaccaaagatctattgaatggccaacttcaagagaaaagatttggtatcataatgttccacatacaaaattggcagaagttaaaggtcatcaaaattgggttaaagttactggtgaatacttgacatttccaggtggtggtactcaattcaaacatggtgctttacattatattgattttattcaacaatctgttccagatattgcttggggtaaaagatctagagttgttttggatgttggttgtggtgttgcttcttttggtggtttcttatttgatagagatgttttgactatgtctttggctccaaaagatgaacatgaagcacaagttcaatttgctttggaaagaggtattccagctatttctgcagttatgggtactaaaagattgccatatccagctggtgcttttgatattgttcattgtgctagatgtagagttccgtggcatattgaaggtggtaaattattgttggaattaaatagattgttgagaccaggtggttactttgtttggtctgctacaccaatttatcaaaagattccagaagatgttgaaatttgggaagctatgaaaaagttaactaaatctatgtgttgggaagttgtttctattactaaagatagagttaatggtgttggtattgctgtttatagaaaaccaattactaatgaatgttatgaacaaagatctcaaaatgaaccaccattgtgtcaagaatcagatgatccaaatgctgcatggtatgttccattgcaagtttgtatgcataaagttccaattgcagaatcagaaagaggttctcaatggccagaattgtggccagctagattggataaaccaccatactggttgtcatcttctcaagttggtgtttacggtaaaccagctccagaagattttgctactgattttcaacattggagaagagttgttactaaatcttacgttaatggtttaggtattaattggtcagctgttagaaatgttatggatatgagagcagtttacggtggttttgctgcagctttgaaagatatgaatgtttgggttatgaatattgtttcaattgatgctccagatactttgccaattatttatgaaagaggtttgtttggtatttaccatgattggtgtgaatcattttcttcttacccaagatcttacgatttgttacatgctgatcatttgttttcaaaaattaaaaagaaatgtaattttaatgcttttgttgcagaagttgatagattattgagaccagaaggtaaaattattgttagagataatgttgaaacaattaatgaattggaatctatttttaaatctttgcattgggatattagaatgacatattctaaaaataaggaaggtttgttatgtgctcaaaagactatgtggagaccaaaagaatttgaaacagttacttatgcaattgcttaa

SEQ ID NO: 256 - DNA, 714 bp, synthetic construct
  source 1..714: mol_type = other DNA
atgtcatccgaagcatgtttgactccattaccagaagtttctgatattaatgaaattgctggtggtccattagctaaatggccagaaagattgactgctgttccaccaagaattgtttctggttcattggatggtattactgctgcaacatttgaagaagatactaatttgtggaaaaagagagttgctcattataaagttttggattcacaattggctgaaccaggtagatatagaaatttgttggatatgaatgcttacttaggtggttttgctgcagctttgactaatgatccagtttgggttatgaatgttgttccagttgaagctaaattgaatactttgggtgctatttacgaaagaggtttgattggtacttaccaaacatggtgtgaagctatgtctacttacccaagaacttacgattttattcatgcagatgctgttttctctttgtaccaagatagatgtgatatggaagatattttgttagaaatggatagaattttaagaccacaaggttctgttattattagagatgatgttgatattttagttaatgttaaatctgttgttgatagattgcaatgggattctagaatggttgatcatgaaggtggtccacatgttagagaaaaattgttaattgctgttaaacaatattggactgcaccagctccatctgaagatcaagaacaatctagaactgcataa

SEQ ID NO: 257 - DNA, 1812 bp, synthetic construct
  source 1..1812: mol_type = other DNA
atgtcatccaaacataaagatggtaaaccatctacacaaactggtaaatcttcaagaactgttccaatggctattatgtttgttgttttgtgtggtttgtctttttacttaggtggtattttctgttctgaaaaagataaatatgcttcagcaaaagttgaagatacagttgaatctccaaaatcaacagcttctggttcattgcaagttaaagatgtttcttttccagaatgtggtttggaataccaagattacacaccatgtactgatccaagaagatggaaaaagtacggtttacatagattaacatttttggaaagacattgtccaccagcttttgaaagaaaagaatgtttggttccattgccagatggttataaaattccaattagatggccaaaatcaagagatcaatgttggtatagaaatgttccatatgattggattaataagcaaaaatctaatcaacattggttgaaaaaggaaggtgacaaatttttgtttccaggtggtggtactatgtttccaaatggtgttggtagatacgttgatttgatgcaagatttgattccaggtatgaaagatggtactattagaactgctattgatacaggttgtggtgttgcttcttggggtggtgacttgttagatagaaatattttgacagtttcattggcaccaagagataatcatgaagctcaagttcaatttgcattggaaagaggtattccagctattttgggtgttatttctactcaaagattaccatttccatctaattcttttgatatggctcattgttcaagatgtttgattccgtggactgaatttggtggtatttacttgttagaaattcatagaattttgagaccaggtggtttttgggttttgtctggtccaccagttaattacgaaaatagatggagaggttggaatactacaattgaagaacaaaaatctgattacgataaattacaagatttgttaacatctttgtgttttaaattttataaaaagaaagatgatatttctgtttggcaaaaatctttggataataattgttacaaaaagtttgattcatctgataattatccaactaaatgtgatgatggtactgaaccagattcagcttggtacacaccattgagaccatgtgttgttgttccaaatcataaatacaaaaatttgggtttaaaatcattgccaaaatggccacaaagattgcatgttgctccagaaagagttgctgatgttagaggtggttctgaaggtgcttttaatcatgatgattctaaatggaaaactagagttaaacattacaaaaagttattgccagctgttggtacagataaaattagaaatgttatggatatgaatactttgtacggtggttttgcagctgctttaattgattatccattgtgggttatgaatgttgtttcatcttacacttctaatactttagctgttgtttttgatagaggtttaattggtacttatcatgattggtgtgaagcattttctacttatccaagaacttatgatttgttacatttggatggtttgtttactgctgaatctcatagatgtgaaattaaatatgttttattggaaatggatagaattttaagaccaaatggttatgctattattagagaatcatcttattacttggatgctgttgctactgttgctaaaagaatgagatggggttgtagaaaagttgaaactgaagataaattggataaagaaaagattttgatttgtcaaaagaaattatggtactctaatactaaaaagtcatcaagataa

SEQ ID NO: 258 - DNA, 1326 bp, synthetic construct
  source 1..1326: mol_type = other DNA
atgtcatccggtagaaaatataaagaatacattactatttcttcttcattattgcattcatttccatctttttcatttttgtcttttccatttttgggtttagttccaatggcaacatgggctaatttgccatattacttaacattgaaaattaaaaagactaatttgtattacatggctatgattacagttttatgttcaatgtcttacttgattggtatttggcaacatggtggtttgaaattagcagctaatacagcaacttctacattaccacaattgccatgtaatccattgaaaaagaaattcacaactattggttttccattagattttactgctcatcataatgctgatgatttggttccattgtcaacatctgcaagagttcaacaatttccaccatgtaaaccagaattgtctgaatacactccatgtgaagatgctaaaagatcattgaaatttgatagagataaaaatatttacagagaaagacattgtccagaaaaagaagaattgttaaaatgtagaataccagcaccatatggttacaaaactccattcagatggccagcttctagagatatggtttggtacgctaatgttccacataaacatttgactgttgaaaaagctagacaaaattgggttcaattcaaaggtgacagattcaaatttccaggtggtggtacaatgtttccaagaggtgctgatgcatatattgatgatattggtaaattgattaatttgagagatggttcaattagaactgctattgatactggttgtggtgttgcttcttggggtgcttacttgttatctagaaatattttgtctgtttcatttgctccaagagatactcatgttgctcaagttcaatttgcattggaaagaggtgttccagctttgattggtgttatggcttcaattagattgccatacccatctagagcttttgatattgctcattgttctagatgtttgattccgtggggtaaatttgatggtttatttttgattgaagttgatagaattttgagaccaggtggttattggattttatctggtccaccaattaattgggaaaatcattggaaaggttgggatagaaaaccagaagatttgaaagctgaacaagatggtattgaaaatgttgcaaaaatgttgtgttggaaaaagttgattcaaaagaatgatattgctatttggcaaaaaccaacaaatcataaacattgtaaaattaatagaaaagtttttaaaaatccaccatttgttagacataaaattcaaactaaacatggtactccagaatggaaaccagcataa

SEQ ID NO: 259 - DNA, 1482 bp, synthetic construct
  source 1..1482: mol_type = other DNA
atgtcatccgatttgaataaggaacaagaaagagaaatgcaaaaatcttattgggttgaacatacagctgatttgacagttgaagctatgatgttggattctaaagctgcagatttggataaagaagaaagaccagaagttttgtctttattgccaccatatgatggtaaatctattttagaattgggtgctggtattggtagattcactggtgaattggctaaaaaggcaggtcatttggttgctttggattttattgaatctgcaattgaaaagaatgaatcttcaaatggtcattacaaaaatgcaaaattcatttgtgctgatgttacttctccagaattgtcttttccagttgaatctatggatttgattttctctaattggttattgatgtacttgtctgatgaagaagttgaagcattggctgaaagaatggttaaatggttgaaagttggtggtcatattttctttagagaatcttgttttcatcaatctggtgaccataaaagaaagaataatccaacacattacagagttccaagattttatactaaagtttttaaagaatgtcatttgagagatggttctggtaattcttttgaattgacattggttggttgtaaatgtattggtgcttacgttagaaataagaaaaatcaaaatcaaatttgttgggtttggcaaaaagttagatctcaagatgatagaggttttcaaagatttttggataatgttcaatacaaatgtaatggtattttgagatatgaaagagtttttggtcatggttttgtttctacaggtggtattgaaacaactagagaatttgtttctaaattggatttgcaaccaggtcaaaaagttttggatgttggttgtggtattggtggtggtgacttttatatggcagatgaatacgatgttcatgttgttggtattgatttgtctattaatatgatttcttttgctttggaaagagctattggtttgaaatgttctgttgaatttgaagttgcagattgtactaaaaagacttatccagatggttcttttgatgttatttattctagagatacaattttgcatattcaagataaaccagctttgtttaaatctttttataaatggttaaaaccaggtggtagagttttgatttctgattactgtaaaagatcagtttctccatcagaagattttgctaaatacattaaacaaagaggttacgatttgcatgatgttgaagcatacagacaaatgttgagagatgctggttttgatgaagttattgctgaagatagaacagatcaattcattaaagttttgcaaagagaattggattctgttgaaaaagatagagaagcattcattaaagatttttctgaagaagattacaatgatattgttggtggttggaaagctaaattgattagatcttcttcaggtgaacaaagatggggtttgtttattgctaaaaagaattaa

SEQ ID NO: 260 - DNA, 1062 bp, synthetic construct
  source 1..1062: mol_type = other DNA
atgtcatccggttctgctgaagaagaagcatcattgtttgctatgcaattggcatctgcttcagttttgccaatggttttgaaatctgctattgaattagatttgttggaattaattgctaaagcaggtccaggtgcttacgtttctccatcaaaattggctgcacaattgccaactcataatccacaagctccagttatgttggatagaattttgagattgttagctacttactctgttttgaattgtaaattgaaaaatttgccagatggtggtgttgaaagattatacggtttggctccagtttgtaaatttttgactaaaaatgctgatggtgtttctatggctccattattgttgatgaatcaagataaagttttgatggaatcttggtaccatttgaaagatgcagttattgatggtggtattccattcaataaggcatatggtatgactgcttttgaatatcatggtacagatccaagattcaataaggtttttaatcaaggtatgtcaaatcattctactattacaatgaaaaagattttggaagtttacaaaggttttgaaggtgttaaaacagttgttgatgttggtggtggtactggtgcaacattgaatatgattattgataaatacccaacaattaaaggtattaattttgaattgccacatgttgttgaagatgctccattttacccaggtgttgaacatgttggtggtgacatgtttgtttctgttccagaaggtgacgctatttttatgaaatggatttgtcatgattggtctgatgatcattgtagaaaattattgaaaaattgttaccatgctttgccagaaggtggtaaagttattttggctgaatgtattttgccagatgctccagatacttctttggctactcaaaatgttgttcatattgatgttattatgttggctcataatccaggtggtaaagaaagaactgaaaaagaatttgaagcattggctaaaggtgcaggttttaaagaatttagaaaaatttgttcagcagttaatacatggattatggaattatgtaaa

SEQ ID NO: 261 - DNA, 1635 bp, synthetic construct
  source 1..1635: mol_type = other DNA
atgtcatccattgatgattcagaatctaatgttaaagaaattaaaccatgtcatccaagatatacagattacactccatgtcaagatcaaaatagagctatgaaatttccaagagaaaatatgaattatagagaaagacattgtccaccacaagaagaaaaattgcattgtttaattccagctccaaaaggttacgttacaccattcagatggccaaaatctagagattatgttccatacgctaatgcaccatacaaatctttgactgttgaaaaagctattcaaaattggattcaatacgaaggtaatgtttttagatttccaggtggtggtacacaatttccacaaggtgctgataaatacattgatcaattagcttctgttattccaattgataatggtattgttagaactgctttagatactggttgtggtgttgcatcttggggtgcatacttgtggaaaagaaaagttattgcaatgtcttttgctccaagagattctcatgaagctcaagttcaatttgcattggaaagaggtgttccagctgttattggtgttttgggtactattaaaatgccatacccatctagagcttttgatatggctcattgttctagatgtttgattccgtggggtgctaatgatggtatgtacatgaaagaagttgatagagttttgagaccaggtggttattgggttttgtctggtccaccaattaattggaaaacaaattacaaagcatggcaaagatcaaaagaagatttggaagaagaacaaagaaaaattgaagatattgctaaacaattgtgttgggaaaagaaatctgaaaatgcagaaattgctatttggcaaaagagaattgatactgatgcttgtagagcagaacaagaaaattctggtgcagctttttgtcaagcagctgatccagatgatgtttggtacaaaaagatggaagtttgtattactccatctgatagaaaaggtgacgaagcatacggtgaaagaattaaaccatttccagaaagattatatgctattccaccaagaattgcttcaggttctgttccaggtttttctgcagaatcttacgaagaagataataagaaatggaaaaagcatgtttctgcttacaaaagaattaataagattttggattctggtagatatagaaatattatggatatgaatgctggtttaggtggttttgctgcagctttggaatctccaaaattgtgggttatgaatgttgttccaacaattgctgaaaagaatactttgggtgttatttacgaaagaggtttgattggtatttaccatgattggtgtgaaggtttttctacatacccaagaacatacgatttgattcatgctaatggtttgttttctttgtacaaagataaatgtgattttgaagatattttattggaaatggatagaattttgagaccagaaggtgcagttattattagagatgaagttgatgttttgaatagagttcaaaagattgttagaggtattagatgggattttaaattgacagatcatgaagatggtccattagttccagaaaagattttaattgcagttaaaaagtactgggttgcaggtgaaaataattcaacttctacacaa

SEQ ID NO: 262 - DNA, 789 bp, synthetic construct
  source 1..789: mol_type = other DNA
atgtcatcctcaggttatcaagctaaagaatactctactggtagaccaacatacccagaagaaatttttcaatttgttgcttctaaaacaccatgtcatgatttggcttgggatgttggtactggttctggtcaagcagctagatctttggctagaatttacaaaaatgttgttgctactgatgtttcaccagaacaattggaatgtgctgttaaattgccaaatattaaatattactgtacaccaccaaaaatgtctatgactgaattggaacaaaatgttgcaactgaatcttcagttgatttggtttctattgctacagcaatgcattggtttgatttgccaactttttaccaacaagttaaatgggttttgaaaaagccagatggtgttattgctgcatggtcttacacattgccacaaattaataagaatgttgatactttattgagaaaattgtttttcgcagatgctgttcaatttttcggtccacaagctaaattgattattgaagaaaattacagaactattgattttccatttgaaccagttgatggtttggaacatactggtccatttgaatctaaaatggaaaaattgattaatttggatggttattttacattgattaaatcatggtctgcttacaaaactgctaaagaaaaaggtgttgaattgttatcagaagatttggttaaagaatttactttggcttggaatgaagatggtaaacaagaaaagattgctacttggccattgtacatgagaattggtaaagttggtaaacaa

SEQ ID NO: 263 - DNA, 714 bp, synthetic construct
  source 1..714: mol_type = other DNA
atgtcatccgatgttgatgttgaagcaaaaatggatgctgaaaaattgaaaagaggtattgctgaattttatgatgaatcatctggtatttgggaagatatttggggtgaccatatgcatcatggtttttacgatccatcttcaactgtttcattattggatcatagatctgctcaaattagaatgattgaagaagcattgcaatttgcttcagtttctgatgctttgaatcaaccatttccagatggtaaatttgatttggtttggtctttggaatcaggtgaacatatgccaaataaggctaaatttgttaatgaattggttagagttgcagctccaggtgcaactattattattgttacttggtgtcatagaaatttgtcatcttctgaacaatctttacatccagatgaaaagaaattgttagataaattgtgtgatgcttactatttgccgccgtggtgttcaactgatgattacgttaaattgttagaatcattttctttgcaagatattaaagcagctgattggactgaatacgttgctccattttggccagctgttgtttcttcagctttgtcttggaaaggtattacttctttaattttgtctggttggaaaactattaaaggtgcattggctatgttattgatgattaaaggttataaaaagggtttgattaaatttgctgttattacttgtagaaaaccagaa

SEQ ID NO: 264 - DNA, 1536 bp, synthetic construct
  source 1..1536: mol_type = other DNA
atgtcatccgctaaagattataatggttctccaaaaccacatcatttggaaactcaaagaaaaagattgacttggattttagctgtttctggtttgtgtgttttgttttatgttttgggtgcttggcaatctacaaatccagctccatcttcacaatctgatgtttgggaaagagttggttgtcaagatggttcaccagatcaaaaagttaataatccagatttgtcaacttctacattgtctttatcatcttcatggtcatctacaccattggattttgaatctcatcatcaattggaagttaattcttcagaagaatttagaaaatttccaccatgtgatacatcttatactgaatacacaccatgtcaagatccaccaagaggtagaaaatttgatagaaatatgttgaaatacagagaaagacattgtccagaaaaagaagaattgttacattgtttgattccagcaccaccaaattacaaaactccattcaaatggccacaaagaagagattacgcttggtatgctaatattccacataaacatttgtctgttgaaaaagctaatcaaaattggattcaatttgaaggtgacagattcaaatttccaggtggtggtacaatgtttccaagaggtgcagatgcttatattgatgatattaatgaattgattccattgtctaatggtactattagaacagctgttgatactggttgtggtgttgcatcttggggtgcttatttgtttaaaagaaatattattgcaatgtcttttgcaccaagagatactcatgaagcacaagtttggtttgctttggaaagaggtgttccagctatgattggtgttatgggttctcaaagattgccatatccagctagagcatttgatatggctcattgttctagatgtttgattccgtggcataaatatggtggtttgcatttgattgaagttgatagaattttaagaccaggtggttattggattttgtctggtccaccaattagatggaaaaattactggagaggttgggaaagaacacaagaagatttgaaacaagaacaagatgctattgaagatgttgctaaaaatatttgttggaaaaaggttattgaaaaagatgatttggcagtttggcaaaaaccaattaatcatattgaatgtattaaatcaagagcaattcatcatgctactccaatgtgtaaatctgataatgctgatgttgcatggtataaagatatggaaacttgtatttcaccattaccagaagtttcaaataaggatgaagttgctggtggttctttagaaaaatggccagaaagagtttttgcagttccaccaagaatttcttcaggttctgttgctggtattactatggaaaattttagagatgataatgatgtttggaaagaaagagttgctcattacaaaagagttgttggtccattggatcaaggtcaatatagaaatgttatggatatgaatgcatttttaggtggtttgcaacaattttggcaaaatattcaatgtggt

SEQ ID NO: 265 - DNA, 1875 bp, synthetic construct
  source 1..1875: mol_type = other DNA
atgtcatccacaagaggtagatctgatggtgctcaaaagaaaagattattgacttcagttggtattattgctattttcttattgtttttgtacgtttattttggttcaaaatctaatggtgaatctgctttggaatatggttctagatctttaagaaaattgggttcatcttacttgggtggtgacgaagatggtgacttgggttcaaaacaagatgatgcttcatctaaatttgctttggatgatggtgaagatggtttggttccaaaatcatttttggtttgtgatgatagacattcagaattgattccatgtttagatagacatttgatttaccaaatgagattgaaattagatttgtctttgatggaacattacgaaagacattgtccattgccagaaagaagattcaattgtttgattccaccaccatctggttacaaagttccaattaaatggccaaaatcaagagatgaagtttggaaagctaatattccacatacacatttggcttcagaaaaatctgatcaaaattggatggttgttaaaggtgaaaagattatttttccaggtggtggtactcattttcattatggtgctgataaatatattgcttcaattgctaatatgttgaatttttctgataataatttgaataatgaaggtagattgagaactgttttggatgttggttgtggtgttgcttcttttggtgcatacttgttatcatctgatattattgctatgtctttggcaccaaatgatgttcatcaaaatcaaattcaatttgcattagaaagaggtattccagcatatttgggtgttttaggtactatgagattgccatacccatcaagatcttttgaattagctcattgttctagatgtagaattgattggttgcaaagagatggtattttattgttagaattggatagattgttaagaccaggtggttactttgcttattcttcaccagaagcatacgctcaagatgaagaagatttgagaatttggagagaaatgtcagctttggttgaaagaatgtgttggaaaattgcagctaaaagaaatcaaactgttatttgggttaaaccattgactaatgattgttatatggaaagagaaccaggtactcaaccaccattgtgtcaatctgataatgatccagatgcagtttggggtgttaaaatggaaggttgtattacaagatactctgatcatgatcatagaacaagaggttcaggtttagctccgtggccagcaagattaacttcaccaccaccaagattggcagattttggttattcttcagaaatgtgggaaaaagatatgcaagtttggagaagaagagttgaaaattattggaatttgttatcaccaaaaatttcttctgattctttaagaaatgttatggatatgaaagctaatttgggttcatttggtgctgcattaaaagaaaaagatgtttgggttatgaatgttgttccagaagatggtccaaatacattaaaattggtttatgatagaggtttgattggtacaattcataattggtgtgaagcattttctacatacccaagaacatatgatttgttacatgcatggtctgttttatcagatattgaaaagaaaggttgttctggtgaagatttgttattggaaatggatagaattttgagaccaacaggttttgttattattagagataaacaaccagttattgattttgttaaaaagtttttgggtgctttacattgggaagctgttggtactgctgattcatcttcagatccagatcaagaaggtgacgaattggtttttgttattcaaaagaaattatggttgacttcaggttcttttagagatacagaa

SEQ ID NO: 266 - DNA, 762 bp, synthetic construct
  source 1..762: mol_type = other DNA
atgtcatccgctactttgcaaccaccagtttctcaattgccacaaaaatctccagctccaaaattgttaaataagccattgtttaatattttgttattgactattttctgttctttgtcatattttattggttcttacactaattctatttactcaatttctccatctccatcattgattagaagagatactaattgtggttttgaagttaaaaataatttgaatacaactgttgaaaatgttcaagaaccattgaattttgaaccattgcatactttgtctttaccaatggaagatactccagaatctcatcaaccatttccatactgtccaaagaattacactaattattgtccatgtcaagatccaaataaggaaagattgtttactattgatagaagatttcatagagaaagacattgtccagaaaaaggtgaagcattgagatgtttggttcaaaaaccagctggttacagaaggccatttccgtggccaaaatctagagattacgcttggtttaaaaatgttccattcaaaagattgactgaatacaaaaagtctcaaaattgggttagacaagaaggtgacaaattatttttcccaggtggtggtacttcttttccaaaaggtgttaaaggttacattgatttgttaaaaagagttgttccattaaattctggtgctattagaactgttttggatgttggttgtggtgtttctattaatggttgttctaaaatttttattgtttttgttagattt

SEQ ID NO: 267 - DNA, 1569 bp, synthetic construct
  source 1..1569: mol_type = other DNA
atgtcatccgaaccatctactggtaaaccatcatttttcagaaatattttggttagattatttttgttttgtgtttttattttgggtgttagatttgcttatgttgttactattagaggtgaatcatgtgatttgggtgacttttgtttcttttctttgccagataatttgaatacaattgtttctggtgttggtcaattgggtactaaatcattttcttcatctgcaattattgctgttggtaaatctgctccagcaaaaccacataaattaccagatttgtgggcaactcaaggttttcaaaaagctgctcaattttattcttcagtttttcaagatttgatttcagaaggtttcttgactccaaattcaaaagcattgtgtgttgatacaccaactggtgctgatgtttttgctttgaaagaaattggtgtttcagattctattggtatttttaaaaagtcttcaaaaccattggttatttcaggtcaagcaattaaacaaccatttggtgacgataaatttgattttgttttctcaggtgctggtatgattgaaaaatctgttaaagcagctgattttgcagctgaagtttgtagaacattaaaaccagaaggtttctttgttgttcatactggttctaaagatacttactcttacaattcatttttagatttgtttaattgttgtaaattagttaaattcagagatattgatggttttgattcaaaattaccattcattagagaaattgttatgcaaaagaaattcaatgaaaatgttgatgatgaaattcaaatgccaggtggtaattctgaagataaatgttttgttccaggttacaaacaagaattgattagaaatgctgaaccattgattgttgaagaaccaaagaaaccgtggattactttgaaaaagaatattcaaaatattaaatatttgccatctatggctgatatttcttttaaacaaagatacgtttatgttgatgttggtgctagatcatacggttcttcaattgtttcatggtttaaaaagcaatacccaaaacaaaatagaacatttgaaatttacgctattgaagctgataaacattttcatgaagaatataaatacaaaaagggtgttaaattgttaccatatgcagcttgggttagaaatgaaacattgtttttcgaaattaatgaagatccaattgataaagatgttgttaaaggtagaggtatgggtagaatacaaccagttcaatcatcttcaggtgtttctgatggtaatgttgatgaaattcaaggttttgattttgctgaatggttgaaatctacagtttcagaaaaagattttgttgttatgaaaatggatgttgaaggtacagaatttgatttgattccaagattgattgaaactggtacaatttgtttgattgatgaagttttcttggaatgtcattacaatagatggcaaaaatgttgtccaggtgaaagaacttctaaataccaaaagacttacgatcaatgtttggaattgtttacttcattaagagaatctggtgttttggttcatcaatggtgg

SEQ ID NO: 268 - DNA, 2115 bp, synthetic construct
  source 1..2115: mol_type = other DNA
atgtcatcctctagaccattgcatagaggtgtttctggtggtggtagattttctccaaattcacatgatttgtgggatgatgatgattcacaaatgaaagataaagcagaaaaagaagatggtgacagaaaatctacaaatgctgatcatacatacttgtcaattagacaaccattcagatttttgttagctgataattcttcatcaaaacatggtgttactaaagatggttttgtttcagatccattttctccatcaccaggtgtttctagaaatagacataaattcattttgttattattgagatttggtttagttgttattgttatgttagctttggcaggttctttttggtggactatttcaattacaacttcttcaagaggtcaaatttatagaggttacagaagattgcaacaaaaattggtttcagatttggttgatgttggtaaattgtcattaggttctacaagattgaaagatttggaatactgttcaccagaatctgaacattacgttccatgttttaatgtttcagaaaatttggctttaggtatttcaggtggtaatgaatacgatagacattgtggtccaggttctagacaatcttgttttgttttgccaccagttaattacaaaactccattgagatggccatggggtagagatttgatttgggttgctaatattaatattactgctcaagaagttagatcttcaggttcattgactaaaagaatgatgatgtacgatgaagatcaaatttcttttagatctgcttcaccaggttacgatgatatggaagattacgctcatcaaattgcagaaatgattggtttgagaaatgaatcaaattttgttcaatctggtgttagaactattttagatattggttgtggttatggtaatttgggtgctcatttgttttcaaatgaattgttaactatttgtattgctaattacgaagcatctggttcacaagttcaattgacattagaaagaggtttaccagctatgatttcttcttttacttctaaacaattgccatacccatctttgtcatttgatatgattcattgtgctggttgtggtattgattgggatcaaaaagatggtattttattggttgaagttgatagagttttgagaccaggtggttattttgtttggacatcacaattgacttcatctcaacaatttttaagaaagaatgaaaatcaaaaagaatggaattttgttcatggttttgctaaatctttgtgttgggaaatgttgtctcaacaagttgatactgttgtttggaaaaagacttctaaaaagaattgttattcagctagaaaatctggttcaggtccatctttatgttcaagaggtcatgatgttgaatctccacattacagaccattgcaaggttgtattggtggtactcaatcacaaagatggattccaattgaagaaagaactaaatggccagctagagctagattgaatgcaaaagaattagctattcatggtttaaattctgatgattttgcagaagatacattgaattggaactctgcagttagaaattattggtcattattgtctccagttattttctcagatcatccaaaaagaccaggtgacgaagatccatctccaccattcaatatggttagaaatgttttagatatgaatgcacattttggtggttttaatgctgcattgttagaagctaaaaagtctgtttgggttatgaatgttattccaactacaggtaataatcatttgccattgattttagatagaggttttgttggtgttttgcatgattggtgtgaagcatttccaacttatccaagaacatacgatttggttcatggtgaaggtttcttgtctttagaatttggtgaagaacaaagatgtgaaatgttggatgtttttactgaaattgatagaattttgagaccagaaggttggtttattttgagagatacaactccattgattgaatctgctagaacattgactgcaagattgaaatgggatgcaagaatgattgaaattgaaaataattcagatgaaagattattgatttgtcaaaaaccatttgctaaaagacaagcaatgtaa

SEQ ID NO: 269 - DNA, 306 bp, synthetic construct
  source 1..306: mol_type = other DNA
atgtcatccaaatggattttgcatgattgggatgatggtcattgtttgaaattattgaaaaattgttacaaagcattgtctgataatggtaaagttattgttgttgatgctttggttccagttattccagatacttctccatgtgttaaaactacatgtcaatctgatttgattatgatgactcaaaatccaggtggtaaagaaagatctgaaccagaatttttggcattagctactgcagctggttttaaaggtattagattggaatgttacgtttgtaatttgtgggttatggaattttacaaa

SEQ ID NO: 270 - DNA, 420 bp, synthetic construct
  source 1..420: mol_type = other DNA
atgtcatccaaaattcattcaagatctttgtgtccatcaagatcttacggtggtcaagaagttgaagtttttcaaattgctagaactatgatgcaatctattattttgtactctattatttgttctttgcattgttttcatgcatttccatcatctaattcatctactccagcatacaatgctttgtggaataagtctttaactttgccaaatgttactaataatagatactttttgtttatttttactccattttctaaacatactaaagttaaacatgctactacaactattgttaaattgaataattacatttcaattgttaatgatgttagaaatttgtcagttattgattttatttttgttatttacattcattcatctagatgtcattacacttatagaatttcttcaaataag

SEQ ID NO: 271 - DNA, 1158 bp, synthetic construct
  source 1..1158: mol_type = other DNA
atgtcatcccaaccacaaagaggtagaaaaagagaaagagaaagagatagagaagaaatggaatctgttcaatctaattcatcttcttcagatcaatttgctatgaaaggtggtgacgatgatttttcatatactaaaaattctacacatcaaagagatgctattcaagctactaaatttttcattcaagaatcaattgctgaaaaattggatgttaataagttttgtggtaaagcattttgtgttgctgatttgggttgttcagttggtccaaatacattgattgctatgcaaaatattgttgaagcagttgaattgaaattcaaaaatagaaaaggttttcattcaccaacaattccagaatttcaagttttctttaatgatcatactgttaatgattttaatactttgtttagatctttgccaactggtcatgataaaagatattacggtgttggtgttccaggttcattttacggtagattgtttccatgtgattctattcatattatgcatacttctttttcaactccatttttgtctcaagttccaaaagaagttatggataaaaattctgcagcttggaataagggtagaatacatcataattacgcaaaagctgatgttttgaaagcatacgaagctcaacatgctgaagatattgattgtttcttgactgcaagagctaaagaattggttcatggtggtttattgatggatgttacttcttttagaccagatggtgttccacatactcatgttttaactaatattggtatggaagttttgggttactgtttaatggatttggctaaaaagggtttaattgatgaagaaaatgttgattcttataatgttccagtttacttgcaatctccagaagaattgaaacaagctgttcaaagaaataagtacttttctattgaaaagatggaatcagttccaatgatgattgattctgatgtttcagcaaaagctcaacaatactctttgggtatgagagctgttatgggtgacgttattagagaacaatttggtgctgaaattgttgataaattgtttgatttgtttaaaaagaaattggaagaacatccaaattttgcaaaaggtgttgttttggatatgtttgttttgttaaaaagaaatgctgaagattaa

SEQ ID NO: 272 - DNA, 1101 bp, synthetic construct
  source 1..1101: mol_type = other DNA
atgtcatccgaatcttcacaatctaataatagattttgtgatcaatttgctactaaaggtggtgacgatgatttttcttatactaaaaattcaattcatcaaagagatgcaattgaagctactaaattattgattcaagaagcaattgctgaaaaattggatattaataagttttgtggtaaaactttttgtattgctcatttgggttgttctacaggtccaactacattgactgctatgcaaaatattattgaagctgttgaattgaaatttcaaaatcaaaaaggttttaattttccaaattttccagaatttcaagttttctttaatgatcatactgttaatgattttaatgctttgtttagatcattgccaacaggtcatgataaaagattttacggtgttggtgttccaggttctttgtacggtagattgtttccatgtaattctattcatattacacatacttctttttcaactccatttttgtctttagttccaaaagaagttatggataaaaattcagcagcttggaatgaaggtaaaattcattacaatcatgctaaagaagatgttttgaaagcatacgaaggtaaacatgctgaagatatggattgtttctttatggctagagcaaaagaattggttcatggtggtttgatgttaattgttacatctttgagaccagatggtattccacatgttttgactaatattggtatggaagttttaggttcttgtttgatggatttggctaaaaagggtttgattgatgaagaaaatgttgattcttttaatgttccagtttacttggaatctccagaagaattgaaacaagctattcaaagaaataagtacttttcaatggaaaaagttgaatctgttccaatgattatggattctgatatttcatgtagagcacaacaatattctttggctattagagcagttatggaagatgtttttagagaacaatttgaagctgaaattttagatcaattgtttgatttgtttaaaaagaaattggaaggtcatccaaattttgcatctgctgttgttttagatttgtttgttttgttaaaaagaaatgcagaagattaa

SEQ ID NO: 273 - DNA, 1107 bp, synthetic construct
  source 1..1107: mol_type = other DNA
atgtcatccgaatcattgccaggtaataatagatcttacgatcaatttgctatgaaaggtggtgacgatgatttttcttacactaaaaattctattcatcaaagagatgctattgaagcaactaaattattgattgaagaagcagttgctgaaaaattggatatttcaaatttttgtggtaaagcattttgtattgctgatttgggttgttctgttggtccaaatactttaattgctatgcaaaatattgttgaagaagttaaattgaaattcagaaatagaaaaggtttgaattttccaaatattccagaatttcaagttttctttaatgatcatactgttaatgattttaatgctttgtttagatcattgccaactggtcatgataaatcttattacggtgttggtgttccaggttctttttacggtagattgtttccatgtgatactattcatattatgcatacttctttttcaactccatttttgtctcaagttccaaaagaagttatggaaaagaactctgcagcttggaataagggtagaatacattacaatcatgctaaagaagatgttttaaaagcatacgaaggtaaacatgctgaagatatggattgtttctttatggctagagcaaaagaattggttcatggtggtttgatgttaattgttacttctttgagaccagatggtgttccacatactagagttttgactaatattggtatggaagttttgggttcttgtttaatggaattggctaaaaagggtttgattgatgaagaaaaagttgattcttttaatgctccagtttacttggaatctccagaagaattaaaacaagcaattcaaaagaataagtacttttcaattgaaaagatggaaacagttccaatgattatggattcagatatttcttgtagagcacaacaatactctttggctattagagcagttatggaagatgtttttagagaacaatttgaagctgaaattttggatcaattatttgatttgtttaaaaagaaattggaaggtcatccaaattttgcttctgcagttgttttagatttgtttgttttgttaaaaagaaatgcagaagattaa

SEQ ID NO: 274 - DNA, 1107 bp, synthetic construct
  source 1..1107: mol_type = other DNA
atgtcatccgaatctgttcaatcaaattcttcatcttcagatcaatttgctatgaaaggtggtgacgatgatttttcttacactaaaaattctactcatcaaagagatgcaattcaagctactaaatttttcattcaagaatctattgctgaaaaattggatgttaataagttttgtggtaaagcattttgtgttgctgatttgggttgttctgttggtccaaatactttgattgctatgcaaaatattgttgaagctgttgaattgaaattcaaaaatagaaaaggttttcattctccaactattccagaatttcaagttttctttaatgatcatactgttaatgattttaatactttgtttagatctttgccaactggtcatgataaaagatattacggtgttggtgttccaggttctttttacggtagattgtttccatgtgattcaattcatattatgcatacttctttttcaacaccatttttgtctcaagttccaaaagaagttatggataaaaattctgcagcttggaataagggtagaatacatcataattatgcaaaagctgatgttttgaaagcatatgaagctcaacatgcagaagatattgattgtttcttaactgctagagcaaaagaattggttcatggtggtttattgatggatgttacatcttttagaccagatggtgttccacatacacatgttttgactaatattggtatggaagttttgggttactgtttgatggatttggctaaaaagggtttgattgatgaagaaaatgttgattcttacaatgttccagtttacttgcaatctccagaagaattgaaacaagcagttcaaagaaataagtacttttctattgaaaagatggaatcagttccaatgatgattgattctgatgtttctgcaaaagctcaacaatattctttgggtatgagagcagttatgggtgacgttattagagaacaatttggtgctgaaattgttgataaattgtttgatttgtttaaaaagaaattggaagaacatccaaattttgctaaaggtgttgttttggatatgtttgttttattgaaaagaaatgctgaagattaa

SEQ ID NO: 275 - DNA, 1155 bp, synthetic construct
  source 1..1155: mol_type = other DNA
atgtcatcccagccccaaaggggccgtaaaagggaacgtgaaagagacagggaggagatggaaagccttcaaaacaacaacagatactgtgaccaattcgccatgatcggaggggatgacgattttagctatacaaaaaactcaatccaccaaagagatgctatcgaagccacgaaatttcttattcagggatctattgcagaaaagttagacatcaataatttttgcggtaaagcgttttgcgtcgccgatttggggtgctccgttggtcctaataccttgatcgcgatgcagaatattgtggacgccgtagaacttaagtttaagaacaggaaaggtttccattcacccacaattccagagttccaagtcttctttaacgatcatacagttaatgacttcaatactcttttcaggtcattgcctaccggacacgataaacgttactacggagtcggagttcccggtttttttcttcgtagtataattagtctagattcaatacacattatgcacacgagcttctctacaccgttcctgagccaagtccctaaggaggttatggataaaaactcagcagcctggaacaaagggcgtatccaccacaattacgctaaggccgatgttctaaaggcctacgaggcgcaacacgccgaggacatcgattgttttttaacagcaagggctaaagagttagttcatgggggtttacttatggatgttacctccttcaggccagacggagtacctcacacccatgtgcttacaaatataggaatggaagttttaggttactgtcttatggacctggctaaaaaaggccttatagacgaggagaatgtagacagttacaatgtcccagtttatctacagagtcctgaggagttgaaacaggctgtccaaagaaacaagtatttctcaatcgaaaaaatggagagtgttccaatgatgattgactctgatgtatcagcaaaagcccagcagtactccttgggaatgagggcggtaatgggggacgtaatcagagagcagttcggtgcggaaatagttgacaaacttttcgatctgttcaaaaagaagcttgaggagcaccctaatttcgctaaaggagttgtcctagatatgttcgtgctattaaagaggaacgcggaggac

SEQ ID NO: 276 - DNA, 1146 bp, synthetic construct
  source 1..1146: mol_type = other DNA
atgtcatcccagccccaaagaggtagaaaacgtgaaagggagagggacagagaggaaatgtataaggcgccctttgtgttgtgcgctgcggtcgcggtcataactgatcatatgaacttgagtggcgcaggcaattatgggagggatgcaattgaggcaacaaaatttctaatacaggagtctatagctgaaaagctggacatcaacaaattctgcgggaaagccttctgtgttgccgatttgggatgcagcgccggccctaacacccttatagcgatgcaaaatatagtagaggcggttgaacttaaattcaagaaccgtaaaggctttcattcacccaatatacccgagtttcaggtttttttcaatgaccacaccgtaaacgattttaacacattattccgttcactacccacggggcacgataaaagatactatggagtaggcgtccccgtattgttcaccgtggattattttttggttactttgtttacacacatcatgcacacttctttttcaacgccttttctatctcaagtacctaaagaagtaatggataaaaactctgcggcgtggaataaggggcgtattcaccacaactatgcaaaggccgatgtcttaaaagcttatgaagcacaacacgccgaggacatagactgctttttaacggcgagagctaaggaattggtgcatggcggtttgttaatggatgtcacatcatttagacccgacggcgtaccgcacactcacgtacttactaatataggtatggaggtattaggctactgtttaatggacttagctaaaaagggactaattgacgaggaaaacgttgattcctataacgttccagtttatctacagagtccagaggagctgaaacaggctgttcaaagaaacaaatacttctccatagaaaagatggaatctgtcccaatgatgattgacagtgacgtctctgcaaaagcacagcagtacagtttggggatgagagccgtgatgggagatgttatccgtgagcagttcggcgccgagatagtggacaagctgttcgatctgtttaaaaagaaacttgaggaacatcctaacttcgcgaagggtgtcgtacttgacatgtttgtcttattaaagcgtaatgctgaagac

SEQ ID NO: 277 - DNA, 873 bp, synthetic construct
  source 1..873: mol_type = other DNA
atgtcatcccaaaatatagtagaagcagtggagttaaaatttaagaatagaaaggggtttcacagtccgacgatccccgagtttcaggtattcttcaacgatcataccgtaaatgatttcaatacattgtttcgtagcttaccgacaggacacgacaagagatactatggcgtgggagtgccaggtagtttctacggtcgtttgttcccatgtgactccatacatattatgcacactagctttagtactcctttcttgagccaagtaccgaaggaggttatggacaaaaactcagctgcctggaataaaggtcgtattcatcataattatgccaaggccgatgtattaaaagcttatgaggcacaacacgcggaagacatcgattgcttcctaacagcacgtgcgaaggaactggtccatgggggattgttgatggacgttaccagttttagacccgatggagttccccacactcacgtcttaactaacattgggatggaggtgctaggctactgtcttatggacctggctaaaaagggtctaattgatgaggagaacgtcgactcctataatgttccagtttatctgcagagcccagaggaattaaaacaagctgttcagaggaacaaatatttttcaatcgagaaaatggagtccgtgccgatgatgatagatagtgacgtaagtgcgaaggcacaacaatactcattgggaatgcgtgctgttatgggtgacgttatcagggaacagtttggtgcagaaatcgtggacaagcttttcgatttgttcaagaaaaaattggaagagcacccaaattttgcgaagggggtcgttctggatatgttcgtcctgctaaagagaaacgcagaggat

SEQ ID NO: 278 - DNA, 1506 bp, synthetic construct
  source 1..1506: mol_type = other DNA
atggcagcattgccattgttgattttctcttctttgtttttggctttgttgttgaaattattgttgaaacataagaacaacagaaattctaaattaccaccatgtccaccaaaattgccaatcatcggtaatttgcatttgttgggtaatatgccacaagaatcattgcatcaattggctaagaaatacggtccagttattttcttgcaattgggtgaagttccaactatcgttatttcttcagctagattggcaaaagaagctatgaaaacattcgatttggcattatcttcaagaccacaaattttctctgctaagcatttgttttataattgtactgatgttgttttctctccatatggtgcttactggagaaacattagaaaaatctgtatcttggaattgttatctgcaaagagagttcaatctttttcatacgttagagaagaagaagttgctcatttggttcaaagagttgctgaagcatatccaggtactacaaatttgacaaagatgttgggtttgtacgcaaacaatttggtttgtagagttgctttcggtaaagatttctcacaaggtggtgacaaccatagacataacttccaaaaattgttagaagaataccaagttttgttaggtggtttttctattggtgactttttcccatcaatggaatttgttcataagtttactggtatggaatctagattaaaagatacttttagaagatttgatcaatttttcgatgaaatcttgcaagaacatagaaacccagaatcaagaaaagaacataaggatttggttgatgttttgttagaaattcaaaagaatggtgacccagaaactccattaactacagataacatcaagtctatcattttggatatgtttgctgcaggtactgatacaaacttcatcacattggattgggctatgactgaattaatgacaaacccaggtgttttgaaaagagcacaagcagaagttagaaatatcgttggtgaaagaagatctgtttcagaaaacgatttgccacaattgccatatgttaaggcagttgttaaggaaacttttagattacatccaccagctccagttttggttccaagagaatctatggaagatgttactattgatggttacgatattccagcaaaaacaagatttttcatcaacgcttgggcaattggtagagatccaaaatcttgggaaaatccagatgtttttgaaccagaaagattcatgaactctcatatcgatttcaagggtcaagattacgaattgattccatttggtgctggtagaagatcttgtccagcaatcactttttcagctgttacagttgaattggctttagcacaattgttgcattctttcgattgggaattaccaccaggtgttgaagcaaaagatttggatatgactgaagtttttggtatcacaatgcataagatctatccattgatggttgttgctaaaccatacttttcacaaggtttgaattaa

SEQ ID NO: 279 - DNA, 1506 bp, synthetic construct
  source 1..1506: mol_type = other DNA
atggcagctttaccattgttgattttctcttctttgtttttggctttgttgttgaaattattgttgaaacatactaaattggttaaattaccaccaggtccaagaccattaccaatcatcggtaatttgcatcaattgaacggttctttgccacatagaattttgactgatttggctaagaaatatggtccattgatgcatttgcaattgggtgaagtttcaacagttattgtttcttcaccagctgttgcaaaggaagttatgaaggatcatgatattattttctgttatagacctttgttattgtctccagcaatcttgaactacggtggtacagatattgcttttgcaccatatggtgaatactggagacaattgagaaaaatctgtactatcgaattgttatcttctaaaagagttcaaacttttagatctattagagaacaagaagtttcaaatttgtttagatctattaaatcagaagaaggttctgttgttaatttgtctaagatgatctattctttgacttactcaattacagctcaagctgcatttggtaaagaatctaagtaccaggataagtttatttcagttatgcaagatgctattaaattaatgggtggttttaatatcgcagatgtttacccatctattaaaatcttggaaaagattactggtattagacataaattggaagaaacacataaccaaatcgatgaaatcgttcaaaacattttgattgaacataaggctaagagagcagaatcaaaattgggtaatagaggtgctaaggaagatttggttgatgttttgttgaacatccaatcttcaggcgaatttgaaccaccattaactgatgataatttgaaagctgttatatatgatattttctctgctggtggtgaaacttcttctactacagttatttgggcaatgtcagaaatgattagaaatccaaaagttatgaagaaagctcaagatgaagttagagaagtttttgctggtaatggtaatgttgatgaatctagattgcatgaattaaaatacttgcaagctgttattaaagaaactatgagattaaatccagcagttccattattgttaccaagagaatgttcagaacattgtcaaattaatggtttcgatgttccagctaaaactagagttattgttaatgcttgggcaattggtagagatccaaaacaatggacagaaccagaaaaattcaaaccagatagatttttggattctgaaatcgatttcagaggtaaaactttttcttttattccatttggtgctggtagaagaatttgtccaggtatttcttttgctttgccattagttgaattgccattagcacaattgttgtaccatttcgattggaaattgccaggtcaatcaaaaccagatgaattagatatgactgaaatcttcggtttgacagttggtagaaagcatgatttgaatttggttccaattccatacagaccatgttctgttttgtaa

SEQ ID NO: 280 - DNA, 1506 bp, synthetic construct
  source 1..1506: mol_type = other DNA
atggcagaattgttgtttactttgtgtgctttgttggttttcttttctttcgcagttaagatcgttagaaaaacaaaaccagttaaattaccaccaggtccaagaccattgccaatcatcggtaatttgcatcaattgaacggttctttaccacatagaattttaagaaatttggctaagaaatatggtccattgatgcatttgcaattgggtgaagtttcaactgttattatttcttcaccagctttggcaaaggaaatcatgaacgaacatgatattgttttctgttacagaccatttttgttatctccatcaatcttgaactacggttgtgctgatattgcttttgcaccatatggtgaatactggagacaattgagaaaaatctgtactatggaattgttaacatctagaagagttcaaacttttagatcaattagagaagttgaagtttctaatttgattagatcaatttcttcagaagaaggttctgttgttaatttgtctaagaaaatatattctttgacttactcaattacagctcaagctgcatttggtaaaagatctaagtaccatgataagtttatttcaatcatgcaagatgttgttaaattgatgggtggttttaaaatcgcagatatgtacccatctttaaaaatcttggaaaaattgactggtattagacataaattagaaatggttcataaccaagttgatgaagttattgaaaacattttgtctgaacataaggttaagagagctgattcaaatttgggtaacagagaagcaaaagaagatttggttgatgttttgttgaacatccaatcttcaggcgaatttggtgttccattaacagatcataacatcaaggctgttatatatgatgttttctctgctggtggtgaaacttcttctactacagttatttggacaatgtcagaaatgattagaaatccaaaagttatgaagaaagctcaagatgaagttagagaagtttttgctggtaatggtaatgttgatgaatctagattgcatgaattaaaatatttgcaagctgttattaaagaatcaatgagattgcatccagctgcaccattgttattgccaagagaatgttctgaacattgtcaaattaatggttacgatattccagctaaaactagagttattgttaatgcttgggcaattggtagagattcaaaagcatggacagaaccagaaaagtttaatccaaacagatttttagattctgaaattgattttagaggtaaaactttttcttttattccatttggtgctggtagaagaatttgtccaggtatttcttttgctttaccattggttgaattaccattggcacaattgttgtaccatttcgattggaaattgccaggtcaatcaaaaccagaagaattagatatgacagaaatcttcggtttggcagttggtagaaagcatgatttgaaattgatcccaatcccatacagaccatgttctagagaataa

SEQ ID NO: 281 - DNA, 672 bp, synthetic construct
  source 1..672: mol_type = other DNA
atggcaattgcaaaagatgttttggttactcatcatccatcttttgcagatagaccaaacaatttggctacagaaatcatcagatacgatcaagaagatatgggtttttcaccatatggtgactactggaagcaaatgagaaagatctgtatgatgcaattgttatctatgactaacgttgcatcattttcttcaatcagacaagatgaaatttctaaattggttgaagctattagatcaacaccaggtgttccagttaatttgactgaaaagattttcgcatgtacatcttcaatcatttgtagagctgcattcggtcaagtttgtcaagataaggaaaacatgattaaaaatttgaaaatcgctttcgttttgttagcaggttttaatttggctgatgtttttccatctttgaagtttattccaatcatcactggttttaaacaaaaattgattaaagttcatcataaaatggatgaaatcttgggtgacgttattaaagaacatatcttgaaacatgaaaacggtaaaaagtgtaacgctgaatcaggtgacgaagatttgatcgatgttttgttgcaacaacctaaaaatggtaatttgcatatcccaatcactgcaagaaatatcaaagcattgattttagtttctacaatttcattgtgtttttaa

SEQ ID NO: 282 - DNA, 1341 bp, synthetic construct
  source 1..1341: mol_type = other DNA
atggcattgcaattgggttctgttaaaacattagttgtttcttcagctgaagctgcacatgaaatcatgaaaactcatgatttgaatttcttgaacagaccagaatcaaaggttaacagaaaattgttgtacgattttaaagatttgtctgttgctccatatggtgaatactggagacaaatgaaatcaatctgtgttttgcatttgttatctaataagagagttcaatcaatcagatacattagagaagaagaaacttcatttttgatcactaagattagagaaggttctttagcttcaccatcttcaggtgcagttaatttgtctgaaatgttcgttactttgacaaatgatgttgtttcaagagctgcatttggtagaaaatattctgagggtgaatcaggtagaaagtttagaaaattgatgtctgaatttgtttcagttttaggtggtttcgatttcggtacagttttgccatgtttgggttggattgatagattaaatggtttgactgctagagttgaaagaatcgcaaaggaaatggataagttcttagaaggtgttttggaagaacatttggattctgatagaaagaataacgaatcaagagttgttatgactagaaaaattcatggtgaaaacggtgaagattttgctgatgttttgttgggtatctataagaataaggctgcaggtatttctatcgatagagattcaattaaaggtttgttattggatatttttgcaggtggtacagatactacatacactgttttagaatgggctatgacagaattattgagacatccaaaagcattaaaagaattgcaagatgaagttagaggtttggttggtggtggtagagaaatcacagaacaagatttggaaaagatgaagtatttgaaagctgttattaaagaaactttaagattgcatccaccaattccattattggttccaagaaaagctattaatgatgcaaagatcatggattacgatatttctgcaggtacaatggttattactaacgctttcgcaattggtagagatccatctttgtgggaagaaccagatgagtttagaccagaaagatttttaaattcatgtatcgatttcagaggtcatgatttccaattgattccatttggttctggtagaagaggttgtccaggtatttcatttgctatggcaacaaacgaacatgttttggctaatttggttttgaaattcgattgggatttgccagatggtgcaaaaggtactgatttggatatgactgaatgtacaggtttgactatccatagaaagttcccaatcttcgctgttgcaacaccagtttaa

SEQ ID NO: 283 - DNA, 1023 bp, synthetic construct
  source 1..1023: mol_type = other DNA
atggcattcatgactttgacaaacgatgttatgtctagagctgcatttggttggatttgttcagagggtgaatctggtagaaagtttagaaaattgatgtcagaatctgctagagtttttggtgtttttgatttcggtacttatttgccatgtttgggtcaaatcgattcagttaacggtttgatggttagagttgaaagaatcgcaaaggaaatggatgaatttttagaaagagttttggaagaacatttggattctgatagaaagaataacgaatcaagagttgttatgacttctaagattcatggtgaaaacggtgaagatttcgctgatgttttatttggtatctataagaataaggctgcaggttcttcaatcgatagagattctattaaagcattgttgttggatatgtttgcaggtggtactgatacaaactacacatttttggaatgggctatcactgaattattgagacatccaaaagcattaaaagaattgcaagatgaagttagaggtttagttgatggtggtagagaaatcacagaacaagattctgaaaagatgaagtatttgaaagctgttattaaagaaactttaagattgcatccaccattaccattgtcagttagaaaagctattaatgatgttaagatcatggattacgatatttctgcaggtactacagttattacaaacgctttcgcaattggtagagatccatcattgtgggaagaaccagatgagtttagacctgaaagatttttaaactgttgtgttgatttcagaggtcatgatttccaattgattccatttggttcaggtagaagaggttgtacaggtatttcttttgctatggcaactaacgaacatgttttggctaatttggttttgaaattcgattgggatttgccagatggtgcaaagggtacagatttgaacatgactaaatctacaggtttgactatccataggaaaaattcttcttcttgttgttgtaattcatctttattgactcaataa

SEQ ID NO: 284 - DNA, 1125 bp, synthetic construct
  source 1..1125: mol_type = other DNA
atggcagaattgttatctccaaagagagttcaaacttttagatcaattagagaaggtgaagttttgaacatgatcaagtctattgctttacatatgggttcttcagttgatttgtctagaaaaattttctctttgccatacggtattacagcacaagctgcattcggtaaaaagggtaaataccaagaacaatacatcatgttgattgatgaaatggttaaattgtctggtggtttttcattagttgatgtttacccatctattaaattgttagaaagaatttcaggtactagaacaaaattagaaaaattgcataaacaaattgatggtattttacaaatcatcgttaacgaacataaaaagaaattggaaaaagaagaattaggttctggtgaaaagaaagttgatttggttgatgttttgttgaacatcggtaaaagaggtgaatttggttcacatttggctgataacaacatcaaggcaatcatctatgatattttctctgctggttcagaaacttcttcaactacagttgaatgggcaatttctgaaatgatcaagtcaagaacaatcatgaagagagcacaagatgaagttagaaatatttttaaggaacgtggtaacgttgatgaatctaatttgcatgaattgaaatacttacaagctgttattaaagaaactatgagattgcatccaggtgcaccattgttgttgccaaaggaatcaatcgaaaagtgtgaagttaagggttacgaaatcccagctaaaacaagagttattattaatgcttgggcattaggtagagatccaatgtactggtctgaagcagaaaagtttaatccagaaagatttttggattcagaattagatttcagaggtaaccatttcgaatatattccatttggtgctggtagaagaatttgtccaggtatttcttttgctttgccaaacactgaattaccattggcacaattattgtttcatttcgattggaaattgcctggtgacttgaaacaagaacaattggatatgactgaagtttttggtgttactgttagaagaaagaatgatttgagattgattccagttccatactctcattcttctttgaataaggatttgtaa

SEQ ID NO: 285 - DNA, 774 bp, synthetic construct
  source 1..774: mol_type = other DNA
atggcattggctaagatcgttatgaaatctaaaaacaaaaattctggtaaaaagttgccaccaggtccaagacaagttccaatcatcggtaacatgcatcaattgtatggtccattaccacatcataaattgagagatttggctaagaaatacggtccattgatgcatttgcaattgggtgaaatttctactatcgttgtttcttcaccagaatttgcaaaggaagtttacagaacacatgatattatcttcgctgaaagagcagaattgttatctatttcaattgtttcatacaatttcttgggtatcgtttttgctccatatgcagattactggagacaattaagaaaaatctgtactatggaattgttatctccaaagagagttcaaacttttagatcaatctgggaaggtgaagttttgaacatgatcaagtctattgctttacaaaagggttcttcagttaatttgtctagaaaaattttctctttaacttatggtattacagcacaagctgcattcggtaaaaagggtaaataccaagaacaatacgttatgttggttgatgaaatggttaaattgtctggtggtttttctttggttgatttgtacccatctattaaattgttagaaagaatttcaggtactagaacaaaattggaaatcttgcataagcaaattgatggtattttgcaaactattgttaatgaacataaaaagaaattggaaaaagaagaattaggttctggtgaaaagaaatctttgtctggttaa

SEQ ID NO: 286 - DNA, 1566 bp, synthetic construct
  source 1..1566: mol_type = other DNA
atggcagcaattttctctaagatcggtatcgttggtgctggtatttctggtattgctgcagctaagcaattgagacattacaagccagttgtttttgaagcaactgattctttaggtggtgtttggagacattgttcttacagatcaactaaattgcaaacaccaagatgtgattacgaattttcagattttccttggacagaaagagataattcttcatttccatcttacatggaaatcttggattacttaaattcttacgcttcaaacttcggtgttttggaattgattaagtttaattcaaaggttgttgaaattaagtttactggtaacagagatcaatctaactactctgattcaggttctaattcaaaggaatacggtaatttgttaggtggtcaaccagtttgggaagttgcagttcaaacttctgattcagatacaatcgaatggtacgctttcgaatttttagttatttgtactggtaaatacggtgacattccaatcatcccaaagttcccaaacaataagggtccagaagtttttagaggtcaagttatgcatacattggattactcaaaattggatgaagaagcatctgttaatttgttgaaagataagaaagttgttgttattggttacaagaaatctgctatcgatttggctgttgaatgtgcaaaagctaatgaaggtccagaaggtaaaccatgtactgttgttgttagaactttgcattggacagttccacattactctatctggggtttgccattctatttgttttattcaacaagagcatctcaattcttgcatgaaagaccaaaccaaggtattttgagaaatatcttgtgtaaattgttatctccagttagaaaagctatgtcaaagatcatcgaatcttatttggtttggaaattgccattgaagaaatacggtttgaaaccagatcatccatttgaagaagattacggttcttgtcaaatggcaattttgccagaagaatttttcgcagaagctgataagggtatgatcgagtttaaaagagcttcacaatggtggttttgtgaaggtggtgttcaattcgatgataactctaaattagaagcagatgttgttttgttagctactggtttcgatggtaaaagaaaattgagatcaatcttgccagaaccttttagatctttgattgaaactgatggtgttatgccattgtacagaggtacaatccatccattgatcccaaacatggcattcgttggttacatcgaatctgtttcaaatttgcatacagctgaaatcagatgtatctggttgtcaagattagttgaaaactacttcaaattgccatctttagaaaacatgttggaacaaactcaaaaggaaatggaaatcatgagaagaactacaagatttttcaaaagatcttgtatctcaacattttctattaatcattctgatgaaatttgtgaagaaatgggttggaaatcatggaggaaaaattcttggttggcagaagcattttctccatacaactctcaagattaccaagaaaagaaagttaattaa

SEQ ID NO: 287 - DNA, 990 bp, synthetic construct
  source 1..990: mol_type = other DNA
atggcagcaatgtcaaaggttttgtctttgtttttcttgcaattcttgtgtgttttgtgtatttcagatttgtctaacggtcaaggtttggatgttaacttctacaagaaaacttgtccatctttagaagatattgttaaggatactacagcaagattcatttctagagcaccatctttggctgcaccattgttaagaatgcatttccatgattgttttgttagaggttgtgatggttcagttttgttaaattctacttcttcaaaccaatcagaaaaggctgcaatcccaaaccaatctttaagaggtttccaagttattgatgcagttaaatcagaattggaaaagaaatgtccaggtgttgtttcttgtgctgatattgttgctttagttgcaagagatgctattacattgattaaaggtccatcatggcaagttgaattaggtagaagagatggtagagtttcaatcgcttctcaagttttgaataagttgccatctccttttatgaacatcgcacaattgaaagcatcttttcaatctttgggtttatcagttaaggatttggcagttttgtctggtggtcatactattggtatttcacattgtgcttctgttaacaacagattatacaacttcactggtaaaggtgacacagatccaactttagatccaaagtacgttgcaactttgaaaagaaagtgtcaaccaggtgacgttactacaattttggaaatggacccaggttctgctaaaacattcgatactgattacttcactacagtttcaaaaagaagaggtttatttcaatctgatgctgcattgttagatgatactgaaacaaatacttacgttcaacaacatttgtcatcttctggttcaggttctttctttgatgattttggtgttccaatggttaaaatgggtagaattggtgttttgacaggtaaccaaggtgaaattagaaaacaatgtgctttggttaattaa

SEQ ID NO: 288 - DNA, 990 bp, synthetic construct
  source 1..990: mol_type = other DNA
atggcagcatttccaagacatttggctgtttttatgttgttgcaaatcatcttgtttccaacagcatttcaacattgtaacgctcaaggtttgaaattaggtttctacaagaaaacttgtccaggtgctgaagcaacagttaagcaaatcatgactggtgttatttctagagcaccaacattggctgcaccattgttaagaatgcatttccatgattgttttgttagaggttgtgatggttctgttttgttaaattcaactaaaaataataagtctgaaaaggaagctatcccaaaccaatcattgagaggtttccaaatcatcgataacatcaagtctgctttggaaaagaaatgtccaggtgttgtttcatgtgctgatattttggctttagttgcaagagatgctgtttctttgttaaatggtccattttggattgttccattaggtagaagagatggtaaagtttcaatcatgaacgaagcattgacaaatttgccaccaccattcgctaacatcactcaattgaaagcatttttctctgctaagggtttaaatacaaaggatttggcagttttatcaggtggtcatactattggtacagctcattgtttgggttttactaacagattatacaactttactggtaaaggtgacactgatccatctttagatccaaactacatcccattgttgaaaagaaagtgtaagccaggtgacgttactacattagttgaaatgactccaggtacttttaaaactttcgatgaagattactacacattgattactaaaagaagaggtttatttcaatctgatgctgcattgttagatgattcacaaacaagagcatacgttaaattgcaagctgcaactcatggttctactttctttaaggatttcgctgtttcaatggttaagatgggtaaagttggtgttttgactggtaaagcaggtgaaattaggagacattgtgctttagttaattaa

SEQ ID NO: 289 - DNA, 951 bp, synthetic construct
  source 1..951: mol_type = other DNA
atggcagcttcttcaattgttccaactattgatttggacccatttttgagagaaggtgacgaagaagctaagaaaaatgcaatcgaagaaatctgtcaagcatgttctgaatacggtttctttcaagttgttaatcatggtgttccattggatttgatgactagagcaacagatatgttcactgaatttttcaattatccaatcgaagaaaagtttaaatctatttcaaagccaggtgtttctccaccaccaggttacttcaactctccagatcattcaaaggagggtaacgaacatttcttgatcttgacaccatcattgggtttaaatgtttacccagaaaatccaccagagtttagaaaggttttggaagaaatcttcccaaatttggctaaattagcattgttaatcgaaagaattgtttctgtttcattgggtttagcaccaaatttcttgaaagaatacaacaacgatagatcttgtgatttcttgatgggtttacattactttccagctgaagaaactgaaaataatggtaaatcagcacatgaagatggtaactgtatcacattcgtttttcaagatgaagttggtggtttggaagttttgaaaaatggtcaatggattccaatcactccagctcaaggtacattggttgttaacatcggtgacgttgttcaagttttatctaacaataagtttaaatcagcaacacatagagttgttagaccaaagggtaaatctagatactcatatgttttctttcataatttgcatggtgacaagtgggttgaaccattaccacaattcactaaggatattggtgaatctgctaagtacagaggtttcgtttacaaggaatacatgcaacatagaattagaaatagaacacatccaccagcaagaccagaagatttgatccatatcactcattactctatttcaacataa

SEQ ID NO: 290 - DNA, 954 bp, synthetic construct
  source 1..954: mol_type = other DNA
atggcaccattgttacaaatgccaactgttgatttgtcatctttctttacaggtgacgatgttggtcaaaagaatgttttgtctaaaatttcacaagcatgttctgattacggtttctttcaaatcgttaatcatggtttgtgtttggatttgatgaacagagctttggaaatttctagagttttctttgaatcaccaactgaagaaaaattgaaatgttctccaagaccaggtgcttcaccaccagcaggttacaacagaaagccaaacccagaatacgaattttctgaatacttcatcatgtttccaccaggttcagatttcaacagatttccaccaaatttgccacaattgagagaagttatggaagaattattttctcaatttttgaaaactgcttcagttatttcttcaatcttgatcgaatctttaggtttgccacaatcaatcgatgaaggttttaatttcgcagatagatcttcagatttgttgattgctttattttatttgtctgcaaaggaaaaggaaaacatgggtgttaacccacataaggatgtttcttgtttcacaatggttttgcaagatgaagttggtggtttgggtgttgaaaaagatggtgaatggttaccattgggtccaactgaaggtgctttgatcgttaacatcgcagatgttttgcaagttttgacaaacgataagtttaaatctccaaatcatagagttttaagaccaaaagctagatcaagatcttcatttgctttctttcatgatttgccaggtaaaacttggttagaaccattgccagagtttactagagaaattggtgaattaccaaagtacagaaagttcttgtacaaggattacaaacaaatgagagttagaaataagtctaacccacattcaagattagaagatgaaatcacaatttctcattacgctatcgcatcaaaataa

SEQ ID NO: 291 - DNA, 1506 bp, synthetic construct
  source 1..1506: mol_type = other DNA
atggcagctttgccattgttaatttttgcttcagtttctttgattgttgttttgaaatttgtttcaaaaccaaagaataatagaaattctaaattgccaccatgtccaccaaaattgccaattattggtaatttgcatttgttaggtaatatgccacaagaatctttgcatcaattggctaaaaagtacggtccagttattttcttgcaattaggtgaagttccaacaattgttatttcttcagcaagattggctaaagaagcaatgaaaacttttgatttggctttatcttcaagaccacaaattttctcagcaaaacatttgttttacaattgtacagatgttgttttctctccatatggtgcttactggagaaatattagaaaaatttgtattttagaattgttgtctgctaaaagagttcaatcattttcttatgttagagaagaagaagttgctcatttggttcaaagagttgctgaagcatatccaggtacaactaatttgacaaaaatgttaggtttatatgctaataatttggtttgtagagttgcttttggtaaagatttttctcaaggtggtgacaatcatagacataattttcaaaaattattggaagaataccaagttttgttaggtggtttttctattggtgactttttcccatctatggaatttgttcataaattcactggtatggaatctagattaaaagatacttttagaagatttgatcaatttttcgatgaaattttacaagaacatagaaatccagaatctagaaaagaacataaagatttggttgatgttttattggaaattcaaaagaatggtgacccagaaactccattgacaactgataatattaaatctattattttggatatgtttgctgcaggtacagatactaattttattactttagattgggctatgactgaattgatgactaatccaggtgttttgaaaagagcacaagctgaagttagaaatattgttggtgaaagaagatcagtttctgaaaatgatttgccacaattgccatatgttaaagcagttgttaaagaaacttttagattgcatccaccagcaccagttttagttccaagagaatctatggaagatgttactattgatggttacgatattccagctaaaacaagatttttcattaatgcatgggctattggtagagatccaaaatcatgggaaaatccagatgtttttgaaccagaaagattcatgaactcacatattgattttaaaggtcaagattatgaattgattccatttggtgctggtagaagatcttgtccagcaattactttttctgctgttactgttgaattagctttggcacaattgttacattcttttgattgggaattaccaccaggtgttgaagctaaagatttggatatgactgaagtttttggtattactatgcataaaatttacccattgatggttgttgctaaaccatacttttcacaaggtttgaattaa

SEQ ID NO: 292 - DNA, 1506 bp, synthetic construct
  source 1..1506: mol_type = other DNA
atggcagcattaccattgtttatttttgcatctatttctttggttccagttttggttattttattgatgaaaaaggataaaagatcagttaaattgccaccatctccaccaaaattgccaattattggtaatttgcatcaaattggtaatatgccacatgaatcatttcataatttggctaaaaagtttggtccaattatgtttttgtggttaggtcaagttccaactgttgttatttcttcagcaagattggctaaagaagcattgaaaacatctgatttggcattgtcatctagaccagaattgttttctgctaaacatttgcattacaattgtacagatttggcattttctccatacggtacttattggagaaatattagaaaaattacaactttacaattgttatctgctaaaagagttcaatcattttcttttgttagaaaagatgaagttgcacatttggttaaaagaattactgaatcttatccatcaactattaatttgtctcatatgttggaagtttatgctaataataatatttgtagagttgcttttggtaaagatttttctcaaggtggtgacaatgttagacataattttcaaaaagttttggaagaataccaaatgttgttaggtggtttttcaattggtgactactttccatctatggaatttttgcataaattcactggtatggaatcaagattgaaagatacttttagaagatttgatcatatttgtgataaaattttggaagaacatagaaaaacagaaaatagaaaagatcaaaaagatttggttgatgttttattggaaattcaacataatggtgacttggaaacaccattaactacagataatattaaagctattattattgatatgtttgttggtggtactgatacaaattttattacattagattggacaatgactgaattgattattaatccaaaagttatgcaaagagcacaagctgaagttagatctattgttggtgaaagaagatttgtttcagaaaatgatttgtctcatttgggttacatgaaagctgttgttaaagaaacttttagattgcatccaccagctccattattggcaccaagagaatctattcaagatgttacaattgatggttacaatattccagctaaaactagatttttcattaatgcatgggctattggtagagattctgaattgtgggaaaatccagaaatgtttgaaccagaaagattcatgaactcacatattgattttaaaggtcaagattttgaattgattccatttggtgcaggtagaagaatttgtccagctatttcttttactgttgctactgttgaattagctttggctcaattattgcatggttttgattgggaattgccaccaggtgttgaagctaaagatttggatatgactgaagcatttggtattacaatgcatagaacttctccattgatggttgttgcaaaaccatatttttctgatcatttgaattaa

SEQ ID NO: 293 - DNA, 1506 bp, synthetic construct
  source 1..1506: mol_type = other DNA
atggcagcattaccattgtttatttttgcatcaatttcattggttttagttttggttattttatcaatgaaaaaggataaaagatctgttaaattaccaccatcaccaccaaaattgccaattattggtaatttgcatcaaattggtaatatgccacatgaatcttttcataatttggctaaaaagtttggtccaattatgtttttatggttgggtcaagttccaactgttgttatttcatctgctagattggctaaagaagcattgaaaacatttgatttggcattatctaatagaccagaattgttttcagctaaacatttgcattataattgtactgatttggcattttctccatatggtacttactggagaaatattagaaaaattacaactttacaattgttatctgctaaaagagttcaatcattttcttttgttagaaaagatgaagttgttcatttggttaaaagaattactgaatcttacccatctactattaatttgtctcatgttttggaagtttacgctaataataatatttgtccagttgcttttggtaaagatttttctcaaggtggtgacaatgttagacataattttcaaaaagttttagaagaataccaaatgttgttaggtggtttttctattggtgactactttccatctatggaatttttgcataaattcactggtatggaatcaagattaaaagatactttgagaagatttgatcatatttgtgataaaattttggaagaacatagaaaaactgaaaatagaaaagatcaaaaagatttggttgatgttttgttagaaattcaacataatggtgacttggaaacaccattgactacagataatattaaagctattattattgatatgtttgttggtggtactgatacaaattctattactttggattggacaatgactgaattgattatgaatccaaaagttatgcaaagagcacaagctgaagttagatcaattgttggtgaaagaagatttgtttctgaaaatgatttgtctcatttgggttacatgaaagctgttgttaaagaaacttttagattacatccaccagcaccattattggctccaagagaatctatgcaagatgttactattgatggttataatattccagctaaaacaagatttttcattaatgcatgggctattggtagagattctgaattgtgggaaaatccagaaatgtttgaaccagaaagattcatgaactctcatattgattttaaaggtcaagattttgaattgattccatttggtgctggtagaagaatttgtccagctatttcttttactgttgctacagttgaattggctttagcacaattgttacattcttttgattgggaattgccaccaggtgttgaagcaaaagatttggatatgacagaagcatttggtattactatgcatagaacttctccattgatggttgttgcaaaaccatacttttctgatcatatgaattaa

SEQ ID NO: 294 - DNA, 1494 bp, synthetic construct
  source 1..1494: mol_type = other DNA
atggcagctttaccattgtttatttttgcatctatttcattggttccagttttggttattttattgatgaaaaaggataaaagatctgttaaattgccaccatctccaccaaaattgccaattattggtaatttgcatcaaattggtaatatgccacatgaatcttttcataatttggctaaaaagtttggtccaattatgtttttgtggttaggtcaagttccaacagttgttatttcatctgcaagattggctaaagaagcattgaaaacttctgatttggctttatcatctagaccagaattgttttctgcaaaacatttgcattacaattgtacagatttggcattttctccatacggtacttattggagaaatattagaaaaattacaactttgcaattattgtctgctaaaagagttcaatcattttcttttgttagaaaagatgaagttgctcatttggttaaaagaattactgaatcttacccatcaacaattaatttgtcacatatgttggaagtttacgctaataataatatttgtagagttgcttttggtaaagatttttctcaaggtggtgacaatgttagacataattttcaaaaagttttggaagaataccaaatgttgttaggtggtttttctattggtgactactttccatcaatggaatttttgcataaattcacaggtatggaatctagattgaaagatacttttagaagatttgatcatatttgtgataaaattttagaagaacatagaaaaacagaaaatagaaaagatcaaaaagatttggttgatgttttattggaaattcaacataatggtgacttggaaactccattgacaactgataatattaaagatatgtttgttggtggtactgatacaaattttattactttggattggacaatgactgaattaattattaatccaaaagttatgcaaagagcacaagctgaagttagatcaattgttggtgaaagaagatttgtttctgaaaatgatttgtctcatttgggttacatgaaagctgttgttaaagaaacttttagattgcatccaccagctccattattggctccaagagaatctattcaagatgttactattgatggttataatattccagctaaaactagatttttcattaatgcatgggctattggtagagattctgaattgtgggaaaatccagaaatgtttgaaccagaaagattcatgaactctcatattgattttaaaggtcaagattttgaattgattccatttggtgctggtagaagaatttgtccagctatttcttttactgttgctacagttgaattggctttagcacaattattgcatggttttgattgggaattgccaccaggtgttgaagcaaaagatttggatatgactgaagcatttggtattactatgcatagaacttctccattgatggttgttgctaaaccatatttttctgatcatttgaattaa

SEQ ID NO: 295 - DNA, 1506 bp, synthetic construct
  source 1..1506: mol_type = other DNA
atggcagctttgccattattgattttctcatctttgtttttagctttgttattgaaattattgttaaaacataaaaataatagaaattctaaattgccaccatgtccaccaaaattgccaattattggtaatttgcatcaaattggtaatatgccacatgaatcttttaataatttggctaaaaagtttggtccaattatgtttttgtggttaggtcaagttccaactgttgttatttcatctgcaagattggctaaagaagcattgaaaacttttgatttggcattatcttcaagaccagaattgttttcagcaaaacatttgcattacaattgtacagatttggctttttctccatatggtacatactggagaaatattagaaaaattactacattgcaattattgtctgctaaaagagttcaatctttttcttttgttagaaaagatgaagttgcacatttggttaaaagaattactgaatcatacccatctactattaatttgtcacatatgttagaagtttacgctaataataatatttgtagagttgcttttggtaaagatttttctcaaggtggtgacaatgttagacataattttcaaaaagttttggaagaatatcaaatgttattgggtggtttttcaattggtgactactttccatctatggaatttttgcataaattcacaggtatggaatctagattgaaagatacttttagatcttttgatcatatttgtgataaaattttggaagaacatagaaaaactgaaaatagaaaagatcaaaaagatttggttgatgttttgttggaaattcaacataatggtgacttggaaactccattgacaactgataatattaaagctattattattgatatgtttgttggtggtactgatactaatttgattacattagattggacaatgactgaattgattattaatccaaaagttatgcaaagagcacaagctgaagttagatctattgttggtgaaagaagatttgtttctgaaaatgatttgtctcatttgggttacatgaaagctgttgttaaagaaacttttagattgcatccaccagcaccattattggttccaagagaatcaattcaagatgttactattgatggttattacattccagcaaaaacaagatttttcattaatgcttgggctattggtagagatccagaattgtgggaaaatccagaaatgtttgatccagaaagattcatgaactctcatattgattttaaaggtcaagattttgaattgattccatttggtgcaggtagaagaatttgtccagctattacattttcaattgctaatgttgaattagctttggcacaattattgcattcatttgattgggaattaccaccaggtgttgaagctaaagatttggatatgacagaagcatttggtattacaatgcatagaatttctccattgatggttgttgctaaaccatacttgtcagattacttgaattaa

SEQ ID NO: 296 - DNA, 1494 bp, synthetic construct
  source 1..1494: mol_type = other DNA
atggcagcattaccattgtttatttttgcttcaatttctttggttttagttttggttattttgtctatgaaaaaggataaaagatctgttaaattgccaccatctccaccaaaattgccaattattggtaatttgcatcaaattggtaatatgccacatgaatcatttcataatttggctaaaaagtttggtccaattatgtttttgtggttgggtcaagttccaactgttgttatttcttcagcaagattagctaaagaagcattgaaaacttttgatttggctttgtctaatagaccagaattattttctgcaaaacatttgcattacaattgtacagatctggctttttctccatacggtacttattggagaaatattagaaaaattactacattacaattgttgtcagcaaaaagagttcaatctttttcatttgttagaaaagatgaagttgttcatttggttaaaagaattactgaatcttacccatcaactattaatttgtcacatgttttggaagtttacgctaataataatatttgtccagttgcatttggtaaagatttttctcaaggtggtgacaatgttagacataattttcaaaaagttttggaagaatatcaaatgttattgggtggtttttcaattggtgactattttccatctatggaatttttacataaattcactggtatggaatcaagattgaaagatacattaagaagatttgatcatatttgtgataaaattttggaagaacatagaaaaactgaaaatagaaaagatcaaaaagatttggttgatgttttgttagaaattcaacataatggtgacttggaaactccattaacaactgataatattaaagatatgtttgttggtggtacagatactaattctattactttagattggactatgacagaattgattatgaatccaaaagttatgcaaagagcacaagctgaagttagatcaattgttggtgaaagaagatttgtttctgaaaatgatttgtctcatttgggttacatgaaagctgttgttaaagaaacttttagattgcatccaccagctccattattggcaccaagagaatctatgcaagatgttactattgatggttacaatattccagctaaaactagatttttcattaatgcttgggcaattggtagagattcagaattgtgggaaaatccagaaatgtttgaaccagaaagattcatgaactcacatattgattttaaaggtcaagattttgaattgattccatttggtgcaggtagaagaatttgtccagctatttcttttacagttgcaacagttgaattggctttagctcaattgttacattcttttgattgggaattgccaccaggtgttgaagctaaagatttggatatgacagaagcatttggtattactatgcatagaacatctccattaatggttgttgcaaaaccatacttttcagatcatatgaattaa

SEQ ID NO: 297 - DNA, 1506 bp, synthetic construct
  source 1..1506: mol_type = other DNA
atggcagcattgccattattgatttttgcttcattgtctttgattgttgttttgaaatttttattgaaaccaaagaataatagaaattctaaattgccaccatgtccaccaaaattgccaattattggtaatttgcatttgttaggtaatatgccacaagaatctttgcatcaattggctaaaaagtatggtccaattattttcttgcaattaggtgaagttccaacagttgttatttcttcagctagattggctaaagaagctatgaaaacttttgatttggctttatcttcaagaccacaaattttctctgctaaacatttgttttataattgtactgatgttgttttctcaccatacggtgcttattggagaaatattagaaaaatttgtattttggaattattgtcagcaaaaagagttcaatcattttcttacgttagagaagaagaagttgctcatttggttcaaagagttgctgaagcatatccaggtacaactaatttgactaaaatgttgggtttatacgctaataatttggtttgtagagttgcatttggtaaagatttttctcaaggtggtgacaatcatagacataattttcaaaaattattggaagaatatcaagttttgttaggtggtttttcaattggtgactttttcccatcaatggaatttgttcataaattcactggtatggaatctagattgaaagatacttttagaagatttgatcaatttttcgatgaaattttgcaagaacatagaaatccagaatctagaaaagaacataaagatttggttgatgttttgttagaaattcaaaagaatggtgacccagaaactccattgactacagataatattaaagctattattttagatatgtttgctgctggtactgatacaaattttattactttggattgggctatgactgaattgatgactaatccaggtgttttgaaaagagcacaagcagaagttagaaatattgttggtgaaagaagatcagtttctgaaaatgatttgccacaattaccatatgttaaagcagttgttaaagaaacttttagattgcatccaccagctccagttttggttccaagagaatctatggaagatgttactattgatggttacgatattccagctaaaactagatttttcattaatgcttgggcaattggtagagatccaaaatcttgggaaaatccagatgtttttgaaccagaaagattcatgaactctcatattgattttaaaggtcaagattacgaattgattccatttggtgctggtagaagatcttgtccagctattactttttctgcagttactgttgaattagcattggctcaattgttacattcttttgattgggaattaccaccaggtgttgaagctaaagatttggatatgactgaagtttttggtattactatgcataaaatttatccattaatggttgttgctaaaccatatttttcacaaggtttaaattaa

SEQ ID NO: 298 - DNA, 1554 bp, synthetic construct
  source 1..1554: mol_type = other DNA
atggcagcattaattgaagatcattctacacaagcatcagtttttattttaatggaattgtttttaactttgtgtgctttattggcatttttctcaattgctttgaaaattgttagaaaaactaaattggttaaattaccaccaggtccaagaccattaccaattattggtaatttgcatcaattaaatggttctttgccacatagaattttgactgatttggctaaaaagtacggtccattgatgcatttgcaattaggtgaagtttctactgttattgtttcatctccagcagttgctaaagaagttatgaaagatcatgatattattttctgttacagaccattgttattgtctccagcaattttgaattacggtggtactgatattgcatttgctccatacggtgaatattggagacaattaagaaaaatttgtactattgaattgttatcatctaaaagagttcaaacttttagatctattagagaacaagaagtttctaatttgtttagatctattaaatcagcagaaggttctgttgttaatttgtctaaaatgatttattctttgacatactctattactgctcaagctgcatttggtaaagaatcaaaataccaagataaattcatttctgttatgcaagatgctattaaattgatgggtggttttaatattgctgatgtttacccatctattaaaattttagaaaagattacaggtattagacataaattagaagaaacacataatcaaattgatgaaattgttcaaaatattttgattgaacataaagcaaaaagagctgaatctaaattaggtaatagaggtgctaaagaagatttggttgatgttttattgaatattcaatcatctggtgaatttgaaccaccattgactgatgataatttgaaagctgttatttacgatattttctctgcaggtggtgaaacatcttcaactacagttatttgggctatgtctgaaatgattagaaatccaaaagttatgaaaaaggctcaagatgaagttagagaagtttttgctggtaatggtaatgttgatgaatctagattgcatgaattaaaatatttgcaagctgttattaaagaaactatgagattaaatccagctgttccattattgttaccaagagaatgttcagaacattgtcaaattaatggttacgatgttccagctaaaacaagagttattgttaatgcatgggctattggtagagatccaaaacaatggactgaaccagaaaaattcaaaccagatagatttttggattctgaaattgattttagaggtaaaactttttcttttattccatttggtgcaggtagaagaatttgtccaggtatttcttttgctttgccattagttgaattaccattggcacaattattgtaccattttgattggaaattgccaggtcaatctaaaccagatgaattggatatgactgaaatttttggtttgactgttggtagaaaacatgatttgaatttggttccaattccatatagaccatgttcagttttgtaa

SEQ ID NO: 299 - DNA, 1524 bp, synthetic construct
  source 1..1524: mol_type = other DNA
atggcagctttgtttgaatgggaaaaagaaggttttccatatccatacttgttaatttttgcttcattatctttgattgttgttttgaaatttttgttaaaaccaaagaataatagaaattctaaattgccaccatgtccaccaaaattgccaattattggtaatttgcatttgttgggtaatatgccacaagaatctttgcatcaattggctaaaaagtacggtccaattattttcttacaattgggtgaagttccaactgttgttatttcttcagctagattagctaaagaagcaatgaaaacttttgatttggcattatcttcaagaccacaaattttctctgctaaacatttgttttacaattgtactgatgttgttttctcaccatatggtgcttactggagaaatattagaaaaatttgtattttagaattgttgtcagctaaaagagttcaatctttttcatacgttagagaagaagaagttgcacatttggttcaaagagttgcagaagcatacccaggtactacaaatttgactaaaatgttgggtttatacgcaaataatttggtttgtagagttgcttttggtaaagatttttctcaaggtggtgacaatcatagacataattttcaaaaattgttagaagaataccaagttttgttaggtggtttttctattggtgactttttcccatctatggaatttgttcataaattcactggtatggaatcaagattgaaagatacttttagaagatttgatcaatttttcgatgaaattttgcaagaacatagaaatccagaatctagaaaagaacataaagatttggttgatgttttattggaaattcaaaagaatggtgacccagaaactccattgactacagataatattaaagctattattttggatatgtttgcagctggtactgatactaattttattactttggattgggcaatgactgaattgatgactaatccaggtgttttgaaaagagcacaagctgaagttagaaatattgttggtgaaagaagatcagtttctgaaaatgatttgccacaattgccatacgttaaagcagttgttaaagaaacttttagattgcatccaccagcaccagttttggttccaagagaatcaatggaagatgttactattgatggttacgatattccagctaaaacaagatttttcattaatgcatgggcaattggtagagatccaaaatcttgggaaaatccagatgtttttgaaccagaaagattcatgaactctcatattgattttaaaggtcaagattacgaattaattccatttggtgcaggtagaagatcttgtccagctattactttttctgctgttactgttgaattggcattggctcaattattgcattcttttgattgggaattgccaccaggtgttgaagcaaaagatttggatatgacagaagtttttggtattacaatgcataaaatttacccattgatggttgttgctaaaccatatttttca

SEQ ID NO: 300 - DNA, 963 bp, synthetic construct
  source 1..963: mol_type = other DNA
atggcagctttgattgaagatcattctacacaagcatctgtttttattttgatggaattgtttttaactttgtgtgctttattggcatttttctcaattgctttgaaaattgttagaaaaacaaaattagttaaattaccaccaggtccaagaccattgccaattattggtaatttgcatcaattaaatggttctttgccacatagaattttgactgatttggctaaaaagtacggtccattaatgcatttgcaattaggtgaagtttctactgttattgtttcttcaccagctgttgcaaaagaagttatgaaagatcatgatattattttctgttacagaccattgttattgtctccagctattttgaattacggtggtactgatattgcttttgcaccatacggtgaatattggagacaattaagaaaaatttgtacaattgaattgttatcttcaaaaagagttcaaacttttagatcaattagagaacaagaagtttctaatttgtttagatctattaaatctgctgaaggttctgttgttaatttgtctaaaatgatttactctttgacttattctattactgctcaagcagcttttggtaaagaatctaaataccaagataaattcatttctgttatgcaagatgctattaaattgatgggtggttttaatattgctgatgtttacccatctattaaaattttagaaaagattacaggtattagacataaattggaagaaacacataatcaaattgatgaaattgttcaaaatattttaattgaacataaagctaaaagagcagaatctaaattgggtaatagaggtgctaaagaagatttggttgatgttttattgaatattcaatcatctggtgaatttgaaccaccattgactgatgataatttgaaagctgttatttacgtttcattgttttactcaccatctcatattaaa

SEQ ID NO: 301 - DNA, 1395 bp, synthetic construct
  source 1..1395: mol_type = other DNA
atggcagctttgttagatccgtggcaaaaagaatcatttttgcatccattcatttttgcttctatttcattggttccagttttggttattttattgatgaaaaaggataaaagatctgttaaattgccaccatctccaccaaaattgccaattattggtaatttgcatcaaattggtaatatgccacatgaatcttttcataatttggctaaaaagtttggtccaattatgtttttgtggttaggtcaagttccaactgttgttatttcatcagctagattagcaaaagaagcattgaaaacttctgatttggctttatcatctagaccagaattgttttctgctaaacatctgcattacaattgtacagatttggctttttcaccatatggtacatattggagaaatattagaaaaattacaacattgcaattattgtctgctaaaagagttcaatctttttcatttgttagaaaagatgaagttgctcatttggttaaaagaattactgaatcatatccatcaactattaatttgtcacatatgttggaagtttatgcaaataataatatttgtagagttgcatttggtaaagatttttcacaaggtggtgacaatgttagacataattttcaaaaagttttagaagaatatcaaatgttgttaggtggtttttctattggtgactattttccatctatggaatttttacataaattcactggtatggaatctagattaaaagatacttttagaagatttgatcatatttgtgataaaattttagaagaacatagaaaaactgaaaatagaaaagatcaaaaagatttggttgatgttttattggaaattcaacataatggtgacttggaaacaccattgactacagataatattaaagctattattattgatatgtttgttggtggtactgatactaattttattactttggattggactatgacagaattgattattaatccaaaagttatgcaaagagcacaagctgaagttagatctattgttggtgaaagaagatttgtttctgaaaatgatttgtctcatttgggttatatgaaagctgttgttaaagaaacttttagattgcatccaccagctccattattggctccaagagaatctattcaagatgttactattgatggttataatattccatttggtgctggtagaagaatttgtccagctatttcttttacagttgctactgttgaattggcattagctcaattgttgcattcttttgattgggaattgccaccaggtgttgaagcaaaagatttggatatgactgaagcatttggtattactatgcatagaacttctccattaatggttgttgctaaaccatacttttctgatcatttgaat

SEQ ID NO: 302 - DNA, 1608 bp, synthetic construct
  source 1..1608: mol_type = other DNA
atggcagctaatgttaaagctaataagcataatattgtttgtattttgtgtttcttatttttgacaatggctaaacaaatttttgttttagatttgccattttcatctttgtttccattggctttgttattgttgactttagttatttggttttacgcttcttcaagaccacaaaagagattgccaccatcaccaccaaaattgccaattgttggtcatttgtttagattgggtatttatccacatagaactttgcaaagattatctaaaaagtacggtccattgatgatgattcatttgggttctaaaccaattttggttgcttcttcagcagatgttgcttgtgaaattatgaaaaatcatgatttggtttttgcaaatagaccaaaaagatctattaattcaaaattattgtacgattgtaaagatattgcaactgctcaatatggtgaatactggagacaaattagatctatttgtgttttgcaattattgtcaaataagagagttcaatcttttagacatgttagagaagaagaaacttcaatgatgattcaaaagattagagaaagatgttcatcttcttcttcttcattgattaatttgtcagatgctttgattactttgacaaatgatgttatttgtagagttgctttgggtagaaaatattcaaatgatgaaaatggtagaaaatctatgaaaattatgaaagaatttggtgaattgttaggtactttttctgttggtgactacttgccgtggttagcttgggttaatagattcaatggtttggatgctaaattagaaaaagttgctaaacatattgatcaatttttggaaggtgttattgaagaacataaattcagaaagaaaggtgaagattacgttgattctactgaaaagaaagcattggatttggttgatattttattggatattcaaaaagataattctgttggtttagctttggaattagattctgttaaagcaattattttggatatgtttgcaggtggtactgatacatcatacactgcaatggaatgggcaatgtctgaattattgagagatccaaaaactttggaaaaattacaagttgaagttagaaaagttgctcaaggtaaatctgaaattactgaagatgatttggataaaatgcattacttaaaagcagttattaaagaaactttgagattacatactccaattccattgttagttccaagagaatcaactcaacatgttaaaattttaggttatgatattccagctggtactgaagttatggttaatgcatgggctattgctagagatccagttcaatggaaaaagccagaagaatttttgccagaaagatttttaaattctactattgattttagaggttttaattttgaatttattccatttggtgctggtagaagaggttgtccaggtgcaacttttgctattgctgttaatgaattggctttggcaaaattgttacatgaatttgattttgcattgccagatggtgctaaaccagaagatttggatatgtctgatgctacaggttttactgttcatagaaaatttccattgttagcagttgctattccacattcttgt

SEQ ID NO: 303 - DNA, 1617 bp, synthetic construct
  source 1..1617: mol_type = other DNA
atggcagctgttagaattttgaaaaagaaaaagtttcaattcacaattttgaatactaaaaaggaaatgtctatttttcaatttgaagaaattacagctggtttatctaaacattttccatctttgttgttattgttattgatttctttattgttgttgagatggaaaaattctacatcagatacaactcaaaatttgccaccatctccaccacaattaccaattattggtaatttgcatcaattgggttctattccacaaagatctttgaaatcattggcacaaaaatacggttctttgatgttattgagattgggttctaaaccagttttgattatttcttcagcagatgctgctgaagaagttttgaaaactcatgatttgatttttgcaaatagaccaaaaccagattttgttggtagattattgtacaattttaaagatattgctttttctccatacggtggttattggagacatattagatctatttgtgttttgcaattattgactaataagagagttcaatcttttagaaatattagagaagaagaagttgaattaatgttggaaaagattagagaatcatgtgcttcttcatctattattttgattggtgaaattttggctactttgacaaataatattgtttctagagttgcaactggtaaaagatactctgaagaagaatctggttcaaaattcaaagatttgtttgaagaattttcaatgttgttaggtgtttttaatgttggtgactacattccgtggttggcatggattaatcatattaatggtttggaagcaaaagttaaaagagttggtaaagattttgatgaatacatggataaaattgttcaagaaggtattattagacaagaaaagagaggttctaatggtggtgaccatgaaaaagaacaacaaaatttggttgatgttttgttagaattgcaaagaactaattctgctggttttgctattggtagagattctattaaagctattattacaaatatgtttgttggtggtactgatacaactttttctttattggaatgggctatttctgaattattgagaaatacaaatgctatgcataaattgcaaaaagaagttagacaatttttgggtagaaaatctagaattactgaagatgatttggaaaatttgcattacttgaaagcagttattaaagaagcattgagattgcatccaccagctccattgttgattccaagagaatcatctaaagctgttaaattaatgggttacaatattgctgcaggtacacaagttattattaatgcatgggcaattggtagagatccaaaattgtgggatgatgctggtgaatttagaccagaaagatttttaaattcatctgttgatattaatggtcaacattttgaattaattccatttggttcaggtagaagatcttgtccaggtactgcatttgctatgattactgctgaattggcattagctaatttgatttgtaattttgattttgaattggcaggtggtgctagaccagaagatttggatatgacagaagcaccaggtattattactccaagaaaagttccattaagattggttgcttctttgccaaat

SEQ ID NO: 304 - DNA, 1545 bp, synthetic construct
  source 1..1545: mol_type = other DNA
atggcagtttcttttccagcttttcaagatgaagctaaaagatggttgtctaatcattctgttgctgtttctttattattgccatcttttatgatttttattttgtttttatctaaatggtggtttttgacaacttctaaaaagaaaaatttgccaccatcaccaccaagattgccaattattggtaatttgcatcaaattggttctgttgcacatagatcttttaaatctttggctgaaaaacatggtccattgatgttgttacatgttggtagacaaaaattgttagttgtttcttcagctgaaactgcaagagaagttttgaaaacacatgatgttgcattttgtggtagaccagattctgaagttacaagaagaattttctataatttgaaaaatatttctttgtcaccatacggtgactactggaaaattgttagatctattgctgttaatcaaattttgaatcaaaagagagttcaatcttttagatctgttagagaagaagaaatttctttgttagttgaaagaattaaagaatcttgtgcatcatcttctgttatttgtatgaatacattattgacaactttagttaatgatattgtttctagaattacaattggtaaaagatcatctggtaaaggtggttctagatttgctgaattgttatttgcatctacagaattgtttggttctttcttagctggtgactttttcccaggtttgggttggttaggttatattactggttttgaagctaaaactaaaaagattgcaaaagatttggatcaatgtttagaaaatttgattgaagaacaaattaatagaaataagagaggtgacaatcaaggtaaagataatcaaaatttcttgcaagttttattggaaattcaaagaactgattcatctggtcatgctttggatagagaatctattaaagctattattatggatatggttgctggtggttttgatacatactctttattggaatgggctacatctttattggttaaacatccagatgttatgaaaaagttgcaaaatgaagttaaagaagttgctggttcaaaatcttttatttcagaagatgatttgtctaaattacaatacttaaaagctgttattaaagaaacttttagattgtgtccaggtgcttttattgttagagtttctacaaaagatgttaaaattatgggttatgatattgctgcaggtacacaagttttggttaatgcattggctattggtagagatccaactttatgggaaaaaccagaagaatttagaccagaaagatttttaaattcttcaattgatctgaaaggtcatcattttgaattgttaccatttggttctggtagaagatcatgtccaggttcaacatttgctttggttattgatgaattggttttagcaaatttggtttgtaattttaattatgctttgccaggtggtgcaaaagctgaagatttggatatgacagaagctccaggtgctatgccaagaagaagaactccattgttattggttccatctttatgt

SEQ ID NO: 305 - DNA, 1536 bp, synthetic construct
  source 1..1536: mol_type = other DNA
atggcagcccttttcgaatgggagaaagaaggattccccagcccatatctattgatctttgcgtccgtctccttaatcgtcgtcctaaagttcgtatcaaaaccaaagaacaatcgtaactcaaaattacctccgtgtcctcctaagttaccgatcattgggaacttacacttgctagggaacatgccacaggagagtctacatcagttagcaaagaaatacgggcccgttatcttcttgcaattgggcgaagtgccgacaatagtcatcagcagtgcaagattagcgaaagaggccatgaagacattcgatctggccctatcaagcagaccacaaatattttccgccaagcaccttttctataattgcactgacgtggtgtttagtccgtacggagcttattggcgtaatataagaaaaatatgtatattggagcttttatctgccaagagagttcaaagtttctcctatgtacgtgaagaggaggtagctcaccttgtgcaacgtgtagctgaagcctaccctggtaccaccaatctgaccaagatgttggggctatatgctaataacctagtgtgcagagtcgcgtttggaaaagacttctcacaagggggagacaatcataggcacaacttccaaaagctactggaagagtatcaagtccttctaggaggttttagtataggtgacttctttccatccatggagttcgtacacaaatttacgggcatggaaagtcgtctgaaggacaccttcagaaggtttgatcagtttttcgatgaaatcctacaagagcacaggaacccggaatcaaggaaggagcacaaggatctggtggatgtacttcttgagatccagaagaacggtgaccccgaaactcccttaactacagacaacataaaatctatcatattagatatgtttgcagcgggtacggacaccaactttattacgttggactgggccatgactgagttgatgacaaaccctggtgtcctgaaaagagcacaggcagaagtgcgtaacatagttggggagcgtagaagcgtgagcgagaatgatcttccccaactgccctatgttaaggcagtggtgaaggagactttcagactacatcctcctgcgccagttcttgttccaagggagtcaatggaggatgtgacgatcgatggttacgacattcccgcaaagaccagatttttcataaacgcgtgggcgatcggtagagatcctaagagctgggagaatcctgatgtgttcgagccagaaagatttatgaactcccacatagacttcaagggacaagactatgaattgatccccttcggggcagggagacgtagctgtcctgcgattaccttctccgccgttacagtagagttagcattagcacagcttttacactcattcgattgggaattgccaccgggagttgaagcgaaggatttggatatgacagaggtttttgggatcaccatgcataagatttaccctctaatggtggttgcgaaaccatacttcagtcagggcttaaat

SEQ ID NO: 306 - DNA, 1503 bp, synthetic construct
  source 1..1503: mol_type = other DNA
atggcagagttatttcttacactttgtgcgctactagctttcttcagcatagcccttaaaatcgtgagaaaaacaaagttagtcaaattaccgccaggaccacgtcctttaccgatcatcggcaacttgcatcaattaaatggatcacttccacatcgtatcttaaccgacctagcgaagaaatatggaccattgatgcacctgcaattaggcgaggtgagcaccgttatcgtgagttctccggcggttgccaaagaggtcatgaaggatcacgacattatcttttgctatagacctttgctattatcacctgcgatactaaactatggtggcacggacatagccttcgctccgtacggagagtactggcgtcagttgaggaaaatatgtaccatcgagcttctttcctccaaaagggtacaaacatttaggagtatccgtgagcaagaggtatcaaatctgtttcgttctataaagagcgcggaaggaagcgtagtgaatttgagcaagatgatctattcattaacatacagtattactgctcaggcggcctttggaaaggagtccaagtatcaggataagttcatctctgttatgcaggatgcgataaaactaatgggtggcttcaacattgccgatgtttacccgtccatcaaaattttagagaagattaccggaatcaggcacaagctagaggagacacacaatcaaatcgatgagattgtgcagaacatactgatagagcacaaggctaaacgtgcggaaagtaaactaggcaatcgtggagctaaagaagacctggttgacgtgcttcttaatatccagtcaagtggcgaatttgagccgccgctgacagatgacaacctgaaggccgtgatttatgacatcttcagtgccggtggggaaacaagctctacaacggttatctgggcgatgtctgagatgattaggaatcccaaagtaatgaaaaaggctcaggacgaagtgagagaagtgtttgcagggaacggaaacgtggacgaaagcagacttcacgagcttaaatacctgcaagcggttataaaagagactatgcgtttgaaccctgctgtaccgctgcttcttccgagggagtgctctgagcactgccaaataaacggatatgatgtaccagccaagacgagggttatcgtcaacgcttgggctatcggcagggacccgaaacagtggacagaacccgagaaattcaaaccggaccgtttcctggacagcgaaatcgattttagaggcaagactttctcctttataccatttggcgccggtagaaggatatgtccgggtatcagttttgccttgcccctggtagaattacctctagcacagcttttgtatcactttgattggaaacttcctggacaatcaaagcccgatgaacttgatatgacagaaatttttgggctaacggttggaaggaaacacgatctgaatctggttccgatcccttacaggccgtgctctgtgcta

SEQ ID NO: 307 - DNA, 1503 bp, synthetic construct
  source 1..1503: mol_type = other DNA
atggcagagttatttctaacactgtgcgcactgctggccttcttttcaatcgcaattaagattgttcgtaagactaagccagttaaacttccgcctggaccaagacctctgccaataatagggaacctacaccaacttaacggctcactaccacacaggatactaactgatttggctaaaaaatatgggccgctaatgcatctacaattgggagaagtgagtacggtaatagtttcctcccctgccgtggccaaggaagtcatgaaggatcacgacattattttctgctacaggcctctgctactatcaccagctatacttaactacggaggaactgacatagccttcgcaccatatggagagtactggagacaacttagaaagatctgcacgatcgagctattgtcctcaaagagggttcaaaccttccgttccatccgtgagagggaagtatccaacctgtttcgtagtattaagtcagaagaggggagtgttgtcaatttatccaaaaggatatactctttgacatattctataaccgcgcaggcagcctttggaaaagagtccaaataccaagacaaattcatatccgtgatgcaagatgccattatgctaatgggtggattcaacatagccgatgtttatccctcaattaaaatcttggagaagattacaggaataagacataagctggaggaagtacataatcaggttgatgaaattgttcagaacatcttaatcgagcacaaagctaaaagggctgaaagcaagttgggtaataggggagctaaagaggacttagtggatgttttgttgaatatccagagcagcggagaatttgaggcgcctttaacagacgataatctaaaagccgtaatttacgacatattttcagctggtggggagaccagtagcacgactgtcatttgggcgatgagcgagatgatcaggaacccgaaagtgatgaaaaaggcccaggacgaagttagagaggtcttcgctggtaacggtaacgttgacgagtccagattacatgagcttaaatatttgcaggcagtaatcaaagaaacaatgagattaaaccctgcagttccactactgcttccgcgtgagtgctcagaacactgtcagattaatgggttcgacgtgcctgctaagacgagagtgatcgtcaatgcatgggccatcggtcgtgatccgaagcaatggacagaaccggagaaatttaaacctgataggtttctagattctgagattgattttcgtggcaagaccttcagttttatcccgtttggcgctggacgtcgtatatgtcctggtatcagtttcgcgctaccgctagttgagctaccccttgcgcagctattatatcatttcgactggaaactaccagggcagttaaaaccagacgagcttgatatgaccgagatcttcggacttactgtcgggagaaaacatgacttaaaccttgtcccgataccctacaggccttgcagcgtgctt

SEQ ID NO: 308 - DNA, 1605 bp, synthetic construct
  source 1..1605: mol_type = other DNA
atggcaataagttctagtatcaagagtatattacttaagttaacaatcgctcagttggcattgttttttttgcagtcttctatggacttcattatattcttcggggcacttttgctgttctatttgatggtggccaagattcacaggaaatctaccggcgaaaacagcaagttaaatttgcctccaggtccgaaaccgttaccgataattggtaatatgcatcagttgcacggctccccgattcatcatcttcttagggacttggcgaagaagtatggtccgctgatgcatttaaagctgggcgaaatatcagctattatagttacaagtccggaaatggcaaaggagatctacaaaacgaacgatttgctatttgcgagcagacccagtacccacctggcctttaagatcattagttacaacttcacgggtatcatatatagcgcgtatggaaatcactggagacaactgcgtaagatttgtacagcagagttgttgtcaccaaaaagagtgcagactttcaagtctattagagaggacgaagtttttaatttaattcagttgatctacagccagaaaggctccataattaatctgagcaggtgtatcttcttactatcatatagcatcactagcagaattgccttcggtaaaagaaacaaatatacagaaagatttatccagttagtcgaggaaattaacaaattggtcagtggtttctctcttgcagacatgtatccttctgtgaagctattacaggttctatcaatcatgcgttataaacttgagatagctcatcgtcaggtagaccaaatactggaaaacatcatcaacgaccacaagcagaagatcacggaagctaagcaagaaggaggtgagcgtaaagaagacttcgtagatgtcctgttaattattcaagaaggaggagacagtgaacctcagcttactgacgctgcaatcaaggctgtcatatttgatatattttccgcagggagcgaaacctccagtactgcaatagagtgggcgctatccgaattaatcagaaatccggaagtaataaagagagcccaggacgaggtgaggaatgtttatgacgagcgtggcaatgttgacgaatcctccctacatgaactgaaatatttacatgctatattcaaagagacattaagattgcatcccagcgcaccacttctacttccgagggaatgtggggagcagtgcgagattaatggatacgtgatccctgcaaaagcaagaatattggtcaatgcttgggccatcggcagagatcctatgtattggaccgaagctgaaaagtttataccggaaagattcttagacagcgagattgactttaaaggtaacaactttgagtacataccatttggtgctggcaggcgtatatgtcctggtatcagcttcagccaacctcacgtcgagctggtgttggctcagttgttattccacttcgactggaaattgccgggtgaattaaagcaagaggagctggatatgactgaagattttggtgtaactaccaagaggtctaatgacttacacttgatcccgattccttataaccgttcttgcttaagaatggatgca

SEQ ID NO: 309 - DNA, 1509 bp, synthetic construct
  source 1..1509: mol_type = other DNA
atggcagagtttataatatttttgggcgctttacttttgttctatttcgtcatatcactgggaaagagttcaaagttaaaactacctcccggaccgaaacaactgccaattatcggcaatatgcatcagctgcatggtagccaactacatcatctattaagggatctggctaaaatttatggtcccctaatgcacctaaaacttggtgaaatatccacgattatcgtgacgagcccggagatggctagagaaatttataaaaccaatgacctgcttttcgcatctaggccgagccattaccttgctttcaaaatcttctcctataactttgcggatatagcattcagcccctatggtaactattggaggcaaatgagaaagatatgtaccttggaacttttgagcaccaaaagggttcaaacatttcagagcatccgtgaagatgaggtcttcaatctgatgaagagtataagtactcagaaagggtctattattaacttatcaaggtctatattcactgtgatttacactataacttcccgtgcagcgttcgggaagagaaataaagatactgaaaggtatatcaggttggtggaggaggtcacgaagctagccagctggttctcacttgccgacgtatacccgagcgtgaaattattccagttgatgtctactgttaggcacaaagctgccatagtgcataaacaagtcgatgaaatcattgaaaacatccttaatgaccacaaggagaagatcgaagaagctaaacaggcgtccggcgaagccaaggaggatttagttgatgtacttctatccattcaaaatagtggagatttcgagccacagttgacagacacgaacataaaagcgattatattcgatatgttcacagctggtggagaaacctctagtacagttgtggaatgggcaatctccgaaatgataaaaaatcccgagactataaagagagcccaaaacgaggtacgtaacgcatttgacgaaaagggcaacgttgacgagtctaaacttcacgaacttaaatatttgaatgctatcattaaagaaaccttaagattacatccttctgctccactgctagttccaagggagtgtactgagcagtgtgagatcaatggctacgtaattcccgccaaagccagattgtttgtcaatgcctgggccatagggagggacccgaaatactggatcgatgcggaaaattttaatcccgagcgtttccttgactctaaggtagactataggggcaatgatttcgagtacatcccttttggtgctggaagacgtatatgccctggcattacattcagtaggcctggaatcgaactggcattagctcagcttctgttccattttgactggaaagtcccgggtgagcttaagcaagaggaacttgatatgacagagaactttggaatgactgtaaggcgtaaaaacgacttgaacttaattcccgttctttacaatagatcttgcctgagcaaggacaac

SEQ ID NO: 310 - DNA, 1503 bp, synthetic construct
  source 1..1503: mol_type = other DNA
atggcagagttgttcctaaccttatgcgctttactagctttcttttccattgccctaaagatagtcaggaaaacaaagctagttaaattgcctcctggtcctaggccactgccaattatagggaaccttcatcagctgaacgggtctctaccccatcgtattttaacagaccttgcgaagaaatatggtccgttaatgcatcttcaattaggcgaggtttctactgtaatcgttagttcacccgcagttgctaaggaggtgatgaaagatcatgatattatcttttgctatcgtccgctgcttttgagtcccgcgatactgaactatggtggaaccgacatagccttcgctccgtatggcgagtattggagacaactacgtaaaatctgtaccatcgaacttcttagtagtaaaagagtgcagacattcaggagtatcagagagcaagaggtctcaaacttatttaggtccatcaagtcagcggaggggagtgttgtaaatttaagcaagatgatctactcccttacctattcaataactgctcaagcggcgtttggtaaagagagtaaatatcaggacaagtttatttccgtcatgcaagacgctatcaaattgatgggaggtttcaacatcgctgatgtctatccgagcattaaaatcttagaaaagataacgggtatcaggcataagttggaagaaacacacaatcaaatagacgagatagttcaaaatatattgatagaacacaaagcaaaacgtgctgagtctaaattaggcaataggggtgcgaaggaagaccttgtggacgttctattaaatatccagagttctggcgagttcgaacctccgttgacagacgataatctaaaagcggtgatctatgacattttttcagccggaggagagacaagtagtacgacggtgatatgggcgatgtcagaaatgattagaaaccccaaggtcatgaaaaaagctcaggacgaagtgagagaggtgtttgcaggtaacggtaatgttgacgaatccaggcttcatgagcttaagtatctgcaagcggtgatcaaagagactatgaggttgaatcccgctgttcctttgcttctgccgagggagtgttctgaacactgccaaatcaacggatacgacgtgccagcaaagactagagtgatagtaaatgcctgggctattggaagagatcctaaacagtggacggagccagagaagtttaagcccgatagatttttggacagcgaaatcgacttcagggggaaaacgttctcattcatcccttttggggcaggccgtagaatctgcccaggaatctcattcgctctgccattggtagaactacctttagcacagctactttaccacttcgattggaagctgccgggacagagcaaaccagatgagctggacatgaccgagatcttcggacttacggtgggcagaaagcacgatcttaatttagtcccaattccatacagaccttgcagcgtctta

SEQ ID NO: 311 - DNA, 1503 bp, synthetic construct
  source 1..1503: mol_type = other DNA
atggcagagttgtttctaaccctttgtgcgttgctagctttctttagtattgcgataaagatggtgaagaactcaaaaccagacaatctgccaccaggtccgatgcaactaccaattattggtaacatacatcagttattcggtagtttagcatacaggattcttacgggacttgcgaagaaacatggacctctgatgcatttgcaattgggtgaaatctcaaccattgtagtatcactggcagaggtggcgaaagaagtaatgaatgaacatgacataattttcgcgtcacgtcctaacctgcttggtaccagaatccactcttacaacgctacaaaaatcgccttctctccctacggcgattattggaggcagttatgcaaaatatgcacgatggaactattgtctcccaaaagggttgagacctttaggtcaatcagggaggaggcagtgcttgacatgataaaaagtatagcgctgcagaggggctctgacgtcaatctgagtaaagagatctttagcttgacctataggattacggcgcaagcggcgttcggaaagagaaataagcaccaagaagagtttatttctgttatggtggacatagtagaactagttgccggtttcaacattgctgatatgtatccctccgtaaaaatacttgagcgtatttcaggcatgaggcaaaagctagaacgtacccacaaacaagtcgatgagatccttgaaaatatacttacggaacataagggcaaacgtgatggtttgcagcccggtaagggcgatagtaaagaagacttggtagatgtgttgttgaaagtgcagaaatcaggagagttcggggcgccactatcaaataataacctaaaggccgtgattcttgacatattctccgctggcgacgaatcaagtagtacaactgcgcaatggacgatgttggaaatgatgaaaaatcccggtaccattaaaaggtcacaagaggaggtgagagaagtgtttggtgacaagggcaatgtagacgagtccaggttacatgagcttaagtacttacaagcggtaattaaggatgcgctgaggttaaggcctccagttccgctgctattgcccagggagtgcgcagagccatgtgagatccatggttacgaaatccccgccaaagctcgtatgatagtaaacgcgtgggcaattggaagagattcaaggaggtggactgaacttgaaaaatttatctccgaaaggtttcttgacagtgactatgacttcaggggtcagaacttttgctatattccgttcggcgccagacgtagaatctgccctggtattgctttcgctttgcctaatgtagagctgctacttgcgcagcttttgtaccattttgattggaaacttccaggggagataaaggaacaacagctggatatgaccgaaaccttcgggctagcggttagacctaaacatgacgtgaatcttgtacctatctcttttagccctagcgatgaaaac

SEQ ID NO: 312 - DNA, 1527 bp, synthetic construct
  source 1..1527: mol_type = other DNA
atggcagacctgatcatattccttagtgctttccttcttttctatctagtggtcgtgaagttgttgcgtaaaccaactggaaagaactccaaacttaacatcccgcctggaccaaaaccattacccataataggtaacatgcaccaattacatgggagtcccatccaccaccttatgcgtgatcttgctaagaaatatggtccgcttatgcatcttaagctgggagagatatccgctataattgtcacctcacctgaaatggcgaaagaaatctacaaaacaaacgacctattatttgcttccagaccttcatctcatctggcgtttaaaattatttcctataacttcgcggacatcatattttcagcgtatggtaatcattggcgtcagttgagaaaaatttgcactgttgaattgctgtcacctaagcgtgtccagactttcaagtcagtacgtgaatacgaggtttttaaattgatccaatcaatcagttcaaagaggggaagtataataaatctttctagaagcatcttcccattatcatatagcatcacttcacgtatcgctttcgggaaaaggaataagtacacagagcgtttcatacagatagttgaggaaattaataagttggtctcaggtttctcacttgcagatatgtacccgagtgtcaaacttctacgtgttatgtctataatgagatataaacttgttatcgctcacaaacaggtcgatcaaatattggagaatattcttaatgatcacaagcagaatgtcacagaagctaagcaggaaggcggagaaaggaaggaggacctagttgacgtgttgttaaatatccaagaaggtggtgatttcgaacccgaacttacaggaacggcaatcaaggctgtgctgtttgacatattcagcgccggatctgaaaccagcagtacagctatagaatgggcgctgtccgagatgatccgtaaccctgaagtgattaggcgtgcccaagatgaggttcgtaacgtttatgacgaaagagggaacgttgatgaatcctcactgcacgaattaaaatacttgagggcgataattaaggagacgcttaggctacacccctcagcaccacttctacttcctagggagtgtagtgaacaatgtgagataaatggttacgtcatcccagctaaagccagaattatagttaatgcttgggctataggtcgtgacccgatgtactgggcagaagcagaaaaatttaatccagaaaggtttcttgacagtgaaattgattttaaaggaaacaatcttgaatatattccctttggtgcaggaagaagaatatgtccggggatcagtttttcacaaccgcacatggagcttgtgctggcgcagctgctttttcactttgattggaaacttcctggcgaactgaaacaggaagagctagatatgactgaagattttggcgtcactattaggaggacgaatgatcttcatctaattccgattccttataatcgtagttgcttacgtatggacgga

SEQ ID NO: 313 - DNA, 1503 bp, synthetic construct
  source 1..1503: mol_type = other DNA
atggcagaactatttcttactctgtgtgcgctgctggcattctttagtattgctataaaaatggtggctatcacattaaagaactccaagccacgtgacttaccaccaggcccgatgcagctaccaattattgggaatatacaccagttatttggttcacttccacacagaatactgacggatcaagcaaaaaagtatggtccacttatgcacttacagttgggcgagatcagcacgtttgtagtatcctctgccgaggtcgctaaagaagtgatgaacgaacacgatataacctttgcgtcaaggccaaatttactgggtacaagaattatctcctacaacgctacagacattgcgttcagtacttacggcgattactggaggcagttaagaaaaatatgtaccatggagttgctgagtccgaaaagagttcaaacattccgttcaattcgtgaagaggccgttttagacatggttaaatcactagcccttcaaaggggttccattgtgaacctatcaaaagaaatattcagtttgacgtatcgtataacagcacaggccgccttcggaaaacagaacaaacatcaagaggagttcatctcagtcatggtcgatattgttgagctagtggctggttttaatatagccgatatgtatcccagcgtcaagatcctagagcgtatcagtgggatgagtcagaaactagagagaacgcacaaacaggtggatgaaattttggaaaatattcttgcggaacataaagggaaaagggacgggctacatccagttaagggggaggtcaaagaggacctagtcgatgttctgctgaaagtacaaaattccggagacttcggtgcacctctatcagattctaatccgaaacgttccagctatgctggtggcgaaacctcctctaccacggcgcaatggagcatgctagagatgatgaaaaatccgggaatcatcaagagggcacaagaagaggtcagagaagtgttcggggacaaagggaatgtggatgagagtagattacacgaacttaaatatctacctgcggtaataaaggagacactgagacttcatccacccgttccgttgcttctaccacgtgaatgcggtgaaccctgtgagatacacggatacgaaatacccgctaaagctcgtgttctggtgaacgcttgggctataggtcgtgatcccaggcattggaccgaacctgacaaattcataccggagagattcctggattccgactatgacttccgtggccaaaacttctgctatataccatttggtgctggacgtaggatctgccctggcatagctttcggattgccaaacgtcgaattgccattggcccagcttctttaccatttcgactggaaccttccaggagagatcggcgagcaacaagtggatatgactgagacgtttggtctggcggtccgtcctaaacacggagtccacttggtgcccttcagcttttctaattcttctgaaaat

SEQ ID NO: 314 - DNA, 2064 bp, synthetic construct
  source 1..2064: mol_type = other DNA
atgactacgctgctaaatccttatttcggagagtttggaggaatgtacgtgcctcaaatcctaatgccggccctaaggcaacttgaggaagcattcgtcagcgcacagaaagaccccgagttccaggcacagttcaatgacctattgaagaattacgccggtagacctactgcattaactaagtgccagaatattaccgcgggaactaacaccacgctatatttgaagagagaggacttactacatggcggggcacacaaaacaaaccaagtacttggccaggctctgctggctaagagaatgggcaaaacagaaattatcgccgagactggggcaggccaacacggcgttgccagcgctctagcatcagcgttactgggactgaaatgtagaatttatatgggcgcgaaggatgtggaacgtcagtcccccaatgtctttaggatgaggctgatgggtgccgaggttattcctgttcatagtggttccgccactcttaaagacgcatgcaacgaggcgcttagagactggagtgggagctacgagacggcccattacatgctagggaccgccgcaggtccccacccgtatccgacaatagttagagaatttcaaagaatgataggagaggaaaccaaggctcagattttagagagggaaggccgtttgccggatgccgtaattgcttgtgtaggtggtggaagcaatgcaataggaatgttcgccgattttattaatgagacgaatgtcggcctaattggtgtggaaccaggcggacacggaatcgaaacgggtgaacatggtgcaccacttaagcatggaagggtgggcatctatttcgggatgaaagccccgatgatgcaaaccgaagatgggcaaatagaggagtcatattctattagcgccggactggacttcccaagcgtaggtccgcaacatgcatacctaaacagtacggggagggctgactacgtgagcataacggacgacgaagcactggaagcttttaaaacactgtgtttgcacgaaggaataattccggcgcttgaatctagtcatgctcttgcccacgccctgaaaatgatgagagagaaccctgataaggaacaattgttggtggtgaacctaagtggtaggggcgataaggatatattcaccgttcacgacatactaaaggcacgtggcgagatcgggggcggttctggtgagggacgtgggagcctattgacttgcggcgatgtcgaggagaatcccgggccaatggaacgttacgagtcactttttgctcaactgaaggagagaaaggagggggccttcgtgccgttcgtgacccttggggacccagggatcgagcaaagccttaagatcattgatacccttattgaggctggggcagacgctctggagttggggataccattttctgaccctttagcggatggccccactattcagaacgctacgttacgtgccttcgcggcgggcgttacaccggcacagtgcttcgaaatgttggctctgataaggcaaaaacacccaaccatccccatagggttactaatgtatgccaacttagtgttcaataaggggattgatgagttctatgcgcaatgtgaaaaggtgggcgtcgattcagtgctggtcgcggacgtacccgtagaggaatccgctcccttcagacaggccgcgcttcgtcataacgttgcaccgatatttatatgccccccaaacgcagacgacgatttgttacgtcaaatcgcctcttacgggcgtggatacacttacttgctaagtagggctggggtaactggggctgagaaccgtgctgctctacccttgaaccatttagtcgctaaactgaaagagtataatgctgctccacccctgcagggctttgggataagtgcaccagatcaggtaaaggcggctatcgatgcaggagcggcgggcgcgatatcaggcagcgcgatagtaaagattatagaacaacacataaatgagcccgaaaagatgctggccgcgttgaaggtattcgttcagcccatgaaggctgccactcgttca

SEQ ID NO: 315 - DNA, 396 bp, synthetic construct
  source 1..396: mol_type = other DNA
atggcagcagaacaaaccacttttactaggagtgaaatcgaaaagaataacgacaaagataaggtcctgattatactgcatgataaggttttcaatgtccatcgtttcctgaacgagcaccctgggggagaagaggtgttattggatcataaaggaaaagacggctctgaagatttcgacgatgtgggccattccaaagacgcaatggaactaatgaagacctaccaggtgggcgtacttgtagaaagtgagcgtagtaataagccgccgaaaaagggttgggtcgcaggatataacacaaagcagagtgaaaagtatgtccaaccaggcgttcctttttatctgctgattgggggtatcgcgttggtagtcgcgcttttcttctatgcttactaa

SEQ ID NO: 316 - DNA, 408 bp, synthetic construct
  source 1..408: mol_type = other DNA
atggcagcaagtgaccagaagttgcacaaattcgacgaggtcagtaagcacaataagaccaaagactgttggctaattatcaacggtaaggtttacgacgttaccccctttatggatgatcaccccggcggggatgaagtgctgttgagtgcaacaggcaaggatgctacaaatgatttcgaggacgttggccatagcgatagtgcgagagagatgatggataagtactatataggcgaaatggatatggcaaccgtgccactgaagagaacttacatacctccccagcaagctcagtataatcctgacaagactccagaatttgtgatcaaaatcctgcaattccttgttcccttgttaatcttagggcttgcgttcgcggtacgtcattatactaaggaaaagtaa

SEQ ID NO: 317 - DNA, 2040 bp, synthetic construct
  source 1..2040: mol_type = other DNA
atggcagaaaagacggaggccatcctggagaccgaacacatatcagaagcctcagattccctatttagttctctcgacatcttcttagttgcacttatagtgggcggcatatcctggtactttatcaacaagtacaagaagactactacagttaccagtgaaagaagttactcaatctccccgagccccatcaccctggagaaccccagtgactcttcattcattaagaaattgcagtcatccggcaggagcctggtagttttctacggtagccaaaccggcaccgcagaagagtttgccggaagacttgccaaggaaggagcgagatatagacttaaaggaatggttgctgacctggaggactacgaaatggaggagctagtgaatctaaagaatatcccgaactcattggctgtgttctgtttagcgtcctacggagagggagagcccacagataatgcacgtgaattttgtgactggcttcagaactccgagccggaccttaaaggcttgacttacactgtcttcgggcttggcaataaatcttatgagtattataatagggttgccattaacgttgataaacgtctaaaagagctgggcgcgacgcgtatatatgacttggggttaggagatgacgacgcaaacattgaagacgattttattacctggaaagatcaattctggcccactgtatgtgagcactttggtattgaacctacggaagaggagattaatatgcaccagtataggctagaagaatttgaggaagcacccgataaagtgtttgttggtgagatggctaagctggatagcttgaagaaccaaaaacctccgcatgatgcaaagaacccattcttagccaaagtcaaagtgaaccgtgatctgaataacagtgatcgttcttgcctgcacatagagttcgacattaaaggcagtaaaatgaggtacgactctggcgaccacatcgcagtgtaccctactaatgactctgagctggtcgaaaagatagggaaactttgtgcaaagaacctggatactattttcaccttgataaataatgacgaagaatccaataaaaagcatccttttccatgcccctgttcttacagaactgctctaacacattacctggatataacacaaaatcccaggacacatgtgcttaaagagttggccgaatattgcactgatgaggcaaacaaggaaaaacttagactaatgtcctccacaacaccagaggggaaagcactgtaccagtcctggatcattgaggataatcgttctgtagttcacgttattgaggacatgccatgctgcagaccatcattggactacctgtgcgaattattgccacgtctgcagccgagattctattccatcagttcttctgccaagttgtacccagatactgtgcacatcagtgcagtcgtagtcgaatatgatacacccacagggaggcataataagggcgttgcgacaacgtggttgaaagagaaagtcccagacccagaaaaagaagcgccagtagtccctgtatttataaggaaatcacagttcagactgcccacaaagccagaaacaccaataattatgattggacccgggaccggactagctcccttcagaggctttattcaagagagaaatatttgcaagagcgagggcaaacctgtcggtgaaacggtactttactttggatgtaggaaacgtagtgaggatttcatttacggtgatgagcttttagaatacgaaaagaatggtttattgaccttacatctagcgttctccagagaccaaccgcataaagtatacgtcactcatctgcttcagcaacatgccgatgaggtctggagggtcataggtgagaataacggccacttatacatttgtggcgatgctaagagtatggccgttgacgttagggttatcttaactaagatattgcaagaaaagggccagatgacggaacaggaggctgtagcgttcctgaagaagatggaaacgcagaggcgttatagtgctgatgtgtggtcttaa

SEQ ID NO: 318 - DNA, 2049 bp, synthetic construct
  source 1..2049: mol_type = other DNA
atggcagaaaacagcgagggggtaaagatcgaatccaccgtggttaccgaggagcctttaatctcagctcttgacattgcactgttcgtcgttataataacagtagcatatttttggtactttaagcgtgataaacagagcagtagtagtgagaaaaagccttacacgatccaaccaagttctctgagttccatagagcaaacatccaacagtagcttcattaaaaagctaaaatcaacgggtcgttctttggtagttttctatggaagtcaaaccggtaccgccgaagaatttgcaggacgtatagcaaaggaaggggcaagatataaaatgaagggtatggtagcagaccccgaggagtgtgacatggccgatttggtggaaatgaaggagattcagaagtctatggcaatcttttgcgtggcaacttatggagagggagacccgacagacaacgcaatggatttctacgagtggttgcaaaatggcgatgcagatttagaaggtttgaattacgccgttttcgggctgggcaataagacctatgaacattataatgagatcgccatttacatcgaccagcgcctagaagagttgggtgctactcgtgtgcatgagattggtttgggagacgacgatgccaatattgaagatgattttgtctcctggaaggaaaaattgtgggaatcagtttgtagtcattacggcattgaagaaacaggggaggagtccaatattaggcagtacaagctagtagactgcagtgagatactgccggagcgtatattcagcggtgagattagtcgtctgaagagctacgagaatcagcgttttcctttcgacgttaagaacccgtatttaagcaagatcgcagtcaatagggaactacataagtctggcgaccgtagctgtatgcacatagagttcgatatcgatggaagcaaaatgcgttacgatacgggagatcatgttgcagtctatcccaaaaactcctcagaactggttgagaaaatcggggaactgctaaatgcagatttggataccgtcttcagtttactaaatactgacgaggagagtagtaagaaacacccgttcccttgtccttgcacataccgtacggcgctgacttattacctggatattacgtcaaacccgagaacgcacatcatgaaagaattgatagaatacgcgtcagaccccaaagaccaggagaagctgaagctaatggcttccagcactcccgaaggtaagaaggagtttcacgactggatattaagggacaatcgtaatattgtgcacattttggaggatttaccgtctgtaaaaccagacctggatcatctttgcgaactgctgcctaggttgcaatgcagatactattcaattagctcaagccccaaagtgtatccgaagtccattcacatcactgcagtgcttgttaactatacaacccctacaaacagggttaacaagggtgtggcaacgaacctactggcgcaattgaaaccaactaatgatgagttaccccagccaactatacctatctatatcagacgtagtcaatttcgtcttccaccgaagagtcaaactcccataattatgattggaccgggtactggactagctccgtttaggggctttatacaggagcgtgattacgctcgtaaggaaggaagagagatcggtgaaatggtcctatactttggttgtaggaagaaagatgaagactttatctatgaaaatgaattgcaggaatatgtagcgaacggaactttaactaagctgcatttggcattctctcgtgatcaaccagaaaagatttacgttacgcatctattagagcaaaacgctgacgaactgtggaatataatcggagaaaagaatggtcacctgtatgtgtgtggggatgctcgttctatggcgaaagacgtgcataacattatagtaaaagttgtcatggagaaaggccagatgacgaactcacaggcattgaactatgtgaaaaagatggaacaacagaagcgttactctgccgatgtatggtcctaa

SEQ ID NO: 319 - DNA, 2148 bp, synthetic construct
  source 1..2148: mol_type = other DNA
atggcagactcttcttcagaaaaactttctccattcgagctaatgagcgcgattctgaaaggtgcgaaacttgacgggtctaattcatctgactcaggagttgcagtgagcccggcagtgatggccatgttgttggagaacaaagaactggttatgatcttgactacatcagttgctgttttgattggatgcgttgtggtcctaatttggcgtcgtagctctgggtccgggaagaaggtcgttgaaccgcctaagttgatagtcccaaagtccgtggtagaacccgaggaaattgatgaaggcaaaaagaagttcactatcttcttcggcacacaaacagggactgctgaagggttcgcgaaggctctagcggaggaagcaaaggcaaggtacgagaaggcagtgattaaagtgatagacattgacgactatgcagctgatgacgaagagtacgaggaaaaattcaggaaggaaactctggcgtttttcattcttgccacctatggtgacggggaacctactgacaacgctgcgcgtttctacaagtggttcgtggaaggaaacgacagaggcgactggctgaagaatttgcagtacggcgtctttggcttaggcaatagacaatatgagcatttcaataaaattgcaaaagtagttgatgaaaaggttgcggaacaaggaggcaaaaggatcgtcccattagttttaggggatgatgatcaatgcattgaagacgattttgcagcatggcgtgagaacgtctggcccgaacttgataacttgcttagagacgaggacgatactactgtttcaacgacctacacagccgccatcccagaatacagagtcgtattccctgacaagagcgacagcttaatatccgaagcaaatggacacgcgaacggttatgcaaatggtaacactgtttacgatgcccagcacccgtgcaggagtaatgtagctgtgagaaaggaattgcatactccagcgtcagatagatcttgcacacatctggattttgatatcgccggaaccggactttcctacggcactggcgaccatgtgggggtgtattgtgataacctgtctgaaacagtggaggaggcagagcgtctactaaacttacctccagagacttactttagcctacacgcggacaaagaggatgggacaccccttgcggggtccagtttaccgcccccatttccaccatgcaccttgagaacggcactaacgaggtatgcggacttactaaatacgcccaagaaatccgctctgcttgccttggcggcgtacgcttcagatcctaacgaggcggataggttgaaatacttagcatccccagctggtaaagatgagtatgctcaaagccttgttgcgaaccagagatctcttcttgaggtgatggccgagttcccctctgcaaagccccctttgggcgtattcttcgcggccatagcgccaagattgcaaccaaggttctatagcattagctcaagtcccaggatggcaccttcacgtatccacgtaacctgtgccctagtttatgagaaaacaccaggcgggcgtatacataagggagtgtgcagtacatggatgaaaaacgctatcccacttgaagaatctagggactgttcttgggcgcccattttcgtgaggcaaagcaacttcaagttacccgctgacccgaaggttccggtcattatgattggccccggaactggactagcaccgttcagaggttttctacaggagaggttggcgctaaaagaagaaggggctgaattaggtaccgccgtcttctttttcggctgtagaaatagaaaaatggattacatatatgaagacgagttgaatcatttcctagagattggcgcactgtccgagcttctagtggctttttcccgtgagggacctacaaaacagtacgttcaacacaagatggccgaaaaggcgtcagatatatggaggatgatcagcgatggggcatacgtgtatgtatgtggcgacgcgaaagggatggcaagagacgtacatagaacgttgcacaccattgcacaggaacagggatctatggactccactcaagcagaaggtttcgtgaaaaatcttcaaatgactggacgttatttgcgtgatgtatggtaa

SEQ ID NO: 320 - DNA, 2094 bp, synthetic construct
  source 1..2094: mol_type = other DNA
atggcacacaacatggatctgcctacagtagacgcaagtcaacccaaggctatccgtatggccgggagcccgatgcttgagaatgagagcggccctgaagacgaacaaccgtttctttctataacagacattatgttgatcctgggtatccttttggcaatgatgttcgtatatgtcaggagtcgtatgaggagagaagcggcgccggcacccaagtcttactctattcaaccgacgtctttcgcggttgcgccttcctctgaaaacagcttcatcaagaaattaaagacgtctgggaggtctctggttgtgttttacgggagtcagaccggtacggctgaagaatttgccggcagactagcaaaggaagggatacgttataagatgaaaggaatggtggcagacccagaggaatgcaacatggaagaattagtacaccttaaaacgataccaaatagcttagctgtattctgtttagcaacctatggtgagggtgacccgacggacaatgctatggagttcattgactggctgaaaaacggtgacgcggatttaacagaactaaattatgcagtgttcggcctgggaaataagacgtatgagcactacaacgaagtaggaatttatgttgaccaccgtttagaacagttaggtgctacgagagttttcgagctgggacttggtgacgacgacgctaacattgaggatgattttatcacgtggaaagataaattttggccggccgtctgtgagtttttcggtatcgagggtgctggcgaagacgttagtatcaggcaatataagttaacggagcatgtcgatatgccagctgagagaatatatacgggggagttcgcgaggcttcattcatttaaaaatcagcgtccgccctatgacgctaaaaatcctttcttagcccccgtcattgtgaacagggagttgcacgggcctacgtctgagagatcatgtatgcacatcgagttctatatcgaagactctaaaatacgttacgatgcaggggatcatctagccgtatatccagtcaacaatgccaagttagttaacaagattggagaacagtgcggggcgaatttagatatagtgttcacgttaacgaacactgatgaggagagtacaaagaagcatccgtttccttgcccttgcacttaccgtaccgcgctcacgtattatttggatataacgtctaacccacgtacccacattcttaaagaactagcagagtacgcaactgatccggccgacaaggagaagctgaagttgatggcttcaacgacggcagagggcaaagccgcctaccagcagtggataattcaggaaaaccgtaacatagtccacattcttgaggatataccgtcactgaagccaccgcttgatcatttgtgcgaaatacttcctcgtttacagtgcagatactatagtatatctagcagtcccaaactacaccctacaactatccatatcacggccgttgtagtggagtataagacccctaccgggcgtatcaatcgtggtgtaacaacctcctggttaaaagaaaaacacccttccgatcctccgtgtttggtcccaatatttcttaggaaaagtcaattccgtcttcctaccaggcctaatattcctatcattatggtcgggcccgggactgggttggccccgttccgtggcttcatccaagagagagactttgcgagaaaggaaggaaaggaggtgggtgacactatactatactttggctgccgtaagagtcaggaggacttcttgtacagggacgagctggaagaatacgtcaagaatgggacgctaactttgcacacggccttctccagggagcagtcacacaaggtctatgtaactcatcttttggagaaaaataaagaagagatctggcacgtcatcggggagcaaaacggccacatatatgtctgcggagacgcaagaaacatggcgagggacgtccacaatatactgctgaaagtggtcatggagcgtggaaatatgtccgaacttgacgccgcgaactacataaagaaaatggagagccagaaaagatattcttctgacgtatggtcctaa

SEQ ID NO: 321 - DNA, 1767 bp, synthetic construct
  source 1..1767: mol_type = other DNA
atggcagaagttgaaaagactgaaaagaatattgctgaagatgatgaatctaaaattatttacagaggttggaaagttatgccattcattattggtaatgaaacatttgaaaaattgggtattgttggttcatcatctaatttggttatttacttaactacagtttttaatatgaaatctattactgctgcaaaagttgttaatatttatggtggtacttctaattttggtactattgttgctgcatttttgtgtgattcttattttggtagatacaaaacattgtcttttgctatgattgcttgtttcttgggttctgttgcaatggatttgactgctgttattcatccattacatccagctcaatgtgctaaagaaattggttctgtttgtaatggtccatctattggtcaaattatgtttttggctggtgcaatggttttgttagttattggtgcaggtggtattagaccatgtaatttgccatttggtgcagatcaatttgatccaaaaactaaagaaggtaaaagaggtattgaatcatttttcaattggtactttttcacttttacttttgctcaaatggtttctttgactttgattgtttacgttcaatctaatgtttcttggtcaattggtttagctattccagcaattttgatgttattgggttgtattattttctttgctggttctaaattgtatgttaaagttaaagcatctggttcaccaattcattcaattactagagttattgttgttgctattaaaaagagaagattgaaaccagttggtccaaatgaattgtacaattatattgcttctgattttaaaaattctaaattgggtcatactgaacaattcagatttttggataaatcagctattcaaactcaagatgataaattgaataaggatggttcaccagttgatgcatggaaattgtgttctatgcaacaagttgaagaagttaaatgtgttattagagttttgccagtttggttgtcagctgcattgttttacttggcttatattcaacaaactacatatactatttttcaatctttgcaatcagatagaagattgggtccaggttcttttcaaattccagctggttcttacactgttttcttaatgttgggtatgactatttttattccaatttacgatagagttttggttccatttttgagaaaatatactggtagagatggtggtattactcaattgcaaagagttggtgcaggtttgtttttatgtattacttctatgatggtttctgcaattgttgaacaatacagaagaaaagttgctttgacaaaaccaactttgggtttggctccaagaaaaggtgctatttcttctatgtctggtatgtggttgattccacaattggttttgatgggtattgctgatgcattggcaggtgttggtcaaatggaattttattacaaacaatttccagaaaatatgagatcttttgcaggttctttgtactattgtggtattggtttggcttcttacttgtctacatttttattgtctgctgttcatgatacaactgaaggtttttctggtggttcatggttaccagaagatttgaataagggtagattggaatatttttactttttagttgctggtatgatgactttaaatttggcttactttttgttagtttctcattggtacagatacaaagatgttgttgctaaagataaagatatggataaaacttcagcagaatttgataaagtttcagtttaa

SEQ ID NO: 322 - DNA, 1791 bp, synthetic construct
  source 1..1791: mol_type = other DNA
atggcagcagaaaaatcatcttcaattgatgctacagaaaatggttttgttaaaattaaagaaccaaatcatagaggtgttaaagctatgccattcattgttggtaatgaaacttttgaaaaattgggtacattaggtacttctgctaatttgttaatttacttgactacagtttttcatatgaaatctattactgctacaaatttgattaatatttttaatggtacttgtaattttggtacattgttaggtgcatttttctctgatacttactttggtagatataaaacattgggttttgcttctgtttcttcaacattaggtatgttaattttgactttgacagctgcaatttcaaaattgcatccaccatcttgtgaaaagaactctaaagcatgttcaggtccaacttcaggtcaatttgcatttttgtttactgcatttttgtttttaattattggtgcttctggtattagaccatgtaatttggcttttggtgcagatcaattcaatccaaatacagaatctggtagaaaaggtacaaattcatttttcaattggtattactttacttttacttttgcaatgatggtttcattgacagttattgtttatgttcaatcatctatttcttgggcattgggtttagctattccaacatttttaatgttgttgtcttgtgttttcttttttgttggttctagaatttacgttaaagttttaccagaaggttcaccattgacatctatgatgcaagttattgttgcagcttttagaaaaagaaaattgaatttgccagatcatcaaccgtgggattcattgtttaattatatttctccatcatctattaattcaaaattgtcttatactgatcaattcagatggttaaataaggctgcaattaaaacatctaaagatcatttgtacgctgaaggttctactgctaatccgtggagattgtgttctatgcaacaagttgaagaagttaaatgtgttattagatctattccaatttggacatctggtgttttatattacgttgttttaaatcaaatgtctacttacttggtttttcaagcattgcaatcagatagaagaattggtaaaacaaattttaaaattccagcagcttcttatacagttttctcaatgttgtctttgtcaatttggattccaatttatgatagaattattgttccatttttaagaaaaattactaaaactgaacaaggtattacaattttgcaaaagatgggttttggtttggttattgctgttattactatggttgtttcagctattattgaagataaaagaagagttattgcattgactagaccaattgttggtattgaagaaagaaaaggtggtatttcttcattgtctggtttttggttggttccacaattagcattggctggtatttctgaagcattcactgttattgctcaagttgaatttttctacaaacaatttccagaaaatatgagatcatttggtggttcatttttgttttgtggttttgcaatttcatcttatttgtcatcatttttgatttcaattgttcataaagttacaagaaattcttcaggtggtaattggttggatgaagatttgaataaggctagattggattacttttactatttggttgctggtttggaagttttaaattttggttactttttggtttgtgctcattggtatagatacaaagaaactcaatctggtaatgaaattgctatggaacaaattttggatgaaccaaagaaaaatttggatgttgtttaa

SEQ ID NO: 323 - DNA, 1770 bp, synthetic construct
  source 1..1770: mol_type = other DNA
atggcagaagaaaatcaagaacataaagctattaaaaatcatgaaccaaattacagaggtattaaagctatgccatttgttattggtaatgaaacttttgaaaaattgggtacaattggtacttctacaaatttgttgatttacttgacttctgtttttcatatgaaaacaattacagcaactaatttggttaatatttttaatggtacttgtaattttggtactttggttggtgcatttttgtgtgatacttactttggtagatacaatactttaggttttgcttctgtttcttcatttttgggtatgttagttttgacattaactgctgctattaaaaatttgcatccagaagcatgtggtaatgaagaaggttctaaatgtatttcaccatctggtggtcaaatggcatttttattgtctggttttgcatttttgattgttggtgcttcaggtattagaccatgtaatttggcatttggtgcagatcaattcaatccaaatactgattctggtagaagaggtattaattcatttttcaattggtactattttacatacacttttgcaatgatggtttcattgactattattgtttacgttcaatctaatatttcttggtcaattggtttggctattccaacatttttaatgtttttgtcatgtgctacatttttcgttggtactagaatttatgttagagttttgccagaaggttcaccattgatttctgttgctagagttattgtttcagcttttaaaaagagaaaattagaattgccagatgaaccaaaattatcattgtttaatcattcagcatcttcaacttcatctattaattcaaaaatttcttacactgatcaattcagatttttgaataaggctgcaattattacttcagaagatgaatttaattcagatggttcagttaaaaatccgtggagaatttgtactattcaacaagttgaagaagttaaatgtttgttgaaattgattccagtttggatttcttgtattatttactatatttcagttgttcaacaacaaaattacgttgtttttcaagcattgcaatctgatagaagattatttggtacatctaaattcaaaattccagctgcatcttacattgttattgctatgttggctttgactatttggattccaatttttgatagaattattgttccatgtttgcaaaagattactggtaaagaaggtggtattactattttacaaagaatgggtattggtatggttttgtcagttttgacaatgattacatcagctttggttgaatctaaaagaagaaaatttgctttgaatagaccaacattgggttttgaaccaagaaaaggtgcaatttcttcaatgtctggtgtttggttaattccacaattggctttatcaggtttgtctgaagcattcactattattggtgaaaatgaattgttttataaagaatgtccagaaaatatgagatcaattgctatggcattggtttttgttggttttgctggttcttcatacttgtcatctttgttatcttcaattgttcaaaagacaactggtggtaatgaatcttggttagctgaagatttgaatagaggtagattggattacttttactatttgattggtgctttagaaacattaaatttgatttactttttagtttgttctaaatggtataaatacaaacaagttgaaaagattccacaagttgctatggaaaaattaaataatgttgaagaaaaatcattagtttaa

SEQ ID NO: 324 - DNA, 1785 bp, synthetic construct
  source 1..1785: mol_type = other DNA
atggcagaaaagaatgaaaaattggttccaaatggtactaaagaagatggtaaagttgcagctaaaaatgaaccaaattatagaggtattaaagcaatgccatttgttttgggtaatgaaacatgtgaaaaattgggtacaattggtacttcttcaaatttgtctgtttacttgtcaacagtttttcatatgaatactatgtctgcaaataatgttattaatatttttaatggtacatgttacttgggttctttggttggtgcatttttgtgtgatacatattttggtagatacaaaactttaggtttgggttctgttgcttcatttttgggtatgttgatgttaacattgactgctgcaatttcaactttacatccaactaattgtgataattctggtgactgtaaaggtccatctgctggtcaaatggcatttttagttggtggttttgttttgatggttattggtgcttcaggtattagaccatgtaatttggcttttggtgctgatcaattcaatccaaatacagaatctggtagaagaggtattaattcatttttcaattggtattactttacttatacatttgctgttatggtttctgctacaattattgtttatgttcaatcagatatttcttggtcaattggttttgctattccaacatcattgatgtttttatcttgtgctttgtttttcgctggtacatggttgtatgttaaagttttgccacaaggttcaccaatgacttctttagttcaagttattgttgtttctgttaaaaagagaagattaaaattgccaggtgaaccatctttattgtttaatcatattccatctaatgcaattaattctaaattaccaagaactaataattttagatttttagataaagctgcaattttgacaccagaagataaagttaatccagatggttctgcagctaataagtggaaattatgttcaattcaacaagttgaagaagttaaatgtgttttgagagttttaccaatttggattgctggtgttatttttcaaatttctgttactcaacaatcaaattatgttccatttcaagcatttcaatcagatagacatttgattaaaggtaataagtttcaaattccagcaggtacatataatgttttctcaatgttagctttgactatttggttgccaatttacgatagaattattattccattgttaagaagatggactaaaaaggaagatggtattacattgttacaaagaatgggtgttggtattgttttatcaattttgtcaatgttggtttcagctttggttgaatctagaagaagaaatttggctttgactagaccaactttaggtattcaagaaggtcaaggttctatttcttcaatgtcagctttatggtttgttccacaattggctttaatgggtttggctgaaggttttacttttattggtgaaaatgaattgttttataaagaatttccagaaaatatgagatctattgcagcttctttggttttgtgtggtgttgctgttgcttcatatttgacatcttttattgctgaaattgttcataaaacaactagatcaggtactagagattggttggctcaagatttgaataaggctagattggattactttttctacatggttgctgttttagaagcattaaatttggtttactttttagtttgtgctaaatggtatagatacaaaggtactggtgacttgggtaaattagaagttgctatggaaaaattggaaacagaaaaacatttggtttaa

SEQ ID NO: 325 - DNA, 2052 bp, synthetic construct
  source 1..2052: mol_type = other DNA
atgttgaatcatccatctcaaggttcagatgatgctcaagatgaaaaacaaggtgactttccagttattgaagaagaaaagacacaagctgttactttgaaagattcatacgtttctgatgatgttgctaattctactgaaagatacaatttgtctccatctccagaagatgaagattttgaagctccaactgaagaagaaatgcaaacattgagacatgttggtggtaaaaatgaaccaaattacagaggtattaaagcaatgccatttgttttgggtaatgaaacttgtgaaaaattgggtacaattggtacttcatctaatttgtcagtttacttatcaactgtttttcatatgaatacaatgtctgctaataatgttattaatatttttaatggtacatgttacttgggttctttggttggtgcatttttatgtgatacatactttggtagatataaaactttgggtttaggttctgttgcatcatttttgggtatgttgatgttaactttgacagcagctatttcaactttgcatccaacaaattgtgataattctggtgactgtaaaggtccatcagcaggtcaaatggcatttttggttggtggttttgttttaatggttattggtgcttctggtattagaccatgtaatttggcatttggtgctgatcaattcaatccaaatacagaatcaggtagaagaggtattaattcatttttcaattggtattactttacatatacttttgctgttatggtttcagctactattattgtttatgttcaatctgatatttcttggtcaattggttttgctattccaacttcattgatgtttttatcttgtgctttatttttcgctggtacatggttatacgttaaagttttaccacaaggttctccaatgacatcattggttcaagttattgttgtttctgttaaaaagagaagattgaaattgccaggtgaaccatcattattgtttaatcatattccatctaatgctattaattctaaattaccaagaacaaataattttagatttttggataaagcagctattttgacaccagaagataaagttaatccagatggttctgctgcaaataagtggaaattatgttcaattcaacaagttgaagaagttaaatgtgttttaagagttttgccaatttggattgcaggtgttatttttcaaatttctgttacacaacaatcaaattacgttccatttcaagcatttcaatctgatagacatttgattaaaggtaataagtttcaaattccagctggtacttacaatgttttctctatgttagcattgactatttggttgccaatttatgatagaattattattccattgttaagaagatggactaaaaaggaagatggtattacattattgcaaagaatgggtgttggtattgttttgtcaattttatctatgttggtttcagctttggttgaatctagaagaagaaatttggctttaactagaccaacattgggtattcaagaaggtcaaggttctatttcatcaatgtctgctttgtggtttgttccacaattagctttgatgggtttggcagaaggttttacttttattggtgaaaatgaattgttttacaaagaatttccagaaaatatgagatctattgcagcttcattagttttgtgtggtgttgcagttgcttcatacttgacttcttttattgcagaaattgttcataaaacaactagatctggtactagagattggttggcacaagatttgaataaggcaagattggattactttttctacatggttgctgttttagaagcattgaatctggtttactttttggtttgtgctaaatggtacagatataaaggtacaggtgacttgggtaaattggaagttgcaatggaaaaattggaaactgaaaaacatttggttgaagaagaagatgaatttgatttgaatccaatttcagcaccaaaagctaatgatattgaaattttggaaccaatggattctttaagatcaacagctaaatattaa

SEQ ID NO: 326 - DNA, 1950 bp, synthetic construct
  source 1..1950: mol_type = other DNA
atgttaaatcatccatctcaaggttcagatgatgctcaagatgaaaaacaaggtgactttccagttattgaagaagaaaagactcaagcagttacattgaaagattcatatgtttcagatgatgttgctaattcaactgaaagatataatttgtcaccatctccagaagatgaagattttgaagcaccaactgaagaagaaatgcaaactttaagacatgttggtggtaaaaatgaaccaaattacagaggtattaaagctatgccatttgttttgggtaatgaaacatgtgaaaaattgggtactattggtacatcatctaatttgtcagtttacttatctactgtttttcatatgaatacaatgtctgctaataatgttattaatatttttaatggtacttgttacttgggttctttagttggtgcatttttgtgtgatacatactttggtagatataaaactttgggtttaggttctgttgcttcatttttgggtatgttaatgttgactttaacagcagctatttctacattgcatccaactaattgtgataattctggtgactgtaaaggtccatctgctggtcaaatggcatttttagttggtggttttgttttaatggttattggtgcttctggtattagaccatgtaatttggcttttggtgcagatcaattcaatccaaatacagaatctggtagaagaggtattaattcatttttcaattggtactactttacatacacatttgctgttatggtttctgctactattattgtttatgttcaatcagatatttcatggtctattggttttgctattccaacatcattgatgtttttgtcatgtgcattgtttttcgctggtacttggttgtacgttaaagttttaccacaaggttctccaatgacatctttggttcaagttattgttgtttcagttaaaaagagaagattaaaattgccaggtgaaccatcattgttgtttaatcatattccatctaatgcaattaattcaaaattaccaagaactaataattttagatttttagataaagctgcaattttaactccagaagataaagttaatccagatggttctgcagctaataagtggaaattgtgttctattcaacaagttgaagaagttaaatgtgttttgagagttttgccaatttggattgctggtgttatttttcaaatttcagttactcaacaatctaattatgttccatttcaagcatttcaatctgatagacatttgattaaaggtaataagtttcaaattccagctggtacttataatgttttctctatgttagctttgacaatttggttaccaatttacgatagaattattattccattgttaagaagatggactaaaaaggaagatggtattacattattgcaaagaatgggtgttggtattgttttgtctattttgtcaatgttggtttcagctttggttgaatcaagaagaagaaatttggcattaacaagaccaacattaggtattcaagaaggtcaaggttcaatttcatcaatgtcagctttatggtttgttccacaattagctttaatgggtttggcagaaggttttacttttattggtgaaaatgaattgttttacaaagaatttccagaaaatatgagatcaattgctgcatctttggttttgtgtggtgttgctgttgcatcttacttaacatcttttattgctgaaattgttcataaaacaactagatcaggtactagagattggttggctcaagatttgaataaggcaagattggattactttttctatatggttgctgttttggaagcattaaatttggtttactttttggtttgtgctaaatggtacagatacaaaggtactggtgacttgggtaaattggaagttgctatggaaaaattggaaacagaaaaacatttggtttaa

SEQ ID NO: 327 - DNA, 1275 bp, synthetic construct
  source 1..1275: mol_type = other DNA
atgggaggtgcgagtgccagcggtgtagacaataaatttaataaggaaagggtgatcgctattggtgagataatgagattaccaaatcttaattctttacaggtcgtcgcattcatcaattcactaagggatgatccgagccaaagcgccaacctacttgccgaagcgaagaaattaaacgacgcccaggctcccaaaggaggtagcagcgcaagttctgcaggaggcagtagtgttgacaacaaatttaacaaggaagcgcaaactgcgggtgtggaaatcatggaactgccaaacctgaatacacggcagctcctggctttcatccagtcattacgtgatgatccgtcccagtcagcgaatttgctggcagaggctaaaaagctcaacgatgcgcaagctcctaaaaccagtggaggcagttccgcctccagcgcaggagggtcaagtgtagacaacaaattcaacaaagaaagagtgattgcgataggtgagatcatgcggttacctaacttaaacagtctccaagtggtggccttcatcaatagtttacgggatgacccaagccaaagcgctaacttactagcagaagctaaaaagctaaatgatgctcaggcaccgaaaggaggttctagtgcgtccagtgcaggagggagcagtgttgataacaagttcaacaaggaggctcagaccgcaggggtggagattatggagctacctaacctaaacactagacaacttttggcgtttatccagtcccttagggacgacccatcccaatccgcaaatctattggcggaggctaagaagctgaacgatgcacaagcccccaagacaagtggtagcggtgcatctggtagtggtgcaggtggaagcagcgtagacaataagtttaataaggagcgtgtaatagccatcggagaaattatgcgtttgccaaatctgaactccctacaggtggtggctttcataaatagtttacgtgatgacccctcacaatccgcgaacttgttagctgaggcaaaaaagttaaacgacgctcaggctccaaaaggttccggtgcctccggaagcggagcaggtggatctagcgttgacaataaatttaacaaagaggcgcagactgcaggcgtggaaattatggaattgccgaacttgaacacccgtcagcttttggccttcatacagagcttacgtgatgacccatctcaatcagctaacctattagcagaggctaagaagttaaacgatgctcaggccccaaagacaagttaa

SEQ ID NO: 328 - DNA, 210 bp, synthetic construct
  source 1..210: mol_type = other DNA
atggtagacaacaaattcaacaaagaactgggttgggcgacctgggagatcttcaacttacctaacttaaacggtgtgcaagtgaaggccttcatcgatagtttacgggatgacccaagccaaagcgctaacttgctggcagaagctaaaaagctaaatgatgctcaggcgccgaaaggttcaagcagctcaggttcgagttcttccggc

SEQ ID NO: 329 - DNA, 210 bp, synthetic construct
  source 1..210: mol_type = other DNA
atggtagacaacaaattcaacaaagaaacaatacaagcgagtcaagagatcagactattacctaacttaaacggtagacaaaagctcgccttcatccacagtttacttgatgacccaagccaaagcgctaacttgctggcagaagctaaaaagctaaatgatgctcaggcgccgaaaggttcaagcagctcaggttcgagttcttccggc

SEQ ID NO: 330 - DNA, 168 bp, synthetic construct
  source 1..168: mol_type = other DNA
atgcaactgctgagatgttttagcatcttttccgttattgcgagcgtgttagctcaggaacttactactatctgcgaacaaatcccaagccccacgctggaatcaaccccttactcactgagtacaacaacgatacttgcgaacggcaaacttgacaagagaggaagt

SEQ ID NO: 331 - DNA, 216 bp, synthetic construct
  source 1..216: mol_type = other DNA
atgaaggtgcttgatttgctaaccgtgctgagtgcttcctcactactgagcaccttcgcagcggcggagtcaacggctactgctgatagcacaactgcagccagtagtacggcatcttgtaatcctttaaaaactactggatgcacacctgacaccgcgttagccactagcttcagcgaagacttctcatcttccagtcttgacaagcgtgggtcc

SEQ ID NO: 332 - DNA, 153 bp, synthetic construct
  source 1..153: mol_type = other DNA
atgttatcactgaagactttgttgtgtactcttctaacagtctcaagcgtgttggcgacgccagttcctgccagggacccctcctccattcaattcgtccatgaagaaaacaagaagaggtactatgactacgaccacgggtcccttggcgaa

SEQ ID NO: 333 - DNA, 99 bp, synthetic construct
  source 1..99: mol_type = other DNA
atgaagtttgcgtatagtttactgcttccccttgccggtgtttcagcctccgtcatcaactataaacgtgacggcgactcattagacaaacgtggaagt

SEQ ID NO: 334 - DNA, 198 bp, synthetic construct
  source 1..198: mol_type = other DNA
atgaagctacagtctcttctagtgtctgcagcggtacttacatccctaacagaaaatgtaaatgcatggagtcctaataattcctatgtcccggctaacgtgacctgcgacgacgacataaatctagtccgtgaggcatctggccttagcgacaacgaaactgagtggctgaagaaacgtgacgcttatacgaaagag

SEQ ID NO: 335 - DNA, 60 bp, synthetic construct
  source 1..60: mol_type = other DNA
atgctattgcaggctttcttgttcttgctggcaggattcgcggcaaaaatcagcgcatct

SEQ ID NO: 336 - DNA, 297 bp, synthetic construct
  source 1..297: mol_type = other DNA
atgaaactgtccgctacgactctaactgcggcaagcctaataggctattctacgattgtgtctgctcttccgtatgcggcggacattgacacaggatgtacgacgactgcgcacggttcccatcagcacaagagagctgtggctgtaacctacgtgtacgagacagttaccgtggataaaaatggtcaaacagtaacgccgaccagtaccgaagccagtagtaccgtcgccagcaccacaacgcttattagtgaatcctccgtcacaaagtccagctccctggataaaaggggttct

SEQ ID NO: 337 - DNA, 276 bp, synthetic construct
  source 1..276: mol_type = other DNA
atgcgttttccttccatctttacggacgtgttgttcgctgcgtcaagcgccttagccacccctgttaacacgaccacagaagacgagacagctcagatccctgccgaggcggttataggttacagtgaccttgaaggggacttcgatgttgccgttttgccgttttctaattccaccaataacgggttgctattcataaataccacaatcgcgtcaatcgcagctaaggaagaaggagtaagtttagagaagagggaagcaacgaatttcggatca

SEQ ID NO: 338 - DNA, 300 bp, synthetic construct
  source 1..300: mol_type = other DNA
atggaagagcaaagagagatattagaacagttgaagaaaactttacagatgctgactgttgagccgtccaagaataatcaaatcgccaacgaagaaaaagagaagaaagaaaacgaaaatagctggtgcatcttggaacacaattacgaggatattgcacaagagtttatcgactttatttataagaaccccacaacctatcacgttgtttctttcttcgcggaattgctggacaagcacaacttcaaatacttatcagagaaaagcaactggcaggattccatcggagaagatggcgga

SEQ ID NO: 339 - DNA, 225 bp, synthetic construct
  source 1..225: mol_type = other DNA
atgttcagaatacaattacgtaccatgtcatctaagacgtgcaagagtgactaccctaaggagtttgtctcattcctgaatagcagtcacagtccatatcataccgttcacaacattaagaaacatttagtaagtaatggattcaaggagctaagcgagcgtgactcatgggctggacatgtggcgcagaaaggcaagtatttcgttacacgtaacgggagtagt

SEQ ID NO: 340 - DNA, 891 bp, synthetic construct
  source 1..891: mol_type = other DNA
atgtctgacagcagtccgactattaactttattaactttaaccaaaccggtacatgtatcagtttaggaacgtccaaaggcttcaagatattcaactgtgaacctttcggcaagttctattcagaagacagtggcggatatgccattgtagagatgctattcagtaccagtcttctagccttagttggcataggtgatcaacccgctcttagccctaggcgtctaaggatcatcaatactaagaaacactctatcatctgtgaggtgacgttcccgacttccattttgtccgttaagatgaataagtcacgtcttgtcgtacttttgcaagagcagatatatatctatgacataaacacaatgaggcttctacacactattgagaccaatccgaacccaagaggtttgatggcgatgtcaccaagtgtagccaacagctatctggtgtacccttctcctcctaaagtaattaacagcgagattaaggctcacgcaacaacgaataatataacactgtctgtcggtggaaacactgagacgagttttaaacgtgaccagcaagacgcgggccatagcgacatctctgatctggaccaatactctagcttcactaagagggacgatgctgaccccacgtcttctaacggtggcaatagcagtattattaagaatggcgacgttatagttttcaacctggagactcttcagcccacgatggtcatcgaggcacataagggtgagatcgcggcaatggcaatttcctttgacggaacacttatggcaacagcgtccgacaaggggactataattagagtatttgatatagaaacgggagacaaaatctatcagttccgtaggggcacatacgcgacaagaatttatagcataagc

SEQ ID NO: 341 - DNA, 171 bp, synthetic construct
  source 1..171: mol_type = other DNA
atgattgcccttcctgtagaaaaagcaccaaggaagtctctttggcagagacacagggccttcatctccgggatcgtcgctctaattattataggtacgtttttcttaacttctggcttgcatcctgctccaccgcatgaagcaaaacgtccccatcatgggaaaggaccc

SEQ ID NO: 342 - DNA, 105 bp, synthetic construct
  source 1..105: mol_type = other DNA
atgtttagcctgaaggcattattgccgctggcgttgttacttgtctctgccaatcaggttgccgccaaggtccacaaagcgaaaatatacaaacacgagttgtcc

SEQ ID NO: 343 - DNA, 297 bp, synthetic construct
  source 1..297: mol_type = other DNA
atgaaattggagaacactttatttacacttggtgcactgggttccatttctgctgcgttagtaattccgaatctggagaatgcagccgatcaccatgaattgataaataaagaggaccaccatgagaggcccaggaaggttgaatttacaaaggatgacgacgaggaaccttctgacagcgaggacaaggaacacgggaaattccacaagaagggtaggaaaggtcaggacaaagagtctcccgaatttaatggtaagagggcctcaggtagccacgggtccgcgcatgaaggagga

SEQ ID NO: 344 - DNA, 492 bp, synthetic construct
  source 1..492: mol_type = other DNA
atgaaagcgttcacatctcttctgtgcggtttgggtttgtcaacgaccttggccaaagccatttccctacaaaggccgttaggcctggacaaagacgtgcttcttcaagcagcggaaaagttcgggttagacttggatctggatcatttgctaaaggaactggacagcaacgtcttggatgcgtgggctcaaatcgaacatctatacccgaatcaagtaatgtctctggaaacgtcaaccaaaccgaaattcccagaggcaatcaagaccaaaaaggactgggacttcgtagttaagaacgacgcaatcgagaactatcaactgcgtgtcaacaaaattaaagatccaaagattttgggaatcgaccccaacgtaactcaatacacgggctacttggatgtcgaagacgaggataagcacttcttcttctggacttttgagtcaagaaatgaccctgcgaaagatccagtgatcttgtggctgaatggtgga

SEQ ID NO: 345 - DNA, 225 bp, synthetic construct
  source 1..225: mol_type = other DNA
atgaccaaaaactttattgttacgttgaagaaaaatacgccggacgtggaagcaaagaaattcttggatagcgtccaccatgctggagggtcaatcgtccataagttcgatataatcaaaggctacacaataaaagtgcccgatgtcctgcacctgaataaactaaaggaaaaacacaatgacgtgatagaaaatgtcgaagaagacaaagaggtacatacgaac

SEQ ID NO: 346 - DNA, 234 bp, synthetic construct
  source 1..234: mol_type = other DNA
atgaaatcttttattactagaaacaaaactgccattttggcgacggttgccgccactggtactgctattggtgcctattactactacaaccaattacagcagcagcaacagcgtggtaagaaaaatacaattaacaaggatgagaagaaggacacaaaggattcacagaaggaaacagaaggcgcaaagaagtctactgcaccttctaacccaccgatatacccagttagctct

SEQ ID NO: 347 - DNA, 351 bp, synthetic construct
  source 1..351: mol_type = other DNA
atggcgatgttctgtaccttctttgagaaacatcatagaaaatgggacatcttactagaaaagagcaccggagtgatggaggcgatgaaagtaacttcagaagagaaagagcagttgtctacagctatcgatagaatgaatgaaggtctggacgcatttattcaactatataacgaatccgagatcgatgaacctttaatccagttggatgacgatacagcagaactaatgaaacaggctagggacatgtacggccaagagaaacttaacgagaaattaaacacaataatcaaacaaatcctgtcaatttctgtctccgaagagggtgagaaagaaggaagcggatcaggc

SEQ ID NO: 348 - DNA, 513 bp, synthetic construct
  source 1..513: mol_type = other DNA
atggctgacagggacaggagtggcatttacggaggtacctatggccaacaccagcagggaggaggtagacccatgggcgaacaggttaaaggaatgattcaggacaagggtcccactgcctctcaggcgcttactgtcgcaaccttattccctctgggcggtctacttttggtcctatctggattggccttagccgcaagcgttgtcggtttagctctggcgacccccgtatttctactattttctcccgttcttgtcccggctgctttactaatcggtaccgctgttacggggtttctgacctcaggtgctctaggactgggaggactttctagccttacttgcttggccaataccgctagacaggcgtttcagaggactcctgactatgtagaggaagcccgtcgtagaatggcggaagccgctgcacatgctggccaaaagacggctcaggcaggacacgccatccagtcaagggctcaagaggctgcatctggaggcgcaggtgctgga

SEQ ID NO: 349 - DNA, 279 bp, synthetic construct
  source 1..279: mol_type = other DNA
atgtctacgctacagagaaggcgtgttaacagagcagactctggtgacacgtccagcatccacagctccgcaaataatacaaaaggtgataagattgcaaacatcgctgtcgatggcgatgacgataacggtacaaacaagaaaatagcatacgatcccgaagagagtaagctaagagataacataaacattcctactttgactttaatggaggaggttcttttgatgggtttacgtgacagagagggatacctatccttctggaatgattccatcagt

SEQ ID NO: 350 - DNA, 264 bp, synthetic construct
  source 1..264: mol_type = other DNA
atgtcacttcccttgagaacgatagtacatttggtaaagccctttgcttgcactgctaggtttagtgcgagatacccaatccacgtcattgttgttgctgttttattgagtgccgctgcttatctatccgtgacacaatcttaccttaacgaatggaagctggactctaatcagtattctacatacttaaacataaagccggatgagttgtttgagaaatgcacacactactataggtctggtggaaacgctgcaattagaagc

SEQ ID NO: 351 - DNA, 87 bp, synthetic construct
  source 1..87: mol_type = other DNA
atggatagcaaagggtcctcccagaagggatcaagattgcttctgctgctagtatccaatcttttattgtgccagggggttgtatca

SEQ ID NO: 352 - DNA, 69 bp, synthetic construct
  source 1..69: mol_type = other DNA
atgaaagcgttcacgtccttattatgtggcttgggtttgagcacgacactggctaaggccatctcactg

SEQ ID NO: 353 - DNA, 219 bp, synthetic construct
  source 1..219: mol_type = other DNA
atgtccgcgaccaaaagcatcgtaggggaggctctggaatacgtcaatatcgggctttcacacttccttgctcttcccttagcccaaagaataagtctgatcattattattccattcatctataatattgtatggcagcttctatattctttgcgtaaggacaggcccccgcttgttttctactggattccctgggtcgggtccgcagttgtgtacggg

SEQ ID NO: 354 - DNA, 138 bp, synthetic construct
  source 1..138: mol_type = other DNA
atgcctttcggtattgataatactgatttcacggtcctagcgggtttggtattagcagttctattatatgtcaagcgtaactccatcaaggagttgcttatgtctgatgacggggatataaccgccgtgtcctctggt

SEQ ID NO: 355 - DNA, 99 bp, synthetic construct
  source 1..99: mol_type = other DNA
atggagttgaccatggcatctacgatgagtctggccttactggtgttgagcgcggcctacgttctagtagcactgagaagaagtagatcttccagcagc

SEQ ID NO: 356 - DNA, 210 bp, synthetic construct
  source 1..210: mol_type = other DNA
atgtcatctgtggcagaaaacataattcagcacgcaacacacaacagtacgctgcaccaacttgcgaaggaccaaccttccgtcggcgttacgaccgcatttagcatcttagacactctaaagagcatgagctatctgaagatattcgctactcttatttgtatcttgcttgtatgggatcaagttgcctaccagattaagaaaggctca

SEQ ID NO: 357 - DNA, 75 bp, synthetic construct
  source 1..75: mol_type = other DNA
atggaagaattattaaaacaagcactacaacaagcacagcaattattgcaacaggcccaagagttggcgaagaag

SEQ ID NO: 358 - DNA, 753 bp, synthetic construct
  source 1..753: mol_type = other DNA
atggtatctaaaggagaagaattgtttacaggcgtggtaccaattctggttgaattggacggtgacgtgaacggacacaaattcagcgtgagtggagaaggcgagggagatgctacctatggcaagttgacgcttaaactgatctgcacaacgggcaaattaccagtgccctggccgacgcttgtaacaactcttggatacgggttacagtgctttgcccgttatccagaccatatgaaacagcatgacttcttcaaatctgcgatgccggagggatatgtacaggaacgtacgattttctttaaggacgatgggaactacaagactcgtgctgaggttaagtttgaaggcgacactctagtcaataggatagaattaaagggtattgattttaaggaggatgggaacatcctgggccataaactagagtacaactacaattcacataatgtctacatcaccgctgataaacagaagaacgggatcaaagctaatttcaagatacgtcataatatcgaagatggtggcgtccagcttgctgaccactaccagcagaacacgcctataggcgacgggccggtgttgctacctgacaatcattatctgtcctatcagtccgccctttcaaaagaccctaatgagaagagggatcatatggtgcttttagaatttgtaaccgcggcagggatcacacttgggatggatgagctgtataaaggaggtagtggaagtgcgggatctggcggtagtagc

SEQ ID NO: 359 - DNA, 1161 bp, synthetic construct
  source 1..1161: mol_type = other DNA
atgaagatcgaagaaggaaagttagtgatctggataaatggtgataaaggctacaatgggttggcggaagtaggaaaaaagttcgagaaagacacaggaatcaaagttacggtcgagcaccccgataaactagaggaaaagtttccacaggtagctgctacgggggacggaccagacattatcttttgggcccacgatagattcgggggttatgctcagtccggacttctggccgagattactccagacaaggccttccaagacaaactttacccgttcacatgggacgcagtcaggtacaatggaaagctgattgcatatccgatagctgtggaggcacttagcctaatttacaacaaggatctactacctaaccctcccaagacttgggaagaaattccagctctggacaaggagttaaaagcaaagggtaagagtgcacttatgttcaatctacaagagccttatttcacatggcccctaatagccgccgacggaggctatgcctttaagtacgaaaacggcaagtatgacataaaggatgttggggtagacaacgcgggagccaaggctggattaactttcctggtggatttaattaagaacaaacacatgaacgcagacactgactactctatcgcagaagcagcgttcaataaaggcgaaacggcgatgacaattaacgggccctgggcttggtcaaacattgacacgagtaaagttaactatggtgtaacggtattgcccacatttaagggacaacccagtaaacctttcgtaggagtcttgtcagccgggatcaatgcagcttccccgaataaagagcttgctaaggaatttcttgaaaattatcttttaaccgatgagggattggaggcggttaacaaggacaagcctcttggtgctgtagccctgaaatcctatgaagaagagttagctaaggacccaagaatcgccgcaacaatggagaatgctcagaagggagaaattatgccaaatataccacaaatgagtgccttctggtatgcggtaaggacggcagttattaatgccgcttcaggtagacaaacagtcgatgaggctttgaaagatgcacagactaacagttcatccaacaataataacaataacaataacaataacctgggtatcgagggccgt

SEQ ID NO: 360 - DNA, 1473 bp, synthetic construct
  source 1..1473: mol_type = other DNA
atgacatcactatccaagtcattcatgcagtctggtaggatttgcgccgcctgcttctaccttcttttcactctgcttagcatccctatatcctttaaagtaggcggtcttgaatgcggactttcttttacggttacattgtttacgctatacttcattacaacaacacttaatgtactggcgagaaggcatggagggcgtttgtacatcttcttcactaattgcctatactacagccaacacttcatcatcgcgagtttgctttatttatttctttccggcttctccaacgacgagttaggcaatgtgttgaagaataagtacaacgagagtgagtcttttttagaagcacttaaaaacagtttgaactcaaaccaaatcaattacgtcctgtactattactactaccgtttcgttgtgcaaccttggcaattcgtactaacaaaatctactccattcttcacactatctgaaggattttttactattttagctattcaagcagtcggcgagaccaataggtggctatctaacgacttgaactccaacacctggatcatctcctcattactaacatccgggggcgtgattaccgcttccttgtattatctgtataggatatatgtcactcctatatggcctcttagtattcagactgcatctttgttgggtttggtcttgagtatggtatgtggactaggactgtacggaattgttagccaaaaggggtcagtgattgagtcttctttgttcttcgcctacattgttcgttgtatatatgaaatcagtccgaagctggcgacaacagctactgatgagattttaaacttgttcaaagatgtatggcagaagcaccagcgtaatctgccgaccgcagacaaccttttgtgctattttcacaatgttatcttgaagaacgcggaggtactttggggtagcttcataccaagaggtagaaagaaaacaggagattttcacgacaagctaatttccattctatcattcgaaaaagtaagtttaatctctaagccattctggaaattcttcaaaaacttcacgttcagtgtaccgttaagtatcaatgaattttgccaagtgaccatcaagatggcgagtgaaagcgtttctcccgcaatagtaataaacctgtgttttagggttctgatgttttacagcgcgaccagaataattccagctttgcaaaggaaaaacgacaaacagttgaggaagagcaggcgtatcatgaaaggattgtactggtacagtccatgcattctgatcgccatgtacactcacttaattcttcaatactcaggcgaactgaagaaagatttatgtatttggggatgcagcgaaaagtggtttggagtcgaccaacccgaaattatagtagactcatggggcttttggaactggtgcaacatattctgtactattttggtgtacgcaactgagctgattgggagcggaagt

SEQ ID NO: 361 - DNA, 912 bp, synthetic construct
  source 1..912: mol_type = other DNA
atgcaacaagcgactggcaatgaattgttaggtatattggatttagataatgacatcgactttgagacggcgtaccaaatgttatcaagtaacttcgatgatcaaatgtccgcacatatacacgaaaatacctttagcgccacctcacctcctttacttactcacgaactaggcatcataccgaacgtggcaaccgtacaaccaagtcatgtggagaccattccggcggacaatcaaactcaccatgctccgttacatactcatgcccactacttgaaccacaatccacaccagccaagcatgggcttcgatcaggcgcttggattaaagctgtccccaagcagcagcggcttgttgtcaaccaacgaaagtaatgcgattgagcagttcttagacaaccttatttcacaagacatgatgagttccaatgcgtctatgaatagtgagagtcacctacacatcagatcaccaaaaaaacagcaccgttacaccgaattgaaccaacgttaccccgagacgcacccccactccaataccggtgagctaccgactaatacggctgatgtcccgactgaattcacaacccgtgagggcccgcatcagcccatcggaaatgaccactataatcctccccccttttctgttcctgagataaggattccagatagcgatattcctgcgaatattgaggatgaccccgtaaaagttaggaagtggaaacatgtgcaaatggaaaagatccgtagaattaatacaaaagaggcgtttgagcgtttgatcaagtctgtgagaactccgccgaaggagaatggcaaaaggattccgaaacatatcctgctaacctgtgttatgaacgacataaaaagtatcagaagcgccaatgaagcattgcagcacattctggacgattca

SEQ ID NO: 362 - DNA, 1152 bp, synthetic construct
  source 1..1152: mol_type = other DNA
atgagtaaaagcaagacattcctgttcacgagcgaatcagttggcgaggggcatcccgataagatatgcgaccaggttagtgatgcgattctggacgcatgtctggaacaggatcctttttcaaaagtggcgtgtgagacggccgcgaagacgggaatgatcatggtgttcggagagatcaccactaaagcccgtttagattatcaacaaattgtacgtgataccataaagaagataggttatgatgactcagcaaaggggtttgattataaaacttgtaacgtcttggtcgctatcgagcaacagtcccctgatattgcacaaggtttacattatgagaagtcccttgaagacttaggagcaggcgatcaaggtattatgtttgggtacgcaaccgacgagacccccgaaggactgcctttaacaatacttcttgctcataagctgaacatggcaatggcagacgcgaggagggatggtagcctgccctggttgcgtccagatactaaaactcaagtgacggttgaatatgaagatgataacgggaggtgggtgccgaagaggattgacacggttgttatctccgcacagcacgccgatgagatctccacggccgacctacgtacacaactacaaaaagatattgtggaaaaagtaattccgaaggatatgttggatgaaaatacaaagtattttatccaaccatctggaaggttcgtcatagggggtccccaaggggacgctggcctaactggcaggaagattattgtagatgcgtatggcggagcaagtagcgttggagggggggctttcagcggcaaagactattccaaggtagataggagcgccgcgtacgcggcgagatgggtggcaaaatccttagtggctgcaggactttgtaagcgtgttcaagtgcaattctcatacgcgatcggtatagcggagcccctttccttacatgttgatacgtatggcacagcaactaaaagtgacgatgagataatagaaatcattaagaaaaacttcgatttgagacctggcgtactagtcaaagagttggacttagcaagaccaatctaccttcccacggcttcttatggacattttaccaaccaggagtactcttgggagaagccgaaaaagctggagttc

SEQ ID NO: 363 - DNA, 666 bp, synthetic construct
  source 1..666: mol_type = other DNA
atgtcctccagcgaatccatccgtatggtgctgattggtcccccaggggcggggaagggtacgcaagccccaaatctgcaagaaagatttcacgctgctcacctagctacaggagatatgttgcgtagtcagattgcaaaaggtacacagctaggcctggaagctaaaaaaattatggaccaaggcggccttgttagtgacgatattatggtaaacatgataaaagatgagcttacgaataacccggcttgcaagaatggattcatcttagatggtttcccgcgtacaattccgcaggccgaaaagttggatcaaatgctgaaagaacaaggcacacctttagagaaggccattgagcttaaagttgatgacgagctactagtggcccgtatcaccgggagacttatccaccccgccagcggaagaagttaccacaagatattcaaccctccgaaagaagacatgaaagacgatgtcaccggagaagcattagttcaaaggtctgatgacaacgcggatgcgctaaaaaagagattggctgcgtaccacgcacagacggaaccgattgtagatttctataagaaaacagggatttgggccggagtcgatgcttcccaaccccctgccactgtatgggctgatatccttaataaactaggtaaggat

SEQ ID NO: 364 - DNA, 1761 bp, synthetic construct
  source 1..1761: mol_type = other DNA
atggacatacttaaaagaggaaatgagagcgacaaatttactaaaatagagacggaatctacaactatacccaacgactcagacagatccggatctctgattagacgtatgaaagactcatttaaacaatcaaacctgcatgttattccagaggatttggaaaacagtgaacagacggaacaggagaaaatacagtggaaactggctagtcaaccgtaccagaaggttttgtctcagagacacttgaccatgatcgctatcgggggcacactaggaacaggactatttattggccttggttactctttggcctctgggccagcagcactactaattggtttccttcttgttgggacctccatgttttgtgtagttcagtccgccgcagaattgtcctgtcagtttcccgtttctggtagctacgccacacacgtgtctaggttcatagacgaatctgtgggtttcacagtagcaacaaactatgccttggcgtggctgatctcctttcctagtgaactgatcggatgtgcgctaacgatttcttactggaaccaaacggtaaatccggcggtgtgggtagcaatattttatgtatttatcatggtgttaaatttgtttggtgttcgtggcttcgcagagacagaattcgcgcttagtataattaaggtaatagcaatctttatatttattatcattgggatcgtgttaatagcaggagggggccctaattcaacaggctatataggggcgaagtactggcacgaccctggagcgttcgctaagccggtgttcaagaatttatgcaatacatttgtctcagcggcgttctcctttggtgggagtgaactagtccttttgacctctactgaaagcaagaacatttcagcgataagtagggcggcaaagggtaccttctggaggattgctatcttctatatcactaccgtagtcataatcggctgcttagtaccatataacgacccaaggctgttaagtggctctaactctgaagacgtatccgcctccccgtttgtcatcgcgctatccaacaccgggagcatgggtgctaaggtgtcaaattttatgaacgtcgtaattctagtagcagtagtatccgtgtgcaattcatgtgtttacgcaagctcaaggctgattcaagcgcttggggcctctggtcaactaccatcagtgtgtagttatatggatagaaaagggagaccgttagtggggatagggattagcggagctttcggcttgttaggattccttgtggcaagtaaaaaggaagacgaagtgtttacctggctttttgccctgtgctcaataagcagcttttttacctggttttgtatatgtatgtcccagatcaggtttaggatggctctaaaggcgcagggcaggagtaacgacgagattgcgtataagagcatccttggggtttacggaggaatattaggttgcgtgctgaatgctttgctgatagcgggggagatctatgtcagcgcggcgcctgtcggatccccaagtagtgcggaggcattttttgagtattgtctatccatccccatcatgatagtcgtttattttgctcatcgtttttatagacgtgactggaaacatttctacattaaaaggtccgagatagatttggacacaggatgctccgtcgaaaatttggagttgtttaaggcccagaaagaagctgaagagcaattgatagccagcaaacctttctactataagatctatagattttggtgt

SEQ ID NO: 365 - DNA, 1722 bp, synthetic construct
  source 1..1722: mol_type = other DNA
atgagcgaaggaagaacgtttttatctcagctaaatgttttcaataaggaaaattaccaattctcttctagcaccacaaaaaaagaagtatcaaactctacagtggacgctgacaacggagcttctgattttgaggccgggcagcagttcgccaccgaattagaccagggggaaaagcagctagggattctaagttgcattgggttgatatgtaataggatgttggggacgggggtgtttgcagtgagttcaacgatatatacgttatgcgggagcgtggggctggctcttatcatgtgggcagttggagctattattgcaatctcaggtttgtacgtctacatggagtttgggaccgctatacctaaaaatggcggcgagaagaattacttggaagcaatcttcagaaaacctaaattctttataacctgtatgtacgccgcatatatttttttcttaggatgggctgccggtaacagtataaacacagcaattatgttcctaacggctgctgacacggaggtgactaagtggaatcagagaggaattggtgtagcagtggtgtttttcgcatttctgataaattccttgaatgttaagattggtctatatttacaaaacatactaggaattttcaagatcggaatagtattattcatctctatcactggttgggtggcgctagggggaggtctaaaagacggataccagagccacaattttagaaatgcgttcgagggaacggagaccgcgaccgcctatggcattgttaatgcattatactcagtaatttggagttttgtcggatactccaacgtcaattatgcgcttggtgaggtgaaaaaccccgttaggacattgaagatcgctgggcctacatccatggttttcttggccatcatatatatatttgttaatatagcttatttcgccgttgtgcccaaagacaagctaatatcttccaagctaattcttgctgcagacttctttgatatagtcttcggaggtcaagctaaaagggcggcggccgctctagtgggcctttccgcgttaggtaatgtcttaagtgtgattttcagccagggaaggattatacaacagttgggacgtgagggggtcttaccattctcaaatttcttcgccagttccaagccgttcaatagccctatggtcgggttatttcagcatttcatagtctgtactgtcacaatattggccccgccacctggcgatgcatacctgttagttcaaaatttaataagctacccgatgaacataataaatttcgccattagtgcgggtctgttgtggatctactggcaacgtaggcagggaaagatagaatggaaccctccaattaaggccggagtattcgtgacagggttcttcaccctttccaatttatatttaatcattgccccgtacgttccaccatctaacggagaatcagtgtatagctccatgccctactggatacattgtgtcattgcatggggtatctttttttttggcggggtctattatgtcgtatgggcacagctgttgccgaggtggggtcactataaacttgtaagtaaagacgttcttggagaggacggtttttggagagttaagattgctaaagtttacgatgatactatcggtgatgtagacacccaggaagacggggtcatagaaacaaatattatcgagcactataaatcagagcaggagaaatccttg

SEQ ID NO: 366 - DNA, 1776 bp, synthetic construct
  source 1..1776: mol_type = other DNA
atgacggaggactttatatccagcgttaagaggagcaacgaagaactaaaggagaggaagagcaacttcggttttgtagaatacaaaagcaaacagcttacaagctcatcttctcacaatagcaattcaagccaccacgatgatgataatcaacacgggaagcgtaatattttccaacgttgtgtagactctttcaagtctcccttggacggatcctttgacacgagtaacctgaaaaggacattaaagccgaggcatcttatcatgatagctataggggggagcattggaactggtttgttcgtggggtcaggtaaggcgatagcggaaggcggtcctcttggagtggttattggctgggctattgctggcagtcagatcattggcacgattcacgggttgggtgaaataactgtacgttttcctgtggtcggggcttttgctaattacggtacaaggtttctggatccgtcaatctcattcgtagtatctacgatttatgtcctacagtggttctttgtactaccgttagagataatagccgccgcgatgaccgtgcaatactggaacagttctatagatccggtaatatgggtagcgatcttctacgcagtaattgttagtatcaacttgtttggggttcgtggattcggggaagcggaattcgctttctcaacaataaaggcgatcacagtctgtggcttcatcattctatgtgtagtgttaatatgtggcggaggacctgaccacgagttcataggggcgaagtactggcatgatccgggttgcctggccaacggttttccaggggttttaagcgtgctggtggttgcctcttacagcctgggtggtattgagatgacttgcttggcgtcaggcgagacggacccaaaggggttgccttcagctatcaaacaggtattctggcgtatattattcttttttctgattagcctgacgcttgtgggttttttggtgccctatacgaatcagaacctgctgggcggcagttcagtggacaactccccttttgtcatcgccataaaattgcaccatatcaaggcattaccatctatcgtaaacgccgtaatcttaatttcagtactaagtgttgggaattcatgcattttcgctagttcaagaacactttgcagtatggcccatcagggactaataccttggtggtttgggtacatcgatagagcgggtcgtccgcttgtcggaatcatggccaattccctatttggcttgcttgcttttttagtcaaatctggttccatgagcgaggtatttaattggttaatggcaattgcgggtttggcgacatgtattgtatggttgtctatcaatttatctcacatacgttttcgtctggcaatgaaagcccagggtaagagtcttgatgaattggaatttgtaagcgcagttggtatctggggctccgcatacagtgcattaataaactgtctgatattaatagcccagttttattgttccctgtggccgatagggggctggaccagtggaaaggagcgtgcaaagatcttcttccaaaactacttatgcgcgctaataatgctattcatttttatagtgcataaaatctactacaagtgtcaaaccggcaaatggtggggcgtgaaggcgctgaaagatatagatcttgagacggataggaaggacatcgacattgaaattgtaaagcaagaaattgccgaaaagaagatgtacctagatagcagaccgtggtatgtaagacagtttcacttctggtgt

SEQ ID NO: 367 - DNA, 1800 bp, synthetic construct
  source 1..1800: mol_type = other DNA
atgattaatgacaaactagtccacttcctgatacaaaattacgacgatatacttagggctccgctgtctggtcagttgaaggatgtatattccctatacatatctggcggctacgatgatgagatgcaaaaattgaggaacgataaagacgaagtgctgcagtttgagcagttttggaatgacctgcaagatataatttttgccacaccacagagcatacaattcgatcagaatctgttggttgcagatcgtccggagaagattgtttatctggatgtgttctcactaaagatcctttataataaattccatgctttctattacactttaaaaagttccagctcttcctgcgaagaaaaggtatcttccttgactaccaagcctgaggctgacagtgagaaggaccagttacttgggagattactgggggtgttaaattgggatgtgaacgtatcaaaccagggcttgccgcgtgaacagctttctaacagactacaaaatcttctgagggaaaaaccctcttccttccaactagcgaaggagcgtgccaaatatacaactgaggttatcgagtatataccaatatgttctgactatagccatgcttctctactttccacctccgtctacatcgtcaataataagatcgtcagtcttcaatggtccaaaatttccgcctgccaagagaatcaccccggtctgatcgagtgcattcaaagcaagatccattttatccctaatatcaaaccgcagacagacattagtcttggcgactgctcttacctagacacttgtcataaattgaacacgtgcagatatatccactatttacaatatattccaagctgccttcaagaacgtgctgatcgtgaaactgcatcagaaaataagagaatccgttcaaatgtctccatccccttttataccttaggaaattgcagcgcgcactgcattaagaaagcattgccggcccagtggatccgttgtgatgtaagaaaattcgacttcagggtccttggcaaattctccgtggtaatcgccgaccctgcctggaacattcacatgaatctaccctacggtacgtgtaacgatatcgaattactggggttgccattacatgaactgcaggacgagggtatcatttttctatgggtaacggggagagcaatcgaattgggaaaggagtctttgaataattggggctacaacgtcatcaatgaagtttcttggataaagacaaatcagttaggccgtaccatcgtcactggaagaacggggcattggttgaaccactcaaaggaacatctactggttggtttaaagggcaatccaaaatggatcaataagcacatagatgtggatcttatagtctccatgaccagagaaacttcaaggaaaccggatgagttatacggaatcgctgaacgtctggcagggacacatgcgagaaagttggagatatttggaagggatcataatacaagacctggctggtttactataggaaatcaactgactggtaactgtatctacgagatggatgtcgagagaaagtatcaggagttcatgaaatctaaaacgggcactagtcatacggggaccaagaagatagacaagaaacagccctctaaactgcaacagcaacaccaacaacagtactggaataacatggacatggggtctggaaagtactacgcggaggctaagcagaatcccatgaatcaaaaacacacccctttcgaaagcaagcagcagcaaaaacaacaattccaaaccctaaacaacttgtatttcgcccag

SEQ ID NO: 368 - DNA, 1527 bp, synthetic construct
  source 1..1527: mol_type = other DNA
atgtcatccccaactttgagatcagcattgagaaatttgccagcagctttattggctttagcagctgcttgtgaagctcaaaatgttggtaaatggggtccaatggttaaatttccagttgttccagttgctgttgcattggttccagaaactggtaatttgttggtttggtcatctggttggccaaatagatggactactgctggtaatggtaaaacttacacttctttgtataatgttaatactggtaatatttctgatgcaattgttcaaaatacacaacatgatatgttttgtccaggtacttctttggatgctgatggtagaattattgttacaggtggttcttcagctgcaaaaacttcagttttggattttaaaaagggtgaatcatctccgtggactccattgtctaatatgcaaatttctagaggttaccaatcatcttgtacaacttctgaaggtaaaatttttgttattggtggttctttttcaggtgctggtactagaaatggtgaagtttacgatccaaaagctaatacatggactaaattagctggttgtccagttaaaccattggttatgcaaagaggtatgtttccagattctcatgcttggttatggtcttggaaaaatggttctgttttgcaagctggtccatctaaaaagatgaattggtatgatactaaaggtacaggttctaatactccagcaggtttgagaggtactgatgaagattctatgtgtggtgtttctgttatgtacgatgcagttgctggtaaaatttttacttacggtggtggtaaaggttatactggttacgattcaacatctaatgctcatattttaacattgggtgaaccaggtcaagctgttcaagttcaaaaattagctaatggtaaatataatagaggttttgcaaatgctgttgttatgccagatggtaaaatttgggttgttggtggtatgcaaaagatgtggttgttttctgatacaactccacaattaactccagaattgtttgatccagcaactggttcttttacaccaactacaccacatactgttccaagaaattaccattctacagcattgttaatggctgatgctacaatttggtctggtggtggtggtttgtgtggtgctaattgtaaagaaaatcattttgatggtcaattttggtcaccaccatacttatttgaagctgatggtgttacaccagctaaaagaccagttattcaatcattgtctgatactgctgttagagcaggtgctccaattacaattactatgcaagatgctggtgcatatactttttcaatgattagagtttctgctactacacatacagttaatacagatcaaagaagaataccattggatggtcaagatggtggtgacggtaaatcttttactgttaatgttccaaatgattatggtgttgcaattccaggttactatatgttatttgctatgaatgaagctggtgttccatgtgttgctcaatttttcaaagttactttgtaa

SEQ ID NO: 369 - DNA, 984 bp, synthetic construct
  source 1..984: mol_type = other DNA
atgtcatccgaatttgttagatcattgtgtgtttttattacatttttaggttgtttgatttcttcagctcatggtcaagctgcagctagaaggccaggtccaatttctggtactagaattggtttttacttgacaacttgtccaacagctgaaattattgttagaaatgctgttagagctggttttaattctgatccaagaattgctccaggtattttgagaatgcattttcatgattgttttgttttaggttgtgatggttctgttttgatttctggttcaaatactgaaagaacagctgttccaaatttgaatttgagaggttttgaagttattgataatgctaaaactcaattggaagctacttgtccaggtgttgtttcttgtgctgatattttagcattggctgcaagagatactgttgttttgactagaggtttgggttggcaagttccaacaggtagaagagatggtagagtttctgttgcttctaatgctaataatttgccaggtccaagagattctgttgctgttcaacaacaaaaattttctgctgttggtttgaatactagagatttggttgttttagcaggtggtcatacaattggtactgctggttgtggtgtttttagagatagattgtttaataatacagatccaaatgttaatcaattgtttttaactcaattgcaaactcaatgtccacaaaatggtgacggtgctgttagagttgatttggatactggttctggtactacatttgataattcttactttattaatttgtcaagaggtagaggtgttttggaatcagatcatgttttgtggactgatccagctacaagaccaattgttcaacaattgatgtctccaagaggtaattttaatgctgaatttgctagatctatggttagaatgtctaatattggtgttgttacaggtgctaatggtgaaattagaagagtttgttctgctgttaattaa